SEQUENCE LISTING (171 sequences)

SEQ ID NO: 1 - protein, 4 aa, synthetic construct
NAMS

SEQ ID NO: 2 - protein, 17 aa, synthetic construct
SISGSGGSTSLARSVKG

SEQ ID NO: 3 - protein, 7 aa, synthetic construct
DLGFMDV

SEQ ID NO: 4 - protein, 11 aa, synthetic construct
DAVITISQELA

SEQ ID NO: 5 - protein, 7 aa, synthetic construct
DASYHES

SEQ ID NO: 6 - protein, 9 aa, synthetic construct
QQYGNWPHT

SEQ ID NO: 7 - protein, 115 aa, synthetic construct
MTQLVESGGGLVQPGGSLRLSCAADHTTEYNAMSWVRQAPGKGLEWVGSISGSGGSTSLARSVKGRFTISRDNSKNTLYLQMNSLRAEDTAVYYCARDLGFMDVWGQGTLVTVSS

SEQ ID NO: 8 - protein, 107 aa, synthetic construct
DIQMTQSPSSLSASVGDRVTITCDAVITISQELAWYQQKPGKAPKLLIYDASYHESGVPSRFSGSGSGTDFTLTISSLQPEDFATYYCQQYGNWPHTFGQGTKVEIK

SEQ ID NO: 9 - protein, 11 aa, synthetic construct
DALITISQELA

SEQ ID NO: 10 - protein, 107 aa, synthetic construct
DIQMTQSPSSLSASVGDRVTITCDALITISQELAWYQQKPGKAPKLLIYDASYHESGVPSRFSGSGSGTDFTLTISSLQPEDFATYYCQQYGNWPHTFGQGTKVEIK

SEQ ID NO: 11 - protein, 7 aa, synthetic construct
DASYHDT

SEQ ID NO: 12 - protein, 107 aa, synthetic construct
DIQMTQSPSSLSASVGDRVTITCDAVITISQELAWYQQKPGKAPKLLIYDASYHDTGVPSRFSGSGSGTDFTLTISSLQPEDFATYYCQQYGNWPHTFGQGTKVEIK

SEQ ID NO: 13 - protein, 5 aa, synthetic construct
EQAMS

SEQ ID NO: 14 - protein, 17 aa, synthetic construct
SISPKGGSKSLNRNFNG

SEQ ID NO: 15 - protein, 7 aa, synthetic construct
DIGFMDG

SEQ ID NO: 16 - protein, 11 aa, synthetic construct
HGHVFISNYLA

SEQ ID NO: 17 - protein, 7 aa, synthetic construct
DGEWRDF

SEQ ID NO: 18 - protein, 9 aa, synthetic construct
QQYSNWPHT

SEQ ID NO: 19 - protein, 116 aa, synthetic construct
ARQLVESGGGLVQPGGSLRLSCAASGFHFEEQAMSWVRQAPGKGLEWVGSISPKGGSKSLNRNFNGRFTISRDNSKNTLYLQMNSLRAEDTAVYYCAKDIGFMDGWGQGTLVTVSS

SEQ ID NO: 20 - protein, 106 aa, synthetic construct
YQMTQSPSSLSASVGDRVTITCHGHVFISNYLAWYQQKPGKAPKLLIYDGEWRDFGVPSRFSGSGSDDSFTLTISSLQPEDFATYYCQQYSNWPHTFGQGTKVEIK

SEQ ID NO: 21 - protein, 4 aa, synthetic construct
NAMS

SEQ ID NO: 22 - protein, 17 aa, synthetic construct
AISGSGGSTYYADSVKG

SEQ ID NO: 23 - protein, 7 aa, synthetic construct
DLGFMDV

SEQ ID NO: 24 - protein, 11 aa, synthetic construct
RASQSISSYLN

SEQ ID NO: 25 - protein, 7 aa, synthetic construct
DASYHEF

SEQ ID NO: 26 - protein, 9 aa, synthetic construct
QQSYSTPWT

SEQ ID NO: 27 - protein, 115 aa, synthetic construct
MTQLVESGGGLVQPGGSLRLSCAADHTTEYNAMSWVRQAPGKGLEWVSAISGSGGSTYYADSVKGRFTISRDNSKNTLYLQMNSLRAEDTAVYYCARDLGFMDVWGQGTLVTVSS

SEQ ID NO: 28 - protein, 107 aa, synthetic construct
DIQMTQSPSSLSASVGDRVTITCRASQSISSYLNWYQQKPGKAPKLLIYDASYHEFGVPSRFSGSGSGTDFTLTISSLQPEDFATYYCQQSYSTPWTFGQGTKVEIK

SEQ ID NO: 29 - protein, 5 aa, synthetic construct
DSWIH

SEQ ID NO: 30 - protein, 17 aa, synthetic construct
WISPYGGSTYYADSVKG

SEQ ID NO: 31 - protein, 9 aa, synthetic construct
RHWPGGFDY

SEQ ID NO: 32 - protein, 11 aa, synthetic construct
RASQDVSTAVA

SEQ ID NO: 33 - protein, 7 aa, synthetic construct
SASFLYS

SEQ ID NO: 34 - protein, 9 aa, synthetic construct
QQYLYHPAT

SEQ ID NO: 35 - protein, 118 aa, synthetic construct [sequence omitted]

SEQ ID NO: 36 - protein, 107 aa, synthetic construct [sequence omitted]

SEQ ID NO: 37 - protein, 437 aa, synthetic construct
DIQMTQSPSSLSASVGDRVTITCRASQSISSYLNWYQQKPGKAPKLLIYDASYHEFGVPSRFSGSGSGTDFTLTISSLQPEDFATYYCQQSYSTPWTFGQGTKVEIKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 38 - protein, 222 aa, synthetic construct
MTQLVESGGGLVQPGGSLRLSCAADHTTEYNAMSWVRQAPGKGLEWVSAISGSGGSTYYADSVKGRFTISRDNSKNTLYLQMNSLRAEDTAVYYCARDLGFMDVWGQGTLVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 39 - protein, 443 aa, synthetic construct
MTQLVESGGGLVQPGGSLRLSCAADHTTEYNAMSWVRQAPGKGLEWVGSISGSGGSTSLARSVKGRFTISRDNSKNTLYLQMNSLRAEDTAVYYCARDLGFMDVWGQGTLVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 40 - protein, 399 aa, synthetic construct
CDLPQTHSLGSRRTLMLLAQMRKISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKEGGSGGGGSGGGGSGGGGSGGDIQMTQSPSSLSASVGDRVTITCDAVITISQELAWYQQKPGKAPKLLIYDASYHESGVPSRFSGSGSGTDFTLTISSLQPEDFATYYCQQYGNWPHTFGQGTKVEIKRTVAAPSVFIFPPSDRKLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 41 - protein, 437 aa, synthetic construct; identical to SEQ ID NO: 37

SEQ ID NO: 42 - protein, 222 aa, synthetic construct; identical to SEQ ID NO: 38

SEQ ID NO: 43 - protein, 443 aa, synthetic construct; identical to SEQ ID NO: 39

SEQ ID NO: 44 - protein, 399 aa, synthetic construct
CDLPQTHSLGSRRTLMLLAQMRKISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKEGGSGGGGSGGGGSGGGGSGGDIQMTQSPSSLSASVGDRVTITCDALITISQELAWYQQKPGKAPKLLIYDASYHESGVPSRFSGSGSGTDFTLTISSLQPEDFATYYCQQYGNWPHTFGQGTKVEIKRTVAAPSVFIFPPSDRKLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 45 - protein, 437 aa, synthetic construct; identical to SEQ ID NO: 37

SEQ ID NO: 46 - protein, 222 aa, synthetic construct; identical to SEQ ID NO: 38

SEQ ID NO: 47 - protein, 443 aa, synthetic construct; identical to SEQ ID NO: 39

SEQ ID NO: 48 - protein, 399 aa, synthetic construct
CDLPQTHSLGSRRTLMLLAQMRKISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKEGGSGGGGSGGGGSGGGGSGGDIQMTQSPSSLSASVGDRVTITCDAVITISQELAWYQQKPGKAPKLLIYDASYHDTGVPSRFSGSGSGTDFTLTISSLQPEDFATYYCQQYGNWPHTFGQGTKVEIKRTVAAPSVFIFPPSDRKLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 49 - protein, 437 aa, synthetic construct
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 50 - protein, 225 aa, synthetic construct
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 51 - protein, 443 aa, synthetic construct; identical to SEQ ID NO: 39

SEQ ID NO: 52 - protein, 399 aa, synthetic construct; identical to SEQ ID NO: 44

SEQ ID NO: 53 - protein, 437 aa, synthetic construct; identical to SEQ ID NO: 49

SEQ ID NO: 54 - protein, 225 aa, synthetic construct; identical to SEQ ID NO: 50

SEQ ID NO: 55 - protein, 443 aa, synthetic construct; identical to SEQ ID NO: 39

SEQ ID NO: 56 - protein, 399 aa, synthetic construct; identical to SEQ ID NO: 48

SEQ ID NO: 57 - protein, 437 aa, synthetic construct
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 58 - protein, 225 aa, synthetic construct; identical to SEQ ID NO: 50

SEQ ID NO: 59 - protein, 444 aa, synthetic construct
ARQLVESGGGLVQPGGSLRLSCAASGFHFEEQAMSWVRQAPGKGLEWVGSISPKGGSKSLNRNFNGRFTISRDNSKNTLYLQMNSLRAEDTAVYYCAKDIGFMDGWGQGTLVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 60 - protein, 398 aa, synthetic construct
CDLPQTHSLGSRRTLMLLAQMRKISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKEGGSGGGGSGGGGSGGGGSGGYQMTQSPSSLSASVGDRVTITCHGHVFISNYLAWYQQKPGKAPKLLIYDGEWRDFGVPSRFSGSGSDDSFTLTISSLQPEDFATYYCQQYSNWPHTFGQGTKVEIKRTVAAPSVFIFPPSDRKLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 61 - protein, 638 aa, synthetic construct
CDLPQTHSLGSRRTLMLLAQMRKISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKEGGSGGGGSGGGGSGGGGSGGQVQLVQSGAEVKKPGASVKVSCKASGGTFSSYAISWVRQAPGQGLEWMGIIDPSVTYTRYAQKFQGRVTMTRDTSTSTVYMELSSLRSEDTAVYYCARSLFPTIFGVEVAFDIWGQGTLVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 62 - protein, 226 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 63 - protein, 214 aa, synthetic construct
DIQMTQSPSSLSASVGDRVTITCQASQDISNYLNWYQQKPGKAPKLLIYGASTLQSGVPSRFSGSGSGTDFTLTISSLQPEDFATYYCQQTYSTPITFGQGTKVEIKRTVAAPSVFIFPPSDRKLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 64 - protein, 638 aa, synthetic construct
CDLPQTHSLGSRRTLMLLAQMRKISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKEGGSGGGGSGGGGSGGGGSGGQVQLVQSGAEVKKPGASVKVSCKASGGTFTGYYMHWVRQAPGQGLEWMGWVNPNSGNTGYAQKFQGRVTMTRDTSTSTVYMELSSLRSEDTAVYYCARSLFPTIFGVEVAFDIWGQGTLVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 65 - protein, 226 aa, synthetic construct; identical to SEQ ID NO: 62

SEQ ID NO: 66 - protein, 214 aa, synthetic construct
DIQMTQSPSSLSASVGDRVTITCQASQDISNYLNWYQQKPGKAPKLLIYAASSLQSGVPSRFSGSGSGTDFTLTISSLQPEDFATYYCQQSYSPPPTFGQGTKLEIKRTVAAPSVFIFPPSDRKLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 67 - protein, 632 aa, synthetic construct
CDLPQTHSLGSRRTLMLLAQMRKISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKEGGSGGGGSGGGGSGGGGSGGQVQLVQSGAEVKKPGASVKVSCKASGYTFSNYYVHWVRQAPGQGLEWMGWMDPNSGGTGYAHQFQGRVTMTRDTSTSTVYMELSSLRSEDTAVYYCAKEVFSGWYDYWGQGTLVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 68 - protein, 226 aa, synthetic construct; identical to SEQ ID NO: 62

SEQ ID NO: 69 - protein, 214 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQSYSTPYTFGQGTKVEIKRTVAAPSVFIFPPSDRKLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 70 - protein, 480 aa, synthetic construct
QVQLVQSGAEVKKPGASVKVSCKASGYTFTKNYMHWVRQAPGQGLEWLGWVSPDSGYTGYAQKFQGRVTMTRDTSTSTVYMELSSLRSEDTAVYYCTTDLLSLELDDAFDIWGQGTMVTVSSASGGGGSGGGGSGGGGSHASDIQMTQSPSSLSASVGDRVTITCRASQSISSWLAWYQQKPGKAPKLLIYAASTLQSGVPSRFSGSGSGTDFTLTISSLQPEDFATYYCQQSYSTPLTFGGGTKLEIKPRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPCEEEMTKKQVTLSCAVTDFMPEDIYVEWTNNGKTELNYKNTEPVLDSDGSYFMVSKLRVEKKNWVERNSYSCSVVHEGLHNHHTTKSFSRTPG

SEQ ID NO: 71 - protein, 632 aa, synthetic construct
CDLPQTHSLGSRRTLMLLAQMRRISLFSCLKDRHDFGFPQEEFGNQFQKAETIPVLHEMIQQIFNLFSTKDSSAAWDETLLDKFCTELYQQLNDLEACVMQEERVGETPLMNADSILAVKKYFRRITLYLTEKKYSPCAWEVVRAEIVRSLSLSTNLQERLRRKEGGGGSGGGGSGGGGSGGGGSQVQLVQSGAEVKKPGASVKVSCKASGYTFSNYYIHWVRQAPGQGLEWMGWMDSNSGGTGYAQKFQGRVTMTRDTSTSTVYMELSSLRSEDTAVYYCAKEVFSGWYDYWGQGTLVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVCVLPPPEEEMTKKQVTLWCMVTDFMPEDIYVEWTNNGKTELNYKNTEPVLDSDGSYFMYSKLRVEKKNWVERNSYSCSVVHEGLHNHHTTKSFSRTPG

SEQ ID NO: 72 - protein, 214 aa, synthetic construct
DIQMTQSPSSLSASVGDRVTITCRASQSISSYLNWYQQKPGKAPKLLIYAASSLESGVPSRFSGSGSGTDFTLTISSLQPEDFATYYCQQSYSTPYTFGQGTKVDIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 73 - protein, 480 aa, synthetic construct; identical to SEQ ID NO: 70

SEQ ID NO: 74 - protein, 632 aa, synthetic construct
CDLPQTHSLGSRRTLMLLAQMRRISLFSCLKDRHDFGFPQEEFGNQFQKAETIPVLHEMIQQIFNLFSTKDSSAAWDETLLDKFCTELYQQLNDLEACVMQEERVGETPLMNADSILAVKKYFRRITLYLTEKKYSPCAWEVVRAEIVRSLSLSTNLQERLRRKEGGGGSGGGGSGGGGSGGGGSQVQLVQSGAEVKKPGASVKVSCKASGYTFSGYYIHWVRQAPGQGLEWMGWMDSNSGGTGYAQKFQGRVTMTRDTSTSTVYMELSSLRSEDTAVYYCAKEVFSGWYDYWGQGTLVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVCVLPPPEEEMTKKQVTLWCMVTDFMPEDIYVEWTNNGKTELNYKNTEPVLDSDGSYFMYSKLRVEKKNWVERNSYSCSVVHEGLHNHHTTKSFSRTPG

SEQ ID NO: 75 - protein, 214 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQSYSTPYTFGQGTKVEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 76 - protein, 765 aa, synthetic construct
CDLPQTHSLGSRRTLMLLAQMRRISLFSCLKDRHDFGFPQEEFGNQFQKAETIPVLHEMIQQIFNLFSTKDSSAAWDETLLDKFCTELYQQLNDLEACVMQEERVGETPLMNADSILAVKKYFRRITLYLTEKKYSPCAWEVVRAEIVRSLSLSTNLQERLRRKEGGGGSGGGGSGGGGSGGGGSQVQLVQSGAEVKKPGASVKVSCKASGGTFSSYAISWVRQAPGQGLEWMGIIDPSVTYTRYAQKFQGRVTMTRDTSTSTVYMELSSLRSEDTAVYYCARSLFPTIFGVEVAFDIWGQGTLVTVSSASGGGGSGGGGSGGGGSHASDIQMTQSPSSLSASVGDRVTITCQASQDISNYLNWYQQKPGKAPKLLIYGASTLQSGVPSRFSGSGSGTDFTLTISSLQPEDFATYYCQQTYSTPITFGQGTKVEIKAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPCEEEMTKKQVTLSCAVTDFMPEDIYVEWTNNGKTELNYKNTEPVLDSDGSYFMVSKLRVEKKNWVERNSYSCSVVHEGLHNHHTTKSFSRTPG

SEQ ID NO: 77 - protein, 447 aa, synthetic construct
QVQLVQSGAEVKKPGASVKVSCKASGYTFSGYYIHWVRQAPGQGLEWMGWMDSNSGGTGYAQKFQGRVTMTRDTSTSTVYMELSSLRSEDTAVYYCAKEVFSGWYDYWGQGTLVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVCVLPPPEEEMTKKQVTLWCMVTDFMPEDIYVEWTNNGKTELNYKNTEPVLDSDGSYFMYSKLRVEKKNWVERNSYSCSVVHEGLHNHHTTKSFSRTPG

SEQ ID NO: 78 - protein, 214 aa, synthetic construct; identical to SEQ ID NO: 75

SEQ ID NO: 79 - protein, 165 aa, Homo sapiens
CDLPQTHSLGSRRTLMLLAQMRKISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 80 - protein, 165 aa, Homo sapiens [sequence omitted]

SEQ ID NO: 81 - protein, 188 aa, Homo sapiens
MALTFALLVALLVLSCKSSCSVGCDLPQTHSLGSRKTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 82 - protein, 188 aa, Homo sapiens
MALTFALLVALLVLSCKSSCSVGCDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 83 - protein, 290 aa, Homo sapiens
MRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET

SEQ ID NO: 84 - protein, 225 aa, Homo sapiens
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 85 - protein, 227 aa, Homo sapiens
ESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSL

SEQ ID NO: 86 - protein, 225 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 87 - protein, 225 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 88 - protein, 225 aa, synthetic construct; identical to SEQ ID NO: 87

SEQ ID NO: 89 - protein, 227 aa, synthetic construct
ESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSL

SEQ ID NO: 90 - protein, 227 aa, synthetic construct
ESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLGSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSL

SEQ ID NO: 91 - protein, 107 aa, Homo sapiens [sequence omitted]

SEQ ID NO: 92 - protein, 105 aa, Homo sapiens
QPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 93 - protein, 328 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 94 - protein, 328 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 95 - protein, 325 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSL

SEQ ID NO: 96 - protein, 5 aa, synthetic construct
  REGION 1..5: note = The entire sequence of amino acids 1-5 can be repeated from one to ten times.
GSGGS

SEQ ID NO: 97 - protein, 4 aa, synthetic construct
  REGION 1..4: note = The entire sequence of amino acids 1-4 can be repeated from one to ten times.
GGGS

SEQ ID NO: 98 - protein, 5 aa, synthetic construct
  REGION 1..5: note = The entire sequence of amino acids 1-5 can be repeated from one to ten times.
GSGGG

SEQ ID NO: 99 - protein, 5 aa, synthetic construct
  REGION 1..5: note = The entire sequence of amino acids 1-5 can be repeated from one to ten times.
GGGSG

SEQ ID NO: 100 - protein, 5 aa, synthetic construct
  REGION 1..5: note = The entire sequence of amino acids 1-5 can be repeated from one to ten times.
GSSSG

SEQ ID NO: 101 - protein, 5 aa, synthetic construct
  REGION 1..5: note = The entire sequence of amino acids 1-5 can be repeated from one to ten times.
GGGGS

SEQ ID NO: 102 - protein, 5 aa, synthetic construct
  REGION 1..5: note = The entire sequence of amino acids 1-5 can be repeated from one to ten times.
GGSGG

SEQ ID NO: 103 - protein, 20 aa, synthetic construct
GGSGGGSGGGSGGGSGGGSG

SEQ ID NO: 104 - protein, 25 aa, synthetic construct
GSGSGGSGSGGSGSGGSGSGGSGSG

SEQ ID NO: 105 - protein, 20 aa, synthetic construct
GSGGGGSGGGGSGGGGSGGG

SEQ ID NO: 106 - protein, 25 aa, synthetic construct
GGGSGGGGSGGGGSGGGGSGGGGSG

SEQ ID NO: 107 - protein, 20 aa, synthetic construct
GSSSGGSSSGGSSSGGSSSG

SEQ ID NO: 108 - protein, 20 aa, synthetic construct
GGGGSGGGGSGGGGSGGGGS

SEQ ID NO: 109 - protein, 20 aa, synthetic construct
GGGGSGGGGSGGGGSGGGGS

SEQ ID NO: 110 - protein, 20 aa, synthetic construct
GGSGGGGSGGGGSGGGGSGG

SEQ ID NO: 111 - protein, 240 aa, synthetic construct
MQQLVESGGGLVQPGGSLRLSCAAKETWSPNAMSWVRQAPGKGLEWVGSISPKGGSKSIHRKFQGRFTISRDNSKNTLYLQMNSLRAEDTAVYYCAKDLGFMDEWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTSPPGSGMASMTGGQQMG

SEQ ID NO: 112 - protein, 214 aa, synthetic construct
KTQMTQSPSSLSASVGDRVTITCDGTYGISNELAWYQQKPGKAPKLLIYDGEYHDFDVPSRFSGSGSGSDFTLTISSLQPEDFATYYCQQYGNWPHTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 113 - protein, 241 aa, synthetic construct
ADQLVESGGGLVQPGGSLRLSCAASGFDFSVQAMSWVRQAPGKGLEWVGSISPKGGSKSLNRNFNGRFTISRDNSKNTLYLQMNSLRAEDTAVYYCAKDLGFMDNWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTSPPGSGMASMTGGQQMG

SEQ ID NO: 114 - protein, 213 aa, synthetic construct
YQMTQSPSSLSASVGDRVTITCHGHVFISNYLAWYQQKPGKAPKLLIYDGEWRDFGVPSRFSGSGSDDSFTLTISSLQPEDFATYYCQQYSNWPHTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 115 - protein, 444 aa, synthetic construct
MSQLVESGGGLVQPGGSLRLSCAANNDIEFNAMSWVRQAPGKGLEWVGSISPKGGSKSIHRKFQGRFTISRDNSKNTLYLQMNSLRAEDTAVYYCARDLGFMDIWGQGTLVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 116 - protein, 214 aa, synthetic construct
KTQMTQSPSSLSASVGDRVTITCDGTYGISNELAWYQQKPGKAPKLLIYDGEYHDFDVPSRFSGSGSGSDFTLTISSLQPEDFATYYCQQYGNWPHTFGQGTKVEIKRTVAAPSVFIFPPSDRKLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 117 - protein, 444 aa, synthetic construct
MTQLVESGGGLVQPGGSLRLSCAADHTTEYNAMSWVRQAPGKGLEWVSAISGSGGSTYYADSVKGRFTISRDNSKNTLYLQMNSLRAEDTAVYYCARDLGFMDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 118 - protein, 214 aa, synthetic construct
DIQMTQSPSSLSASVGDRVTITCRASQSISSYLNWYQQKPGKAPKLLIYDASYHESGVPSRFSGSGSGTDFTLTISSLQPEDFATYYCQQSYSTPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 119 - protein, 444 aa, synthetic construct; identical to SEQ ID NO: 117

SEQ ID NO: 120 - protein, 214 aa, synthetic construct
DIQMTQSPSSLSASVGDRVTITCRASQSISSYLNWYQQKPGKAPKLLIYDASSHEFGVPSRFSGSGSGTDFTLTISSLQPEDFATYYCQQSYSTPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 121 - protein, 438 aa, synthetic construct
DIQMTQSPSSLSASVGDRVTITCRASQSISSYLNWYQQKPGKAPKLLIYDASYHEFGVPSRFSGSGSGTDFTLTISSLQPEDFATYYCQQSYSTPWTFGQGTKVEIKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 122 - protein, 222 aa, synthetic construct; identical to SEQ ID NO: 38

SEQ ID NO: 123 - protein, 438 aa, synthetic construct
DIQMTQSPSSLSASVGDRVTITCRASQSISSYLNWYQQKPGKAPKLLIYDAEYHDFGVPSRFSGSGSGTDFTLTISSLQPEDFATYYCQQSYSTPWTFGQGTKVEIKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 124 - protein, 222 aa, synthetic construct; identical to SEQ ID NO: 38

SEQ ID NO: 125 - protein, 444 aa, synthetic construct
MTQLVESGGGLVQPGGSLRLSCAADHTTEYNAMSWVRQAPGKGLEWVGSISGSGGSTSLARSVKGRFTISRDNSKNTLYLQMNSLRAEDTAVYYCARDLGFMDVWGQGTLVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 126 - protein, 214 aa, synthetic construct
DIQMTQSPSSLSASVGDRVTITCDAVITISQELAWYQQKPGKAPKLLIYDAEYHDTGVPSRFSGSGSGTDFTLTISSLQPEDFATYYCQQYGNWPHTFGQGTKVEIKRTVAAPSVFIFPPSDRKLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 127 - protein, 444 aa, synthetic construct; identical to SEQ ID NO: 125

SEQ ID NO: 128 - protein, 214 aa, synthetic construct
DIQMTQSPSSLSASVGDRVTITCDAVITISQELAWYQQKPGKAPKLLIYDASYHESGVPSRFSGSGSGTDFTLTISSLQPEDFATYYCQQYGNWPHTFGQGTKVEIKRTVAAPSVFIFPPSDRKLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 129 - protein, 444 aa, synthetic construct; identical to SEQ ID NO: 125

SEQ ID NO: 130 - protein, 214 aa, synthetic construct
DIQMTQSPSSLSASVGDRVTITCDAVIVISQELAWYQQKPGKAPKLLIYDASYHDTGVPSRFSGSGSGTDFTLTISSLQPEDFATYYCQQYGNWPHTFGQGTKVEIKRTVAAPSVFIFPPSDRKLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 131 - protein, 444 aa, synthetic construct
EVQLVESGGGLVQPGGSLRLSCAADHTTEYNAMSWVRQAPGKGLEWVGSISGSGGSTSLARSVKGRFTISRDNSKNTLYLQMNSLRAEDTAVYYCARDLGFMDVWGQGTLVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 132 - protein, 214 aa, synthetic construct
DIQMTQSPSSLSASVGDRVTITCDAVITISQELAWYQQKPGKAPKLLIYDASYHEFGVPSRFSGSGSGTDFTLTISSLQPEDFATYYCQQYGNWPHTFGQGTKVEIKRTVAAPSVFIFPPSDRKLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 133 - protein, 444 aa, synthetic construct; identical to SEQ ID NO: 125

SEQ ID NO: 134 - protein, 214 aa, synthetic construct
DIQMTQSPSSLSASVGDRVTITCDAVITISQELAWYQQKPGKAPKLLIYDASYHDTGVPSRFSGSGSGTDFTLTISSLQPEDFATYYCQQYGNWPHTFGQGTKVEIKRTVAAPSVFIFPPSDRKLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 135 - protein, 444 aa, synthetic construct; identical to SEQ ID NO: 125

SEQ ID NO: 136 - protein, 214 aa, synthetic construct
DIQMTQSPSSLSASVGDRVTITCDAVITISQELAWYQQKPGKAPKLLIYDASSHEFGVPSRFSGSGSGTDFTLTISSLQPEDFATYYCQQYGNWPHTFGQGTKVEIKRTVAAPSVFIFPPSDRKLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 137 - protein, 444 aa, synthetic construct; identical to SEQ ID NO: 125

SEQ ID NO: 138 - protein, 214 aa, synthetic construct
DIQMTQSPSSLSASVGDRVTITCDALITISQELAWYQQKPGKAPKLLIYDASYHESGVPSRFSGSGSGTDFTLTISSLQPEDFATYYCQQYGNWPHTFGQGTKVEIKRTVAAPSVFIFPPSDRKLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 139 - protein, 246 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGGGSGGGLNDIFEAQKIEWHE

SEQ ID NO: 140 - protein, 246 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGSGGGLNDIFEAQKIEWHE

SEQ ID NO: 141 - protein, 226 aa, synthetic construct; identical to SEQ ID NO: 62

SEQ ID NO: 142 - protein, 443 aa, synthetic construct
MTQLVESGGGLVQPGGSLRLSCAADHTTEYNAMSWVRQAPGKGLEWVSAISGSGGSTYYADSVKGRFTISRDNSKNTLYLQMNSLRAEDTAVYYCARDLGFMDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 143 - protein, 214 aa, synthetic construct
DIQMTQSPSSLSASVGDRVTITCRASQSISSYLNWYQQKPGKAPKLLIYDASYHEFGVPSRFSGSGSGTDFTLTISSLQPEDFATYYCQQSYSTPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 144 - protein, 214 aa, synthetic construct
FTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLA

SEQ ID NO: 145 - protein, 220 aa, Macaca fascicularis
FTVTVPKDLYVVEYGSNMTIECKFPVEKQLDLTSLIVYWEMEDKNIIQFVHGEEDLKVQHSNYRQRAQLLKDQLSLGNAALRITDVKLQDAGVYRCMISYGGADYKRITVKVNAPYNKINQRILVVDPVTSEHELTCQAEGYPKAEVIWTSSDHQVLSGKTTTTNSKREEKLLNVTSTLRINTTANEIFYCIFRRLDPEENHTAELVIPELPLALPPNER

SEQ ID NO: 146 - protein, 165 aa, synthetic construct
CDLPQTHSLGSRRTLMLLAQMRKISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMASFSLSTNLQESLRSKE

SEQ ID NO: 147 - protein, 217 aa, Homo sapiens
ISYDSPDYTDESCTFKISLRNFRSILSWELKNHSIVPTHYTLLYTIMSKPEDLKVVKNCANTTRSFCDLTDEWRSTHEAYVTVLEGFSGNTTLFSCSHNFWLAIDMSFEPPEFEIVGFTNHINVMVKFPSIVEEELQFDLSLVIEEQSEGIVKKHKPEIKGNMSGNFTYIIDKLIPNTNYCVSVYLEHSDEQAVIKSPLKCTLLPPGQESESAESAK

SEQ ID NO: 148 - protein, 137 aa, Homo sapiens
ARSEDRYRNPKGSACSRIWQSPRFIARKRGFTVKMHCYMNSASGNVSWLWKQEMDENPQQLKLEKGRMEESQNESLATLTIQGIRFEDNGIYFCQQKCNNTSEVYQGSGTELRVMGFSTLAQLKQRNTLKDGGNSVT

SEQ ID NO: 149 - protein, 24 aa, synthetic construct
GLNDIFEAQKIEWHEARAHHHHHH

SEQ ID NO: 150 - protein, 225 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 151 - protein, 252 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNRFTQKSLSLSPGGSGLNDIFEAQKIEWHEARAHHHHHH

SEQ ID NO: 152 - protein, 240 aa, synthetic construct
GLNDIFEAQKIEWHEDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 153 - protein, 225 aa, synthetic construct
DKTHTCPPCPAPEAAGGPSVFLFPPKPKDTAMASRTPEVTCVVVDVSHEDPEVKFNWYVDGVEVHNAKTKPREEQYNSTYRVVSVLTVLHQDWANGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 154 - protein, 252 aa, synthetic construct; identical to SEQ ID NO: 151

SEQ ID NO: 155 - protein, 227 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 156 - protein, 23 aa, synthetic construct
HHHHHHGSGLNDIFEAQKIEWHE

SEQ ID NO: 157 - protein, 225 aa, synthetic construct; identical to SEQ ID NO: 84

SEQ ID NO: 158 - protein, 226 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 159 - protein, 246 aa, synthetic construct; identical to SEQ ID NO: 140

SEQ ID NO: 160 - protein, 444 aa, synthetic construct; identical to SEQ ID NO: 125

SEQ ID NO: 161 - protein, 670 aa, synthetic construct
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGSGGGGSGGGGSGGGGSGGDIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 162 - protein, 399 aa, synthetic construct; identical to SEQ ID NO: 40

SEQ ID NO: 163 - protein, 225 aa, synthetic construct; identical to SEQ ID NO: 50

SEQ ID NO: 164 - protein, 676 aa, synthetic construct
MTQLVESGGGLVQPGGSLRLSCAADHTTEYNAMSWVRQAPGKGLEWVGSISGSGGSTSLARSVKGRFTISRDNSKNTLYLQMNSLRAEDTAVYYCARDLGFMDVWGQGTLVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCGGSGGGGSGGGGSGGGGSGGDIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 165 - protein, 438 aa, synthetic construct
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 166 - protein, 225 aa, synthetic construct; identical to SEQ ID NO: 50

SEQ ID NO: 167 - protein, 399 aa, synthetic construct; identical to SEQ ID NO: 40

SEQ ID NO: 168 - protein, 438 aa, synthetic construct
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 169 - protein, 444 aa, synthetic construct; identical to SEQ ID NO: 115

SEQ ID NO: 170 - protein, 399 aa, synthetic construct
CDLPQTHSLGSRRTLMLLAQMRKISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKEGGSGGGGSGGGGSGGGGSGGKTQMTQSPSSLSASVGDRVTITCDGTYGISNELAWYQQKPGKAPKLLIYDGEYHDFDVPSRFSGSGSGSDFTLTISSLQPEDFATYYCQQYGNWPHTFGQGTKVEIKRTVAAPSVFIFPPSDRKLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 171 - protein, 225 aa, synthetic construct; identical to SEQ ID NO: 50

(19) United States
(12) Patent Application Publication
(10) Pub. No.: US 2024/0376205 A1
(43) Pub. Date: Nov. 14, 2024

(54) TARGETED INTERFERON ALPHA FUSION PROTEINS AND METHODS OF USE

(71) Applicant: Hoffmann-La Roche Inc., Little Falls, NJ (US)

(72) Inventors: Jack Anthony BATES, Muenchen (DE); Davide BERTOLDO, Muenchen (DE); Ulrich BRINKMANN, Weilheim (DE); Harald DUERR, Starnberg (DE); Moritz RAPP, Neubeuern (DE); Jasmin SYDOW-ANDERSEN, Muenchen (DE); Pablo UMAÑA, Lachen (CH); Monika WOLF, Penzberg (DE)

(73) Assignee: Hoffmann-La Roche Inc., Little Falls, NJ (US)

(21) Appl. No.: 18/657,083

(22) Filed: May 7, 2024

(30) Foreign Application Priority Data
May 8, 2023 (EP) 23172135.8

(51) Int. Cl. C07K 16/28; A61K 39/00; A61P 35/00; C07K 14/56; C07K 16/24

(52) U.S. Cl. CPC C07K 16/2827; A61P 35/00; C07K 14/56; C07K 16/249; A61K 2039/505; C07K 2317/31; C07K 2317/52; C07K 2317/55; C07K 2317/565; C07K 2319/30

(57) ABSTRACT

The invention provides fusion proteins that comprise a human IFN-α2, a first antigen-binding domain capable of binding to human PD-L1, a second antigen-binding domain capable of binding to human PD-L1 and to human IFN-α2, and methods of using the same.

### CROSS-REFERENCE TO RELATED APPLICATIONS

This application claims priority to European Patent Application No. 23172135.8, filed May 8, 2023, which is incorporated herein by reference in its entirety.

### SEQUENCE LISTING

This application contains a Sequence Listing, which has been submitted electronically in XML format and is hereby incorporated by reference in its entirety. Said XML copy, created on Apr. 23, 2024, is named P38419-US-SequenceListing.xml and is 190,525 bytes in size.

### TECHNICAL FIELD

The present invention relates to fusion proteins comprising a human IFN-α2, a first antigen-binding domain capable of binding to human PD-L1, and a second antigen-binding domain capable of binding to human PD-L1 and to human IFN-α2, and methods of using the same.

### BACKGROUND

The type-I interferons (IFN) are a family of cytokines that play a key role in inflammation, immunoregulation, tumor cell recognition, and T-cell response. Interferon alpha (IFN-α) is a type-I interferon that is naturally produced by the immune system to attack viruses and cancer cells. It has been extensively studied and found to be effective in the treatment of certain types of cancer, such as melanoma, renal cell carcinoma, and chronic myeloid leukemia. The mechanism of action of IFN-α involves inhibition of the tumor cell growth and boosting of the immune system's ability to recognize and destroy cancer cells.

The term “IFN-α” stands for a family of cytokines that comprises multiple subtypes, including IFN-α1, IFN-α2 and several others. IFN-α1 and IFN-α2 differ in their amino acid sequence, but both IFN-α1 and IFN-α2 have similar biological activities and are used clinically for the treatment of cancer and viral infections. IFN-α2 has been shown to be more potent than IFN-α1 in terms of its antiviral and antiproliferative activities.

The mode of action of IFN-α involves binding to its receptor IFNAR, which is composed of the IFNAR1 and IFNAR2 subunits, a crucial step in the activation of the JAK-STAT signaling pathway. This leads to the transcription of genes involved in immune cell activation and apoptosis of cancer cells. The binding of IFN-α to its receptor is a key molecular event that underlies the therapeutic efficacy of IFN-α in the treatment of cancer.

The therapeutic use of IFN-α can have significant side effects, including flu-like symptoms such as fever, chills, and fatigue, however. For this reason, the targeting of IFN-α to tumor cells and to immune cells in the tumor microenvironment (TME) is a highly promising approach. Targeted therapies use agents such as antibodies or antigen-binding domains to specifically target cancer cells based on their unique molecular characteristics, such as overexpression of certain tumor markers on their surface. By selectively targeting cancer cells and immune cells in the TME, damage of healthy cells and/or tissues is avoided, reducing the incidence and severity of undesirable side effects. Furthermore, due to their specificity, targeted therapies can achieve high response rates and improved outcomes in patients. Overall, the benefits of targeted tumor therapy suggest that this approach has the potential to significantly improve outcomes for patients with cancer, while minimizing the toxicity and inconvenience associated with traditional chemotherapy.

In the case of IFN-α, however, targeting alone may not be sufficient to prevent IFN-α from exercising its activity also in the periphery, i.e. in healthy cells and tissues. Thus, novel therapeutic agents are needed that are capable of preventing IFN-α from acting on its receptor unless the molecule is in the proximity of a tumor cell.

WO 2021/231773 provides protein complexes comprising a sensor domain and a therapeutic domain linked by a linker, and methods of use thereof. The therapeutic domain may be IFN-α. Activity of the therapeutic domain comprises a dependence on sensor domain binding to target markers, such as PD-L1.

### SUMMARY

The invention provides fusion proteins that comprise a human IFN-α2 (huIFN-α2) or variant thereof, a first antigen-binding domain capable of binding to human PD-L1 (huPD-L1) and a second antigen-binding domain capable of binding to human PD-L1 and to human IFN-α2, and methods of using the same. These fusion proteins provided herein are characterized in that the second antigen-binding domain is a bispecific Fab and the human IFN-α2 is fused at its C-terminus to the N-terminus of the heavy chain or the light chain of the second antigen-binding domain.

The human IFN-α2 fusion proteins provided herein have been found to work in a self-regulated manner, that is, they show a therapeutic effect predominantly in the presence of the target molecule PD-L1 and remain inactive in its absence. Moreover, the fusion proteins described herein comprise a bispecific antigen-binding domain that is capable of binding to human PD-L1 and to human IFN-α2, i.e. it has affinity for human IFN-α2 and for human PD-L1, such that human IFN-α2 and the human PD-L1 compete for binding to the bispecific antigen-binding domain. Moreover, binding of the bispecific antigen-binding domain to IFN-α2 prevents IFN-α2 from binding to IFNAR. As long as there is no human PD-L1 present, the bispecific antigen-binding domain binds to the fhuman IFN-α2 moiety of the fusion protein, preventing it from binding to its receptor IFNAR and thus from activating the IFNα pathway. In the presence of human PD-L1, the second antigen-binding domain binds to human PD-L1, and the human IFN-α2 moiety is no longer bound to the bispecific antigen-binding domain, thus now being able to bind to the IFNAR receptor and to trigger IFN-α pathway activation in the target cell.

One aspect of the invention is a fusion protein that comprises
a. a first antigen-binding domain capable of binding to human PD-L1;
b. a second antigen-binding domain capable of binding to human PD-L1 and to human IFN-α2; and
c. a human IFN-α2;
   wherein
i. the second antigen-binding domain is a bispecific Fab; and
ii. the human IFN-α2 is fused at its C-terminus to the N-terminus of the heavy chain or the light chain of the second antigen-binding domain.

In one embodiment of the invention, the first antigen-binding domain capable of binding to human PD-L1 is monospecific for human PD-L1.

In one embodiment, the second antigen-binding domain capable of binding to human PD-L1 and to human IFN-α2 is capable of blocking binding of human IFN-α2 to IFNAR. In one particular embodiment, the second antigen-binding domain is capable of blocking binding of huIFN-α2 to the IFNAR2 subunit of the IFNAR.

One embodiment of the invention is a fusion protein that comprises
a. a first antigen-binding domain capable of binding to human PD-L1;
b. a second antigen-binding domain capable of binding to human PD-L1 and to human IFN-α2; and
c. a human IFN-α2;
   wherein
i. the second antigen-binding domain is a bispecific Fab; and
ii. the human IFN-α2 is fused at its C-terminus to the N-terminus of the heavy chain or the light chain of the second antigen-binding domain;
   wherein the bispecific Fab is blocked from binding to PD-L1 when it is bound to human IFN-α2 and is blocked from binding to human IFN-α2 when it is bound to PD-L1
   and/or
   wherein the human IFN-α2 is blocked from binding to IFNAR when it is bound to the bispecific Fab and is able to bind to IFNAR when the bispecific Fab is bound to PD-L1.

In one aspect of the invention, the first antigen-binding domain of the fusion protein capable of binding to human PD-L1 is monospecific for human PD-L1. In one embodiment, the first antigen-binding domain of the fusion protein capable of binding to human PD-L1 is a Fab. In one embodiment, the affinity of the first antigen-binding domain to PD-L1 is characterized by a K_{D} of 1.1 nM or lower as measured using a BIACORE® surface plasmon resonance assay at 25° C.

In one embodiment of the fusion protein provided herein, the first antigen-binding domain or the second antigen-binding domain is a crossover Fab molecule wherein either the variable or the constant regions of the Fab light chain and the Fab heavy chain are exchanged.

In one embodiment, the amino acid at position 124 in the constant domain CL of one of the Fab fragments of the fusion protein is substituted independently by lysine (K), arginine (R) or histidine (H) (numbering according to Kabat EU Index), and in the constant domain CH1 the amino acids at positions 147 and 213 are substituted independently by glutamic acid (E) or aspartic acid (D) (numbering according to Kabat EU index).

In one embodiment, the amino acid at position 123 (EU numbering) in the constant domain CL of one of the Fab fragments of the fusion protein has been replaced by arginine (R) and the amino acid at position 124 (EU numbering) has been substituted by lysine (K) and the amino acids at position 147 (EU numbering) and at position 213 (EU numbering) in one of the CH1 domains have been substituted by glutamic acid (E).

In one embodiment, the amino acid at position 124 in the constant domain CL of the Fab fragment comprising the second antigen binding domain capable of binding to human PD-L1 and to human IFN-α2 is substituted independently by lysine (K), arginine (R) or histidine (H) (numbering according to Kabat EU Index), and the amino acids at positions 147 and 213 in the constant domain CH1 of the Fab fragment comprising the second antigen binding domain capable of binding to human PD-L1 and to human IFN-α2 are substituted independently by glutamic acid (E) or aspartic acid (D) (numbering according to Kabat EU index).

In one embodiment, the amino acid at position 123 (EU numbering) in the constant domain CL of the Fab fragment comprising the second antigen binding domain capable of binding to human PD-L1 and to human IFN-α2 has been replaced by arginine (R) and the amino acid at position 124 (EU numbering) has been substituted by lysine (K) and the amino acids at position 147 (EU numbering) and at position 213 (EU numbering) in one of the CH1 domains of the Fab fragment comprising the antigen binding domain capable of binding to human PD-L1 and to human IFN-α2 have been substituted by glutamic acid (E).

In one embodiment, the fusion protein described herein comprises not more than one human IFN-α2. In one embodiment, the fusion protein described herein comprises exactly one human IFN-α2. In one embodiment, the human IFN-α2 comprised in the fusion protein is selected from human IFN-α2a, human IFN-α2b, or functional variants thereof. In one embodiment, the human IFN-α2 comprised in the fusion protein comprises an amino acid sequence selected from SEQ ID NO:79, SEQ ID NO:80, SEQ ID NO:81, and SEQ ID NO: 82, preferably selected from SEQ ID NO:79 and SEQ ID NO:80. In one embodiment, the human IFN-α2 comprised in the fusion protein provided herein comprises the amino acid sequence of SEQ ID NO:79. In one embodiment, the human IFN-α2 comprised in the fusion protein comprises one or more mutations which modify the binding of human IFN-α2 to the IFNAR1/2 receptor.

In one embodiment, the human IFN-α2 comprised in the fusion protein provided herein is fused at its N-terminus or its C-terminus to the bispecific Fab via a peptidic linker. In one embodiment, the human IFN-α2 comprised in the fusion protein provided herein is fused at its C-terminus to the N-terminus of the light chain of the bispecific Fab. In certain embodiments, the peptidic linker has a length of 16 to 24 amino acids, particularly of 20 amino acids. In one embodiment, the peptidic linker is a glycine serine (GS) linker comprising an amino acid sequence selected from the group consisting of (GS)ₙ, (GSGGS)ₙ (SEQ ID NO:96), (GGGS)ₙ (SEQ ID NO:97), (GSGGG)ₙ (SEQ ID NO:98), (GGGSG)ₙ (SEQ ID NO:99), (GSSSG)ₙ (SEQ ID NO:100), (GGGGS)ₙ (SEQ ID NO:101), (GGSGG)ₙ (SEQ ID NO:102), where n represents an integer of at least 1, preferably from 4 to 6. In another embodiment, the peptidic linker comprises an amino acid sequence selected from GGSGGGSGGGSGGGSGGGSG (SEQ ID NO: 103), GSGSGGSGSGGSG SGGSGSGGSGSG (SEQ ID NO:104), GSGGGG SGGGGSGGGGSGGG (SEQ ID NO: 105), GGGSGGGGSGGGGSGGGGSGGGGSG (SEQ ID NO:106), GSSS GGSSSGGSSSGGSSSG (SEQ ID NO:107), GGGGSGGGGSGGGGSGGGGS (SEQ ID NO: 108), GGGGSGGGGSGGGGSGGGGS (SEQ ID NO:109), and GGSGGGG SGGGGSGGGGSGG (SEQ ID NO:110).

In one particular embodiment, the bispecific Fab comprised in the second antigen-binding domain of the fusion protein provided herein is a DutaFab.

In one embodiment, the affinity of the bispecific Fab to human PD-L1 is from 1-fold to 10-fold, in particular from 2-fold to 5-fold of the affinity to human IFN-α2.

In one embodiment, the affinity of the bispecific Fab to human PD-L1 is characterized by a K_{D} from 1 nM to 10 nM and the affinity to human IFN-α2 is characterized by a K_{D} from 10 nM to 20 nM, as measured using a BIACORE® surface plasmon resonance assay at 25° C. In one embodiment, the affinity of the bispecific Fab to human PD-L1 is characterized by a K_{D} from 1 nM to 10 nM and the affinity to human IFN-α2 is characterized by a K_{D} from 10 nM to 20 nM, wherein the affinity to human PD-L1 is measured using a BIACORE® surface plasmon resonance assay at 25° C. as described in Example 8 g) and wherein the affinity to human IFN-α2 is measured using a BIACORE® surface plasmon resonance assay at a Biacore 8K or 8K+ instrument at 25° C. using untagged huIFNa2a (SEQ ID NO:79), as described in Example 8 h).

In one embodiment, the second antigen-binding domain capable of binding to human PD-L1 and to human IFN-α2 comprises a human IFN-α2 paratope and a human PD-L1 paratope within one cognate pair of a variable light chain domain (VL domain) and a variable heavy chain domain (VH domain), wherein
a) the human IFN-α2 paratope comprises amino acid residues from CDR-H2, CDR-L1 and CDR-L3 of the antigen-binding domain, and wherein the human PD-L1 paratope comprises amino acid residues from the CDR-H1, CDR-H3 and CDR-L2 of the antigen-binding domain, or
b) the human PD-L1 paratope comprises amino acid residues from CDR-H2, CDR-L1 and CDR-L3 of the antigen-binding domain, and wherein the human IFN-α2 paratope comprises amino acid residues from the CDR-H1, CDR-H3 and CDR-L2 of the antigen-binding domain.

In one embodiment, the second antigen-binding domain capable of binding to human PD-L1 and to human IFN-α2 comprises a human IFN-α2 paratope and a human PD-L1 paratope within one cognate pair of a variable light chain domain (VL domain) and a variable heavy chain domain (VH domain), wherein the human IFN-α2 paratope comprises amino acid residues from CDR-H2, CDR-L1 and CDR-L3 of the antigen-binding domain, and wherein the human PD-L1 paratope comprises amino acid residues from the CDR-H1, CDR-H3 and CDR-L2 of the antigen-binding domain.

In one aspect, the fusion protein further comprises an Fc domain composed of a first and a second subunit. In a further aspect, the Fc domain is an IgG Fc domain, particularly an IgG1 Fc domain or an IgG4 Fc domain. In another aspect, the Fc domain comprises one or more amino acid substitutions that reduce binding to an Fc receptor, in particular towards Fcγ receptor. In certain embodiments, the Fc domain is an Fc domain of human IgG1 subclass with the amino acid mutations L234A, L235A and P329G (numbering according to Kabat EU index). In some embodiments, the first subunit of the Fc domain comprises the amino acid substitutions Y349C, T366S, L368A and Y407V (numbering according to Kabat EU index) and the second subunit of the Fc domain comprises the amino acid substitutions S354C and T366W (numbering according to Kabat EU index).

In one embodiment, the fusion protein further comprises an Fc domain composed of a first and a second subunit, and the first antigen-binding domain comprised in the fusion protein is a Fab and is fused at the C-terminus of the Fab heavy chain to the N-terminus of the first subunit of the Fc domain, and the second antigen-binding domain is a bispecific Fab, in particular a DutaFab, and is fused at the C-terminus of the Fab heavy chain to the N-terminus of the second subunit of the Fc domain, and the human IFN-α2 is fused at its C-terminus to the N-terminus of the light chain of the bispecific Fab of the second antigen-binding domain.

In one embodiment, the invention is a fusion protein that comprises
a. a first antigen-binding domain capable of binding to human PD-L1;
b. a second antigen-binding domain capable of binding to human PD-L1 and to human IFN-α2; and
c. a human IFN-α2;
   wherein
i. the second antigen-binding domain is a bispecific Fab; and
ii. the human IFN-α2 is fused at its C-terminus to the N-terminus of the heavy chain or the light chain of the second antigen-binding domain; and
   wherein the second antigen-binding domain capable of binding to human PD-L1 and to human IFN-α2 comprises
a. a heavy chain variable domain (VH) comprising (a) CDR-H1 comprising the amino acid sequence of SEQ ID NO:1, (b) CDR-H2 comprising the amino acid sequence of SEQ ID NO:2, and (c) CDR-H3 comprising the amino acid sequence of SEQ ID NO:3, and a light chain variable domain (VL) comprising (d) CDR-L1 comprising the amino acid sequence of SEQ ID NO:4, (e) CDR-L2 comprising the amino acid sequence of SEQ ID NO:5, and (f) CDR-L3 comprising the amino acid sequence of SEQ ID NO:6; or
b. a heavy chain variable domain (VH) comprising (a) CDR-H1 comprising the amino acid sequence of SEQ ID NO: 1, (b) CDR-H2 comprising the amino acid sequence of SEQ ID NO:2, and (c) CDR-H3 comprising the amino acid sequence of SEQ ID NO:3, and a light chain variable domain (VL) comprising (d) CDR-L1 comprising the amino acid sequence of SEQ ID NO:9, (e) CDR-L2 comprising the amino acid sequence of SEQ ID NO:5, and (f) CDR-L3 comprising the amino acid sequence of SEQ ID NO:6; or
c. a heavy chain variable domain (VH) comprising (a) CDR-H1 comprising the amino acid sequence of SEQ ID NO:1, (b) CDR-H2 comprising the amino acid sequence of SEQ ID NO:2, and (c) CDR-H3 comprising the amino acid sequence of SEQ ID NO:3, and a light chain variable domain (VL) comprising (d) CDR-L1 comprising the amino acid sequence of SEQ ID NO:4, (e) CDR-L2 comprising the amino acid sequence of SEQ ID NO:11, and (f) CDR-L3 comprising the amino acid sequence of SEQ ID NO:6; or
d. a heavy chain variable domain (VH) comprising (a) CDR-H1 comprising the amino acid sequence of SEQ ID NO:13, (b) CDR-H2 comprising the amino acid sequence of SEQ ID NO: 14, and (c) CDR-H3 comprising the amino acid sequence of SEQ ID NO: 15, and a light chain variable domain (VL) comprising (d) CDR-L1 comprising the amino acid sequence of SEQ ID NO:16, (e) CDR-L2 comprising the amino acid sequence of SEQ ID NO:17, and (f) CDR-L3 comprising the amino acid sequence of SEQ ID NO:18.

In one embodiment, the invention is a fusion protein that comprises a first antigen-binding domain capable of binding to human PD-L1, a second antigen-binding domain capable of binding to human PD-L1 and to human IFN-α2, and a human IFN-α2, wherein the second antigen-binding domain is a bispecific Fab and the human IFN-α2 is fused at its C-terminus to the N-terminus of the heavy chain or the light chain of the second antigen-binding domain, wherein the second antigen-binding domain capable of binding to human PD-L1 and to human IFN-α2 comprises
a. a VH domain comprising the amino acid sequence of SEQ ID NO:7 and a VL domain comprising the amino acid sequence of SEQ ID NO:8;
b. a VH domain comprising the amino acid sequence of SEQ ID NO:7 and a VL domain comprising the amino acid sequence of SEQ ID NO:10;
c. a VH domain comprising the amino acid sequence of SEQ ID NO:7 and a VL domain comprising the amino acid sequence of SEQ ID NO: 12; or
d. a VH domain comprising the amino acid sequence of SEQ ID NO: 19 and a VL domain comprising the amino acid sequence of SEQ ID NO:20.

In one embodiment, the invention is a fusion protein that comprises a first antigen-binding domain capable of binding to human PD-L1, a second antigen-binding domain capable of binding to human PD-L1 and to human IFN-α2, and a human IFN-α2, wherein the second antigen-binding domain is a bispecific Fab and the human IFN-α2 is fused at its C-terminus to the N-terminus of the heavy chain or the light chain of the second antigen-binding domain, wherein the first antigen binding domain capable of binding to human PD-L1 comprises
a. a heavy chain variable domain (VH) comprising (a) CDR-H1 comprising the amino acid sequence of SEQ ID NO:21, (b) CDR-H2 comprising the amino acid sequence of SEQ ID NO:22, and (c) CDR-H3 comprising the amino acid sequence of SEQ ID NO:23, and a light chain variable domain (VL) comprising (d) CDR-L1 comprising the amino acid sequence of SEQ ID NO:24, (e) CDR-L2 comprising the amino acid sequence of SEQ ID NO:25, and (f) CDR-L3 comprising the amino acid sequence of SEQ ID NO:26; or
b. a heavy chain variable domain (VH) comprising (a) CDR-H1 comprising the amino acid sequence of SEQ ID NO:29, (b) CDR-H2 comprising the amino acid sequence of SEQ ID NO:30, and (c) CDR-H3 comprising the amino acid sequence of SEQ ID NO:31, and a light chain variable domain (VL) comprising (d) CDR-L1 comprising the amino acid sequence of SEQ ID NO:32, (e) CDR-L2 comprising the amino acid sequence of SEQ ID NO:33, and (f) CDR-L3 comprising the amino acid sequence of SEQ ID NO:34.

In one embodiment, the invention is a fusion protein that comprises a first antigen-binding domain capable of binding to human PD-L1, a second antigen-binding domain capable of binding to human PD-L1 and to human IFN-α2, and a human IFN-α2, wherein the second antigen-binding domain is a bispecific Fab and the human IFN-α2 is fused at its C-terminus to the N-terminus of the heavy chain or the light chain of the second antigen-binding domain, wherein the first antigen binding domain capable of binding to human PD-L1 comprises
a. a VH domain comprising the amino acid sequence of SEQ ID NO:27 and a VL domain comprising the amino acid sequence of SEQ ID NO:28; or
b. a VH domain comprising the amino acid sequence of SEQ ID NO:35 and a VL domain comprising the amino acid sequence of SEQ ID NO:36.

In one embodiment, the invention provides a fusion protein that comprises a first antigen-binding domain capable of binding to human PD-L1, a second antigen-binding domain capable of binding to human PD-L1 and to human IFN-α2, and a human IFN-α2, wherein the second antigen-binding domain is a bispecific Fab and the human IFN-α2 is fused at its C-terminus to the N-terminus of the heavy chain or the light chain of the second antigen-binding domain, wherein
a. the first antigen-binding domain that is capable of binding to human PD-L1 is a crossover Fab molecule wherein either the variable or the constant regions of the Fab light chain and the Fab heavy chain are exchanged, and comprises
   i. a heavy chain variable domain (VH) comprising (a) CDR-H1 comprising the amino acid sequence of SEQ ID NO:21, (b) CDR-H2 comprising the amino acid sequence of SEQ ID NO:22, and (c) CDR-H3 comprising the amino acid sequence of SEQ ID NO:23, and a light chain variable domain (VL) comprising (d) CDR-L1 comprising the amino acid sequence of SEQ ID NO:24, (e) CDR-L2 comprising the amino acid sequence of SEQ ID NO:25, and (f) CDR-L3 comprising the amino acid sequence of SEQ ID NO:26; or
   ii. a heavy chain variable domain (VH) comprising (a) CDR-H1 comprising the amino acid sequence of SEQ ID NO:29, (b) CDR-H2 comprising the amino acid sequence of SEQ ID NO:30, and (c) CDR-H3 comprising the amino acid sequence of SEQ ID NO:31, and a light chain variable domain (VL) comprising (d) CDR-L1 comprising the amino acid sequence of SEQ ID NO:32, (e) CDR-L2 comprising the amino acid sequence of SEQ ID NO:33, and (f) CDR-L3 comprising the amino acid sequence of SEQ ID NO:34; and
b. the second antigen-binding domain that is capable of binding to human PD-L1 and to human IFN-α2 comprises
   i. a heavy chain variable domain (VH) comprising (a) CDR-H1 comprising the amino acid sequence of SEQ ID NO:1, (b) CDR-H2 comprising the amino acid sequence of SEQ ID NO:2, and (c) CDR-H3 comprising the amino acid sequence of SEQ ID NO:3, and a light chain variable domain (VL) comprising (d) CDR-L1 comprising the amino acid sequence of SEQ ID NO:4, (e) CDR-L2 comprising the amino acid sequence of SEQ ID NO: 5, and (f) CDR-L3 comprising the amino acid sequence of SEQ ID NO: 6;
   ii. a heavy chain variable domain (VH) comprising (a) CDR-H1 comprising the amino acid sequence of SEQ ID NO:1, (b) CDR-H2 comprising the amino acid sequence of SEQ ID NO:2, and (c) CDR-H3 comprising the amino acid sequence of SEQ ID NO:3, and a light chain variable domain (VL) comprising (d) CDR-L1 comprising the amino acid sequence of SEQ ID NO:9, (e) CDR-L2 comprising the amino acid sequence of SEQ ID NO: 5, and (f) CDR-L3 comprising the amino acid sequence of SEQ ID NO: 6;
   iii. a heavy chain variable domain (VH) comprising (a) CDR-H1 comprising the amino acid sequence of SEQ ID NO:1, (b) CDR-H2 comprising the amino acid sequence of SEQ ID NO:2, and (c) CDR-H3 comprising the amino acid sequence of SEQ ID NO:3, and a light chain variable domain (VL) comprising (d) CDR-L1 comprising the amino acid sequence of SEQ ID NO:4, (e) CDR-L2 comprising the amino acid sequence of SEQ ID NO: 11, and (f) CDR-L3 comprising the amino acid sequence of SEQ ID NO: 6; or
   iv. a heavy chain variable domain (VH) comprising (a) CDR-H1 comprising the amino acid sequence of SEQ ID NO:13, (b) CDR-H2 comprising the amino acid sequence of SEQ ID NO: 14, and (c) CDR-H3 comprising the amino acid sequence of SEQ ID NO:15, and a light chain variable domain (VL) comprising (d) CDR-L1 comprising the amino acid sequence of SEQ ID NO:16, (e) CDR-L2 comprising the amino acid sequence of SEQ ID NO: 17, and (f) CDR-L3 comprising the amino acid sequence of SEQ ID NO: 18; and
c. the fusion protein further comprises an Fc domain composed of a first and a second subunit.

In a particular embodiment of the fusion protein provided herein, the first antigen-binding domain capable of binding to human PD-L1 is fused at the C-terminus of its Fab heavy chain to the N-terminus of the first subunit of the Fc domain, and the second antigen-binding domain capable of binding to human PD-L1 and to human IFN-α2 is fused at the C-terminus of its Fab heavy chain to the N-terminus of the second subunit of the Fc domain, and the human IFN-α2 is fused at its C-terminus to the N-terminus of the Fab light chain of the second antigen-binding domain.

In one embodiment, the invention is a fusion protein that comprises a first antigen-binding domain capable of binding to human PD-L1, a second antigen-binding domain capable of binding to human PD-L1 and to human IFN-α2, and a human IFN-α2, wherein the second antigen-binding domain is a bispecific Fab and the human IFN-α2 is fused at its C-terminus to the N-terminus of the heavy chain or the light chain of the second antigen-binding domain, wherein
a. the first antigen-binding domain that is capable of binding to human PD-L1 comprises
   a heavy chain variable domain (VH) comprising (a) CDR-H1 comprising the amino acid sequence of SEQ ID NO:21, (b) CDR-H2 comprising the amino acid sequence of SEQ ID NO:22, and (c) CDR-H3 comprising the amino acid sequence of SEQ ID NO:23, and a light chain variable domain (VL) comprising (d) CDR-L1 comprising the amino acid sequence of SEQ ID NO:24, (e) CDR-L2 comprising the amino acid sequence of SEQ ID NO:25, and (f) CDR-L3 comprising the amino acid sequence of SEQ ID NO:26; and
b. the second antigen-binding domain that is capable of binding to human PD-L1 and to human IFN-α2 comprises
   a heavy chain variable domain (VH) comprising (a) CDR-H1 comprising the amino acid sequence of SEQ ID NO:1, (b) CDR-H2 comprising the amino acid sequence of SEQ ID NO:2, and (c) CDR-H3 comprising the amino acid sequence of SEQ ID NO:3, and a light chain variable domain (VL) comprising (d) CDR-L1 comprising the amino acid sequence of SEQ ID NO:4, (e) CDR-L2 comprising the amino acid sequence of SEQ ID NO:5, and (f) CDR-L3 comprising the amino acid sequence of SEQ ID NO:6; and
c. the fusion protein further comprises an Fc domain composed of a first and a second subunit.

In a particular embodiment of the fusion protein provided herein, the first antigen-binding domain is fused at the C-terminus of its Fab heavy chain to the N-terminus of the first subunit of the Fc domain, and the second antigen-binding domain is fused at the C-terminus of its Fab heavy chain to the N-terminus of the second subunit of the Fc domain, and the Interferon alpha is fused at its C-terminus to the N-terminus of the Fab light chain of the second antigen-binding domain.

In one embodiment, the fusion protein comprises a first antigen-binding domain capable of binding to human PD-L1, a second antigen-binding domain capable of binding to human PD-L1 and to human IFN-α2, and a human IFN-α2, wherein the second antigen-binding domain is a bispecific Fab and the human IFN-α2 is fused at its C-terminus to the N-terminus of the heavy chain or the light chain of the second antigen-binding domain, wherein
the second antigen-binding domain capable of binding to human PD-L1 and to human IFN-α2 comprises
   a. a VH domain comprising the amino acid sequence of SEQ ID NO:7 and a VL domain comprising the amino acid sequence of SEQ ID NO:8;
   b. a VH domain comprising the amino acid sequence of SEQ ID NO:7 and a VL domain comprising the amino acid sequence of SEQ ID NO:10;
   c. a VH domain comprising the amino acid sequence of SEQ ID NO:7 and a VL domain comprising the amino acid sequence of SEQ ID NO: 12; or
   d. a VH domain comprising the amino acid sequence of SEQ ID NO: 19 and a VL domain comprising the amino acid sequence of SEQ ID NO:20; and
the first antigen-binding domain capable of binding to human PD-L1 comprises
   a. a VH domain comprising the amino acid sequence of SEQ ID NO:27 and a VL domain comprising the amino acid sequence of SEQ ID NO:28; or
   b. a VH domain comprising the amino acid sequence of SEQ ID NO:35 and a VL domain comprising the amino acid sequence of SEQ ID NO:36.

In one embodiment, the invention is a fusion protein comprising a first antigen-binding domain capable of binding to human PD-L1, a second antigen-binding domain capable of binding to human PD-L1 and to human IFN-α2, and a human IFN-α2, wherein the second antigen-binding domain is a bispecific Fab and the human IFN-α2 is fused at its C-terminus to the N-terminus of the heavy chain or the light chain of the second antigen-binding domain,

wherein the second antigen-binding domain capable of binding to human PD-L1 and to human IFN-α2 comprises a VH domain comprising the amino acid sequence of SEQ ID NO: 7 and a VL domain comprising the amino acid sequence of SEQ ID NO:8, and
wherein the first antigen-binding domain capable of binding to human PD-L1 comprises a VH domain comprising the amino acid sequence of SEQ ID NO:27 and a VL domain comprising the amino acid sequence of SEQ ID NO:28.

In one embodiment, the invention is a fusion protein comprising
a. a first antigen-binding domain capable of binding to human PD-L1;
b. a second antigen-binding domain capable of binding to human PD-L1 and to human IFN-α2;
c. a human IFN-α2; and
d. an Fc domain composed of a first and a second subunit;
   wherein the first antigen-binding domain is a Fab and the second antigen-binding domain is a bispecific Fab, preferably a DutaFab; wherein the first and the second antigen-binding domain are each composed of a heavy chain and a light chain; and
   wherein the fusion protein is composed of
(a) a first polypeptide, comprising (a1) the heavy chain of the first antigen binding domain, fused at its C-terminus to the N-terminus of one of the subunits (e.g. the first subunit) of the Fc domain, and (a2) one of the subunits (e.g. the first subunit) of the Fc domain;
(b) a second polypeptide, comprising the light chain of the first antigen-binding domain;
(c) a third polypeptide, comprising (c1) the heavy chain of the second antigen-binding domain, fused at its C-terminus to the N-terminus of the other one of the subunits (e.g. the second subunit) of the Fc domain, and (c2) the other one of the subunits (e.g. the second subunit) of the Fc domain; and
(d) a fourth polypeptide, comprising (d1) the human IFN-α2, fused at its C-terminus to the N-terminus of the light chain of the second antigen-binding domain, and (d2) the light chain of the second antigen-binding domain.

In one embodiment, the fusion protein comprises
a first polypeptide comprising an amino acid sequence that is at least about 95%, 96%, 97%, 98%, 99% or 100% identical to the amino acid sequence of SEQ ID NO: 37;
a second polypeptide comprising an amino acid sequence that is at least about 95%, 96%, 97%, 98%, 99% or 100% identical to the amino acid sequence of SEQ ID NO: 38;
a third polypeptide comprising an amino acid sequence that is at least about 95%, 96%, 97%, 98%, 99% or 100% identical to the amino acid sequence of SEQ ID NO: 39; and
a fourth polypeptide comprising an amino acid sequence that is at least about 95%, 96%, 97%, 98%, 99% or 100% identical to the amino acid sequence of SEQ ID NO: 40.

In one embodiment, the fusion protein comprises four polypeptides wherein
the first polypeptide comprises an amino acid sequence that is at least about 95%, 96%, 97%, 98%, 99% or 100% identical to the amino acid sequence of SEQ ID NO:41;
the second polypeptide comprises an amino acid sequence that is at least about 95%, 96%, 97%, 98%, 99% or 100% identical to the amino acid sequence of SEQ ID NO:42;
the third polypeptide comprises an amino acid sequence that is at least about 95%, 96%, 97%, 98%, 99% or 100% identical to the amino acid sequence of SEQ ID NO:43; and
the fourth polypeptide comprises an amino acid sequence that is at least about 95%, 96%, 97%, 98%, 99% or 100% identical to the amino acid sequence of SEQ ID NO:44.

In one embodiment, the fusion protein comprises four polypeptides wherein
the first polypeptide comprises an amino acid sequence that is at least about 95%, 96%, 97%, 98%, 99% or 100% identical to the amino acid sequence of SEQ ID NO:45;
the second polypeptide comprises an amino acid sequence that is at least about 95%, 96%, 97%, 98%, 99% or 100% identical to the amino acid sequence of SEQ ID NO:46;
the third polypeptide comprises an amino acid sequence that is at least about 95%, 96%, 97%, 98%, 99% or 100% identical to the amino acid sequence of SEQ ID NO:47; and
the fourth polypeptide comprises an amino acid sequence that is at least about 95%, 96%, 97%, 98%, 99% or 100% identical to the amino acid sequence of SEQ ID NO:48.

In one embodiment, the fusion protein comprises four polypeptides wherein
the first polypeptide comprises an amino acid sequence that is at least about 95%, 96%, 97%, 98%, 99% or 100% identical to the amino acid sequence of SEQ ID NO:49;
the second polypeptide comprises an amino acid sequence that is at least about 95%, 96%, 97%, 98%, 99% or 100% identical to the amino acid sequence of SEQ ID NO:50;
the third polypeptide comprises an amino acid sequence that is at least about 95%, 96%, 97%, 98%, 99% or 100% identical to the amino acid sequence of SEQ ID NO:51; and
the fourth polypeptide comprises an amino acid sequence that is at least about 95%, 96%, 97%, 98%, 99% or 100% identical to the amino acid sequence of SEQ ID NO:52.

In one embodiment, the fusion protein comprises four polypeptides wherein
the first polypeptide comprises an amino acid sequence that is at least about 95%, 96%, 97%, 98%, 99% or 100% identical to the amino acid sequence of SEQ ID NO:53;
the second polypeptide comprises an amino acid sequence that is at least about 95%, 96%, 97%, 98%, 99% or 100% identical to the amino acid sequence of SEQ ID NO:54;
the third polypeptide comprises an amino acid sequence that is at least about 95%, 96%, 97%, 98%, 99% or 100% identical to the amino acid sequence of SEQ ID NO:55; and
the fourth polypeptide comprises an amino acid sequence that is at least about 95%, 96%, 97%, 98%, 99% or 100% identical to the amino acid sequence of SEQ ID NO:56.

In one embodiment, the fusion protein comprises four polypeptides wherein
the first polypeptide comprises an amino acid sequence that is at least about 95%, 96%, 97%, 98%, 99% or 100% identical to the amino acid sequence of SEQ ID NO:57;
the second polypeptide comprises an amino acid sequence that is at least about 95%, 96%, 97%, 98%, 99% or 100% identical to the amino acid sequence of SEQ ID NO:58;
the third polypeptide comprises an amino acid sequence that is at least about 95%, 96%, 97%, 98%, 99% or 100% identical to the amino acid sequence of SEQ ID NO:59; and
the fourth polypeptide comprises an amino acid sequence that is at least about 95%, 96%, 97%, 98%, 99% or 100% identical to the amino acid sequence of SEQ ID NO:60.

Such fusion proteins have highly valuable properties like their binding properties, in particular the high affinity of the second antigen-binding domain to human IFN-α2a, and their blocking properties, which ensure that the IFN-α2a moiety is reliably prevented (“masked”) from activating the IFNAR receptor as long there is no or not sufficient PD-L1-target molecule present. Another valuable property is the ability of the first antigen-binding domain and the second antigen-binding domain in some embodiments of the fusion protein provided herein to bind to the C-terminal domain of PD-L1, thus not competing with other therapeutic anti-PD-L1 antibodies such as Atezolizumab for PD-L1 binding, but still being able to block PD-L1/PD1 interaction. This effect can be observed particularly at elevated concentrations and is likely caused by steric hindrance. Due to this property, the fusion proteins of the invention may be able to achieve a therapeutic effect in addition to IFNα activation, by blocking PD-L1/PD1 interaction. They are also ideally suited to be used in combination therapy with known therapeutic anti-PD-L1 antibodies that bind to the N-terminus of PD-L1.

Further valuable properties of the fusion proteins of the invention are their comparatively low tendency to form undesirable dimers, their high thermostability, their low degradation propensity, and their ability to inhibit tumor growth, particularly in PD-L1 expressing tumors. Another valuable property of the fusion proteins of the invention is their high potency, i.e. they achieve a high level of IFN-α2a pathway activation at PD-L1 high expressing cells at a low dose, whereas the level of IFN-α2a activity at PD-L1 low expressing cells at the same dose is very low in comparison, as e.g. measured in a HEK-blue human IFNAR1/2 reporter cell assay as described herein. The first and the second antigen-binding domains further show crossreactivity with cynomolgus PD-L1. Another highly valuable property is their ability to inhibit tumor cell proliferation and tumor growth (including induction of tumor regression), in particular in PD-L1 expressing tumors or tumor cell lines. Moreover, the fusion proteins described herein have good tolerability and a beneficial side effect profile, particularly in comparison to untargeted, unattenuated IFN-α2, because the second antigen-binding domain that remains bound to IFN-α2 in the absence of PD-L1 expressing target cells prevents undesirable activity of IFN-α2 in peripheral tissue and directs the activity of the IFN-α2 in the fusion protein to the target tissue.

Provided herein is also an antibody that binds to human PD-L1, wherein the antibody comprises
a heavy chain variable domain (VH) comprising (a) CDR-H1 comprising the amino acid sequence of SEQ ID NO:21, (b) CDR-H2 comprising the amino acid sequence of SEQ ID NO:22, and (c) CDR-H3 comprising the amino acid sequence of SEQ ID NO: 23; and
a light chain variable domain (VL) comprising (d) CDR-L1 comprising the amino acid sequence of SEQ ID NO:24, (e) CDR-L2 comprising the amino acid sequence of SEQ ID NO:25, and (f) CDR-L3 comprising the amino acid sequence of SEQ ID NO: 26.

In one embodiment, the antibody that binds to PD-L1 comprises a sequence selected from the group consisting of
(a) a VH sequence having at least 95% sequence identity to the amino acid sequence of SEQ ID NO:27;
(b) a VL sequence having at least 95% sequence identity to the amino acid sequence of SEQ ID NO:28; and
(c) a VH sequence as defined in (a) and a VL sequence as defined in (b).

In one embodiment, the antibody that binds to PD-L1 comprises a VH sequence of SEQ ID NO: 27 and a VL sequence of SEQ ID NO:28.

In one embodiment, the antibody that binds to PD-L1 comprises a heavy chain of SEQ ID NO: 142 and a light chain of SEQ ID NO:143.

Such antibodies have highly valuable properties like their binding properties, in particular their high affinity to PD-L1, and their ability to bind to the C-terminal domain of PD-L1, thus not competing with other therapeutic anti-PD-L1 antibodies such as Atezolizumab for PD-L1 binding, but surprisingly still being able to block PD-L1/PD1 interaction, particularly at elevated concentrations. Due to this property, the antibodies of the invention may be able to achieve an improved therapeutic effect by blocking PD-L1/PD1 interaction. They are also ideally suited to be used in combination therapy with known therapeutic anti-PD-L1 antibodies that bind to the N-terminus of PD-L1. They are also well suited for diagnostic purposes, e.g. for detecting PD-L1 in assays testing the effect of blocking anti-PD-L1 antibodies, as these generally bind to the N-terminal domain of PD-L1. The anti-PD-L1 antibodies of the invention also show crossreactivity with cynomolgus PD-L1.

In one embodiment, the antibody binding to PD-L1 binds specifically to PD-L1. In one embodiment, the antibody that binds to PD-L1 is a monoclonal antibody. In one embodiment, the antibody that binds to PD-L1 is a humanized or chimeric antibody. In one embodiment, the antibody that binds to PD-L1 is a full-length IgG1 antibody.

In certain embodiments, the antibody that binds to PD-L1 is an antibody fragment that binds to PD-L1. In one embodiment, the antibody that binds PD-L1 is a DutaFab.

In one embodiment, the antibody binds to PD-L1 with an affinity of 1.1 nM or lower, preferably of 0.6 nM or lower, as measured using a BIACORE® surface plasmon resonance assay at 25° C. In one embodiment, the antibody binds to the C-terminal domain of PD-L1. In one embodiment, the binding of the antibody to PD-L1 blocks the interaction of PD-L1 and PD1 as measured by SPR. In one embodiment, the antibody that binds to PD-L1 is a multispecific antibody.

The invention provides an isolated nucleic acid encoding the fusion protein or the antibody described herein.

The invention provides a host cell comprising such a nucleic acid.

The invention provides a method of producing a fusion protein or an antibody described herein comprising culturing the host cell under conditions suitable for the expression of the fusion protein or the antibody. The invention provides such a method of producing a fusion protein or an antibody, further comprising recovering the fusion protein or the antibody from the host cell. The invention also provides a fusion protein or antibody produced by such method.

The invention provides a pharmaceutical composition comprising the fusion protein or the antibody described herein and a pharmaceutically acceptable carrier.

The invention provides the fusion protein, the antibody or the pharmaceutical composition described herein for use as a medicament.

The invention provides the fusion protein, the antibody or the pharmaceutical composition described herein for use in treating cancer.

The invention provides the use of the fusion protein, the antibody or the pharmaceutical composition described herein in the manufacture of a medicament for treatment of cancer.

The invention provides the use of the fusion protein, the antibody or the pharmaceutical composition described herein in the manufacture of a medicament for targeting a therapeutically active agent to a tumor cell and/or an immune cell in the tumor microenvironment.

The invention provides a method of treating an individual having cancer comprising administering to the individual an effective amount of the fusion protein, the antibody or the pharmaceutical composition described herein.

The invention provides a method of inhibiting cell proliferation and/or tumor growth in an individual comprising administering to the individual an effective amount of the fusion protein, the antibody or the pharmaceutical composition described herein to inhibit cell proliferation and/or tumor growth.

The invention provides a method of modulating the immune system by directly or indirectly inducing proliferation and/or activation of immune cells in an individual comprising administering to the individual an effective amount of the fusion protein, the antibody or the pharmaceutical composition described herein to modulate the immune system by directly or indirectly inducing proliferation and/or activation of immune cells.

### BRIEF DESCRIPTION OF THE FIGURES

FIG. 1A-1B: Schematic illustration of the PD-L1-targeted IFN-α2a fusion protein used in the Examples. The molecule comprises a CrossFab as a first antigen-binding domain capable of binding huPD-L1 and a DutaFab as a second antigen-binding domain capable of binding to huPD-L1 and to huIFN-α2a. Both antigen-binding domains fused via the C-termini of their heavy chains to the N-terminus of one of the two subunits of an Fc domain. The fusion protein further comprises a huIFN-α2a moiety which is fused via its C-terminus to the N-terminus of the light chain of the second antigen-binding domain. FIGS. 1A and 1 B illustrate the principle of the “Dutaflip” as a molecular switch: FIG. 1A shows the PD-L1-targeted human Interferon-α2 fusion protein in a form where the fused huIFN-α2a molecule is bound to the bispecific anti-PD-L1/anti-IFN-α2a DutaFab. In this form, the binding to the bispecific DutaFab would prevent the huIFN-α2a moiety from binding to its natural receptor IFNAR1/2. FIG. 1B shows the molecule in a form where the fused huIFN-α2a molecule is not bound to the bispecific anti-PD-L1/anti-IFN-α2a Fab, as would be the case, for instance, if the bispecific anti-PD-L1/anti-IFN-α2a binder had bound to PD-L1, e.g. on the surface of a target cell, thus preventing the huIFN-α2a moiety from binding to the bispecific DutaFab. (Black dot: modification in the Fc domain promoting heterodimerization. *: amino acids of opposite charges introduced in the CH and CL domains)

FIG. 2: Results of an SPR assay demonstrating lack of competitive binding of various anti-PD-L1 Fabs with Atezolizumab. The tested anti-PD-L1 Fabs were derived from the anti-PD-L1 binding CDRs (H-side) of the anti-PD-L1/anti-IFN-α2a bispecific DutaFab M14HH17L (parental), as described in Example 7. The sensorgram shows that the tested Fabs did not compete with Atezolizumab binding to PD-L1.

FIG. 3: Results of an SPR assay demonstrating the inhibition of PD1/PD-L1 interaction by various anti-PD-L1 Fabs. The two tested anti-PD-L1 Fabs (P1AI4475 “M14” and P1AI4392) were derived from the anti-PD-L1 binding CDRs (H-side) of the anti-PD-L1/anti-IFN-α2a bispecific DutaFab M14HH17L (parental), as described in Example 7. The sensorgrams show that the two M14HH17L (parental)-derived anti-PD-L1 Fabs block binding of PD-L1 to PD1, like the Atezolizumab-based anti-PD-L1 Fab (P1AI4841) which was used as a positive control. P1AH0183 is a PD-L1-specific antibody derived from bispecific DutaFab E06HP7L, which does not block PD-L1/PD1 interaction.

FIG. 4: The diagram shows the results of the PD1/PD-L1 blockade bioassay, demonstrating that M14HH17L (parental)-derived anti-PD-L1 Fabs can block PD1/PD-L1 interaction also in a cellular context.

FIG. 5A-5F: Schematic illustration of various reference molecules that were used in the examples. FIG. 5A shows fusion proteins wherein IFN-α2a is fused to the N-terminal end of the light chain of the DutaFab binding to PD-L1/IFN-α2a (P1AH1108, P1AH1111). FIG. 5 B shows fusion proteins wherein IFN-α2a is fused to the C-terminal end of the light chain of the DutaFab binding to PD-L1/IFN-α2a (PIAG9500, PIAG9456, PIAH1122, P1AH1123). FIG. 5 C shows a fusion protein with IFN-α2a fused to the N-terminus of the heavy chain of the domain binding to PD-L1/IFN-α2a. FIG. 5 D shows fusions proteins wherein the antigen-binding domain binding to PD-L1/IFN-α2a was fused to the C-terminus of one subunit of the Fc domain and IFN-α2a was fused to the C-terminus of the other subunit of the Fc domain (P1AG4739 and PAG4740). FIG. 5 E and FIG. 5 F show two different 2+1 formats wherein IFN-α2a is fused to the N-terminus of the light chain of a DutaFab binding to PD-L1/IFN-α2a.

FIG. 6A-6C: Schematic illustration of the reference molecules P1AI0336, P1AI0337 and P1AI0338 (FIG. 6A), P1AI1297, P1AI1298 (FIG. 6 B) and P1AI1326 (FIG. 6 C).

FIG. 7A-7B: Mass photometry measurement of molecule P1AI4295 at a concentration of 250 nM (FIG. 7A) and 3 μM (FIG. 7 B) revealed a mass of approximately 172 kDa, demonstrating that the fusion protein forms primarily monomers and does not have a tendency to dimerize.

FIG. 8A-8B: Schematic illustration of the activity assay of PD-L1-targeted IFN-α2a Dutaflip fusion proteins on low and high expressing PD-L1 HEK-Blue IFNα/β reporter cells. When not enough PD-L1 molecules are present on the cell surface (FIG. 8A), IFN-α2 continues to be bound by the DutaFab (“Dutaflip”) and cannot bind to its cell surface receptor IFNAR1/2. No activation of the reporter cells and consequently also no reporter gene expression is achieved. When sufficient PD-L1 is expressed on the cell surface (FIG. 8 B), the “Dutaflip” binds to PD-L1 on the cell surface and no longer masks IFN2α. IFN-α2 is now free to bind to the IFNAR1/2 receptor on the cell surface and triggers detectable SEAP production of the reporter cells (the figure was created with BioRender.com).

FIG. 9A-9B: In vitro activation of parental HEK-Blue IFN-α/β cells (FIG. 9A) or PD-L1 transfected HEK-Blue IFN-α/β cells (FIG. 9 B) by PD-L1-targeted IFN-α2a Dutaflip fusion proteins or recombinant IFN-α2a after 24 hours of incubation. Figures are showing the mean percentage of reporter cell activation relative to the activation in response to 10 nM recombinant IFN-α2a. n=4.

FIG. 10: EC50 values calculated from dose-response curves of HEK-Blue IFN-α/β cells transfected with PD-L1 and treated with different concentrations of PD-L1-targeted IFN-α2a Dutaflip fusion proteins or recombinant IFN-α2a for 24 h. n=4.

FIG. 11A-11E: FIGS. 11A-D show direct comparisons of the in vitro activation of parental HEK-Blue IFN-α/β cells versus PD-L1 transfected (“Clone 045”) HEK-Blue IFN-α/β cells for each tested PD-L1-targeted IFN-α2 Dutaflip fusion protein or reference molecule after 24 hours of incubation in a single graph. Values were normalized to activation by setting the response to 10 nM recombinant IFN-α2a to 100%. n=4. FIG. 11 E shows the difference (A) between IFNα activation in PD-L1 low- and high-expressing cells of each molecule plotted against the concentration of the molecule used in the assay. The graph shows that the fusion proteins of the invention show already significant difference in activation at around 104 nM and generally reach their maximum window between IFNα activation in PD-L1 high-expressing cells and PD-L1 low-expressing cells at a lower concentration than the reference molecules.

FIG. 12A-12C: FIGS. 12A-C show the inhibition of human tumor cell proliferation in vitro by different PD-L1-targeted IFN-α2a Dutaflip fusion proteins (P1AI4295, P1AI4798, P1AI4797, P1AI4748), reference molecule (P1AI1297) or recombinant IFN-α2a in different tumor cell lines. The confluence of the cells was measured every 4 hours for up to ten days, n=2.

FIG. 13A-13C: CXCL10 (IP-10) induction by PD-L1-targeted IFN-α2a Dutaflip fusion proteins, reference molecule or recombinant IFN-α2a in vitro in different human tumor cell lines. Error bars represent standard deviation. FIG. 13A: SK-BR3: 100,000 cells; FIG. 13B: BT-20:200,000 cells; FIG. 13C: HCC1954: 30,000 cells). The levels of CXCL10 in the supernatant were measured by ELISA after 24 hours incubation, n=2.

FIG. 14A-14B: Induction of MHCI (FIG. 14A) and PD-L1 (FIG. 14B) expression of HCC1954 over time with PD-L1-targeted IFN-α2a Dutaflip fusion proteins and recombinant human IFN-α2. FIG. 14A shows the red (MHCI) and FIG. 14 B shows the green (PD-L1) object count/image.

FIG. 15: In vitro IFN-γ secretion induced by PD-L1-targeted IFN-α2a Dutaflip fusion proteins, reference molecule or recombinant IFN-α2a in human NK-92 cells after 48 hours incubation. The figure shows pg/ml IFN-y secretion dependent on the antibody concentration in nM, n=2; error bars represent S.D.

FIG. 16A-16C: In vitro human CD80 expression of pDCs (FIG. 16A), DC1 (FIG. 16B) and DC2 (FIG. 16C) by PD-L1-targeted IFN-α2a Dutaflip fusion proteins or recombinant IFN-α2a after 24h incubation. The figures are showing the geometric mean of CD80 expression on different DC subsets treated with 10 nM of indicated antibodies or recombinant IFN-α2a.

FIG. 17A-17B: T-cell activation after 24 hours incubation with 10 nM PD-L1-targeted IFN-α2a Dutaflip fusion proteins or recombinant IFN-α2a. The figures show the percentage of CD69 expressing CD4+ (FIG. 17A) or CD8+ (FIG. 17B) T cells. Error bars represent standard deviation.

FIG. 18A-18B: In vitro CXCL10 (FIG. 18A) and IL6 (FIG. 18B) secretion in pg/ml of fresh human whole blood (low PD-L1 expression) incubated with indicated PD-L1-targeted IFN-α2a molecules or recombinant human IFN-α2a at a concentration of 4 nM for 24 h. Shown are three independent experiments with a total number of 11 donors. Error bars represent standard deviation.

FIG. 19: Internalization of 10 nM PD-L1-targeted IFN-α2a Dutaflip fusion proteins, reference molecule and control, which were conjugated to human/mouse Fabfluor pH Red Labeling reagent, into the PD-L1 expressing human tumor cell line HCC1954. The figure shows the red object count per image within 48 h incubation. Error bars represent standard deviation.

FIG. 20: FIG. 20 shows the results of the efficacy study of PD-L1-targeted IFN-α2a Dutaflip fusion proteins in tumor-bearing huNSG mice. Tumor volume was measured by caliper 2 times a week. FIG. 20 shows a direct comparison of the results of all subgroups, over a period from day 22 to day 51. Data is shown as tumor volume (Median+/−IQR).

FIG. 21A-21C: FIGS. 21A-21C show the inhibition of human tumor cell proliferation (measured as cell confluence) in vitro by the PD-L1-targeted IFN-α2a Dutaflip fusion proteins P1AI4295 or recombinant huIFN-α2a in tumor cell line COR-L105 at different protein concentrations, 0.1 nM (FIG. 21 A), 1 nM (FIG. 21 B) and 10 nM (FIG. 21 C). Untreated cells were used as negative control. The confluence of the cells was measured every 4 hours for up to ten days, n=2.

FIG. 22A-22C: FIGS. 22A-22C show the inhibition of human tumor cell proliferation in vitro (measured as cell confluence) by the PD-L1-targeted huIFN-α2a Dutaflip fusion proteins P1AI4295 or recombinant huIFN-α2a in tumor cell line HDLM-2 at different protein concentrations, 0.1 nM (FIG. 22 A), 1 nM (FIG. 22 B) and 10 nM (FIG. 22 C). Untreated cells were used as negative control. The confluence of the cells was measured every 4 hours for up to ten days, n=2.

FIG. 23A-23B: FIGS. 23A and 23 B show the results of the PD1/PD-L1 blockade bioassay, demonstrating that M14HH17L (parental)-derived anti-PD-L1 Fabs retain their ability to block PD1/PD-L1 interaction also when used as targeting arms in PD-L1 targeted IFN-α2a fusion proteins.

FIG. 24A-24B: FIGS. 24A and 24 B show the cytokine response elicited by PD-L1-targeted IFN-α2a Dutaflip fusion proteins (labelled “Duta Flip”) (as defined by log 2 x-fold change compared to untreated) for IP-10, ISG-15, and IFNγ in tumor samples obtained from NSCLC, BLCA, ccRCC and TNBC patients. Recombinant huIFN-α2a (labelled “rec. IFNα”) was used as a positive control.

FIG. 25: This figure shows a heat map illustrating the correlation between the frequency of PD-L1 positive cells and PD-L1 positive CD45 negative cells of all samples, respectively, and elevated cytokine response levels for IP-10, ISG-15, IFNγ, GrnzB, IL-6, MIP-1, pan IFNα, CXCL13 in tumor samples obtained from NSCLC, BLCA, ccRCC and TNBC patients treated with the PD-L1-targeted IFN-α2a Dutaflip fusion protein. Recombinant huIFN-α2a (labelled “rec. IFNα”) was used as a positive control.

FIG. 26A-26B: The diagrams of FIGS. 26A and 26 B show the correlation between the frequency of PD-L1 expressing CD45 negative cells and the ISG-15 response within the study cohort (n=13 patients) for the P1AI4295 treated (“Duta Flip”) condition (FIG. 26A) and for the condition treated with recombinant human Interferon Alpha 2a (control, FIG. 26 B), as a specific instance to illustrate the correlation between two continuous variables.

FIG. 27A-27B: UMAP analysis on the snRNA-seq data obtained from the tumor samples post 3D cultivation and treatment. UMAP visualization revealed 44 clusters representing different cellular subpopulations, based on 351,005 cells derived from 39 samples originating from 7 patients (FIG. 27A). FIG. 27 B shows the cellular composition of the sample, analyzed via the expression of known marker genes within each UMAP cluster.

FIG. 28: This diagram illustrates the examination of an inflammatory gene signature (ISG15, MX1, XAF1, HERC5, STAT1, IFIT1, IFI6, CXCL11, ISG20, IL10) by visualizing the IFN-α-targeted cis-activation pattern in PD-L1-positive and -negative tumor cells. In the untreated conditions were used as negative control. Treatment with recombinant IFN-α2a was used as positive control group. Heightened frequencies of positive cells is indicated by the size of the bubble and upregulation in the mean expression levels of the respective markers is indicated by the escalation of the gray scale.

FIG. 29: Comparison of PD-L1 expression levels across the three test conditions (treated with P1AI4295, treated with recombinant IFN-α2a and untreated).

FIG. 30A-30B: In an Immunocompetent Hematotoxicity Assay, P1AI4295 showed less toxicity towards erythroid-lineage (FIG. 30A) and granulocyte/monocyte cells (FIG. 30 B) than recombinant IFN-α2a (control).

FIG. 31A-31D: FIGS. 31A-31 D show the results of the PBMC Immunotoxicity Assay. PD-L1-targeted IFN-α2a fusion protein P1AI4295 shows a reduced % CD69 activation profile in PBMC-derived CD4 T-cells (FIG. 31A), CD8 T-cells (FIG. 31 B), B-cells (FIG. 31 C) and Monocytes (FIG. 31 D), as compared to recombinant IFN-α2a as control.

### DETAILED DESCRIPTION

### I. Definitions

Unless otherwise defined herein, scientific and technical terms used in connection with the present invention shall have the meanings that are commonly understood by those of ordinary skill in the art. Further, unless otherwise required by context, singular terms shall include pluralities and plural terms shall include the singular. The methods and techniques of the present disclosure are generally performed according to conventional methods well known in the art. Generally, nomenclatures used in connection with, and techniques of biochemistry, enzymology, molecular, and cellular biology, microbiology, genetics and protein and nucleic acid chemistry and hybridization described herein are those well-known and commonly used in the art.

Unless otherwise defined herein the term “comprising of” shall include the term “consisting of”.

The term “about” as used herein in connection with a specific value (e.g. temperature, concentration, time and others) shall refer to a variation of +/−1% of the specific value that the term “about” refers to.

An “acceptor human framework” for the purposes herein is a framework comprising the amino acid sequence of a light chain variable domain (VL) framework or a heavy chain variable domain (VH) framework derived from a human immunoglobulin framework or a human consensus framework, as defined below. An acceptor human framework “derived from” a human immunoglobulin framework or a human consensus framework may comprise the same amino acid sequence thereof, or it may contain amino acid sequence changes. In some aspects, the number of amino acid changes are 10 or less, 9 or less, 8 or less, 7 or less, 6 or less, 5 or less, 4 or less, 3 or less, or 2 or less. In some aspects, the VL acceptor human framework is identical in sequence to the VL human immunoglobulin framework sequence or human consensus framework sequence.

“Affinity” refers to the strength of the sum total of noncovalent interactions between a single binding site of a molecule (e.g., an antibody, an antibody fragment or an antigen-binding domain) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (K_{D}). Affinity can be measured by common methods known in the art, including those described herein. Specific illustrative and exemplary methods for measuring binding affinity are described in the following.

An “affinity matured” antibody refers to an antibody with one or more alterations in one or more complementary determining regions (CDRs), compared to a parent antibody which does not possess such alterations, such alterations resulting in an improvement in the affinity of the antibody for antigen.

The terms “anti-huPD-L1 antibody”, “an antibody that binds human PD-L1” and “an antibody that is capable of binding to human PD-L1” refer to an antibody that is capable of binding human PD-L1 with sufficient affinity such that the antibody is useful as a diagnostic and/or therapeutic agent in targeting human PD-L1. In one aspect, the extent of binding of an anti-huPD-L1 antibody to an unrelated, non-huPD-L1 protein is less than about 10% of the binding of the antibody to human PD-L1 as measured, e.g., by surface plasmon resonance (SPR). In certain aspects, an antibody that binds to human PD-L1 has a dissociation constant (K_{D}) of ≤1 μM, ≤100 nM, ≤10 nM, ≤1 nM, ≤0.1 nM, ≤0.01 nM, or ≤0.001 nM (e.g., 10⁻⁸ M or less, e.g., from 10⁻⁸ M to 10⁻¹³ M, e.g., from 10⁻⁹ M to 10⁻¹³ M). An antibody is said to “specifically bind” to human PD-L when the antibody has a K_{D} of 1 μM or less. In certain aspects, an anti-PD-L1 antibody binds to an epitope of human PD-L1 that is conserved among human PD-L1 from different species. As used herein, the term antibody “monospecific for human PD-L1” means an antibody that binds, in particular binds specifically, to one antigen or one epitope on human PD-L1, while a bispecific antibody as referred to herein binds two distinct antigens or two distinct epitopes, in particular two distinct antigens or two distinct epitopes on two distinct target molecules. The terms “human PD-L1” or “anti-human-PD-L1” may for conciseness be referred to simply as “huPD-L1” or “anti-huPD-L1” herein, respectively. Similarly, other human proteins, such as “human PD1” may be referred to as “huPD1”.

The terms “anti-huPD-L1 antigen-binding domain”, “an antigen-binding domain that binds to human PD-L1” and “an antigen-binding domain that is capable of binding to human PD-L1” refer to an antigen-binding domain that is capable of binding human PD-L1 with sufficient affinity such that the antigen-binding domain is useful as a diagnostic and/or therapeutic agent in targeting huPD-L1. In one aspect, the extent of binding of an anti-huPD-L1 antigen-binding domain to an unrelated, non-PD-L1 protein is less than about 10% of the binding of the antigen-binding domain to PD-L1 as measured, e.g., by surface plasmon resonance (SPR). In certain aspects, an antigen-binding domain that binds to huPD-L1 has a dissociation constant (K_{D}) of ≤1 μM, ≤100 nM, ≤10 nM, ≤1 nM, ≤0.1 nM, ≤0.01 nM, or ≤0.001 nM (e.g., 10⁻⁸ M or less, e.g., from 10⁻⁸ M to 10⁻¹³ M, e.g., from 10⁻⁹ M to 10⁻¹³ M). An antigen-binding domain is said to “specifically bind” to human PD-L1 when the antigen-binding domain has a K_{D} of 1 μM or less. In certain aspects, an anti-huPD-L1 antigen-binding domain binds to an epitope of PD-L1 that is conserved among huPD-L1 from different species. As used herein, the term “monospecific” antigen-binding domain as used herein means an antigen-binding domain that binds only one antigen or one epitope.

The terms “an anti-PD-L1/anti-IFN-α2a antigen-binding domain”, “an anti-huPD-L1/anti-huIFN-α2a antigen-binding domain”, and “an antigen-binding domain capable of binding to human PD-L1 and to human IFN-α2a” refer to an antigen-binding domain that is capable of binding human PD-L1 and to human IFN-α2a with sufficient affinity such that the antigen-binding domain is useful as a diagnostic and/or therapeutic agent in targeting huPD-L1 and/or huIFN-α2a. “anti-human-PD-L1/anti-human-IFN-α2” is for case of reading sometimes also referred to simply as “anti-huPD-L1/anti-huIFN-α2” herein. In one aspect, the extent of binding of an anti-huPD-L1/anti-huIFN-α2a antigen-binding domain to an unrelated, non-PD-L1 or non-IFN-α2a protein is less than about 10% of the binding of the antigen-binding domain to PD-L1 or to IFN-α2a as measured, e.g., by surface plasmon resonance (SPR). In certain aspects, an anti-huPD-L1/anti-huIFN-α2a antigen-binding domain has a dissociation constant (K_{D}) with regard to PD-L1 binding of ≤1 μM, ≤100 nM, ≤10 nM, ≤1 nM, ≤0.1 nM, ≤0.01 nM, or ≤0.001 nM (e.g., 10⁻⁸ M or less, e.g., from 10⁻⁸ M to 10⁻¹³ M, e.g., from 10⁻⁹ M to 10⁻¹³ M). An antigen-binding domain is said to “specifically bind” to human PD-L1 when the antibody has a K_{D} of 1 μM or less. In certain aspects, an anti-huPD-L1/anti-huIFN-α2a antigen-binding domain binds to an epitope of huPD-L1 that is conserved among PD-L1 from different species. In other aspects, an anti-huPD-L1/anti-huIFN-α2a antigen-binding domain has a dissociation constant (K_{D}) with regard to huIFN-α2a binding of ≤1 μM, ≤100 nM, ≤10 nM, ≤1 nM, ≤ 0.1 nM, ≤0.01 nM, or ≤0.001 nM (e.g., 10⁻⁸ M or less, e.g., from 10⁻⁸ M to 10⁻¹³ M, e.g., from 10⁻⁹ M to 10⁻¹³ M). An antibody is said to “specifically bind” to huIFN-α2 when the antibody has a K_{D} of 1 μM or less. In certain aspects, an anti-huPD-L1/anti-huIFN-α2 antigen-binding domain binds to an epitope of huIFN-α2 that is conserved among IFN-α2 from different species. The anti-PD-L1/anti-IFN-α2a antigen-binding domains described herein bind to PD-L1 and anti-IFN-α2a in a mutually exclusive manner. They are further capable of blocking huIFN-α2 from binding to IFNAR. In one aspect, they are capable of blocking huIFN-α2 from binding to the IFNAR2 subunit of the IFNAR.

The term “antibody” herein is used in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity.

An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)₂; diabodies; linear antibodies; single-chain antibody molecules (e.g., scFv, and scFab); single domain antibodies (dAbs); and multispecific antibodies formed from antibody fragments. For a review of certain antibody fragments, see Holliger and Hudson, Nature Biotechnology 23:1126-1136 (2005).

The term “antigen binding domain” as used herein refers to a sequence of amino acids in an antibody, an antibody fragment or a fusion protein described herein comprising at least one CDR and being of a conformation to recognize a target antigen or epitope.

A “DutaFab” as used herein is a bispecific antibody as disclosed in WO2012/163520. In a DutaFab, a single pair of a VH domain and a VL domain specifically binds to two different epitopes, wherein one paratope comprises amino acid residues from CDR-H2, CDR-L1 and CDR-L3 and the other paratope comprises amino residues from CDR-H1, CDR-H3 and CDR-L2. DutaFabs comprise two non-overlapping paratopes within a cognate VH/VL pair. DutaFabs and methods for their generation by screening of libraries comprising monospecific Fab fragments are disclosed in WO2012/163520. Herein, the term “DutaFab” may particularly refer to a (bispecific) Fab which comprises a single pair of a VH domain and a VL domain and which specifically binds to two different epitopes, wherein one paratope comprises amino acid residues from CDR-H2, CDR-L1 and CDR-L3 and the other paratope comprises amino residues from CDR-H1, CDR-H3 and CDR-L2. Such DutaFabs may be comprised in the fusion proteins described herein.

Herein, a “bispecific Fab” or a “DutaFab” that is capable of binding to antigen 1 and to antigen 2 in a mutually exclusive manner may for ease of reading be referred to simply as ‘a Dutaflip’, “a Dutaflip arm”, “a Dutaflip moiety” or a Dutaflip domain”. Herein, a “bispecific Fab” or a “DutaFab” that is capable of binding to human PD-L1 and to human IFN-α2 in a mutually exclusive manner may for conciseness be referred to simply as an “anti-huPD-L1/anti-huIFN-α2 Dutaflip”, an “anti-huPD-L1/anti-huIFN-α2 Dutaflip arm”, an “anti-huPD-L1/anti-huIFN-α2 Dutaflip moiety” or an “anti-huPD-L1/anti-huIFN-α2Dutaflip domain”. DutaFabs have been known in the art for being engineered therapeutic Fab fragments that may bind two targets simultaneously (Beckmann R et al. Nat Commun. 2021 Jan. 29; 12 (1): 708. doi: 10.1038/s41467-021-20949-3). The inventors have surprisingly found that DutaFabs could also be engineered to allow for mutually exclusive binding of two target epitopes. This can be made use of for the fusion proteins described herein wherein a human IFN-α2 is bound to a bispecific anti-huPD-L1/anti-huIFN-α2 Fab (DutaFab) and is thereby blocked from binding to its receptor IFNAR1/2 when the number of PD-L1 molecules in the environment/on the surface of the target cell is low. In the presence of a high number of PD-L1 molecules, the anti-huPD-L1/anti-huIFN-α2 Dutaflip will start to bind to PD-L1 and the equilibrium is strongly shifted from masked huIFN-α2 to unmasked, active huIFN-α2, resulting in the release of the human IFN-α2 from the bispecific Fab (DutaFab). The anti-huPD-L1/anti-huIFN-α2 Dutaflip is thus acting as a molecular switch in the fusion proteins provided herein, releasing the huIFN-α2 moiety in the presence of sufficient amounts of target antigen, PD-L1.

The term “blocking” as used herein refers to a reduction or elimination of signaling by a protein, particularly in the presence of a fusion protein or antibody described herein. The binding of a blocking antibody/antigen-binding domain directly interferes with the protein's function in a signaling pathway, such as blocking the binding between a receptor and its ligand. When a protein is blocked, it means that its signaling is reduced or eliminated because it has been bound by a blocking antibody or antigen-binding domain. For instance, when used in the context of blocking huPD-L1 binding to huPD1, blocking means that the huPD1/huPD-L1 signaling level in the presence of the fusion protein or the anti-huPD-L1 antibody described herein is lower than the level of huPD1/huPD-L1 signaling in the absence of the fusion protein or antibody), and the magnitude of the decrease is greater than or equal to 5%, 10%, 20%, 25%, 30%, 40%, 50%, 60%, 70%, 75%, 80%, 90%, 95%, 99% or 100%. When used in the context of blocking huIFN-α2 signaling, blocking of huIFN-α2 signaling means that the huIFN-α2 signaling level in the presence of the fusion proteins described herein is lower than the control level (i.e. the level of signaling in the absence of fusion protein), and the magnitude of the decrease is greater than or equal to 5%, 10%, 20%, 25%, 30%, 40%, 50%, 60%, 70%, 75%, 80%, 90%, 95%, 99% or 100%. Both huPD1/huPD-L1 and huIFN-α2 signaling levels can be measured using a variety of standard techniques, such as cell-based luciferase reporter assays. Those skilled in the art will appreciate that a variety of assays can be used to measure signaling levels, including, for example, commercially available kits.

The term “epitope” denotes the site on an antigen, either proteinaceous or non-proteinaceous, to which an antibody, e.g. an anti-PD-L1 antibody, an anti-IFN-α2 antibody, or an antigen-binding domain comprised in a fusion protein described herein, binds. Epitopes can be formed both from contiguous amino acid stretches (linear epitope) and comprise non-contiguous amino acids (conformational epitope), e.g., coming in spatial proximity due to the folding of the antigen, i.e. by the tertiary folding of a proteinaceous antigen. Linear epitopes are typically still bound by an antibody or antigen-binding domain after exposure of the proteinaceous antigen to denaturing agents, whereas conformational epitopes are typically destroyed upon treatment with denaturing agents. An epitope comprises at least 3, at least 4, at least 5, at least 6, at least 7, or 8-10 amino acids in a unique spatial conformation.

Screening for antibodies binding to a particular epitope (i.e., those binding to the same epitope) can be done using methods routine in the art such as, e.g., without limitation, alanine scanning, peptide blots (see Meth. Mol. Biol. 248 (2004) 443-463), peptide cleavage analysis, epitope excision, epitope extraction, chemical modification of antigens (see Prot. Sci. 9 (2000) 487-496), and cross-blocking (see “Antibodies”, Harlow and Lane (Cold Spring Harbor Press, Cold Spring Harb., NY).

Antigen Structure-based Antibody Profiling (ASAP), also known as Modification-Assisted Profiling (MAP), allows to bin a multitude of monoclonal antibodies specifically binding to PD-L1 or IFN-α2 based on the binding profile of each of the antibodies from the multitude to chemically or enzymatically modified antigen surfaces (see, e.g., US 2004/0101920). The antibodies in each bin bind to the same epitope which may be a unique epitope either distinctly different from or partially overlapping with epitope represented by another bin.

Also, competitive binding can be used to easily determine whether an antibody or antigen-binding domain binds to the same epitope of PD-L1 or IFN-α2 as, or competes for binding with, a reference anti-PD-L1 or anti-IFN-α2 antibody or antigen-binding domain, respectively. For example, an “antibody or antigen-binding domain that binds to the same epitope” as a reference anti-PD-L1 or anti-IFN-α2 antibody or antigen-binding domain refers to an antibody or antigen-binding domain that blocks binding of the reference anti-PD-L1 or anti-IFN-α2 antibody or antigen-binding domain to its antigen in a competition assay by 50% or more, and conversely, the reference antibody or antigen-binding domain blocks binding of the antibody or antigen-binding domain to its antigen in a competition assay by 50% or more. Also for example, to determine if an antibody binds to the same epitope as a reference anti-PD-L1 or anti-IFN-α2 antibody or antigen-binding domain, the reference antibody or antigen-binding domain is allowed to bind to PD-L1 or anti-IFN-α2, respectively, under saturating conditions. After removal of the excess of the reference anti-PD-L1 or anti-IFN-α2 antibody or antigen-binding domain, the ability of an anti-PD-L1 or anti-IFN-α2 antibody or antigen-binding domain in question to bind to PD-L1 or IFN-α2, respectively, is assessed. If the anti-PD-L1 or anti-IFN-α2 antibody or antigen-binding domain is able to bind to PD-L1 or IFN-α2, respectively, after saturation binding of the reference anti-PD-L1 or anti-IFN-α2 antibody, it can be concluded that the anti-PD-L1 or anti-IFN-α2 antibody or antigen-binding domain in question binds to a different epitope than the reference anti-PD-L1 or anti-IFN-α2 antibody or antigen-binding domain. But, if the anti-PD-L1 or anti-IFN-α2 antibody or antigen-binding domain in question is not able to bind to PD-L1 or IFN-α2, respectively, after saturation binding of the reference anti-PD-L1 or anti-IFN-α2 antibody or antigen-binding domain, then the anti-PD-L1 or anti-IFN-α2 antibody or antigen-binding domain in question may bind to the same epitope as the epitope bound by the reference anti-PD-L1 or anti-IFN-α2 antibody or antigen-binding domain. To confirm whether the antibody in question binds to the same epitope or is just hampered from binding by steric reasons routine experimentation can be used (e.g., peptide mutation and binding analyses using ELISA, RIA, surface plasmon resonance, flow cytometry or any other quantitative or qualitative antibody-binding assay available in the art). This assay should be carried out in two set-ups, i.e. with both of the antibodies being the saturating antibody. If, in both set-ups, only the first (saturating) antibody or antigen-binding domain is capable of binding to PD-L1 or IFN-α2, respectively, then it can be concluded that the anti-PD-L1 or anti-IFN-α2 antibody or antigen-binding domain in question and the reference anti-PD-L1 or anti-IFN-α2 antibody or antigen-binding domain compete for binding to PD-L1 or IFN-α2, respectively.

In some aspects, two antibodies or antigen-binding domains are deemed to bind to the same or an overlapping epitope if a 1-, 5-, 10-, 20- or 100-fold excess of one antibody inhibits binding of the other by at least 50%, at least 75%, at least 90% or even 99% or more as measured in a competitive binding assay (see, e.g., Junghans et al., Cancer Res. 50 (1990) 1495-1502).

In some aspects, two antibodies or antigen-binding domains are deemed to bind to the same epitope if essentially all amino acid mutations in the antigen that reduce or eliminate binding of one antibody also reduce or eliminate binding of the other. Two antibodies are deemed to have “overlapping epitopes” if only a subset of the amino acid mutations that reduce or eliminate binding of one antibody reduce or eliminate binding of the other.

The term “chimeric” antibody refers to an antibody in which a portion of the heavy and/or light chain is derived from a particular source or species, while the remainder of the heavy and/or light chain is derived from a different source or species.

The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g., IgG₁, IgG₂, IgG₃, IgG₄, IgA₁, and IgA₂. In certain aspects, the antibody is of the IgG₁ isotype. In certain aspects, the antibody is of the IgG₁ isotype with the P329G, L234A and L235A mutation to reduce Fc-region effector function. In other aspects, the antibody is of the IgG₂ isotype. In certain aspects, the antibody is of the IgG₄ isotype with the S228P mutation in the hinge region to improve stability of IgG₄ antibody. The heavy chain constant domains that correspond to the different classes of immunoglobulins are called α, δ, ε, γ, and μ, respectively. The light chain of an antibody may be assigned to one of two types, called kappa (κ) and lambda (λ), based on the amino acid sequence of its constant domain.

The terms “constant region derived from human origin” or “human constant region” as used in the current application denotes a constant heavy chain region of a human antibody of the subclass IgG₁, IgG₂, IgG₃, or IgG₄ and/or a constant light chain kappa or lambda region. Such constant regions are well known in the state of the art and e.g. described by Kabat, E. A., et al., Sequences of Proteins of Immunological Interest, 5th ed., Public Health Service, National Institutes of Health, Bethesda, MD (1991) (see also e.g. Johnson, G., and Wu, T. T., Nucleic Acids Res. 28 (2000) 214-218; Kabat, E. A., et al., Proc. Natl. Acad. Sci. USA 72 (1975) 2785-2788). Unless otherwise specified herein, numbering of amino acid residues in the constant region is according to the EU numbering system, also called the EU index of Kabat, as described in Kabat, E. A. et al., Sequences of Proteins of Immunological Interest, 5th ed., Public Health Service, National Institutes of Health, Bethesda, MD (1991), NIH Publication 91-3242.

“Effector functions” refer to those biological activities attributable to the Fc region of an antibody, which vary with the antibody isotype. Examples of antibody effector functions include C1q binding and complement dependent cytotoxicity (CDC); Fc receptor binding; antibody-dependent cell-mediated cytotoxicity (ADCC); phagocytosis; down regulation of cell surface receptors (e.g., B cell receptor); and B cell activation.

An “effective amount” of an agent, e.g., a pharmaceutical composition, refers to an amount effective, at dosages and for periods of time necessary, to achieve the desired therapeutic or prophylactic result.

A “Fab molecule” refers to a protein consisting of the VH and CH1 domain of the heavy chain (the “Fab heavy chain”) and the VL and CL domain of the light chain (the “Fab light chain”) of an immunoglobulin.

The term “crossover” Fab molecule (also termed “CrossFab”) as used herein refers a Fab molecule wherein the variable domains or the constant domains of the Fab heavy and light chain are exchanged (i.e. replaced by each other), i.e. the crossover Fab molecule comprises a peptide chain composed of the light chain variable domain VL and the heavy chain constant domain 1 CH1 (VL-CH1, in N-to C-terminal direction), and a peptide chain composed of the heavy chain variable domain VH and the light chain constant domain CL (VH-CL, in N-to C-terminal direction). For clarity, in a crossover Fab molecule wherein the variable domains of the Fab light chain and the Fab heavy chain are exchanged, the peptide chain comprising the heavy chain constant domain 1 CH1 is referred to herein as the “heavy chain” of the (crossover) Fab molecule. Conversely, in a crossover Fab molecule wherein the constant domains of the Fab light chain and the Fab heavy chain are exchanged, the peptide chain comprising the heavy chain variable domain VH is referred to herein as the “heavy chain” of the (crossover) Fab molecule.

In contrast thereto, by a “conventional” Fab molecule is meant a Fab molecule in its natural format, i.e. comprising a heavy chain composed of the heavy chain variable and constant domains (VH-CH1, in N-to C-terminal direction), and a light chain composed of the light chain variable and constant domains (VL-CL, in N-to C-terminal direction).

The terms “Fc region” and “Fc domain” herein are used interchangeably and are used to define a C-terminal region of an immunoglobulin heavy chain that contains at least a portion of the constant region. The term includes native sequence Fc regions and variant Fc regions. In one aspect, a human IgG heavy chain Fc region extends from Cys226, or from Pro230, to the carboxyl-terminus of the heavy chain. However, antibodies or fusion proteins produced by host cells may undergo post-translational cleavage of one or more, particularly one or two, amino acids from the C-terminus of the heavy chain. Therefore, an antibody or fusion protein produced by a host cell by expression of a specific nucleic acid molecule encoding a full-length heavy chain may include the full-length heavy chain, or it may include a cleaved variant of the full-length heavy chain. This may be the case where the final two C-terminal amino acids of the heavy chain are glycine (G446) and lysine (K447, EU numbering system). Therefore, the C-terminal lysine (Lys447), or the C-terminal glycine (Gly446) and lysine (Lys447), of the Fc region may or may not be present. Amino acid sequences of heavy chains including an Fc region are denoted herein without C-terminal glycine-lysine dipeptide if not indicated otherwise. In one aspect, a heavy chain including an Fc region as specified herein, comprised in an antibody or fusion protein described herein, comprises an additional C-terminal glycine-lysine dipeptide (G446 and K447, EU numbering system). In one aspect, a heavy chain including an Fc region as specified herein, comprised in an antibody or fusion protein described herein, comprises an additional C-terminal glycine residue (G446, numbering according to EU index). Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, MD, 1991. A “subunit” of an Fc domain as used herein refers to one of the two polypeptides forming the dimeric Fc domain, i.e. a polypeptide comprising C-terminal constant regions of an immunoglobulin heavy chain, capable of stable self-association. For example, a subunit of an IgG Fc domain comprises an IgG CH2 and an IgG CH3 constant domain.

“Framework” or “FR” refers to variable domain residues other than complementary determining regions (CDRs). The FR of a variable domain generally consists of four FR domains: FR1, FR2, FR3, and FR4. Accordingly, the CDR and FR sequences generally appear in the following sequence in VH (or VL): FR1-CDR-H1(CDR-L1)-FR2-CDR-H2(CDR-L2)-FR3-CDR-H3(CDR-L3)-FR4.

The terms “full length antibody”, “intact antibody”, and “whole antibody” are used herein interchangeably to refer to an antibody having a structure substantially similar to a native antibody structure or having heavy chains that contain an Fc region as defined herein.

The term “fusion protein” as used herein refers to a hybrid polypeptide which comprises entire proteins (e.g. human IFN-α2, IgG-type antibodies) or protein domains (e.g. a Fab, a DutaFab or an Fc region) from at least two different proteins that are fused to each other. One protein may be located at the amino-terminal (N-terminal) portion of the fusion protein or at the carboxy-terminal (C-terminal) protein, thus forming an “N-terminal fusion (protein)” or a “C-terminal fusion (protein),” respectively. The term “fusion protein” as used herein encompasses also fusion proteins which are composed of more than one polypeptide chain. In such a fusion protein composed of more than one polypeptide chains, one or more polypeptide chain may have originated from fusing two or more individual polypeptide chains to each other. In some aspects, the polypeptide chains in such a protein may be covalently linked by disulfide bonds. The term “fusion protein” encompasses for instance proteins obtained by fusing a cytokine to an antibody. It also encompasses IgGs or IgG-like molecules (comprising two heavy chains and two light chains) wherein a non-antibody protein is fused to only one polypeptide chain, or to several polypeptide chains, of the IgG or IgG-like molecule. The term “fusion protein” herein also encompasses molecules wherein different (naturally occurring or engineered) antigen binding domains (e.g. Fabs, CrossFabs, DutaFabs), antibody domains (e.g. Fc domains) and non-antibody proteins (including cytokines, such as human IFN-α2) are fused to each other, including molecules wherein different antigen-binding domains are fused to an Fc domain, resulting in a molecule resembling an IgG in structure. The term “fusion protein” as used herein particularly encompasses fusion proteins wherein
a) a (crossover) Fab is fused at the C-terminus of its heavy chain (directly or via a peptidic linker) to the N-terminus of one of the two subunits of the Fc domain;
b) a DutaFab is fused at the C-terminus of its heavy chain (directly or via a peptidic linker) to the N-terminus of the other of the two subunits of the Fc domain; and
c) a cytokine such as human IFN-α2 is fused at its C-terminus directly or via a peptidic linker to the N-terminus of the light chain of the second antigen-binding domain.

By “fused” is meant that the components (e.g. a Fab molecule and an Fc domain subunit) are linked by peptide bonds, either directly or via one or more peptidic linkers. Proteins may be readily fused using conventional procedures or produced by recombinant methods or obtained by chemical synthesis (e.g., by introducing direct peptide bonds or by using peptide linkers). Any of the fusion proteins provided herein may be produced by any method known in the art. For example, the fusion proteins provided herein may be produced via recombinant protein expression and purification, which is especially suited for fusion proteins comprising a peptide linker, but can also be used for fusion proteins wherein domains fused to each other directly. By merging the coding sequences that encode for separate protein domains (and optionally peptide linkers) on the DNA level into a single coding sequence, a mRNA transcript can be produced which can then be translated into a single polypeptide containing all the desired protein domains (and linkers). Methods for recombinant protein expression and purification are well known, and include those described by Green and Sambrook, Molecular Cloning: A Laboratory Manual (4th ed., Cold Spring Harbor Laboratory Press, Cold Spring Harbor, N.Y. (2012)).

Glutamine or glutamate residues at the N-terminus of antibody heavy or light chains may be converted to pyro-glutamate spontaneously (see e.g. Liu et al., Journal of Pharmaceutical Sciences 97, 2426-2447 (2008), Rehder et al., Journal of Chromatography A 1102, 164-175 (2006), Chelius et al., Anal Chem 78, 2370-2376 (2006)). Hence, variable domains disclosed herein which comprise either a glutamine (Q) or a glutamate (E) amino acid residue at the N-terminus of an the antibody heavy or light chain, may comprise an N-terminal pyro-glutamate (pyroE) residue instead of the N-terminal Q or E residue. Likewise, antibody heavy chains or light chains disclosed herein which comprise either a glutamine (Q) or a glutamate (E) amino acid residue at the N-terminus, may comprise an N-terminal pyro-glutamate (pyroE) residue instead of the N-terminal Q or E residue. Accordingly, for each antibody heavy chain, light chain, or variable domain sequence disclosed herein that contains an N-terminal Q or E residue, the corresponding sequence with an N-terminal pyroE residue is also encompassed.

The terms “host cell”, “host cell line”, and “host cell culture” are used interchangeably and refer to cells into which exogenous nucleic acid has been introduced, including the progeny of such cells. Host cells include “transformants” and “transformed cells”, which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein.

A “human antibody” is one which possesses an amino acid sequence which corresponds to that of an antibody produced by a human or a human cell or derived from a non-human source that utilizes human antibody repertoires or other human antibody-encoding sequences. This definition of a human antibody specifically excludes a humanized antibody comprising non-human antigen-binding residues.

A “human consensus framework” is a framework which represents the most commonly occurring amino acid residues in a selection of human immunoglobulin VL or VH framework sequences. Generally, the selection of human immunoglobulin VL or VH sequences is from a subgroup of variable domain sequences. Generally, the subgroup of sequences is a subgroup as in Kabat et al., Sequences of Proteins of Immunological Interest, Fifth Edition, NIH Publication 91-3242, Bethesda MD (1991), vols. 1-3. In one aspect, for the VL, the subgroup is subgroup kappa I as in Kabat et al., supra. In one aspect, for the VH, the subgroup is subgroup III as in Kabat et al., supra.

A “humanized” antibody refers to a chimeric antibody comprising amino acid residues from non-human CDRs and amino acid residues from human FRs. In certain aspects, a humanized antibody will comprise substantially all of at least one, and typically two, variable domains, in which all or substantially all of the CDRs correspond to those of a non-human antibody, and all or substantially all of the FRs correspond to those of a human antibody. A humanized antibody optionally may comprise at least a portion of an antibody constant region derived from a human antibody. A “humanized form” of an antibody, e.g., a non-human antibody, refers to an antibody that has undergone humanization.

The term “hypervariable region” or “HVR” as used herein refers to each of the regions of an antibody variable domain which are hypervariable in sequence and which determine antigen binding specificity, for example “complementarity determining regions” (“CDRs”).

Generally, antibodies or antigen-binding domains comprise six CDRs: three in the VH (CDR-H1, CDR-H2, CDR-H3), and three in the VL (CDR-L1, CDR-L2, CDR-L3). Exemplary CDRs herein include:
(a) hypervariable loops occurring at amino acid residues 26-32 (L1), 50-52 (L2), 91-96 (L3), 26-32 (H1), 53-55 (H2), and 96-101 (H3) (Chothia and Lesk, J. Mol. Biol. 196:901-917 (1987));
(b) CDRs occurring at amino acid residues 24-34 (L1), 50-56 (L2), 89-97 (L3), 31-35b (H1), 50-65 (H2), and 95-102 (H3) (Kabat et al., Sequences of Proteins of Immunological Interest, 5th Ed. Public Health Service, National Institutes of Health, Bethesda, MD (1991)); and
(c) antigen contacts occurring at amino acid residues 27c-36 (L1), 46-55 (L2), 89-96 (L3), 30-35b (H1), 47-58 (H2), and 93-101 (H3) (MacCallum et al. J. Mol. Biol. 262:732-745 (1996)).

Unless otherwise indicated, the CDRs are determined according to Kabat et al., supra. One of skill in the art will understand that the CDR designations can also be determined according to Chothia, supra, McCallum, supra, or any other scientifically accepted nomenclature system.

An “immunoconjugate” is an antibody conjugated to one or more heterologous molecule(s), including but not limited to a cytotoxic agent.

An “individual” or “subject” is a mammal. Mammals include, but are not limited to, domesticated animals (e.g., cows, sheep, cats, dogs, and horses), primates (e.g., humans and non-human primates such as monkeys), rabbits, and rodents (e.g., mice and rats). In certain aspects, the individual or subject is a human.

The term “Interferon alpha”, “Interferon α”, “IFN alpha” or “IFNα”, as used herein, refers to any native Interferon alpha from any vertebrate source, including mammals such as primates (e.g., humans) and rodents (e.g., mice and rats), unless otherwise indicated. The term encompasses “full-length”, unprocessed Interferon alpha as well as any form of Interferon alpha that results from processing in the cell, unless otherwise indicated. The term also encompasses naturally occurring variants of Interferon alpha, e.g., splice variants or allelic variants. Interferons (IFNs) are a family of proteins that were originally named for their ability to interfere with viral replication and propagation. To date, it is known that interferons are also involved in combating bacterial and parasitic infections, inhibit cell division, and promote or impede the differentiation of cells. The interferons are classified based on their receptor specificity into three types of interferons: type I, type II and type III. The interferons of type I are monomeric proteins and include IFN alpha, IFN beta and IFN omega that are products of leukocytes and fibroblasts, IFN kappa that is expressed by human keratinocytes, IFN epsilon that is exclusively expressed in lung, brain, small intestine and the reproductive tissue and IFN tau that has been described only in ruminants. The term “type I interferon” as used herein is intended to refer to members of the type I interferon family of molecules that are ligands for IFNAR-I (i.e., members of the type I interferon family of molecules that are capable of binding IFNAR-I). Examples of type I interferon ligands are interferon alpha 1, 2a, 2b, 4, 5, 6, 7, 8, 10, 14, 16, 17, 21, interferon beta and interferon omega.

The interferon alpha family is composed of 13 intron-less fully translated genes (excluding pseudogenes). Each member includes mature proteins of 165 or 166 amino acid residues, with two conserved disulfide bonds: Cys1-Cys98 and Cys29-Cys138. A high level of sequence homology (70-99%) is displayed among the various interferon alpha subtypes, and about 35% homology exists between these subtypes and IFN beta. Despite the high homology, a shared 3D core structure and a shared receptor of the different subtypes, their biological activities, among them anti-proliferative, antiviral, and immunomodulation, differ notably.

Of the known IFN alpha subtypes, only interferon alpha 2 (IFN-α2) has been extensively studied for its pharmaceutical potential. IFN-α2 is known to have anti-cancer effects. It is mainly used in second line adjunct therapy of hematopoietic cancers. However, this treatment is not always effective and sometimes results in intolerable side effects related to the dosage and duration of therapy. Three alleles exist: Interferon alpha-2A, Interferon alpha-2B and Interferon alpha-2C. In nature, allele alpha-2B is the predominant allele while allele alpha-2A is less predominant and alpha-2C only a minor allelic variant.

The terms “human IFNα” or “human IFNα-2” may for ease of reading also be referred to as “huIFNα” or “huIFNα-2” herein. The terms “human Interferon-alpha 2”, “human Interferon-α 2”, “human IFN-alpha 2”, or “human IFN-α2”, as used herein, relate to mature human interferon alpha 2 either of the human IFN-α2a allele, having the amino acid sequence set forth in SEQ ID NO:79, or of the human IFN-α2b allele, having the amino acid sequence set forth in SEQ ID NO:80. They relate preferably to human IFN-α2 of the human IFN-α2a allele having the amino acid sequence set forth in SEQ ID NO:79. They may also relate to the full-length precursors of the respective human IFN-α2 alleles, i.e. IFN-α2a or IFN-α2b comprising an N-terminal signal peptide. In one aspect, the mature human IFN-α2 has the sequence of the human IFN-α2a precursor of SEQ ID NO:81. In one aspect, the human IFN-α2 has the sequence of the human IFN-α2b precursor of SEQ ID NO: 82. The terms may further relate to functional variants of these proteins.

Such functional variants may be homologues of human IFN-α2 that have typically at least one amino acid exchange that does not significantly impair functionality of the protein, i.e. binding to and/or activating of the human IFNAR1/2 receptor. The sequence identity for such variants is thus typically higher than 95%, often more than 98%. Functional variants of human IFN-α2 may also comprise one or more amino acid exchanges that prevent glycosylation of the human IFN-α2 as disclosed in WO2016/065409A1. It is understood that the polypeptides described herein may also comprise additional amino acid sequences on the N- or C-terminus, such as a signal peptide, which is also present in naturally occurring human IFN-α2 prior to post-translational processing. Other elements that may be present include various tags or markers that facilitate expression, purification and/or detection, as well as protease recognition sites that allow cleavage of such additional sequence elements.

The terms “human IFNα” or “human IFNα-2” may further encompass mutated variants of human IFNα-2 which have amino acid substitutions which reduce their affinity for the IFNAR1 and IFNAR2 receptor complex (IFNAR) and reduced or abolished ability to activate IFNAR expressing cells as isolated molecules but retain the ability to bind IFNAR and the ability to bind and activate the IFNAR receptor complex, particularly when fused to a targeting moiety. These variants with reduced affinity for IFNAR may also be referred to as “attenuated huIFNα-2” herein. In some aspects, the attenuated huIFNα-2 has a biological activity selected from less than 70% less than 60% less than 50% less than 40% less than 30% less than 20% or less than 10% of the biological activity of the wild-type huIFNα-2 of which it is deduced (i.e., the wild-type huIFNα-2 of which the coding sequence has been mutated to obtain the mutant IFN). Attenuated huIFNα-2 variants confer reduced biological activity, and thus reduced off-target activity and off-target toxicity, to the fusion proteins described herein. The targeting of the mutated huIFNα-2 variants achieved by the first and second antigen-binding domains restores the activity of the mutated ligand with the degree of activity restoration apparently correlated with the level of targeting biologic on the cells. On the other hand, introducing mutations into the native huIFNα-2 polypeptide sequence may increase immunogenicity. Thus, in one aspect of the fusion proteins herein, the huIFNα-2 comprised in the fusion protein is native huIFNα-2. In one particular aspect, the huIFNα-2 comprised in the fusion protein described herein is huIFNα-2 of SEQ ID NO: 79.

In an embodiment, the human IFN-α2 binds to and/or activates the IFN-α/β receptor (IFNAR), i.e., IFNAR1 and/or IFNAR2. The terms “IFNAR” and “IFNAR 1/2” are used interchangeably herein and refer to the interferon-α/β receptor, a membrane receptor which binds endogenous type I interferons and consists of the two subunits IFNAR1 and IFNAR2.

An “isolated” antibody is one which has been separated from a component of its natural environment. In some aspects, an antibody is purified to greater than 95% or 99% purity as determined by, for example, electrophoretic (e.g., SDS-PAGE, isoelectric focusing (IEF), capillary electrophoresis) or chromatographic (e.g., ion exchange or reverse phase HPLC) methods. For a review of methods for assessment of antibody purity, see, e.g., Flatman et al., J. Chromatogr. B 848:79-87 (2007).

The term “nucleic acid molecule” or “polynucleotide” includes any compound and/or substance that comprises a polymer of nucleotides. Each nucleotide is composed of a base, specifically a purine- or pyrimidine base (i.e. cytosine (C), guanine (G), adenine (A), thymine (T) or uracil (U)), a sugar (i.e. deoxyribose or ribose), and a phosphate group. Often, the nucleic acid molecule is described by the sequence of bases, whereby said bases represent the primary structure (linear structure) of a nucleic acid molecule. The sequence of bases is typically represented from 5′ to 3′. Herein, the term nucleic acid molecule encompasses deoxyribonucleic acid (DNA) including e.g., complementary DNA (cDNA) and genomic DNA, ribonucleic acid (RNA), in particular messenger RNA (mRNA), synthetic forms of DNA or RNA, and mixed polymers comprising two or more of these molecules. The nucleic acid molecule may be linear or circular. In addition, the term nucleic acid molecule includes both, sense and antisense strands, as well as single stranded and double stranded forms. Moreover, the herein described nucleic acid molecule can contain naturally occurring or non-naturally occurring nucleotides. Examples of non-naturally occurring nucleotides include modified nucleotide bases with derivatized sugars or phosphate backbone linkages or chemically modified residues. Nucleic acid molecules also encompass DNA and RNA molecules which are suitable as a vector for direct expression of an antibody of the invention in vitro and/or in vivo, e.g., in a host or patient. Such DNA (e.g., cDNA) or RNA (e.g., mRNA) vectors can be unmodified or modified. For example, mRNA can be chemically modified to enhance the stability of the RNA vector and/or expression of the encoded molecule so that mRNA can be injected into a subject to generate the antibody in vivo (see e.g., Stadler et al, Nature Medicine 2017, published online 12 Jun. 2017, doi: 10.1038/nm.4356 or EP 2 101 823 B1).

An “isolated” nucleic acid refers to a nucleic acid molecule that has been separated from a component of its natural environment. An isolated nucleic acid includes a nucleic acid molecule contained in cells that ordinarily contain the nucleic acid molecule, but the nucleic acid molecule is present extrachromosomally or at a chromosomal location that is different from its natural chromosomal location.

“Isolated nucleic acid encoding a fusion protein” refers to one or more nucleic acid molecules encoding the polypeptides that make up the fusion protein (or fragments thereof), including such nucleic acid molecule(s) in a single vector or separate vectors, and such nucleic acid molecule(s) present at one or more locations in a host cell. “Isolated nucleic acid encoding an anti-PD-L1 antibody” refers to one or more nucleic acid molecules encoding anti-PD-L1 antibody heavy and light chains (or fragments thereof), including such nucleic acid molecule(s) in a single vector or separate vectors, and such nucleic acid molecule(s) present at one or more locations in a host cell.

The term “monoclonal antibody” as used herein refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method. For example, the monoclonal antibodies in accordance with the present invention may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein.

A “naked antibody” refers to an antibody that is not conjugated to a heterologous moiety (e.g., a cytotoxic moiety) or radiolabel. The naked antibody may be present in a pharmaceutical composition.

“Native antibodies” refer to naturally occurring immunoglobulin molecules with varying structures. For example, native IgG antibodies are heterotetrameric glycoproteins of about 150,000 Daltons, composed of two identical light chains and two identical heavy chains that are disulfide-bonded. From N- to C-terminus, each heavy chain has a variable domain (VH), also called a variable heavy domain or a heavy chain variable region, followed by three constant heavy domains (CH1, CH2, and CH3). Similarly, from N- to C-terminus, each light chain has a variable domain (VL), also called a variable light domain or a light chain variable region, followed by a constant light (CL) domain.

The term “package insert” is used to refer to instructions customarily included in commercial packages of therapeutic products, that contain information about the indications, usage, dosage, administration, combination therapy, contraindications and/or warnings concerning the use of such therapeutic products.

The terms “peptide linker” or “peptidic linker” are used herein to refer to a peptide comprising one or more amino acids, typically about 2 to 20 amino acids. Peptide linkers are known in the art or are described herein. Suitable, non-immunogenic peptide linkers are, for example, (G₃S)ₙ (SEQ ID NO:97) or (G₄S)ₙ (SEQ ID NO:101) peptide linkers, wherein “n” is generally a number between 1 and 10, typically between 2 and 4, in particular 2. Peptide linkers of particular interest are (GSGGS)ₙ (SEQ ID NO:96), (GGGS)ₙ (SEQ ID NO:97), (GSGGG)ₙ (SEQ ID NO:98), (GGGSG)ₙ (SEQ ID NO:99), (GSSSG)ₙ (SEQ ID NO: 100), (GGGGS)ₙ (SEQ ID NO:101) and (GGSGG)ₙ (SEQ ID NO: 102), where n represents an integer of at least 1, preferably from 4 to 6.

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity for the purposes of the alignment. Alignment for purposes of determining percent amino acid sequence identity can be achieved in various ways that are within the skill in the art, for instance, using publicly available computer software such as BLAST, BLAST-2, Clustal W, Megalign (DNASTAR) software or the FASTA program package. Those skilled in the art can determine appropriate parameters for aligning sequences, including any algorithms needed to achieve maximal alignment over the full length of the sequences being compared. Alternatively, the percent identity values can be generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087 and is described in WO 2001/007611.

Unless otherwise indicated, for purposes herein, percent amino acid sequence identity values are generated using the ggsearch program of the FASTA package version 36.3.8c or later with a BLOSUM50 comparison matrix. The FASTA program package was authored by W. R. Pearson and D. J. Lipman (1988), “Improved Tools for Biological Sequence Analysis”, PNAS 85:2444-2448; W. R. Pearson (1996) “Effective protein sequence comparison” Meth. Enzymol. 266:227-258; and Pearson et. al. (1997) Genomics 46:24-36 and is publicly available from www.fasta.bioch.virginia.edu/fasta_www2/fasta_down.shtml or www. ebi.ac.uk/Tools/sss/fasta. Alternatively, a public server accessible at fasta.bioch.virginia.edu/fasta_www2/index.cgi can be used to compare the sequences, using the ggsearch (global protein: protein) program and default options (BLOSUM50; open: −10; ext: −2; Ktup=2) to ensure a global, rather than local, alignment is performed. Percent amino acid identity is given in the output alignment header.

The term “pharmaceutical composition” or “pharmaceutical formulation” refers to a preparation which is in such form as to permit the biological activity of an active ingredient contained therein to be effective, and which contains no additional components which are unacceptably toxic to a subject to which the pharmaceutical composition would be administered.

A “pharmaceutically acceptable carrier” refers to an ingredient in a pharmaceutical composition or formulation, other than an active ingredient, which is nontoxic to a subject. A pharmaceutically acceptable carrier includes, but is not limited to, a buffer, excipient, stabilizer, or preservative.

The term “PD-L1”, as used herein, refers to any native PD-L1 from any vertebrate source, including mammals such as primates (e.g., humans) and rodents (e.g., mice and rats), unless otherwise indicated. The term encompasses “full-length”, unprocessed PD-L1 as well as any form of PD-L1 that results from processing in the cell. The term also encompasses naturally occurring variants of PD-L1, e.g., splice variants or allelic variants. The amino acid sequence of an exemplary human PD-L1 is shown in SEQ ID NO:83.

The terms “target cell” and “target tissue” herein refer to a specific type of cell or tissue, respectively, which is the intended recipient of the fusion proteins, antibodies or treatments described herein. In particular, they may be a cell or tissue that exhibits a particular receptor or characteristic that makes it susceptible or responsive to the intended effect of the fusion protein, antibody or treatment. In the context of the fusion proteins and antibodies described herein, the receptor present on the target cell or in the target tissue may particularly be PD-L1.

As used herein, “treatment” (and grammatical variations thereof such as “treat” or “treating”) refers to clinical intervention in an attempt to alter the natural course of a disease in the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In some aspects, antibodies of the invention are used to delay development of a disease or to slow the progression of a disease.

The term “variable region” or “variable domain” refers to the domain of an antibody heavy or light chain that is involved in binding the antibody to antigen. The variable domains of the heavy chain and light chain (VH and VL, respectively) of a native antibody generally have similar structures, with each domain comprising four conserved framework regions (FRs) and three complementary determining regions (CDRs). (See, e.g., Kindt et al. Kuby Immunology, 6^{th} ed., W.H. Freeman and Co., page 91 (2007).) A single VH or VL domain may be sufficient to confer antigen-binding specificity. Furthermore, antibodies that bind a particular antigen may be isolated using a VH or VL domain from an antibody that binds the antigen to screen a library of complementary VL or VH domains, respectively. See, e.g., Portolano et al., J. Immunol. 150:880-887 (1993); Clarkson et al., Nature 352:624-628 (1991).

The term “vector”, as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors”.

### II. Compositions and Methods

In one aspect, the invention is based, in part, on the finding that the PD-L1-targeted IFN-α2 fusion proteins described herein that exert IFN-α2 activity once the fusion protein is bound to the target antigen PD-L1 surprisingly show improved anti-tumor activity, in particular with regard to their ability to inhibit tumor cell proliferation and tumor growth. They further show improved anti-tumor activity with regard to directly or indirectly inducing proliferation and/or activation of immune cells. They also show an improved side effect profile and better tolerability, in particular compared to recombinant IFN-α2a or other untargeted and/or unattenuated IFN-α2 variants. In certain aspects, fusion proteins comprising a human IFN-α2, a first antigen-binding domain capable of binding to human PD-L1 and a second antigen-binding domain capable of binding to human PD-L1 and human IFN-α2 are provided. Fusion proteins of the invention are useful, e.g., for the treatment of cancer.

The invention is further based, in part, on the finding that the anti-PD-L1 antibodies provided herein—in contrast to other PD1/PD-L1 blocking antibodies which bind to the N-terminus of PD-L1—are surprisingly capable of blocking the interaction of PD-L1 and PD1 in an SPR binding assay even though they bind to the C-terminal domain of PD-L1. This property has the technical effect that these antibodies do not compete with other therapeutic anti-PD-L1 antibodies that block PD-L1/PD1 interaction (e.g. Atezolizumab, BMS-936559, Avelumab, Durvalumab) for PD-L1 binding because these bind to the N-terminal domain of PD-L1. As a consequence, the fusion proteins and antibodies according to the invention can be used in combination (either concomitantly or sequentially) with these other anti-PD-L1 binding antibodies to treat PD-L1 mediated diseases. Antibodies of the invention are thus useful, e.g., for the diagnosis or treatment of cancer.

### A. Exemplary Molecules of the Invention

### 1. Exemplary Fusion Proteins

In one aspect, the invention provides PD-L1-targeted IFN-α2 fusion proteins comprising a human IFN-α2, a first antigen-binding domain capable of binding to human PD-L1 and a second antigen-binding domain capable of binding to human PD-L1 and to human IFN-α2. In one aspect, provided are isolated PD-L1-targeted IFN-α2 fusion proteins comprising a human IFN-α2, a first antigen-binding domain capable of binding to human PD-L1 and a second antigen-binding domain capable of binding to human PD-L1 and to human IFN-α2. In one aspect, the invention provides fusion proteins comprising a human IFN-α2, a first antigen-binding domain capable of binding to human PD-L1 and a second antigen-binding domain capable of binding to human PD-L1 and to human IFN-α2, wherein the second antigen-binding domain is a bispecific Fab. In one aspect, the invention provides fusion proteins comprising a human IFN-α2, a first antigen-binding domain capable of binding to human PD-L1 and a second antigen-binding domain capable of binding to human PD-L1 and to human IFN-α2, wherein the human IFN-α2 is further fused at its C-terminus to the N-terminus of the heavy chain or of the light chain of the second antigen-binding domain. In one aspect, the human IFN-α2 is fused at its C-terminus to the N-terminus of the light chain of the second antigen-binding domain.

In certain aspects of the fusion proteins comprising a human IFN-α2, a first antigen-binding domain capable of binding to human PD-L1 and a second antigen-binding domain capable of binding to human PD-L1 and to human IFN-α2, the second antigen-binding domain binds human PD-L1 and human IFN-α2 in a mutually exclusive manner, that is, it binds to only one of its two target antigens at the same time, but not to both at once. In certain aspects of the fusion proteins comprising a human IFN-α2, a first antigen-binding domain capable of binding to human PD-L1 and a second antigen-binding domain capable of binding to human PD-L1 and to human IFN-α2, the second antigen-binding domain capable of binding to human PD-L1 and to human IFN-α2 is no longer able to bind to human IFN-α2, when it is bound to human PD-L1. In certain aspects, in the fusion proteins comprising a human IFN-α2, a first antigen-binding domain capable of binding to human PD-L1 and a second antigen-binding domain capable of binding to human PD-L1 and to human IFN-α2, the second antigen-binding domain capable of binding to human PD-L1 and to human IFN-α2 is no longer able to bind human PD-L1, when it is bound to human IFN-α2.

In certain aspects of the fusion proteins described herein, the second antigen-binding domain is blocked from binding PD-L1 when it is bound to human IFN-α2 and is blocked from binding human IFN-α2 when it has bound to PD-L1. In a particular aspect, the affinity of the second antigen-binding domain for human PD-L1 is higher than its affinity for human IFN-α2. In certain aspects, the second antigen-binding domain will bind preferably to human PD-L1, so that the human IFN-α2 moiety will no longer bind to the second antigen-binding domain in the presence of the target antigen, i.e. human PD-L1. In some aspects, the activity of the human IFN-α2 is reduced and/or blocked when it is bound to the second antigen-binding domain. In some aspects, the human IFN-α2 is inactive when it is bound to the second antigen-binding domain. In certain aspects, the second antigen-binding domain blocks the activity of the human IFN-α2 when bound to the human IFN-α2. In other aspects, the human IFN-α2 is active when the second antigen-binding domain is bound to huPD-L1 on the target cell. In one aspect, the second antigen-binding domain has affinity for human IFN-α2 and human PD-L1, such that the marker and the therapeutic domain compete for binding to the second antigen-binding domain. In one aspect, the equilibrium between the second antigen-binding domain being bound to human IFN-α2 and the second antigen-binding domain being bound to human PD-L1 will be shifted strongly towards bound human PD-L1 and towards an unbound and active human IFN-α moiety in the presence of PD-L1 molecules, in particular in the presence of a high number of PD-L1 molecules, particularly on the surface of a target cell.

In certain aspects, in a fusion protein comprising a human IFN-α2, a first antigen-binding domain capable of binding to human PD-L1 and a second antigen-binding domain capable of binding to human PD-L1 and to human IFN-α2, the human IFN-α2 moiety is bound to the second antigen-binding domain, in particular a bispecific anti-PD-L1/anti-IFN-α2 Fab, and thereby blocked from binding to its receptor IFNAR1/2 when the number of PD-L1 molecules in the environment or on the surface of the target cell is low (e.g. below a defined threshold) and the human IFN-α2 is released from the second antigen-binding domain when the number of PD-L1 molecules on the surface of the target cell is high (e.g. above a defined threshold). In one aspect, in a fusion protein comprising a human IFN-α2, a first antigen-binding domain capable of binding to human PD-L1 and a second antigen-binding domain capable of binding to human PD-L1 and to human IFN-α2, the human IFN-α2 is bound to the second antigen-binding domain (e.g. a bispecific anti-PD-L1/anti-IFN-α2 Fab). The human IFN-α2 is thereby blocked from binding to its receptor IFNAR1/2 in the presence of cells that express little or no PD-L1 molecules in their surface, in particular in the proximity of non-tumor cells. The huIFN-α2 or variant thereof is released from the second antigen-binding domain and binds to its receptor IFNAR1/2 in the presence of PD-L1 high expressing cells like tumor cells or immune cells in the tumor microenvironment.

The fusion proteins provided herein may also be provided in an asymmetric form with a domain crossover in one or more antigen-binding domains of the same antigen specificity, i.e. by exchanging the VH/VL domains (see e.g., WO 2009/080252 and WO 2015/150447), the CH1/CL domains (see e.g., WO 2009/080253) or the complete Fab arms (see e.g., WO 2009/080251, WO 2016/016299, also see Schaefer et al, PNAS, 108 (2011) 1187-1191, and Klein at al., MAbs 8 (2016) 1010-20). In one aspect, the fusion protein as described herein comprises a CrossFab. The term “CrossFab” or “xFab” or “crossover Fab” refers to a Fab fragment, wherein either the variable regions or the constant regions of the heavy and light chain are exchanged. A crossover Fab comprises a polypeptide chain composed of the light chain variable region (VL) and the heavy chain constant region 1 (CH1), and a polypeptide chain composed of the heavy chain variable region (VH) and the light chain constant region (CL). Asymmetrical Fab arms can also be engineered by introducing charged or non-charged amino acid mutations into domain interfaces to direct correct Fab 30 pairing. See e.g., WO 2016/172485. This way of protein engineering advantageously promotes the correct assembly of the fusion proteins described herein when being produced in a host cell.

For ease of reading, the fusion proteins provided herein are sometimes also referred to herein as “PD-L1-targeted IFN-α2a fusion protein”, “PD-L1-targeted IFN-α2a Dutaflip fusion protein”, “huPD-L1-targeted human IFN-α2a fusion protein” or “PD-L1-targeted human IFN-α2a fusion protein” or variations thereof.

### Interferon Alpha 2a

A fusion protein of the invention comprises a human IFN-α2, that is, the amino acid sequence of the human IFN-α2 is fused to another domain of the fusion protein cither directly (e.g. via a peptide bond) or via a peptidic linker. In one aspect, the human IFN-α2 has the sequence of the mature full length human IFN-α2, that is, it lacks an N-terminal signal peptide. In one aspect, the human IFN-α2 has the sequence of mature human IFN-α2 of the human IFN-α2a allele (SEQ ID NO:79). In one aspect, the human IFN-α2 has the sequence of mature human IFN-α2 of the human IFN-α2b allele (SEQ ID NO:80). In one aspect, the human IFN-α2 has the sequence of the precursor human IFN-α2, that is, it comprises an N-terminal signal peptide. In one aspect, the precursor human IFN-α2 has the sequence of the human IFN-α2a allele (SEQ ID NO:81). In one aspect, the human IFN-α2 has the sequence of the human IFN-α2b allele (SEQ ID NO:82). In one aspect, the human IFN-α2 is fused at its C-terminus to the N-terminus of the heavy chain or the light chain of the second antigen-binding domain capable of binding to human PD-L1 and to human IFN-α2, either directly or via a peptidic liker. In one aspect, the human IFN-α2 is fused at its C-terminus to the N-terminus of the light chain of the second antigen-binding domain capable of binding to human PD-L1 and to human IFN-α2.

### First Antigen Binding Domain Capable of Binding to Human PD-L1

A fusion protein of the invention comprises a first antigen-binding domain, in particular a Fab, that is capable of binding to human PD-L1. In one aspect of the fusion protein provided herein, the first antigen-binding domain is monospecific for human PD-L1, i.e. it is capable of binding solely to an antigen or an epitope on human PD-L1. In certain aspects, the first antigen-binding domain is not capable of binding to any antigen or epitope other than an antigen or epitope on human PD-L1. In one aspect, the first antigen-binding domain is capable of specifically binding to human PD-L1. In particular embodiments, the first antigen-binding domain which is capable of binding to human PD-L1 is a crossover Fab molecule, i.e. a Fab molecule wherein the variable domains VH and VL or the constant domains CH1 and CL of the Fab heavy and light chains are exchanged/replaced by each other. In such embodiments, the second antigen-binding domain that is capable of binding to human PD-L1 and to human IFN-α2 has a conventional Fab domain structure (i.e. without domain crossover) as described herein.

In alternative embodiments, the first antigen-binding domain which is capable of binding to human PD-L1 is a conventional Fab molecule (i.e. without domain crossover). In such embodiments, the second antigen-binding domain that is capable of binding to human PD-L1 and to human IFN-α2 is a crossover Fab molecule as described herein, i.e. a Fab molecule wherein the variable domains VH and VL or the constant domains CH1 and CL of the Fab heavy and light chains are exchanged/replaced by each other.

The first antigen-binding domain which is capable of binding to human PD-L1 is able to direct the fusion protein to a target site, for example to a specific type of tumor cell or immune cell in the tumor microenvironment that expresses human PD-L1. Such a cell expressing PD-L1 is herein sometimes also referred to as “target cell”.

The first antigen-binding domain of the fusion protein may incorporate any of the features, singly or in combination, described herein in relation to the fusion protein and the antigen-binding domains contained therein, unless scientifically clearly unreasonable or impossible.

Thus, in one aspect, the invention provides a fusion protein comprising a human IFN-α2, a first antigen-binding domain capable of binding to human PD-L1 and a second antigen-binding domain capable of binding to human PD-L1 and to human IFN-α2, wherein the second antigen-binding domain is a bispecific Fab, particularly a DutaFab, and the human IFN-α2 is fused at its C-terminus to the N-terminus of the heavy chain or the light chain of the second antigen-binding domain, wherein the

the first antigen-binding domain capable of binding to human PD-L1 comprises
(i) (a) CDR-H1 comprising the amino acid sequence of SEQ ID NO:21; (b) CDR-H2 comprising the amino acid sequence of SEQ ID NO:22; and (c) CDR-H3 comprising the amino acid sequence of SEQ ID NO:23, or
(ii) (a) CDR-H1 comprising the amino acid sequence of SEQ ID NO:29; (b) CDR-H2 comprising the amino acid sequence of SEQ ID NO:30; and (c) CDR-H3 comprising the amino acid sequence of SEQ ID NO:31.

In another aspect, the invention provides a fusion protein comprising a human IFN-α2, a first antigen-binding domain capable of binding to human PD-L1 and a second antigen-binding domain capable of binding to human PD-L1 and to human IFN-α2, wherein the first antigen-binding domain capable of binding to human PD-L1 comprises
(i) (a) CDR-L1 comprising the amino acid sequence of SEQ ID NO:24; (b) CDR-L2 comprising the amino acid sequence of SEQ ID NO:25; and (c) CDR-L3 comprising the amino acid sequence of SEQ ID NO:26, or
(ii) (a) CDR-L1 comprising the amino acid sequence of SEQ ID NO:32; (b) CDR-L2 comprising the amino acid sequence of SEQ ID NO:33; and (c) CDR-L3 comprising the amino acid sequence of SEQ ID NO:34.

In certain aspects, the first antigen-binding domain of the fusion protein described herein comprises
(i) (a) CDR-H1 comprising the amino acid sequence of SEQ ID NO:21; (b) CDR-H2 comprising the amino acid sequence of SEQ ID NO:22; (c) CDR-H3 comprising the amino acid sequence of SEQ ID NO:23, (d) CDR-L1 comprising the amino acid sequence of SEQ ID NO:24; (e) CDR-L2 comprising the amino acid sequence of SEQ ID NO:25; and (f) CDR-L3 comprising the amino acid sequence of SEQ ID NO:26; or
(ii) (a) CDR-H1 comprising the amino acid sequence of SEQ ID NO:29; (b) CDR-H2 comprising the amino acid sequence of SEQ ID NO:30; (c) CDR-H3 comprising the amino acid sequence of SEQ ID NO:31, (d) CDR-L1 comprising the amino acid sequence of SEQ ID NO:32; (e) CDR-L2 comprising the amino acid sequence of SEQ ID NO:33; and (f) CDR-L3 comprising the amino acid sequence of SEQ ID NO:34.

In one aspect, a fusion protein of the invention comprises a first antigen binding domain comprising (a) a VH domain comprising at least one, at least two, or all three VH CDR sequences selected from (i) CDR-H1 comprising the amino acid sequence of SEQ ID NO: 21, (ii) CDR-H2 comprising the amino acid sequence of SEQ ID NO:22, and (iii) CDR-H3 comprising the amino acid sequence of SEQ ID NO:23; and (b) a VL domain comprising at least one, at least two, or all three VL CDR sequences selected from (i) CDR-L1 comprising the amino acid sequence of SEQ ID NO:24, (ii) CDR-L2 comprising the amino acid sequence of SEQ ID NO:25, and (iii) CDR-L3 comprising the amino acid sequence of SEQ ID NO:26.

In another aspect, a fusion protein of the invention comprises a first antigen binding domain comprising (a) a VH domain comprising at least one, at least two, or all three VH CDR sequences selected from (i) CDR-H1 comprising the amino acid sequence of SEQ ID NO: 29, (ii) CDR-H2 comprising the amino acid sequence of SEQ ID NO:30, and (iii) CDR-H3 comprising the amino acid sequence of SEQ ID NO:31; and (b) a VL domain comprising at least one, at least two, or all three VL CDR sequences selected from (i) CDR-L1 comprising the amino acid sequence of SEQ ID NO:32, (ii) CDR-L2 comprising the amino acid sequence of SEQ ID NO:33, and (iii) CDR-L3 comprising the amino acid sequence of SEQ ID NO:34.

In one aspect, the invention provides a fusion protein comprising a first antigen-binding domain comprising (a) CDR-H1 comprising the amino acid sequence of SEQ ID NO:21; (b) CDR-H2 comprising the amino acid sequence of SEQ ID NO:22; (c) CDR-H3 comprising the amino acid sequence of SEQ ID NO:23; (d) CDR-L1 comprising the amino acid sequence of SEQ ID NO:24; (c) CDR-L2 comprising the amino acid sequence of SEQ ID NO: 25; and (f) CDR-L3 comprising the amino acid sequence of SEQ ID NO:26.

In another aspect, the invention provides a fusion protein comprising a first antigen-binding domain comprising (a) CDR-H1 comprising the amino acid sequence of SEQ ID NO:29; (b) CDR-H2 comprising the amino acid sequence of SEQ ID NO:30; (c) CDR-H3 comprising the amino acid sequence of SEQ ID NO:31; (d) CDR-L1 comprising the amino acid sequence of SEQ ID NO:32; (e) CDR-L2 comprising the amino acid sequence of SEQ ID NO: 33; and (f) CDR-L3 comprising the amino acid sequence of SEQ ID NO:34.

In any of the aspects provided herein, a fusion protein is humanized, i.e. the domains of the fusion protein that are derived from immunoglobulins are humanized. In one aspect, the first antigen-binding domain of a fusion protein further comprises an acceptor human framework, e.g. a human immunoglobulin framework or a human consensus framework.

In one aspect, the first antigen-binding domain of a fusion protein described herein comprises one or more of the CDR sequences of the VH of SEQ ID NO:27 or of the VH of SEQ ID NO:35. In another aspect, the first antigen-binding domain of a fusion protein as described herein comprises one or more of the CDR sequences of the VL of SEQ ID NO: 28 or of the VL of SEQ ID NO:36. In certain aspects, the first antigen-binding domain of a fusion protein described herein comprises (i) the CDR sequences of the VH of SEQ ID NO: 27 and the CDR sequences of the VL of SEQ ID NO:28 or (ii) the CDR sequences of the VH of SEQ ID NO:35 and the CDR sequences of the VL of SEQ ID NO:36.

In a further aspect, a fusion protein as described herein comprises a first antigen-binding domain comprising
i. the CDR-H1, CDR-H2 and CDR-H3 amino acid sequences of the VH domain of SEQ ID NO:27 and the CDR-L1, CDR-L2 and CDR-L3 amino acid sequences of the VL domain of SEQ ID NO:28, or
ii. the CDR-H1, CDR-H2 and CDR-H3 amino acid sequences of the VH domain of SEQ ID NO:35 and the CDR-L1, CDR-L2 and CDR-L3 amino acid sequences of the VL domain of SEQ ID NO:36.

In one aspect, the first antigen-binding domain of a fusion protein as described herein comprises (a) CDR-H1 comprising the amino acid sequence of SEQ ID NO:21; (b) CDR-H2 comprising the amino acid sequence of SEQ ID NO:22; (c) CDR-H3 comprising the amino acid sequence of SEQ ID NO:23; (d) CDR-L1 comprising the amino acid sequence of SEQ ID NO:24; (e) CDR-L2 comprising the amino acid sequence of SEQ ID NO:25; and (f) CDR-L3 comprising the amino acid sequence of SEQ ID NO:26, and a VH domain having at least 90%, 91%, 92%, 93%, 94%, 95%, 96%, 97%, 98%, 99%, or 100% sequence identity to the amino acid sequence of SEQ ID NO:27, and a VL domain having at least 90%, 91%, 92%, 93%, 94%, 95%, 96%, 97%, 98%, 99%, or 100% sequence identity to the amino acid sequence of SEQ ID NO:28. In one aspect, the VH domain has at least 95% sequence identity to the amino acid sequence of SEQ ID NO:27. In one aspect, the VL domain has at least 95% sequence identity to the amino acid sequence of SEQ ID NO:28.

In one aspect, the first antigen-binding domain of a fusion protein as described herein comprises (a) CDR-H1 comprising the amino acid sequence of SEQ ID NO:29; (b) CDR-H2 comprising the amino acid sequence of SEQ ID NO:30; (c) CDR-H3 comprising the amino acid sequence of SEQ ID NO:31; (d) CDR-L1 comprising the amino acid sequence of SEQ ID NO:32; (e) CDR-L2 comprising the amino acid sequence of SEQ ID NO:33; and (f) CDR-L3 comprising the amino acid sequence of SEQ ID NO:34, and a VH domain having at least 90%, 91%, 92%, 93%, 94%, 95%, 96%, 97%, 98%, 99%, or 100% sequence identity to the amino acid sequence of SEQ ID NO:35, and a VL domain having at least 90%, 91%, 92%, 93%, 94%, 95%, 96%, 97%, 98%, 99%, or 100% sequence identity to the amino acid sequence of SEQ ID NO:36. In one aspect, the VH domain has at least 95% sequence identity to the amino acid sequence of SEQ ID NO:35. In one aspect, the VL domain has at least 95% sequence identity to the amino acid sequence of SEQ ID NO:36.

In another aspect, the first antigen-binding domain of a fusion protein as described herein comprises a heavy chain variable domain (VH) sequence having at least 90%, 91%, 92%, 93%, 94%, 95%, 96%, 97%, 98%, 99%, or 100% sequence identity to the amino acid sequence of SEQ ID NO:27 or to the amino acid sequence of SEQ ID NO:35. In one aspect, the first antigen-binding domain of a fusion protein comprises a heavy chain variable domain (VH) sequence having at least 95%, sequence identity to the amino acid sequence of SEQ ID NO:27 or to the amino acid sequence of SEQ ID NO:35. In certain aspects, a VH sequence having at least 90%, 91%, 92%, 93%, 94%, 95%, 96%, 97%, 98%, or 99% identity contains substitutions (e.g., conservative substitutions), insertions, or deletions relative to the reference sequence, but an anti-huPD-L1 antigen-binding domain comprising that sequence retains the ability to bind to human PD-L1. In certain aspects, a total of 1 to 10 amino acids have been substituted, inserted and/or deleted in SEQ ID NO:27 or SEQ ID NO:35. In certain aspects, substitutions, insertions, or deletions occur in regions outside the CDRs (i.e., in the FRs). Optionally, the first antigen-binding domain of a fusion protein as described herein comprises the VH sequence in SEQ ID NO:27 or SEQ ID NO: 35, including post-translational modifications of that sequence. In a particular aspect, the VH comprises (i) (a) CDR-H1, comprising the amino acid sequence of SEQ ID NO:21, (b) CDR-H2, comprising the amino acid sequence of SEQ ID NO:22, and (c) CDR-H3, comprising the amino acid sequence of SEQ ID NO:23 or (ii) (a) CDR-H1, comprising the amino acid sequence of SEQ ID NO:29, (b) CDR-H2, comprising the amino acid sequence of SEQ ID NO:30, and (c) CDR-H3, comprising the amino acid sequence of SEQ ID NO: 31.

In another aspect, a fusion protein as described herein comprising a human IFN-α2, a first antigen-binding domain capable of binding to human PD-L1 and a second antigen-binding domain capable of binding to human PD-L1 and to human IFN-α2 is provided, wherein the first antigen-binding domain comprises a light chain variable domain (VL) sequence having at least 90%, 91%, 92%, 93%, 94%, 95%, 96%, 97%, 98%, 99%, or 100% sequence identity to the amino acid sequence of SEQ ID NO:28 or to the amino acid sequence of SEQ ID NO:36. In one aspect, the first antigen-binding domain of the fusion protein comprises a light chain variable domain (VL) sequence having at least 95% sequence identity to the amino acid sequence of SEQ ID NO:28 or to the amino acid sequence of SEQ ID NO:36. In certain aspects, a VL sequence having at least 90%, 91%, 92%, 93%, 94%, 95%, 96%, 97%, 98%, or 99% identity contains substitutions (e.g., conservative substitutions), insertions, or deletions relative to the reference sequence, but an anti-PD-L1 antigen-binding domain comprising that sequence retains the ability to bind to PD-L1. In certain aspects, a total of 1 to 10 amino acids have been substituted, inserted and/or deleted in SEQ ID NO:28 or SEQ ID NO:36. In certain aspects, the substitutions, insertions, or deletions occur in regions outside the CDRs (i.e., in the FRs). Optionally, the first antigen-binding domain of a fusion protein comprises the VL sequence in SEQ ID NO: 28 or SEQ ID NO:36, including post-translational modifications of that sequence. In a particular aspect, the VL comprises (i) (a) CDR-L1, comprising the amino acid sequence of SEQ ID NO:24, (b) CDR-L2, comprising the amino acid sequence of SEQ ID NO:25, and (c) CDR-L3, comprising the amino acid sequence of SEQ ID NO:26 or (ii) (a) CDR-L1, comprising the amino acid sequence of SEQ ID NO:32, (b) CDR-L2, comprising the amino acid sequence of SEQ ID NO:33, and (c) CDR-L3, comprising the amino acid sequence of SEQ ID NO:34.

In certain aspects, a fusion protein comprising a human IFN-α2, a first antigen-binding domain capable of binding to human PD-L1 and a second antigen-binding domain capable of binding to human PD-L1 and to human IFN-α2 is provided, wherein the first antigen-binding domain comprises a VH sequence as in any of the aspects provided above, and a VL sequence as in any of the aspects provided above. In one particular aspect, the first antigen-binding domain comprises the VH and VL sequences in SEQ ID NO:27 and SEQ ID NO: 28, respectively, including post-translational modifications of those sequences. In another aspect, the first antigen-binding domain comprises the VH and VL sequences in SEQ ID NO:35 and SEQ ID NO:36, respectively, including post-translational modifications of those sequences.

### Second Antigen Binding Domain Capable of Binding to Human PD-L1 and to Human IFN-α2

The fusion protein of the invention comprises a second antigen-binding domain, particularly a bispecific Fab, that is capable of binding to human PD-L1 and to human IFN-α2. In one aspect, the second antigen-binding domain is capable of specifically binding to human PD-L1 and to human IFN-α2. In one aspect, the second antigen-binding domain is capable of binding to human IFN-α2 so that human IFNα2 is blocked from binding its receptor IFNAR. In one aspect, the second antigen-binding domain is capable of binding to human IFN-α2 so that human IFN-α2 is blocked from binding its receptor IFNAR and from activating the IFNα signaling pathway. In one aspect, the second antigen-binding domain binds to human PD-L1 and to human IFN-α2 in a mutually exclusive manner, that is, it binds only to one of its two target antigens at the same time, but not to both at once. In other words, while the second antigen-binding domain capable of binding to human PD-L1 and to human IFN-α2 has bound to human PD-L1, it is not able to bind to human IFN-α2, and while it has bound to human IFN-α2, it is not able to bind human PD-L1. In one aspect of the invention, the second antigen-binding domain capable of binding to human PD-L1 and to human IFN-α2 binds to human IFN-α2, thus preventing IFN-α2 from binding to its receptor. In some embodiments, the second antigen-binding domain releases the human IFN-α2 in the presence of human PD-L1, binding instead preferably to human PD-L1 and thus enabling the human IFN-α2 in the fusion protein to bind to its natural receptor.

In certain aspects, the second antigen-binding domain capable of binding to human PD-L1 and to human IFN-α2 is a DutaFab. In one aspect, the second antigen-binding domain capable of binding to human PD-L1 and to human IFN-α2 comprises a human IFN-α2 paratope and a human PD-L1 paratope within one cognate pair of a variable light chain domain (VL domain) and a variable heavy chain domain (VH domain), wherein (a) the human IFN-α2 paratope comprises amino acid residues from CDR-H2, CDR-L1 and CDR-L3 of the antigen-binding domain, and wherein the human PD-L1 paratope comprises amino acid residues from the CDR-H1, CDR-H3 and CDR-L2 of the antigen-binding domain, or (b) the human PD-L1 paratope comprises amino acid residues from CDR-H2, CDR-L1 and CDR-L3 of the antigen-binding domain, and wherein the human IFN-α2 paratope comprises amino acid residues from the CDR-H1, CDR-H3 and CDR-L2 of the antigen-binding domain. In a particular aspect, the second antigen-binding domain capable of binding to human PD-L1 and to human IFN-α2 comprises a human IFN-α2 paratope and a PD-L1 paratope within one cognate pair of a variable light chain domain (VL domain) and a variable heavy chain domain (VH domain), wherein the human IFN-α2 paratope comprises amino acid residues from CDR-H2, CDR-L1 and CDR-L3 of the antigen-binding domain, and wherein the PD-L1 paratope comprises amino acid residues from the CDR-H1, CDR-H3 and CDR-L2 of the antigen-binding domain.

In certain aspects, the second antigen-binding domain is blocked from binding human PD-L1 when it is bound to the human IFN-α2 and is blocked from binding it when it is bound to human PD-L1. In a particular aspect, the affinity of the second antigen-binding domain for human PD-L1 is higher than its affinity for human IFN-α2. In certain aspects, the second antigen-binding domain will bind preferably to human PD-L1, so that the human IFN-α2 moiety bound to the second antigen-binding domain will be released in the presence of the target antigen human PD-L1. In some aspects, the human IFN-α2 is blocked from binding to IFNAR when it is bound to the bispecific Fab and is able to bind to IFNAR when the bispecific Fab is bound to PD-L1. In certain aspects, the second antigen-binding domain capable of binding to human PD-L1 and to human IFN-α2 is bound to the IFN-α2 within the fusion protein in the absence of PD-L1 in the vicinity. In certain aspects, second antigen-binding domain capable of binding to human PD-L1 and to human IFN-α2 releases the IFN-α2 of the fusion protein in the presence human PD-L1 on the target cell/in the target tissue. In certain aspects, the human IFN-α2 of the fusion proteins described herein is bound to the second antigen-binding domain capable of binding to human PD-L1 and to human IFN-α2 in the absence of human PD-L1 in the vicinity. In certain aspects, the human IFN-α2 of the fusion proteins described herein is released from the second antigen-binding domain capable of binding to human PD-L1 and to human IFN-α2 and/or is activated in the presence human PD-L1 on the target cell/in the target tissue.

In certain aspects, in the fusion proteins comprising a human IFN-α2, a first antigen-binding domain capable of binding to human PD-L1 and a second antigen-binding domain capable of binding to human PD-L1 and to human IFN-α2, the human IFN-α2 is bound to the bispecific anti-PD-L1/anti-IFN-α2 Fab and blocked thereby from binding to its receptor IFNAR 1/2 when the number of PD-L1 molecules in the environment/on the surface of the target cell is below a defined threshold and human IFN-α2 is released from the bispecific Fab when the number of PD-L1 molecules on the surface of the target cell is above a defined threshold. In one aspect, in the fusion proteins of the invention comprising a human IFN-α2, a first antigen-binding domain capable of binding to human PD-L1 and a second antigen-binding domain capable of binding to human PD-L1 and to human IFN-α2, the human IFN-α2 is bound to the bispecific anti-PD-L1/anti-IFN-α2 Fab and blocked thereby from binding to its receptor IFNAR 1/2 in the proximity of cells that express few or no PD-L1 molecules on their surface, particularly in the proximity of non-tumor cells. When there is a sufficient number of PD-L1 molecules present (e.g. on PD-L1 expressing tumor tissue), the human IFN-α2 is released from the bispecific Fab and binds to its receptor IFNAR1/2 on tumor cells and/or PD-L1 expressing immune cells in the tumor micro environment.

In particular embodiments, the second antigen-binding domain capable of binding to human PD-L1 and to human IFN-α2 has the domain structure of a conventional Fab molecule, i.e. it has the domain structure of a native Fab (i.e. without domain crossover). In particular embodiments, the second antigen-binding domain capable of binding to human PD-L1 and to human IFN-α2 is not a CrossFab. In such embodiments, the first antigen-binding domain capable of binding to human PD-L1 is a crossover Fab molecule as described herein, i.e. a Fab molecule wherein the variable domains VH and VL or the constant domains CH1 and CL of the Fab heavy and light chains are exchanged/replaced by each other.

In alternative embodiments, the second antigen-binding domain capable of binding to human PD-L1 and to human IFN-α2, is a crossover Fab molecule as described herein, i.e. a Fab molecule wherein the variable domains VH and VL or the constant domains CH1 and CL of the Fab heavy and light chains are exchanged/replaced by each other. In such embodiments, the first antigen-binding domain capable of binding to human PD-L1 is preferably a conventional Fab molecule.

In one aspect, the invention provides a fusion protein comprising a human IFN-α2, a first antigen-binding domain capable of binding to human PD-L1 and a second antigen-binding domain capable of binding to human PD-L1 and to human IFN-α2, wherein the second antigen-binding domain comprises
(i) at least one, at least two, at least three, at least four, at least five, or all six CDRs selected from (a) CDR-H1 comprising the amino acid sequence of SEQ ID NO: 1; (b) CDR-H2 comprising the amino acid sequence of SEQ ID NO:2; (c) CDR-H3 comprising the amino acid sequence of SEQ ID NO:3; (d) CDR-L1 comprising the amino acid sequence of SEQ ID NO:4; (e) CDR-L2 comprising the amino acid sequence of SEQ ID NO:5; and (f) CDR-L3 comprising the amino acid sequence of SEQ ID NO:6,
(ii) at least one, at least two, at least three, at least four, at least five, or all six CDRs selected from (a) CDR-H1 comprising the amino acid sequence of SEQ ID NO: 1; (b) CDR-H2 comprising the amino acid sequence of SEQ ID NO:2; (c) CDR-H3 comprising the amino acid sequence of SEQ ID NO:3; (d) CDR-L1 comprising the amino acid sequence of SEQ ID NO:9; (e) CDR-L2 comprising the amino acid sequence of SEQ ID NO:5; and (f) CDR-L3 comprising the amino acid sequence of SEQ ID NO:6, or
(iii) at least one, at least two, at least three, at least four, at least five, or all six CDRs selected from (a) CDR-H1 comprising the amino acid sequence of SEQ ID NO: 1; (b) CDR-H2 comprising the amino acid sequence of SEQ ID NO:2; (c) CDR-H3 comprising the amino acid sequence of SEQ ID NO:3; (d) CDR-L1 comprising the amino acid sequence of SEQ ID NO:4; (e) CDR-L2 comprising the amino acid sequence of SEQ ID NO:11; and (f) CDR-L3 comprising the amino acid sequence of SEQ ID NO:6.

In another aspect, a fusion protein of the invention comprises a second antigen binding domain comprising
(a) a VH domain comprising at least one, at least two, or all three VH CDR sequences selected from (i) CDR-H1 comprising the amino acid sequence of SEQ ID NO:1, (ii) CDR-H2 comprising the amino acid sequence of SEQ ID NO: 2, and (iii) CDR-H3 comprising the amino acid sequence of SEQ ID NO:3; and (b) a VL domain comprising at least one, at least two, or all three VL CDR sequences selected from (i) CDR-L1 comprising the amino acid sequence of SEQ ID NO:4, (ii) CDR-L2 comprising the amino acid sequence of SEQ ID NO: 5, and (c) CDR-L3 comprising the amino acid sequence of SEQ ID NO:6,
(b) a VH domain comprising at least one, at least two, or all three VH CDR sequences selected from (i) CDR-H1 comprising the amino acid sequence of SEQ ID NO:1, (ii) CDR-H2 comprising the amino acid sequence of SEQ ID NO: 2, and (iii) CDR-H3 comprising the amino acid sequence of SEQ ID NO:3; and (b) a VL domain comprising at least one, at least two, or all three VL CDR sequences selected from (i) CDR-L1 comprising the amino acid sequence of SEQ ID NO:9, (ii) CDR-L2 comprising the amino acid sequence of SEQ ID NO: 5, and (c) CDR-L3 comprising the amino acid sequence of SEQ ID NO:6, or
(c) a VH domain comprising at least one, at least two, or all three VH CDR sequences selected from (i) CDR-H1 comprising the amino acid sequence of SEQ ID NO:1, (ii) CDR-H2 comprising the amino acid sequence of SEQ ID NO: 2, and (iii) CDR-H3 comprising the amino acid sequence of SEQ ID NO:3; and (b) a VL domain comprising at least one, at least two, or all three VL CDR sequences selected from (i) CDR-L1 comprising the amino acid sequence of SEQ ID NO:4, (ii) CDR-L2 comprising the amino acid sequence of SEQ ID NO: 11, and (c) CDR-L3 comprising the amino acid sequence of SEQ ID NO:6.

In another aspect, the invention provides a fusion protein comprising a second antigen-binding domain comprising
(a) CDR-H1 comprising the amino acid sequence of SEQ ID NO:1; (b) CDR-H2 comprising the amino acid sequence of SEQ ID NO:2; (c) CDR-H3 comprising the amino acid sequence of SEQ ID NO:3; (d) CDR-L1 comprising the amino acid sequence of SEQ ID NO:4; (e) CDR-L2 comprising the amino acid sequence of SEQ ID NO:5; and (f) CDR-L3 comprising the amino acid sequence of SEQ ID NO:6,
(b) CDR-H1 comprising the amino acid sequence of SEQ ID NO:1; (b) CDR-H2 comprising the amino acid sequence of SEQ ID NO:2; (c) CDR-H3 comprising the amino acid sequence of SEQ ID NO:3; (d) CDR-L1 comprising the amino acid sequence of SEQ ID NO:9; (e) CDR-L2 comprising the amino acid sequence of SEQ ID NO:5; and (f) CDR-L3 comprising the amino acid sequence of SEQ ID NO:6, or
(c) CDR-H1 comprising the amino acid sequence of SEQ ID NO:1; (b) CDR-H2 comprising the amino acid sequence of SEQ ID NO:2; (c) CDR-H3 comprising the amino acid sequence of SEQ ID NO:3; (d) CDR-L1 comprising the amino acid sequence of SEQ ID NO:4; (e) CDR-L2 comprising the amino acid sequence of SEQ ID NO:11; and (f) CDR-L3 comprising the amino acid sequence of SEQ ID NO:6.

In any of the aspects provided herein, a fusion protein provided which is humanized, i.e. the domains of the fusion protein that are derived from immunoglobulins are humanized. In one aspect, the second antigen-binding domain of a fusion protein further comprises an acceptor human framework, e.g. a human immunoglobulin framework or a human consensus framework.

In another aspect, an second antigen-binding domain of a fusion protein as described herein comprises one or more of the CDR sequences of the VH of SEQ ID NO:7. In another embodiment, a second antigen-binding domain comprises one or more of the CDR sequences of the VL of SEQ ID NO:8, SEQ ID NO:10 or SEQ ID NO:12. In another embodiment, a second antigen-binding domain comprises the CDR sequences of the VH of SEQ ID NO:7 and the CDR sequences of the VL of SEQ ID NO:8, SEQ ID NO: 10 or SEQ ID NO:12. In a particular aspect, a second antigen-binding domain comprises the CDR sequences of the VH of SEQ ID NO:7 and the CDR sequences of the VL of SEQ ID NO: 8.

In a further aspect, the second antigen-binding domain of a fusion protein according to the invention comprises the CDR-H1, CDR-H2 and CDR-H3 amino acid sequences of the VH domain of SEQ ID NO:7 and the CDR-L1, CDR-L2 and CDR-L3 amino acid sequences of the VL domain of SEQ ID NO:8, SEQ ID NO: 10 or SEQ ID NO:12. In another aspect, the second antigen-binding domain of a fusion protein as described herein comprises the CDR-H1, CDR-H2 and CDR-H3 amino acid sequences of the VH domain of SEQ ID NO:7 and the CDR-L1, CDR-L2 and CDR-L3 amino acid sequences of the VL domain of SEQ ID NO: 8.

In one aspect, the second antigen-binding domain of a fusion protein comprises one or more of the heavy chain CDR amino acid sequences of the VH domain of SEQ ID NO:7 and a framework of at least 85%, 86%, 87%, 88%, 89%, 90%, 91%, 92%, 93%, 94%, 95%, 96%, 97%, 98%, 99% sequence identity to the framework amino acid sequence of the VH domain of SEQ ID NO:7. In one aspect, the second antigen-binding domain of a fusion protein comprises the three heavy chain CDR amino acid sequences of the VH domain of SEQ ID NO:7 and a framework of at least 85%, 86%, 87%, 88%, 89%, 90%, 91%, 92%, 93%, 94%, 95%, 96%, 97%, 98%, 99% sequence identity to the framework amino acid sequence of the VH domain of SEQ ID NO:7. In one aspect, the second antigen-binding domain of a fusion protein comprises the three heavy chain CDR amino acid sequences of the VH domain of SEQ ID NO:7 and a framework of at least 95% sequence identity to the framework amino acid sequence of the VH domain of SEQ ID NO:7. In another aspect, the second antigen-binding domain of a fusion protein comprises the three heavy chain CDR amino acid sequences of the VH domain of SEQ ID NO:7 and a framework of at least of at least 98% sequence identity to the framework amino acid sequence of the VH domain of SEQ ID NO:7.

In one aspect, the second antigen-binding domain of a fusion protein comprises one or more of the light chain CDR amino acid sequences of the VL domain of SEQ ID NO:8, SEQ ID NO: 10 or SEQ ID NO: 12 and a framework of at least 85%, 86%, 87%, 88%, 89%, 90%, 91%, 92%, 93%, 94%, 95%, 96%, 97%, 98%, 99% sequence identity to the framework amino acid sequence of the VL domain of SEQ ID NO:8, SEQ ID NO: 10 or SEQ ID NO:12. In one aspect, the second antigen-binding domain of a fusion protein comprises the three light chain CDR amino acid sequences of the VL domain of SEQ ID NO: 8, SEQ ID NO:10 or SEQ ID NO: 12 and a framework of at least 85%, 86%, 87%, 88%, 89%, 90%, 91%, 92%, 93%, 94%, 95%, 96%, 97%, 98%, 99% sequence identity to the framework amino acid sequence of the VL domain of SEQ ID NO:8, SEQ ID NO:10 or SEQ ID NO:12. In one aspect, the second antigen-binding domain of a fusion protein comprises the three light chain CDR amino acid sequences of the VL domain of SEQ ID NO: 8, SEQ ID NO:10 or SEQ ID NO:12 and a framework of at least 95% sequence identity to the framework amino acid sequence of the VL domain of SEQ ID NO:8, SEQ ID NO:10 or SEQ ID NO: 12. In another aspect, the second antigen-binding domain of a fusion protein comprises the three light chain CDR amino acid sequences of the VL domain of SEQ ID NO: 8, SEQ ID NO:10 or SEQ ID NO:12 and a framework of at least particularly of at least 98% sequence identity to the framework amino acid sequence of the VH domain of SEQ ID NO: 8, SEQ ID NO: 10 or SEQ ID NO: 12.

In one aspect, the second antigen-binding domain of a fusion protein comprises one or more of the light chain CDR amino acid sequences of the VL domain of SEQ ID NO:8 and a framework of at least 85%, 86%, 87%, 88%, 89%, 90%, 91%, 92%, 93%, 94%, 95%, 96%, 97%, 98%, 99% sequence identity to the framework amino acid sequence of the VL domain of SEQ ID NO:8. In one aspect, the second antigen-binding domain of a fusion protein comprises the three light chain CDR amino acid sequences of the VL domain of SEQ ID NO:8 and a framework of at least 85%, 86%, 87%, 88%, 89%, 90%, 91%, 92%, 93%, 94%, 95%, 96%, 97%, 98%, 99% sequence identity to the framework amino acid sequence of the VL domain of SEQ ID NO:8. In one aspect, the second antigen-binding domain of a fusion protein comprises the three light chain CDR amino acid sequences of the VL domain of SEQ ID NO:8 and a framework of at least 95% sequence identity to the framework amino acid sequence of the VL domain of SEQ ID NO:8. In another aspect, the second antigen-binding domain of a fusion protein comprises the three light chain CDR amino acid sequences of the VL domain of SEQ ID NO:8 and a framework of at least particularly of at least 98% sequence identity to the framework amino acid sequence of the VH domain of SEQ ID NO:8.

In one aspect, the second antigen-binding domain of a fusion protein comprises (a) CDR-H1 comprising the amino acid sequence of SEQ ID NO:1; (b) CDR-H2 comprising the amino acid sequence of SEQ ID NO:2; (c) CDR-H3 comprising the amino acid sequence of SEQ ID NO:3; (d) CDR-L1 comprising the amino acid sequence of SEQ ID NO:4; (e) CDR-L2 comprising the amino acid sequence of SEQ ID NO:5; and (f) CDR-L3 comprising the amino acid sequence of SEQ ID NO:6, and a VH domain having at least 90%, 91%, 92%, 93%, 94%, 95%, 96%, 97%, 98%, 99%, or 100% sequence identity to the amino acid sequence of SEQ ID NO:7, and a VL domain having at least 90%, 91%, 92%, 93%, 94%, 95%, 96%, 97%, 98%, 99%, or 100% sequence identity to the amino acid sequence of SEQ ID NO:8. In one aspect, the VH domain has at least 95% sequence identity to the amino acid sequence of SEQ ID NO:7. In one aspect, the VL domain has at least 95% sequence identity to the amino acid sequence of SEQ ID NO:8.

In one aspect, the second antigen-binding domain of a fusion protein comprises (a) CDR-H1 comprising the amino acid sequence of SEQ ID NO:1; (b) CDR-H2 comprising the amino acid sequence of SEQ ID NO:2; (c) CDR-H3 comprising the amino acid sequence of SEQ ID NO:3; (d) CDR-L1 comprising the amino acid sequence of SEQ ID NO:9; (c) CDR-L2 comprising the amino acid sequence of SEQ ID NO:5; and (f) CDR-L3 comprising the amino acid sequence of SEQ ID NO:6, and a VH domain having at least 90%, 91%, 92%, 93%, 94%, 95%, 96%, 97%, 98%, 99%, or 100% sequence identity to the amino acid sequence of SEQ ID NO:7, and a VL domain having at least 90%, 91%, 92%, 93%, 94%, 95%, 96%, 97%, 98%, 99%, or 100% sequence identity to the amino acid sequence of SEQ ID NO:10. In one aspect, the VH domain has at least 95% sequence identity to the amino acid sequence of SEQ ID NO:7. In one aspect, the VL domain has at least 95% sequence identity to the amino acid sequence of SEQ ID NO:10.

In one aspect, the second antigen-binding domain of a fusion protein comprises (a) CDR-H1 comprising the amino acid sequence of SEQ ID NO:1; (b) CDR-H2 comprising the amino acid sequence of SEQ ID NO:2; (c) CDR-H3 comprising the amino acid sequence of SEQ ID NO:3; (d) CDR-L1 comprising the amino acid sequence of SEQ ID NO:4; (e) CDR-L2 comprising the amino acid sequence of SEQ ID NO:11; and (f) CDR-L3 comprising the amino acid sequence of SEQ ID NO:6, and a VH domain having at least 90%, 91%, 92%, 93%, 94%, 95%, 96%, 97%, 98%, 99%, or 100% sequence identity to the amino acid sequence of SEQ ID NO:7, and a VL domain having at least 90%, 91%, 92%, 93%, 94%, 95%, 96%, 97%, 98%, 99%, or 100% sequence identity to the amino acid sequence of SEQ ID NO:12. In one aspect, the VH domain has at least 95% sequence identity to the amino acid sequence of SEQ ID NO:7. In one aspect, the VL domain has at least 95% sequence identity to the amino acid sequence of SEQ ID NO:12.

In one aspect, the second antigen-binding domain of a fusion protein comprises (a) CDR-H1 comprising the amino acid sequence of SEQ ID NO:1; (b) CDR-H2 comprising the amino acid sequence of SEQ ID NO:2; (c) CDR-H3 comprising the amino acid sequence of SEQ ID NO:3; (d) CDR-L1 comprising the amino acid sequence of SEQ ID NO:4; (c) CDR-L2 comprising the amino acid sequence of SEQ ID NO:5; and (f) CDR-L3 comprising the amino acid sequence of SEQ ID NO:6, and a VH domain having at least 90%, 91%, 92%, 93%, 94%, 95%, 96%, 97%, 98%, 99%, or 100% sequence identity to the amino acid sequence of SEQ ID NO:7, and a VL domain having at least 90%, 91%, 92%, 93%, 94%, 95%, 96%, 97%, 98%, 99%, or 100% sequence identity to the amino acid sequence of SEQ ID NO:8; wherein the second antigen-binding domain of a fusion protein is capable of binding to human PD-L1 and to human IFN-α2. In one aspect, the VH domain has at least 95% sequence identity to the amino acid sequence of SEQ ID NO:7. In one aspect, the VL domain has at least 95% sequence identity to the amino acid sequence of SEQ ID NO:8. In one aspect, the second antigen-binding domain of a fusion protein capable of binding to human PD-L1 and to human IFN-α2 has a dissociation constant (K_{D})) that is up to 10 fold reduced or up to 10 fold increased when compared to the dissociation constant (K_{D}) of an antigen-binding domain comprising a VH sequence of SEQ ID NO:7 and a VL sequence of SEQ ID NO:8.

In another aspect, the second antigen-binding domain of a fusion protein as described herein comprises a heavy chain variable domain (VH) sequence having at least 90%, 91%, 92%, 93%, 94%, 95%, 96%, 97%, 98%, 99%, or 100% sequence identity to the amino acid sequence of SEQ ID NO:7. In one aspect, the second antigen-binding domain of a fusion protein comprises a heavy chain variable domain (VH) sequence having at least 95%, sequence identity to the amino acid sequence of SEQ ID NO:7. In certain aspects, a VH sequence having at least 90%, 91%, 92%, 93%, 94%, 95%, 96%, 97%, 98%, or 99% identity contains substitutions (e.g., conservative substitutions), insertions, or deletions relative to the reference sequence, but an anti-huPD-L1/huIFN-α2 antigen-binding domain comprising that sequence retains the ability to bind to human PD-L1 and to human IFN-α2. In certain aspects, a total of 1 to 10 amino acids have been substituted, inserted and/or deleted in SEQ ID NO:7. In certain aspects, substitutions, insertions, or deletions occur in regions outside the CDRs (i.e., in the FRs). Optionally, the second antigen-binding domain of a fusion protein comprises the VH sequence in SEQ ID NO:7, including post-translational modifications of that sequence. In a particular aspect, the VH comprises one, two or three CDRs selected from: (a) CDR-H1, comprising the amino acid sequence of SEQ ID NO:1, (b) CDR-H2, comprising the amino acid sequence of SEQ ID NO:2, and (c) CDR-H3, comprising the amino acid sequence of SEQ ID NO:3. In another aspect, a fusion protein comprising a human IFN-α2, a first antigen-binding domain capable of binding to human PD-L1 and a second antigen-binding domain capable of binding to human PD-L1 and to human IFN-α2 is provided, wherein the second antigen-binding domain comprises a light chain variable domain (VL) sequence having at least 90%, 91%, 92%, 93%, 94%, 95%, 96%, 97%, 98%, 99%, or 100% sequence identity to the amino acid sequence of SEQ ID NO:8. In one aspect, the second antigen-binding domain of the fusion protein comprises a light chain variable domain (VL) sequence having at least 95% sequence identity to the amino acid sequence of SEQ ID NO:8. In certain aspects, a VL sequence having at least 90%, 91%, 92%, 93%, 94%, 95%, 96%, 97%, 98%, or 99% identity contains substitutions (e.g., conservative substitutions), insertions, or deletions relative to the reference sequence, but an anti-huPD-L1/huIFN-α2 antigen-binding domain comprising that sequence retains the ability to bind to human PD-L1 and to human IFN-α2. In certain aspects, a total of 1 to 10 amino acids have been substituted, inserted and/or deleted in SEQ ID NO:8. In certain aspects, the substitutions, insertions, or deletions occur in regions outside the CDRs (i.e., in the FRs). Optionally, the second antigen-binding domain of a fusion protein comprises the VL sequence in SEQ ID NO:8, including post-translational modifications of that sequence. In a particular aspect, the VL comprises one, two or three CDRs selected from: (a) CDR-L1, comprising the amino acid sequence of SEQ ID NO:4 or SEQ ID NO:9, (b) CDR-L2, comprising the amino acid sequence of SEQ ID NO: 5 or SEQ ID NO:11, and (c) CDR-L3, comprising the amino acid sequence of SEQ ID NO: 6. In a very particular aspect, the VL comprises one, two or three CDRs selected from: (a) CDR-L1, comprising the amino acid sequence of SEQ ID NO:4, (b) CDR-L2, comprising the amino acid sequence of SEQ ID NO:5, and (c) CDR-L3, comprising the amino acid sequence of SEQ ID NO:6.

In another aspect, a fusion protein comprising a human IFN-α2, a first antigen-binding domain capable of binding to human PD-L1 and a second antigen-binding domain capable of binding to human PD-L1 and to human IFN-α2 is provided, wherein the second antigen-binding domain comprises a VH sequence as in any of the aspects provided above, and a VL sequence as in any of the aspects provided above. In one aspect, the second antigen-binding domain comprises the VH and VL sequences in SEQ ID NO:7 and SEQ ID NO:8, SEQ ID NO: 10 or SEQ ID NO: 12, respectively, including post-translational modifications of those sequences. In one aspect, the second antigen-binding domain comprises the VH and VL sequences in SEQ ID NO:7 and SEQ ID NO:8, respectively, including post-translational modifications of those sequences.

In a further aspect of the invention, a fusion protein according to any of the above aspects comprises domains derived from a monoclonal antibody, including a chimeric, humanized or human antibody. In one aspect, a fusion protein according to any of the above aspects comprises at least one antibody fragment, e.g., a Fv, Fab, Fab′, scFv, diabody, or F(ab′)₂ fragment.

In a further aspect, the fusion protein according to any of the above aspects comprises antigen-binding domains derived from antibodies that are of IgG1 isotype/subclass and comprises an Fc domain comprising two subunits having the amino acid sequence of SEQ ID NO: 84, SEQ ID NO:86, SEQ ID NO:87 or SEQ ID NO:88, or parts of the amino acid sequence of SEQ ID NO:84, SEQ ID NO:86, SEQ ID NO:87 or SEQ ID NO:88. In another aspect, the fusion protein according to any of the above aspects comprises domains derived from antibodies that are of IgG1 isotype/subclass and comprise a constant heavy chain domain of SEQ ID NO:93 or SEQ ID NO:94, or the constant parts of the heavy chain amino acid sequence of SEQ ID NO:93 or SEQ ID NO:94. In one aspect, additionally the C-terminal glycine (Gly446) is present. In one aspect, additionally the C-terminal glycine (Gly446) and the C-terminal lysine (Lys447) is present.

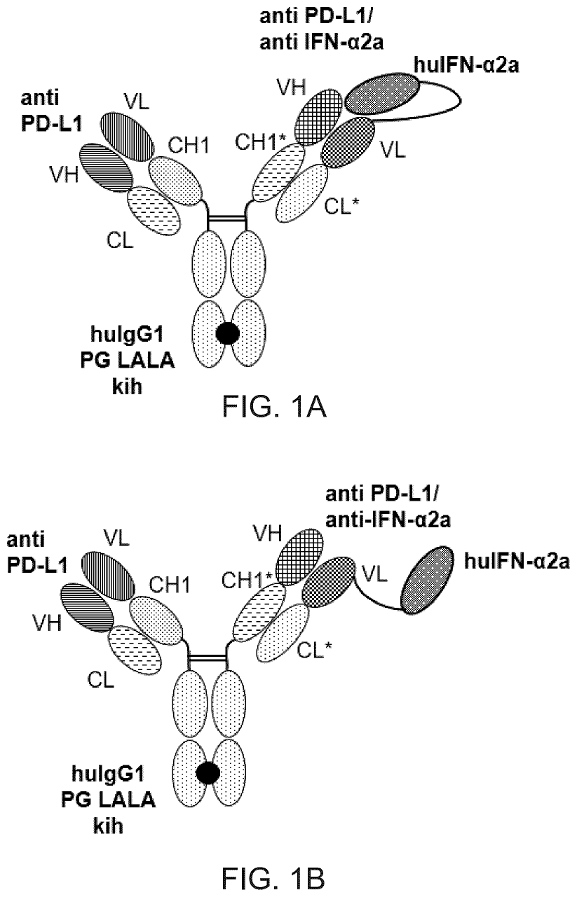

In particular aspects, the invention provides PD-L1-targeted IFN-α2 fusion proteins comprising a human IFN-α2, a first antigen-binding domain capable of binding to human PD-L1, a second antigen-binding domain capable of binding to human PD-L1 and to human IFN-α2, and an Fc domain composed of a first and a second subunit (as shown in FIG. 1A-1B), wherein a) the first antigen-binding domain is a crossover Fab and is fused at the C-terminus of its heavy chain to the N-terminus of one of the two subunits of the Fc domain, b) the second antigen-binding domain is a DutaFab and is fused at the C-terminus of its heavy chain to the N-terminus of the other of the two subunits of the Fc domain, and c) the human IFN-α2 is wildtype human IFN-α2 and is fused at its C-terminus to the N-terminus of the light chain of the second antigen-binding domain.

In a further aspect of the invention, a fusion protein is provided that comprises a first antigen-binding domain capable of binding to human PD-L1, a second antigen-binding domain capable of binding to human PD-L1 and to human IFN-α2, a human IFN-α2, and an Fc domain composed of a first and a second subunit, wherein the second antigen-binding domain is a bispecific Fab, and

wherein the fusion protein is composed of
(a) a first polypeptide, comprising (a1) the heavy chain of the first antigen binding domain, fused at its C-terminus to the N-terminus of one of the subunits (e.g. the first subunit) of the Fc domain, and (a2) one of the subunits (e.g. the first subunit) of the Fc domain;
(b) a second polypeptide, comprising the light chain of the first antigen-binding domain;
(c) a third polypeptide, comprising (c1) the heavy chain of the second antigen-binding domain, fused at its C-terminus to the N-terminus of the other one of the subunits (e.g. the second subunit) of the Fc domain, and (c2) the other one of the subunits (e.g. the second subunit) of the Fc domain; and
(d) a fourth polypeptide, comprising (d1) the human IFN-α2, fused at its C-terminus to the N-terminus of the light chain of the second antigen-binding domain, and (d2) the light chain of the second antigen-binding domain.

In one aspect, a fusion protein as described herein comprises a first polypeptide comprising an amino acid sequence that is at least about 95%, 96%, 97%, 98%, 99% or 100% identical to the amino acid sequence of SEQ ID NO:37, a second polypeptide comprising an amino acid sequence that is at least about 95%, 96%, 97%, 98%, 99% or 100% identical to the amino acid sequence of SEQ ID NO:38, a third polypeptide comprising an amino acid sequence that is at least about 95%, 96%, 97%, 98%, 99% or 100% identical to the amino acid sequence of SEQ ID NO:39, and a fourth polypeptide comprising an amino acid sequence that is at least about 95%, 96%, 97%, 98%, 99% or 100% identical to the amino acid sequence of SEQ ID NO:40. In a further aspect, the fusion protein comprises a first polypeptide comprising an amino acid sequence of SEQ ID NO:37, a second polypeptide comprising an amino acid sequence of SEQ ID NO:38, a third polypeptide comprising an amino acid sequence of SEQ ID NO:39, and a fourth polypeptide comprising an amino acid sequence of SEQ ID NO:40.

In a further aspect, a fusion protein as described herein comprises a first polypeptide comprising an amino acid sequence that is at least about 95%, 96%, 97%, 98%, 99% or 100% identical to the amino acid sequence of SEQ ID NO:41, a second polypeptide comprising an amino acid sequence that is at least about 95%, 96%, 97%, 98%, 99% or 100% identical to the amino acid sequence of SEQ ID NO:42, a third polypeptide comprising an amino acid sequence that is at least about 95%, 96%, 97%, 98%, 99% or 100% identical to the amino acid sequence of SEQ ID NO:43, and a fourth polypeptide comprising an amino acid sequence that is at least about 95%, 96%, 97%, 98%, 99% or 100% identical to the amino acid sequence of SEQ ID NO:44. In a further aspect, the fusion protein comprises a first polypeptide comprising an amino acid sequence of SEQ ID NO: 41, a second polypeptide comprising an amino acid sequence of SEQ ID NO:42, a third polypeptide comprising an amino acid sequence that is of SEQ ID NO:43, and a fourth polypeptide comprising an amino acid sequence that is of SEQ ID NO:44.

In a further aspect, a fusion protein as described herein comprises a first polypeptide comprising an amino acid sequence that is at least about 95%, 96%, 97%, 98%, 99% or 100% identical to the amino acid sequence of SEQ ID NO:45, a second polypeptide comprising an amino acid sequence that is at least about 95%, 96%, 97%, 98%, 99% or 100% identical to the amino acid sequence of SEQ ID NO:46, a third polypeptide comprising an amino acid sequence that is at least about 95%, 96%, 97%, 98%, 99% or 100% identical to the amino acid sequence of SEQ ID NO:47, and a fourth polypeptide comprising an amino acid sequence that is at least about 95%, 96%, 97%, 98%, 99% or 100% identical to the amino acid sequence of SEQ ID NO:48. In a further aspect, the fusion protein comprises a first polypeptide comprising an amino acid sequence of SEQ ID NO: 45, a second polypeptide comprising an amino acid sequence of SEQ ID NO:46, a third polypeptide comprising an amino acid sequence of SEQ ID NO:47, and a fourth polypeptide comprising an amino acid sequence of SEQ ID NO:48.

In a further aspect, a fusion protein as described herein comprises a first polypeptide comprising a first polypeptide comprising an amino acid sequence that is at least about 95%, 96%, 97%, 98%, 99% or 100% identical to the amino acid sequence of SEQ ID NO: 49, a second polypeptide comprising an amino acid sequence that is at least about 95%, 96%, 97%, 98%, 99% or 100% identical to the amino acid sequence of SEQ ID NO:50, a third polypeptide comprising an amino acid sequence that is at least about 95%, 96%, 97%, 98%, 99% or 100% identical to the amino acid sequence of SEQ ID NO:51, and a fourth polypeptide comprising an amino acid sequence that is at least about 95%, 96%, 97%, 98%, 99% or 100% identical to the amino acid sequence of SEQ ID NO:52. In a further aspect, the fusion protein comprises a first polypeptide comprising a first polypeptide comprising an amino acid sequence of SEQ ID NO:49, a second polypeptide comprising an amino acid sequence of SEQ ID NO:50, a third polypeptide comprising an amino acid sequence of SEQ ID NO:51, and a fourth polypeptide comprising an amino acid sequence of SEQ ID NO: 52.

In a further aspect, a fusion protein as described herein comprises a first polypeptide comprising an amino acid sequence that is at least about 95%, 96%, 97%, 98%, 99% or 100% identical to the amino acid sequence of SEQ ID NO:53, a second polypeptide comprising an amino acid sequence that is at least about 95%, 96%, 97%, 98%, 99% or 100% identical to the amino acid sequence of SEQ ID NO:54, a third polypeptide comprising an amino acid sequence that is at least about 95%, 96%, 97%, 98%, 99% or 100% identical to the amino acid sequence of SEQ ID NO:55, and a fourth polypeptide comprising an amino acid sequence that is at least about 95%, 96%, 97%, 98%, 99% or 100% identical to the amino acid sequence of SEQ ID NO:56. In a further aspect, the fusion protein comprises a first polypeptide comprising an amino acid sequence of SEQ ID NO: 53, a second polypeptide comprising an amino acid sequence of SEQ ID NO:54, a third polypeptide comprising an amino acid sequence of SEQ ID NO:55, and a fourth polypeptide comprising an amino acid sequence of SEQ ID NO:56.

In a further aspect, a fusion protein as described herein comprises a first polypeptide comprising an amino acid sequence that is at least about 95%, 96%, 97%, 98%, 99% or 100% identical to the amino acid sequence of SEQ ID NO:57, a second polypeptide comprising an amino acid sequence that is at least about 95%, 96%, 97%, 98%, 99% or 100% identical to the amino acid sequence of SEQ ID NO:58, a third polypeptide comprising an amino acid sequence that is at least about 95%, 96%, 97%, 98%, 99% or 100% identical to the amino acid sequence of SEQ ID NO:59, and a fourth polypeptide comprising an amino acid sequence that is at least about 95%, 96%, 97%, 98%, 99% or 100% identical to the amino acid sequence of SEQ ID NO:60. In a further aspect, the fusion protein comprises a first polypeptide comprising an amino acid sequence of SEQ ID NO: 57, a second polypeptide comprising an amino acid sequence of SEQ ID NO:58, a third polypeptide comprising an amino acid sequence of SEQ ID NO:59, and a fourth polypeptide comprising an amino acid sequence of SEQ ID NO:60.

In one particular aspect, the invention provides a fusion protein that comprises a) a first antigen-binding domain capable of binding to human PD-L1, b) a second antigen-binding domain capable of binding to human PD-L1 and to human IFN-α2 and capable of blocking the human IFN-α2 from binding to IFNAR, c) a human IFN-α2, and d) an Fc domain composed of a first and a second subunit,

wherein
i. the first antigen-binding domain is a Fab and is fused at the C-terminus of its Fab heavy chain to the N-terminus of the first subunit of the Fc domain;
ii. the second antigen-binding domain is a bispecific Fab (in particular a DutaFab) and is fused at the C-terminus of its Fab heavy chain to the N-terminus of the second subunit of the Fc domain, and
iii. the human IFN-α2 is fused at its C-terminus to the N-terminus of the light chain of the second antigen-binding domain via a peptidic linker,
   and wherein
A. the first antigen-binding domain capable of binding to human PD L1 comprises a heavy chain variable domain (VH) comprising (a) CDR-H1 comprising the amino acid sequence of SEQ ID NO:21, (b) CDR-H2 comprising the amino acid sequence of SEQ ID NO:22, and (c) CDR-H3 comprising the amino acid sequence of SEQ ID NO:23, and a light chain variable domain (VL) comprising (d) CDR-L1 comprising the amino acid sequence of SEQ ID NO:24, (e) CDR-L2 comprising the amino acid sequence of SEQ ID NO:25, and (f) CDR-L3 comprising the amino acid sequence of SEQ ID NO:26; and
B. the second antigen-binding domain capable of binding to human PD-L1 and to human IFN-α2 comprises a heavy chain variable domain (VH) comprising (a) CDR-H1 comprising the amino acid sequence of SEQ ID NO:1, (b) CDR-H2 comprising the amino acid sequence of SEQ ID NO:2, and (c) CDR-H3 comprising the amino acid sequence of SEQ ID NO:3, and a light chain variable domain (VL) comprising (d) CDR-L1 comprising the amino acid sequence of SEQ ID NO:4, (e) CDR-L2 comprising the amino acid sequence of SEQ ID NO:5, and (f) CDR-L3 comprising the amino acid sequence of SEQ ID NO: 6.

In a further aspect, the invention provides a fusion protein that comprises a) a first antigen-binding domain capable of binding to human PD-L1, b) a second antigen-binding domain capable of binding to human PD-L1 and to human IFN-α2 and capable of blocking the human IFN-α2 from binding to IFNAR, c) a human IFN-α2, and d) an Fc domain composed of a first and a second subunit,
wherein
i. the first antigen-binding domain is a Fab and is fused at the C-terminus of its Fab heavy chain to the N-terminus of the first subunit of the Fc domain;
ii. the second antigen-binding domain is a bispecific Fab (in particular a DutaFab) and is fused at the C-terminus of its Fab heavy chain to the N-terminus of the second subunit of the Fc domain, and
iii. the human IFN-α2 is fused at its C-terminus to the N-terminus of the light chain of the second antigen-binding domain via a peptidic linker,
   and wherein
A, wherein the first antigen-binding domain capable of binding to human PD-L1 comprises a VH domain comprising the amino acid sequence of SEQ ID NO:27 and a VL domain comprising the amino acid sequence of SEQ ID NO:28 and
B. the second antigen-binding domain capable of binding to human PD-L1 and to human IFN-α2 comprises a VH domain comprising the amino acid sequence of SEQ ID NO:7 and a VL domain comprising the amino acid sequence of SEQ ID NO: 8.

In a further aspect, a fusion protein according to any of the above aspects may incorporate any of the features, singly or in combination, as described in Sections 2-8 below.

### 2. Exemplary Anti-PD-L1 Antibodies

In one aspect, the invention provides antibodies that bind to human PD-L1. In one aspect, provided are isolated antibodies that bind to human PD-L1. In one aspect, the invention provides antibodies that specifically bind to human PD-L1. In certain aspects, an anti-huPD-L1 antibody binds to the C-terminal domain of human PD-L1. In certain aspects, an anti-huPD-L1 antibody does not compete with Atezolizumab for PD-L1 binding. In one aspect, an anti-huPD-L1 antibody blocks binding of PD-L1 to PD1. In one aspect, an anti-huPD-L1 antibody blocks binding of PD-L1 to PD1, in particular at a concentration of at least 1-10 nM. In one aspect, an anti-huPD-L1 antibody blocks PD1/PD-L1 interaction, in particular at a concentration of at least 1-10 nM. In another aspect, an anti-huPD-L1 antibody binds to human PD-L1 with an affinity of ≤1.1 nM, in particular of ≤0.8 nM.

In one aspect, the invention provides an anti-huPD-L1 antibody comprising
(i) a VH domain comprising a) CDR-H1 comprising the amino acid sequence of SEQ ID NO: 21; (b) CDR-H2 comprising the amino acid sequence of SEQ ID NO:22; and (c) CDR-H3 comprising the amino acid sequence of SEQ ID NO:23; and
(ii) a VL domain comprising (a) CDR-L1 comprising the amino acid sequence of SEQ ID NO: 24; (b) CDR-L2 comprising the amino acid sequence of SEQ ID NO:25; and (c) CDR-L3 comprising the amino acid sequence of SEQ ID NO:26.

In any of the aspects provided herein, an anti-huPD-L1 antibody is humanized. In one aspect, an anti-huPD-L1 antibody further comprises an acceptor human framework, e.g. a human immunoglobulin framework or a human consensus framework.

In another aspect, an anti-huPD-L1 antibody comprises one or more of the CDR sequences of the VH of SEQ ID NO:27. In another embodiment, an anti-huPD-L1 antibody comprises one or more of the CDR sequences of the VL of SEQ ID NO:28. In another embodiment, an anti-huPD-L1 antibody comprises the CDR sequences of the VH of SEQ ID NO:27 and the CDR sequences of the VL of SEQ ID NO:28.

In a further aspect, an anti-huPD-L1 antibody comprises the CDR-H1, CDR-H2 and CDR-H3 amino acid sequences of the VH domain of SEQ ID NO:27 and the CDR-L1, CDR-L2 and CDR-L3 amino acid sequences of the VL domain of SEQ ID NO:28.

In one aspect, an anti-huPD-L1 antibody comprises one or more of the heavy chain CDR amino acid sequences of the VH domain of SEQ ID NO:27 and a framework of at least 85%, 86%, 87%, 88%, 89%, 90%, 91%, 92%, 93%, 94%, 95%, 96%, 97%, 98%, 99% sequence identity to the framework amino acid sequence of the VH domain of SEQ ID NO: 27. In one aspect, the anti-huPD-L1 antibody comprises the three heavy chain CDR amino acid sequences of the VH domain of SEQ ID NO:27 and a framework of at least 85%, 86%, 87%, 88%, 89%, 90%, 91%, 92%, 93%, 94%, 95%, 96%, 97%, 98%, 99% sequence identity to the framework amino acid sequence of the VH domain of SEQ ID NO: 27. In one aspect, the anti-huPD-L1 antibody comprises the three heavy chain CDR amino acid sequences of the VH domain of SEQ ID NO:27 and a framework of at least 95% sequence identity to the framework amino acid sequence of the VH domain of SEQ ID NO: 27. In another aspect, the anti-huPD-L1 antibody comprises the three heavy chain CDR amino acid sequences of the VH domain of SEQ ID NO:27 and a framework of at least of at least 98% sequence identity to the framework amino acid sequence of the VH domain of SEQ ID NO:27.

In one aspect, an anti-huPD-L1 antibody comprises one or more of the light chain CDR amino acid sequences of the VL domain of SEQ ID NO:28 and a framework of at least 85%, 86%, 87%, 88%, 89%, 90%, 91%, 92%, 93%, 94%, 95%, 96%, 97%, 98%, 99% sequence identity to the framework amino acid sequence of the VL domain of SEQ ID NO: 28. In one aspect, the anti-huPD-L1 antibody comprises the three light chain CDR amino acid sequences of the VL domain of SEQ ID NO:28 and a framework of at least 85%, 86%, 87%, 88%, 89%, 90%, 91%, 92%, 93%, 94%, 95%, 96%, 97%, 98%, 99% sequence identity to the framework amino acid sequence of the VL domain of SEQ ID NO: 28. In one aspect, the anti-huPD-L1 antibody comprises the three light chain CDR amino acid sequences of the VL domain of SEQ ID NO:28 and a framework of at least 95% sequence identity to the framework amino acid sequence of the VL domain of SEQ ID NO: 28. In another aspect, the anti-huPD-L1 antibody comprises the three light chain CDR amino acid sequences of the VL domain of SEQ ID NO:28 and a framework of at least particularly of at least 98% sequence identity to the framework amino acid sequence of the VH domain of SEQ ID NO:28.

In one aspect, the anti-huPD-L1 antibody comprises (a) CDR-H1 comprising the amino acid sequence of SEQ ID NO:21; (b) CDR-H2 comprising the amino acid sequence of SEQ ID NO: 22; (c) CDR-H3 comprising the amino acid sequence of SEQ ID NO:23; (d) CDR-L1 comprising the amino acid sequence of SEQ ID NO:24; (c) CDR-L2 comprising the amino acid sequence of SEQ ID NO:25; and (f) CDR-L3 comprising the amino acid sequence of SEQ ID NO:26, and a VH domain having at least 90%, 91%, 92%, 93%, 94%, 95%, 96%, 97%, 98%, 99%, or 100% sequence identity to the amino acid sequence of SEQ ID NO: 27, and a VL domain having at least 90%, 91%, 92%, 93%, 94%, 95%, 96%, 97%, 98%, 99%, or 100% sequence identity to the amino acid sequence of SEQ ID NO:28. In one aspect, the VH domain has at least 95% sequence identity to the amino acid sequence of SEQ ID NO:27. In one aspect, the VL domain has at least 95% sequence identity to the amino acid sequence of SEQ ID NO:28. In one aspect, the anti-huPD-L1 antibody comprises a VH domain that has at least 95% sequence identity to the amino acid sequence of SEQ ID NO:27 and a VL domain that has at least 95% sequence identity to the amino acid sequence of SEQ ID NO:28.

In one aspect, the anti-huPD-L1 antibody comprises (a) CDR-H1 comprising the amino acid sequence of SEQ ID NO:21; (b) CDR-H2 comprising the amino acid sequence of SEQ ID NO: 22; (c) CDR-H3 comprising the amino acid sequence of SEQ ID NO:23; (d) CDR-L1 comprising the amino acid sequence of SEQ ID NO:24; (e) CDR-L2 comprising the amino acid sequence of SEQ ID NO:25; and (f) CDR-L3 comprising the amino acid sequence of SEQ ID NO:26, and a VH domain having at least 90%, 91%, 92%, 93%, 94%, 95%, 96%, 97%, 98%, 99%, or 100% sequence identity to the amino acid sequence of SEQ ID NO: 27, and a VL domain having at least 90%, 91%, 92%, 93%, 94%, 95%, 96%, 97%, 98%, 99%, or 100% sequence identity to the amino acid sequence of SEQ ID NO:28; wherein the antibody specifically binds to human PD-L1. In one aspect, the VH domain has at least 95% sequence identity to the amino acid sequence of SEQ ID NO:27. In one aspect, the VL domain has at least 95% sequence identity to the amino acid sequence of SEQ ID NO:28. In one aspect, the antibody binds to PD-L1 having a dissociation constant (K_{D}) that is up to 10 fold reduced or up to 10 fold increased when compared to the dissociation constant (K_{D}) of an antibody comprising a VH sequence of SEQ ID NO:27 and a VL sequence of SEQ ID NO:28.

In another aspect, an anti-huPD-L1 antibody comprises a heavy chain variable domain (VH) sequence having at least 90%, 91%, 92%, 93%, 94%, 95%, 96%, 97%, 98%, 99%, or 100% sequence identity to the amino acid sequence of SEQ ID NO:27. In one aspect, an anti-huPD-L1 antibody comprises a heavy chain variable domain (VH) sequence having at least 95%, sequence identity to the amino acid sequence of SEQ ID NO:27. In certain aspects, a VH sequence having at least 90%, 91%, 92%, 93%, 94%, 95%, 96%, 97%, 98%, or 99% identity contains substitutions (e.g., conservative substitutions), insertions, or deletions relative to the reference sequence, but an anti-huPD-L1 antibody comprising that sequence retains the ability to bind to human PD-L1. In certain aspects, a total of 1 to 10 amino acids have been substituted, inserted and/or deleted in SEQ ID NO:27. In certain aspects, substitutions, insertions, or deletions occur in regions outside the CDRs (i.e., in the FRs). Optionally, the anti-PD-L1 antibody comprises the VH sequence in SEQ ID NO: 27, including post-translational modifications of that sequence. In a particular aspect, the VH comprises one, two or three CDRs selected from: (a) CDR-H1, comprising the amino acid sequence of SEQ ID NO:21, (b) CDR-H2, comprising the amino acid sequence of SEQ ID NO:22, and (c) CDR-H3, comprising the amino acid sequence of SEQ ID NO: 23. In another aspect, an anti-huPD-L1 antibody is provided, wherein the antibody comprises a light chain variable domain (VL) sequence having at least 90%, 91%, 92%, 93%, 94%, 95%, 96%, 97%, 98%, 99%, or 100% sequence identity to the amino acid sequence of SEQ ID NO:28. In one aspect, an anti-huPD-L1 antibody comprises a light chain variable domain (VL) sequence having at least 95% sequence identity to the amino acid sequence of SEQ ID NO:28. In certain aspects, a VL sequence having at least 90%, 91%, 92%, 93%, 94%, 95%, 96%, 97%, 98%, or 99% identity contains substitutions (e.g., conservative substitutions), insertions, or deletions relative to the reference sequence, but an anti-huPD-L1 antibody comprising that sequence retains the ability to bind to PD-L1. In certain aspects, a total of 1 to 10 amino acids have been substituted, inserted and/or deleted in SEQ ID NO:28. In certain aspects, the substitutions, insertions, or deletions occur in regions outside the CDRs (i.e., in the FRs). Optionally, the anti-huPD-L1 antibody comprises the VL sequence in SEQ ID NO:28, including post-translational modifications of that sequence. In a particular aspect, the VL comprises one, two or three CDRs selected from: (a) CDR-L1, comprising the amino acid sequence of SEQ ID NO:24, (b) CDR-L2, comprising the amino acid sequence of SEQ ID NO:25, and (c) CDR-L3, comprising the amino acid sequence of SEQ ID NO:26.

In another aspect, an anti-huPD-L1 antibody is provided, wherein the antibody comprises a VH sequence as in any of the aspects provided above, and a VL sequence as in any of the aspects provided above. In one aspect, the antibody comprises the VH and VL sequences in SEQ ID NO:27 and SEQ ID NO:28, respectively, including post-translational modifications of those sequences.

In a further aspect of the invention, an anti-huPD-L1 antibody according to any of the above aspects is a monoclonal antibody, including a chimeric, humanized or human antibody. In one aspect, an anti-huPD-L1 antibody is an antibody fragment, e.g., an Fv, Fab, Fab′, scFv, diabody, or F(ab′)₂ fragment.

In one aspect, an anti-huPD-L1 antibody according to any of the above aspects is a DutaFab. In one aspect, an anti-huPD-L1 antibody comprises a human PD-L1 paratope and a non-binding paratope (i.e. a paratope that binds to no epitope) within one cognate pair of a variable light chain domain (VL domain) and a variable heavy chain domain (VH domain), wherein the non-binding paratope comprises amino acid residues from CDR-H2, CDR-L1 and CDR-L3 of the antigen-binding domain, and wherein the PD-L1 paratope comprises amino acid residues from the CDR-H1, CDR-H3 and CDR-L2 of the antigen-binding domain.

In another aspect, the antibody is a full-length antibody, e.g., an intact IgG1 antibody or other antibody class or isotype as defined herein.

In a further aspect, the antibody as described herein is of IgG1 isotype/subclass and comprises a constant heavy chain domain of SEQ ID NO:93 or the constant parts of the heavy chain amino acid sequence of SEQ ID NO:142. In another aspect, the antibody according to any of the above aspects comprises a constant heavy chain domain of SEQ ID: 93 and of the heavy chain amino acid sequence of SEQ ID NO:142. In one aspect, additionally the C-terminal glycine (Gly446) is present. In one aspect, additionally the C-terminal glycine (Gly446) and the C-terminal lysine (Lys447) is present.

In one aspect, an anti-huPD-L1 antibody is provided that comprises a heavy chain having the amino acid sequence of SEQ ID NO:142 and a light chain having the amino acid sequence of SEQ ID NO:143.

In a further aspect, an anti-huPD-L1 antibody according to any of the above aspects may incorporate any of the features, singly or in combination, as described in Sections 3-8 below:

### 3. Antibody Affinity

In certain aspects, an antibody or an antigen-binding domain provided herein has a dissociation constant (K_{D}) of ≤1 μM, ≤100 nM, ≤10 nM, ≤1 nM or ≤0.1 nM, (e.g., 10⁻⁸ M or less, e.g., from 10⁻⁸ M to 10⁻¹⁰ M, e.g., from 10⁻⁹ M to 10⁻¹⁰ M).

In one aspect, K_{D} is measured using a BIACORE® surface plasmon resonance assay. For example, an assay using a BIACORE®-2000 or a BIACORE®-3000 (BIAcore, Inc., Piscataway, NJ) is performed at 25° C. with immobilized antigen CM5 chips at ˜10 response units (RU). In one aspect, carboxymethylated dextran biosensor chips (CM5, BIACORE, Inc.) are activated with N-ethyl-N′-(3-dimethylaminopropyl)-carbodiimide hydrochloride (EDC) and N-hydroxysuccinimide (NHS) according to the supplier's instructions. Antigen is diluted with 10 mM sodium acetate, pH 4.8, to 5 μg/ml (˜0.2 μM) before injection at a flow rate of 5 μl/minute to achieve approximately 10 response units (RU) of coupled protein. Following the injection of antigen, 1 M ethanolamine is injected to block unreacted groups. For kinetics measurements, two-fold serial dilutions of Fab (0.78 nM to 500 nM) are injected in PBS with 0.05% polysorbate 20 (TWEEN-20™) surfactant (PBST) at 25° C. at a flow rate of approximately 25 μl/min. Association rates (kₒₙ) and dissociation rates (k_{off}) are calculated using a simple one-to-one Langmuir binding model (BIACORE® Evaluation Software version 3.2) by simultaneously fitting the association and dissociation sensorgrams. The equilibrium dissociation constant (K_{D}) is calculated as the ratio k_{off}/kₒₙ. See, e.g., Chen et al., J. Mol. Biol. 293:865-881 (1999). If the on-rate exceeds 10⁶ M⁻¹ s⁻¹ by the surface plasmon resonance assay above, then the on-rate can be determined by using a fluorescent quenching technique that measures the increase or decrease in fluorescence emission intensity (excitation=295 nm; emission=340 nm, 16 nm band-pass) at 25° C. of a 20 nM anti-antigen antibody (Fab form) in PBS, pH 7.2, in the presence of increasing concentrations of antigen as measured in a spectrometer, such as a stop-flow equipped spectrophometer (Aviv Instruments) or a SLM-AMINCO™ spectrophotometer (ThermoSpectronic) with a stirred cuvette.

In one aspect, the K_{D} for binding of an anti-PD-L1/anti-IFN-α2 antigen-binding domain to IFN-α2 is measured using a BIACORE® surface plasmon resonance assay. For example, an assay using a Biacore 8K or 8K+ instrument (Cytiva) is performed at 25° C. Anti-human Fab antibody (Cytiva; Catalog number 28958325) is immobilized on a CM5 chip according to the manufacturer's instructions. 100 nM anti-PD-L1/anti-IFN-α2a DutaFabs are captured (10 μl/min, 60 sec) and 0 nM, 10 nM, 50 nM and 150 nM of untagged huIFN-α2a (SEQ ID NO: 79) are flown at 30 μl/min for 120 sec followed by a 240 second dissociation window at a flow rate of 30 μl/min. The surface is regenerated by injecting 10 mM glycine, pH 2, for 60 seconds at a flow rate of 30 μl/min. Binding curves are evaluated using Biacore 8K evaluation software (Cytiva) and for the calculation of binding properties a 1:1 Langmuir binding model is used.

### 4. Antibody Fragments

In certain aspects, a fusion protein provided herein comprises one or more antibody fragments. In certain aspects, an antibody provided herein is an antibody fragment.

In one aspect, the antibody fragment is a Fab, Fab′, Fab′-SH, or F(ab′)₂ fragment, in particular a Fab fragment. Papain digestion of intact antibodies produces two identical antigen-binding fragments, called “Fab” fragments containing each the heavy- and light-chain variable domains (VH and VL, respectively) and also the constant domain of the light chain (CL) and the first constant domain of the heavy chain (CH1). The term “Fab fragment” thus refers to an antibody fragment comprising a light chain comprising a VL domain and a CL domain, and a heavy chain fragment comprising a VH domain and a CH1 domain. “Fab′ fragments” differ from Fab fragments by the addition of residues at the carboxy terminus of the CH1 domain including one or more cysteines from the antibody hinge region. Fab′-SH are Fab′ fragments in which the cysteine residue(s) of the constant domains bear a free thiol group. Pepsin treatment yields an F(ab′)₂ fragment that has two antigen-binding sites (two Fab fragments) and a part of the Fc region. For discussion of Fab and F(ab′)₂ fragments comprising salvage receptor binding epitope residues and having increased in vivo half-life, see U.S. Pat. No. 5,869,046.

In certain aspects, the antibody fragment is a DutaFab. A DutaFab is a Fab wherein a single pair of a VH domain and a VL domain is capable of specifically binding to two different epitopes, wherein one paratope comprises amino acid residues from CDR-H2, CDR-L1 and CDR-L3 and the other paratope comprises amino residues from CDR-H1, CDR-H3 and CDR-L2. In one aspect, the DutaFab comprises two non-overlapping paratopes within a cognate VH/VL pair and binds to the two different epitopes in a mutually exclusive manner (“Dutaflip”).

In another aspect, the antibody fragment is a diabody, a triabody or a tetrabody. “Diabodies” are antibody fragments with two antigen-binding sites that may be bivalent or bispecific. See, for example, EP 404,097; WO 1993/01161; Hudson et al., Nat. Med. 9:129-134 (2003); and Hollinger et al., Proc. Natl. Acad. Sci. USA 90:6444-6448 (1993). Triabodies and tetrabodies are also described in Hudson et al., Nat. Med. 9:129-134 (2003).

In a further aspect, the antibody fragment is a single chain Fab fragment. A “single chain Fab fragment” or “scFab” is a polypeptide consisting of an antibody heavy chain variable domain (VH), an antibody heavy chain constant domain 1 (CH1), an antibody light chain variable domain (VL), an antibody light chain constant domain (CL) and a linker, wherein said antibody domains and said linker have one of the following orders in N-terminal to C-terminal direction: a) VH-CH1-linker-VL-CL, b) VL-CL-linker-VH-CH1, c) VH-CL-linker-VL-CH1 or d) VL-CH1-linker-VH-CL. In particular, said linker is a polypeptide of at least 30 amino acids, preferably between 32 and 50 amino acids. Said single chain Fab fragments are stabilized via the natural disulfide bond between the CL domain and the CH1 domain. In addition, these single chain Fab fragments might be further stabilized by generation of interchain disulfide bonds via insertion of cysteine residues (e.g., position 44 in the variable heavy chain and position 100 in the variable light chain according to Kabat numbering).

In another aspect, the antibody fragment is single-chain variable fragment (scFv). A “single-chain variable fragment” or “scFv” is a fusion protein of the variable domains of the heavy (VH) and light chains (VL) of an antibody, connected by a linker. In particular, the linker is a short polypeptide of 10 to 25 amino acids and is usually rich in glycine for flexibility, as well as serine or threonine for solubility, and can either connect the N-terminus of the VH with the C-terminus of the VL, or vice versa. This protein retains the specificity of the original antibody, despite removal of the constant regions and the introduction of the linker. For a review of scFv fragments, see, e.g., Plückthun, in The Pharmacology of Monoclonal Antibodies, vol. 113, Rosenburg and Moore eds., (Springer-Verlag, New York), pp. 269-315 (1994); see also WO 93/16185; and U.S. Pat. Nos. 5,571,894 and 5,587,458.

In another aspect, the antibody fragment is a single-domain antibody. “Single-domain antibodies” are antibody fragments comprising all or a portion of the heavy chain variable domain or all or a portion of the light chain variable domain of an antibody. In certain aspects, a single-domain antibody is a human single-domain antibody (Domantis, Inc., Waltham, MA; see, e.g., U.S. Pat. No. 6,248,516 B1).

Antibody fragments can be made by various techniques, including but not limited to proteolytic digestion of an intact antibody as well as recombinant production by recombinant host cells (e.g., E. coli), as described herein.

### 5. Chimeric and Humanized Antibodies

In certain aspects, an antibody provided herein is a chimeric antibody. In certain aspects, a fusion protein provided herein comprises a chimeric antibody or a fragment of a chimeric antibody. Certain chimeric antibodies are described, e.g., in U.S. Pat. No. 4,816,567; and Morrison et al., Proc. Natl. Acad. Sci. USA, 81:6851-6855 (1984)). In one example, a chimeric antibody comprises a non-human variable region (e.g., a variable region derived from a mouse, rat, hamster, rabbit, or non-human primate, such as a monkey) and a human constant region. In a further example, a chimeric antibody is a “class switched” antibody in which the class or subclass has been changed from that of the parent antibody. Chimeric antibodies include antigen-binding fragments thereof.

In certain aspects, a chimeric antibody is a humanized antibody. Typically, a non-human antibody is humanized to reduce immunogenicity to humans, while retaining the specificity and affinity of the parental non-human antibody. Generally, a humanized antibody comprises one or more variable domains in which the CDRs (or portions thereof) are derived from a non-human antibody, and FRs (or portions thereof) are derived from human antibody sequences. A humanized antibody optionally will also comprise at least a portion of a human constant region. In some aspects, some FR residues in a humanized antibody are substituted with corresponding residues from a non-human antibody (e.g., the antibody from which the CDR residues are derived), e.g., to restore or improve antibody specificity or affinity.

Humanized antibodies and methods of making them are reviewed, e.g., in Almagro and Fransson, Front. Biosci. 13:1619-1633 (2008), and are further described, e.g., in Riechmann et al., Nature 332:323-329 (1988); Queen et al., Proc. Nat'l Acad. Sci. USA 86:10029-10033 (1989); U.S. Pat. Nos. 5,821,337, 7,527,791, 6,982,321, and 7,087,409; Kashmiri et al., Methods 36:25-34 (2005) (describing specificity determining region (SDR) grafting); Padlan, Mol. Immunol. 28:489-498 (1991) (describing “resurfacing”); Dall'Acqua et al., Methods 36:43-60 (2005) (describing “FR shuffling”); and Osbourn et al., Methods 36:61-68 (2005) and Klimka et al., Br. J. Cancer, 83:252-260 (2000) (describing the “guided selection” approach to FR shuffling).

Human framework regions that may be used for humanization include but are not limited to: framework regions selected using the “best-fit” method (see, e.g., Sims et al. J. Immunol. 151:2296 (1993)); framework regions derived from the consensus sequence of human antibodies of a particular subgroup of light or heavy chain variable regions (see, e.g., Carter et al. Proc. Natl. Acad. Sci. USA, 89:4285 (1992); and Presta et al. J. Immunol., 151:2623 (1993)); human mature (somatically mutated) framework regions or human germline framework regions (see, e.g., Almagro and Fransson, Front. Biosci. 13:1619-1633 (2008)); and framework regions derived from screening FR libraries (see, e.g., Baca et al., J. Biol. Chem. 272:10678-10684 (1997) and Rosok et al., J. Biol. Chem. 271:22611-22618 (1996)).

### 6. Human Antibodies

In certain aspects, an antibody provided herein is a human antibody. In other aspects, a fusion protein provided herein comprises a human antibody or a fragment of a human antibody. Human antibodies can be produced using various techniques known in the art. Human antibodies are described generally in van Dijk and van de Winkel, Curr. Opin. Pharmacol. 5:368-74 (2001) and Lonberg, Curr. Opin. Immunol. 20:450-459 (2008).

Human antibodies may be prepared by administering an immunogen to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or which are present extrachromosomally or integrated randomly into the animal's chromosomes. In such transgenic mice, the endogenous immunoglobulin loci have generally been inactivated. For review of methods for obtaining human antibodies from transgenic animals, see Lonberg, Nat. Biotech. 23:1117-1125 (2005). See also, e.g., U.S. Pat. Nos. 6,075,181 and 6,150,584 describing XENOMOUSE™ technology; U.S. Pat. No. 5,770,429 describing HUMAB® technology; U.S. Pat. No. 7,041,870 describing K-M MOUSE® technology, and U.S. Patent Application Publication No. US 2007/0061900, describing VELOCIMOUSE® technology). Human variable regions from intact antibodies generated by such animals may be further modified, e.g., by combining with a different human constant region.

Human antibodies can also be made by hybridoma-based methods. Human myeloma and mouse-human heteromyeloma cell lines for the production of human monoclonal antibodies have been described. (See, e.g., Kozbor J. Immunol., 133:3001 (1984); Brodeur et al., Monoclonal Antibody Production Techniques and Applications, pp. 51-63 (Marcel Dekker, Inc., New York, 1987); and Boerner et al., J. Immunol., 147:86 (1991).) Human antibodies generated via human B-cell hybridoma technology are also described in Li et al., Proc. Natl. Acad. Sci. USA, 103:3557-3562 (2006). Additional methods include those described, for example, in U.S. Pat. No. 7,189,826 (describing production of monoclonal human IgM antibodies from hybridoma cell lines) and Ni, Xiandai Mianyixue, 26 (4): 265-268 (2006) (describing human-human hybridomas). Human hybridoma technology (Trioma technology) is also described in Vollmers and Brandlein, Histology and Histopathology, 20 (3): 927-937 (2005) and Vollmers and Brandlein, Methods and Findings in Experimental and Clinical Pharmacology, 27 (3): 185-91 (2005).

Human antibodies may also be generated by isolating variable domain sequences selected from human-derived phage display libraries. Such variable domain sequences may then be combined with a desired human constant domain. Techniques for selecting human antibodies from antibody libraries are described below.

### 7. Library-Derived Antibodies

In certain aspects, an antibody or antigen-binding domain as described herein is derived from a library. In other aspects, a fusion protein provided herein comprises an antibody, in particular an antibody fragment, such as a Fab or a DutaFab derived from a library. Antibodies of the invention may be isolated by screening combinatorial libraries for antibodies with the desired activity or activities. Methods for screening combinatorial libraries are reviewed, e.g., in Lerner et al. in Nature Reviews 16:498-508 (2016). For example, a variety of methods is known in the art for generating phage display libraries and screening such libraries for antibodies possessing the desired binding characteristics. Such methods are reviewed, e.g., in Frenzel et al. in mAbs 8:1177-1194 (2016); Bazan et al. in Human Vaccines and Immunotherapeutics 8:1817-1828 (2012) and Zhao et al. in Critical Reviews in Biotechnology 36:276-289 (2016) as well as in Hoogenboom et al. in Methods in Molecular Biology 178:1-37 (O'Brien et al., ed., Human Press, Totowa, NJ, 2001) and in Marks and Bradbury in Methods in Molecular Biology 248:161-175 (Lo, ed., Human Press, Totowa, NJ, 2003).

In certain phage display methods, repertoires of VH and VL genes are separately cloned by polymerase chain reaction (PCR) and recombined randomly in phage libraries, which can then be screened for antigen-binding phage as described in Winter et al. in Annual Review of Immunology 12:433-455 (1994). Phage typically display antibody fragments, either as single-chain Fv (scFv) fragments or as Fab fragments. Libraries from immunized sources provide high-affinity antibodies to the immunogen without the requirement of constructing hybridomas. Alternatively, the naive repertoire can be cloned (e.g., from human) to provide a single source of antibodies to a wide range of non-self and also self-antigens without any immunization as described by Griffiths et al. in EMBO Journal 12:725-734 (1993). Furthermore, naive libraries can also be made synthetically by cloning unrearranged V-gene segments from stem cells, and using PCR primers containing random sequence to encode the highly variable CDR3 regions and to accomplish rearrangement in vitro, as described by Hoogenboom and Winter in Journal of Molecular Biology 227:381-388 (1992). Patent publications describing human antibody phage libraries include, for example: U.S. Pat. Nos. 5,750,373; 7,985,840; 7,785,903 and 8,679,490 as well as US Patent Publication Nos. 2005/0079574, 2007/0117126, 2007/0237764 and 2007/0292936.

Further examples of methods known in the art for screening combinatorial libraries for antibodies with a desired activity or activities include ribosome and mRNA display, as well as methods for antibody display and selection on bacteria, mammalian cells, insect cells or yeast cells. Methods for yeast surface display are reviewed, e.g., in Scholler et al. in Methods in Molecular Biology 503:135-56 (2012) and in Cherf et al. in Methods in Molecular biology 1319:155-175 (2015) as well as in Zhao et al. in Methods in Molecular Biology 889:73-84 (2012). Methods for ribosome display are described, e.g., in He et al. in Nucleic Acids Research 25:5132-5134 (1997) and in Hanes et al. in PNAS 94:4937-4942 (1997).

Antibodies or antibody fragments isolated from human antibody libraries are considered human antibodies or human antibody fragments herein.

### 8. Multispecific Antibodies

In certain aspects, an antibody provided herein is a multispecific antibody, e.g., a bispecific antibody. “Multispecific antibodies” are monoclonal antibodies that have binding specificities for at least two different sites, i.e., different epitopes on different antigens or different epitopes on the same antigen. In certain aspects, the multispecific antibody has three or more binding specificities. In certain aspects, one of the binding specificities is for human PD-L1 and the other specificity is for any other antigen. In certain aspects, bispecific antibodies may bind to two (or more) different epitopes of human PD-L1. Multispecific (e.g., bispecific) antibodies may also be used to localize cytotoxic agents or cells to cells which express human PD-L1. Multispecific antibodies may be prepared as full-length antibodies or antibody fragments.

Techniques for making multispecific antibodies include, but are not limited to, recombinant co-expression of two immunoglobulin heavy chain-light chain pairs having different specificities (see Milstein and Cuello, Nature 305:537 (1983)) and “knob-in-hole” engineering (see, e.g., U.S. Pat. No. 5,731,168, and Atwell et al., J. Mol. Biol. 270:26 (1997)). Multi-specific antibodies may also be made by engineering electrostatic steering effects for making antibody Fc-heterodimeric molecules (see, e.g., WO 2009/089004); cross-linking two or more antibodies or fragments (see, e.g., U.S. Pat. No. 4,676,980, and Brennan et al., Science, 229:81 (1985)); using leucine zippers to produce bi-specific antibodies (see, e.g., Kostelny et al., J. Immunol., 148 (5): 1547-1553 (1992) and WO 2011/034605); using the common light chain technology for circumventing the light chain mis-pairing problem (see, e.g., WO 98/50431); using “diabody” technology for making bispecific antibody fragments (see, e.g., Hollinger et al., Proc. Natl. Acad. Sci. USA, 90:6444-6448 (1993)); and using single-chain Fv (scFv) dimers (see, e.g., Gruber et al., J. Immunol., 152:5368 (1994)); and preparing trispecific antibodies as described, e.g., in Tutt et al. J. Immunol. 147:60 (1991).

Engineered antibodies with three or more antigen binding sites, including for example, “Octopus antibodies”, or DVD-Ig are also included herein (see, e.g., WO 2001/77342 and WO 2008/024715). Other examples of multispecific antibodies with three or more antigen binding sites can be found in WO 2010/115589, WO 2010/112193, WO 2010/136172, WO 2010/145792, and WO 2013/026831. The bispecific antibody or antigen-binding fragment thereof also includes a “Dual Acting Fab” or “DAF” comprising an antigen-binding site that binds to human PD-L1 as well as another different antigen, or two different epitopes of human PD-L1 (see, e.g., US 2008/0069820 and WO 2015/095539).

Multi-specific antibodies may also be provided in an asymmetric form with a domain crossover in one or more binding arms of the same antigen specificity, i.e. by exchanging the VH/VL domains (see e.g., WO 2009/080252 and WO 2015/150447), the CH1/CL domains (see e.g., WO 2009/080253) or the complete Fab arms (see e.g., WO 2009/080251, WO 2016/016299, also see Schaefer et al, PNAS, 108 (2011) 1187-1191, and Klein at al., MAbs 8 (2016) 1010-20). In one aspect, the multispecific antibody comprises a CrossFab. The term “CrossFab” or “xFab” or “crossover Fab” refers to a Fab, wherein either the variable regions or the constant regions of the heavy and light chain arc exchanged. A CrossFab comprises a polypeptide chain composed of the light chain variable region (VL) and the heavy chain constant region 1 (CH1), and a polypeptide chain composed of the heavy chain variable region (VH) and the light chain constant region (CL). Asymmetrical Fab arms can also be engineered by introducing charged or non-charged amino acid mutations into domain interfaces to direct correct Fab pairing. See e.g., WO 2016/172485.

Various further molecular formats for multispecific antibodies are known in the art and are included herein (see e.g., Spiess et al., Mol Immunol 67 (2015) 95-106).

A particular type of multispecific antibodies, also included herein, are bispecific antibodies designed to simultaneously bind to a surface antigen on a target cell, e.g., a tumor cell and/or an immune cell in the tumor micro environment, and to an activating, invariant component of the T cell receptor (TCR) complex, such as CD3, for retargeting of T cells to kill target cells. Hence, in certain aspects, an antibody provided herein is a multispecific antibody, particularly a bispecific antibody, wherein one of the binding specificities is for human PD-L1 and the other is for CD3.

Examples of bispecific antibody formats that may be useful for this purpose include, but are not limited to, the so-called “BITE” (bispecific T cell engager) molecules wherein two scFv molecules are fused by a flexible linker (see, e.g., WO 2004/106381, WO 2005/061547, WO 2007/042261, and WO 2008/119567, Nagorsen and Bäuerle, Exp Cell Res 317, 1255-1260 (2011)); diabodies (Holliger et al., Prot Eng 9, 299-305 (1996)) and derivatives thereof, such as tandem diabodies (“TandAb”; Kipriyanov et al., J Mol Biol 293, 41-56 (1999)); “DART” (dual affinity retargeting) molecules which are based on the diabody format but feature a C-terminal disulfide bridge for additional stabilization (Johnson et al., J Mol Biol 399, 436-449 (2010)), and so-called triomabs, which are whole hybrid mouse/rat IgG molecules (reviewed in Seimetz et al., Cancer Treat Rev 36, 458-467 (2010)). Particular T cell bispecific antibody formats included herein are described in WO 2013/026833, WO 2013/026839, WO 2016/020309; Bacac et al., Oncoimmunology 5 (8) (2016) c1203498.

### 9. Antibody Variants

In certain aspects, amino acid sequence variants of the antibodies and antigen-binding domains provided herein are contemplated. For example, it may be desirable to alter the binding affinity and/or other biological properties of the antibody. Amino acid sequence variants of an antibody may be prepared by introducing appropriate modifications into the nucleotide sequence encoding the antibody, or by peptide synthesis. Such modifications include, for example, deletions from, and/or insertions into and/or substitutions of residues within the amino acid sequences of the antibody. Any combination of deletion, insertion, and substitution can be made to arrive at the final construct, provided that the final construct possesses the desired characteristics, e.g., antigen binding.

### a) Substitution, Insertion, and Deletion Variants

In certain aspects, antibody variants and variants of antigen-binding domains having one or more amino acid substitutions are provided. Sites of interest for substitutional mutagenesis include the CDRs and FRs. Conservative substitutions are shown in Table 1 under the heading of “preferred substitutions”. More substantial changes are provided in Table 1 under the heading of “exemplary substitutions”, and as further described below in reference to amino acid side chain classes. Amino acid substitutions may be introduced into an antibody of interest and the products screened for a desired activity, e.g., retained/improved antigen binding, decreased immunogenicity, or improved ADCC or CDC.

TABLE 1

| Original | Exemplary | Preferred |
|---|---|---|
| Residue | Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; Norleucine | Leu |
| Leu (L) | Norleucine; Ile; Val; Met; Ala; Phe | Ile |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; Norleucine | Leu |

Amino acids may be grouped according to common side-chain properties:
(1) hydrophobic: Norleucine, Met, Ala, Val, Leu, Ile;
(2) neutral hydrophilic: Cys, Scr, Thr, Asn, Gln;
(3) acidic: Asp, Glu;
(4) basic: His, Lys, Arg;
(5) residues that influence chain orientation: Gly, Pro;
(6) aromatic: Trp, Tyr, Phe.

Non-conservative substitutions will entail exchanging a member of one of these classes for a member of another class.

One type of substitutional variant involves substituting one or more hypervariable region residues of a parent antibody (e.g., a humanized or human antibody). Generally, the resulting variant(s) selected for further study will have modifications (e.g., improvements) in certain biological properties (e.g., increased affinity, reduced immunogenicity) relative to the parent antibody and/or will have substantially retained certain biological properties of the parent antibody. An exemplary substitutional variant is an affinity-matured antibody, which may be conveniently generated, e.g., using phage display-based affinity maturation techniques such as those described herein. Briefly, one or more. CDR residues are mutated and the variant antibodies displayed on phage and screened for a particular biological activity (e.g., binding affinity).

Alterations (e.g., substitutions) may be made in CDRs, e.g., to improve antibody affinity. Such alterations may be made in CDR “hotspots”, i.e., residues encoded by codons that undergo mutation at high frequency during the somatic maturation process (see, e.g., Chowdhury, Methods Mol. Biol. 207:179-196 (2008)), and/or residues that contact antigen, with the resulting variant VH or VL being tested for binding affinity. Affinity maturation by constructing and reselecting from secondary libraries has been described, e.g., in Hoogenboom et al. in Methods in Molecular Biology 178:1-37 (O'Brien et al., ed., Human Press, Totowa, NJ, (2001).) In some aspects of affinity maturation, diversity is introduced into the variable genes chosen for maturation by any of a variety of methods (e.g., error-prone PCR, chain shuffling, or oligonucleotide-directed mutagenesis). A secondary library is then created. The library is then screened to identify any antibody variants with the desired affinity. Another method to introduce diversity involves CDR-directed approaches, in which several CDR residues (e.g., 4-6 residues at a time) are randomized. CDR residues involved in antigen binding may be specifically identified, e.g., using alanine scanning mutagenesis or modeling. CDR-H3 and CDR-L3 in particular are often targeted.

In certain aspects, substitutions, insertions, or deletions may occur within one or more CDRs so long as such alterations do not substantially reduce the ability of the antibody to bind antigen. For example, conservative alterations (e.g., conservative substitutions as provided herein) that do not substantially reduce binding affinity may be made in the CDRs. Such alterations may, for example, be outside of antigen contacting residues in the CDRs. In certain variant VH and VL sequences provided above, each CDR either is unaltered, or contains no more than one, two or three amino acid substitutions.

A useful method for identification of residues or regions of an antibody or antigen-binding domain that may be targeted for mutagenesis is called “alanine scanning mutagenesis” as described by Cunningham and Wells (1989) Science, 244:1081-1085. In this method, a residue or group of target residues (e.g., charged residues such as Arg, Asp, His, Lys, and Glu) are identified and replaced by a neutral or negatively charged amino acid (e.g., alanine or polyalanine) to determine whether the interaction of the antibody with antigen is affected. Further substitutions may be introduced at the amino acid locations demonstrating functional sensitivity to the initial substitutions. Alternatively, or additionally, a crystal structure of an antigen-antibody complex may be used to identify contact points between the antibody and antigen. Such contact residues and neighboring residues may be targeted or eliminated as candidates for substitution. Variants may be screened to determine whether they contain the desired properties.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue. Other insertional variants of the antibody molecule include the fusion to the N- or C-terminus of the antibody to an enzyme (e.g., for ADEPT (antibody directed enzyme prodrug therapy)) or a polypeptide which increases the serum half-life of the antibody.

### b) Glycosylation Variants

In certain aspects, a fusion protein or antibody provided herein is altered to increase or decrease the extent to which the antibody is glycosylated. Addition or deletion of glycosylation sites to an antibody may be conveniently accomplished by altering the amino acid sequence such that one or more glycosylation sites is created or removed.

Where the fusion protein or antibody comprises an Fc region, the oligosaccharide attached thereto may be altered. Native antibodies produced by mammalian cells typically comprise a branched, biantennary oligosaccharide that is generally attached by an N-linkage to Asn297 of the CH2 domain of the Fc region. See, e.g., Wright et al. TIBTECH 15:26-32 (1997). The oligosaccharide may include various carbohydrates, e.g., mannose, N-acetyl glucosamine (GlcNAc), galactose, and sialic acid, as well as a fucose attached to a GlcNAc in the “stem” of the biantennary oligosaccharide structure. In some aspects, modifications of the oligosaccharide in an antibody of the invention may be made in order to create antibody variants with certain improved properties.

In one aspect, antibody and fusion protein variants are provided having a non-fucosylated oligosaccharide, i.e. an oligosaccharide structure that lacks fucose attached (directly or indirectly) to an Fc region. Such non-fucosylated oligosaccharide (also referred to as “afucosylated” oligosaccharide) particularly is an N-linked oligosaccharide which lacks a fucose residue attached to the first GlcNAc in the stem of the biantennary oligosaccharide structure. In one aspect, antibody variants are provided having an increased proportion of non-fucosylated oligosaccharides in the Fc region as compared to a native or parent antibody. For example, the proportion of non-fucosylated oligosaccharides may be at least about 20%, at least about 40%, at least about 60%, at least about 80%, or even about 100% (i.e. no fucosylated oligosaccharides are present). The percentage of non-fucosylated oligosaccharides is the (average) amount of oligosaccharides lacking fucose residues, relative to the sum of all oligosaccharides attached to Asn 297 (e. g. complex, hybrid and high mannose structures) as measured by MALDI-TOF mass spectrometry, as described in WO 2006/082515, for example. Asn297 refers to the asparagine residue located at about position 297 in the Fc region (EU numbering of Fc region residues); however, Asn297 may also be located about ±3 amino acids upstream or downstream of position 297, i.e., between positions 294 and 300, due to minor sequence variations in antibodies. Such antibodies having an increased proportion of non-fucosylated oligosaccharides in the Fc region may have improved FcγRIIIa receptor binding and/or improved effector function, in particular improved ADCC function. Sec, e.g., US 2003/0157108; US 2004/0093621.

Examples of cell lines capable of producing antibodies with reduced fucosylation include Lec13 CHO cells deficient in protein fucosylation (Ripka et al. Arch. Biochem. Biophys. 249:533-545 (1986); US 2003/0157108; and WO 2004/056312, especially at Example 11), and knockout cell lines, such as alpha-1,6-fucosyltransferase gene, FUT8, knockout CHO cells (see, e.g., Yamane-Ohnuki et al. Biotech. Bioeng. 87:614-622 (2004); Kanda, Y. et al., Biotechnol. Bioeng., 94 (4): 680-688 (2006); and WO 2003/085107), or cells with reduced or abolished activity of a GDP-fucose synthesis or transporter protein (see, e.g., US2004259150, US2005031613, US2004132140, US2004110282).

In a further aspect, antibody variants are provided with bisected oligosaccharides, e.g., in which a biantennary oligosaccharide attached to the Fc region of the antibody is bisected by GlcNAc. Such antibody variants may have reduced fucosylation and/or improved ADCC function as described above. Examples of such antibody variants are described, e.g., in Umana et al., Nat Biotechnol 17, 176-180 (1999); Ferrara et al., Biotechn Bioeng 93, 851-861 (2006); WO 99/54342; WO 2004/065540, WO 2003/011878.

Antibody variants with at least one galactose residue in the oligosaccharide attached to the Fc region are also provided. Such antibody variants may have improved CDC function. Such antibody variants are described, e.g., in WO 1997/30087; WO 1998/58964; and WO 1999/22764.

### c) Fc Region Variants

In certain aspects, one or more amino acid modifications may be introduced into the Fc region of a fusion protein or antibody provided herein, thereby generating an Fc region variant. The Fc region variant may comprise a human Fc region sequence (e.g., a human IgG₁, IgG₂, IgG₃ or IgG₄ Fc region) comprising an amino acid modification (e.g., a substitution) at one or more amino acid positions.

In certain aspects, the invention contemplates a fusion protein or antibody variant that possesses some but not all effector functions, which make it a desirable candidate for applications in which the half-life of the antibody in vivo is important yet certain effector functions (such as complement-dependent cytotoxicity (CDC) and antibody-dependent cell-mediated cytotoxicity (ADCC)) are unnecessary or deleterious. In vitro and/or in vivo cytotoxicity assays can be conducted to confirm the reduction/depletion of CDC and/or ADCC activities. For example, Fc receptor (FcR) binding assays can be conducted to ensure that the antibody lacks FcγR binding (hence likely lacking ADCC activity), but retains FcRn binding ability. The primary cells for mediating ADCC, NK cells, express FcγRIII only, whereas monocytes express FcγRI, FcγRII and FcγRIII. FcR expression on hematopoietic cells is summarized in Table 3 on page 464 of Ravetch and Kinet, Annu. Rev. Immunol. 9:457-492 (1991). Non-limiting examples of in vitro assays to assess ADCC activity of a molecule of interest is described in U.S. Pat. No. 5,500,362 (see, e.g., Hellstrom, I. et al. Proc. Nat'l Acad. Sci. USA 83:7059-7063 (1986)) and Hellstrom, I et al., Proc. Nat'l Acad. Sci. USA 82:1499-1502 (1985); 5,821,337 (see Bruggemann, M. et al., J. Exp. Med. 166:1351-1361 (1987)). Alternatively, non-radioactive assays methods may be employed (see, for example, ACTI™ non-radioactive cytotoxicity assay for flow cytometry (CellTechnology, Inc. Mountain View, CA; and CytoTox 96® non-radioactive cytotoxicity assay (Promega, Madison, WI). Useful effector cells for such assays include peripheral blood mononuclear cells (PBMC) and Natural Killer (NK) cells. Alternatively, or additionally, ADCC activity of the molecule of interest may be assessed in vivo, e.g., in an animal model such as that disclosed in Clynes et al. Proc. Nat'l Acad. Sci. USA 95:652-656 (1998). C1q binding assays may also be carried out to confirm that the antibody is unable to bind C1q and hence lacks CDC activity. See, e.g., C1q and C3c binding ELISA in WO 2006/029879 and WO 2005/100402. To assess complement activation, a CDC assay may be performed (see, for example, Gazzano-Santoro et al., J. Immunol. Methods 202:163 (1996); Cragg, M. S. et al., Blood 101:1045-1052 (2003); and Cragg, M. S. and M. J. Glennie, Blood 103:2738-2743 (2004)). FcRn binding and in vivo clearance/half-life determinations can also be performed using methods known in the art (see, e.g., Petkova, S. B. et al., Int'l. Immunol. 18 (12): 1759-1769 (2006); WO 2013/120929 A1).

Antibodies or fusion proteins with reduced effector function include those with substitution of one or more of Fc region residues 238, 265, 269, 270, 297, 327 and 329 (U.S. Pat. No. 6,737,056). Such Fc mutants include Fc mutants with substitutions at two or more of amino acid positions 265, 269, 270, 297 and 327, including the so-called “DANA” Fc mutant with substitution of residues 265 and 297 to alanine (U.S. Pat. No. 7,332,581).

Certain antibody variants with improved or diminished binding to FcRs are described. (Sec, e.g., U.S. Pat. No. 6,737,056; WO 2004/056312, and Shields et al., J. Biol. Chem. 9 (2): 6591-6604 (2001).)

In certain aspects, a fusion protein or antibody variant comprises an Fc region with one or more amino acid substitutions which improve ADCC, e.g., substitutions at positions 298, 333, and/or 334 of the Fc region (EU numbering of residues).

In certain aspects, a fusion protein or antibody variant comprises an Fc region with one or more amino acid substitutions which diminish FcγR binding, e.g., substitutions at positions 234 and 235 of the Fc region (EU numbering of residues). In one aspect, the substitutions are L234A and L235A (LALA). In certain aspects, the antibody variant further comprises D265A and/or P329G in an Fc region derived from a human IgG₁ Fc region. In one aspect, the substitutions are L234A, L235A and P329G (LALA-PG) in an Fc region derived from a human IgG₁ Fc region. (See, e.g., WO 2012/130831). In another aspect, the substitutions are L234A, L235A and D265A (LALA-DA) in an Fc region derived from a human IgG₁ Fc region.

In some aspects, alterations are made in the Fc region that result in altered (i.e., either improved or diminished) C1q binding and/or Complement Dependent Cytotoxicity (CDC), e.g., as described in U.S. Pat. No. 6,194,551, WO 99/51642, and Idusogie et al. J. Immunol. 164:4178-4184 (2000).

Antibodies with increased half-lives and improved binding to the neonatal Fc receptor (FcRn), which is responsible for the transfer of maternal IgGs to the fetus (Guyer et al., J. Immunol. 117:587 (1976) and Kim et al., J. Immunol. 24:249 (1994)), are described in US2005/0014934 (Hinton et al.). Those antibodies comprise an Fc region with one or more substitutions therein which improve binding of the Fc region to FcRn. Such Fc variants include those with substitutions at one or more of Fc region residues: 238, 252, 254, 256, 265, 272, 286, 303, 305, 307, 311, 312, 317, 340, 356, 360, 362, 376, 378, 380, 382, 413, 424 or 434, e.g., substitution of Fc region residue 434 (See, e.g., U.S. Pat. No. 7,371,826; Dall'Acqua, W. F., et al. J. Biol. Chem. 281 (2006) 23514-23524).

Fc region residues critical to the mouse Fc-mouse FcRn interaction have been identified by site-directed mutagenesis (see e.g. Dall'Acqua, W. F., et al. J. Immunol 169 (2002) 5171-5180). Residues 1253, H310, H433, N434, and H435 (EU numbering of residues) are involved in the interaction (Medesan, C., et al., Eur. J. Immunol. 26 (1996) 2533; Firan, M., et al., Int. Immunol. 13 (2001) 993; Kim, J. K., et al., Eur. J. Immunol. 24 (1994) 542). Residues 1253, H310, and H435 were found to be critical for the interaction of human Fc with murine FcRn (Kim, J. K., et al., Eur. J. Immunol. 29 (1999) 2819). Studies of the human Fc-human FcRn complex have shown that residues 1253, S254, H435, and Y436 are crucial for the interaction (Firan, M., et al., Int. Immunol. 13 (2001) 993; Shields, R. L., et al., J. Biol. Chem. 276 (2001) 6591-6604). In Yeung, Y. A., et al. (J. Immunol. 182 (2009) 7667-7671) various mutants of residues 248 to 259 and 301 to 317 and 376 to 382 and 424 to 437 have been reported and examined.

In certain aspects, a fusion protein or antibody variant comprises an Fc region with one or more amino acid substitutions, which reduce FcRn binding, e.g., substitutions at positions 253, and/or 310, and/or 435 of the Fc-region (EU numbering of residues). In certain aspects, the fusion protein or antibody variant comprises an Fc region with the amino acid substitutions at positions 253, 310 and 435. In one aspect, the substitutions are I253A, H310A and H435A in an Fc region derived from a human IgG1 Fc-region. Sec, e.g., Grevys, A., et al., J. Immunol. 194 (2015) 5497-5508.

In certain aspects, a fusion protein or antibody variant comprises an Fc region with one or more amino acid substitutions, which reduce FcRn binding, e.g., substitutions at positions 310, and/or 433, and/or 436 of the Fc region (EU numbering of residues). In certain aspects, the antibody variant comprises an Fc region with the amino acid substitutions at positions 310, 433 and 436. In one aspect, the substitutions are H310A, H433A and Y436A in an Fc region derived from a human IgG1 Fc-region. (Sec, e.g., WO 2014/177460 A1).

In certain aspects, a fusion protein or antibody variant comprises an Fc region with one or more amino acid substitutions which increase FcRn binding, e.g., substitutions at positions 252, and/or 254, and/or 256 of the Fc region (EU numbering of residues). In certain aspects, the fusion protein or antibody variant comprises an Fc region with amino acid substitutions at positions 252, 254, and 256. In one aspect, the substitutions are M252Y, S254T and T256E in an Fc region derived from a human IgG1 Fc-region. See also Duncan & Winter, Nature 322:738-40 (1988); U.S. Pat. Nos. 5,648,260; 5,624,821; and WO 94/29351 concerning other examples of Fc region variants.

In certain aspects, the invention further contemplates fusion proteins or antibody variants that comprise different antigen binding moieties, which may be fused to one or the other of the two subunits of the Fc domain, so that the two subunits of the Fc domain will typically be comprised in two non-identical polypeptide chains. Recombinant co-expression of these polypeptides and subsequent dimerization may lead to several possible combinations of the two polypeptides. To improve the yield and purity of fusion proteins or antibody variants in recombinant production, it will thus be advantageous to introduce a modification in the Fc domain of the respective fusion protein or antibody variant which promotes the association of the desired polypeptides.

Accordingly, in particular aspects, the Fc domain of the fusion protein or antibody variant described herein comprises a modification promoting the association of the first and the second subunit of the Fc domain. The site of most extensive protein-protein interaction between the two subunits of a human IgG Fc domain is in the CH3 domain of the Fc domain. Thus, in one aspect said modification is in the CH3 domain of the Fc domain.

Several approaches for modifications in the CH3 domain of the Fc domain in order to enforce heterodimerization exist, which are well described e.g. in WO 96/27011, WO 98/050431, EP 1870459, WO 2007/110205, WO 2007/147901, WO 2009/089004, WO 2010/129304, WO 2011/90754, WO 2011/143545, WO 2012058768, WO 2013157954, WO 2013096291. Typically, in all such approaches the CH3 domain of the first subunit of the Fc domain and the CH3 domain of the second subunit of the Fc domain are both engineered in a complementary manner so that each CH3 domain (or the heavy chain comprising it) can no longer homodimerize with itself but is forced to heterodimerize with the complementarily engineered other CH3 domain (so that the first and second CH3 domain heterodimerize and no homodimers between the two first or the two second CH3 domains are formed). These different approaches for improved heavy chain heterodimerization are contemplated as different alternatives in combination with the heavy-light chain modifications (e.g. VH and VL exchange/replacement in one binding arm and the introduction of substitutions of charged amino acids with opposite charges in the CH1/CL interface) in the fusion protein or antibody variant described herein which reduce heavy/light chain mispairing and Bence Jones-type side products.

In a certain aspect, said modification promoting the association of the first and the second subunit of the Fc domain is a so-called “knob-into-hole” modification, comprising a “knob” modification in one of the two subunits of the Fc domain and a “hole” modification in the other one of the two subunits of the Fc domain.

The knob-into-hole technology is described e.g. in U.S. Pat. Nos. 5,731,168; 7,695,936; Ridgway et al., Prot Eng 9, 617-621 (1996) and Carter, J Immunol Meth 248, 7-15 (2001). Generally, the method involves introducing a protuberance (“knob”) at the interface of a first polypeptide and a corresponding cavity (“hole”) in the interface of a second polypeptide, such that the protuberance can be positioned in the cavity so as to promote heterodimer formation and hinder homodimer formation. Protuberances are constructed by replacing small amino acid side chains from the interface of the first polypeptide with larger side chains (e.g. tyrosine or tryptophan). Compensatory cavities of identical or similar size to the protuberances are created in the interface of the second polypeptide by replacing large amino acid side chains with smaller ones (e.g. alanine or threonine).

Accordingly, in certain aspects, in the CH3 domain of one subunit of the Fc domain of the fusion protein or antibody variant described herein, an amino acid residue is replaced with an amino acid residue having a larger side chain volume, thereby generating a protuberance within the CH3 domain of this subunit which is positionable in a cavity within the CH3 domain of the other subunit, and in the CH3 domain of the other subunit of the Fc domain an amino acid residue is replaced with an amino acid residue having a smaller side chain volume, thereby generating a cavity within the CH3 domain of this subunit within which the protuberance within the CH3 domain of the first subunit is positionable.

In one aspect, said amino acid residue having a larger side chain volume is selected from the group consisting of arginine (R), phenylalanine (F), tyrosine (Y), and tryptophan (W). In one aspect, said amino acid residue having a smaller side chain volume is selected from the group consisting of alanine (A), serine(S), threonine (T), and valine (V). The protuberance and cavity can be made by altering the nucleic acid encoding the polypeptides, e.g. by site-specific mutagenesis, or by peptide synthesis.

In certain aspects, in (the CH3 domain of) the first subunit of the Fc domain (the “hole” subunit) of the fusion proteins described herein, the tyrosine residue at position 407 is replaced with a valine residue (Y407V), and in (the CH3 domain of) the second subunit of the Fc domain (the “knobs” subunit) the threonine residue at position 366 is replaced with a tryptophan residue (T366W).

In one aspect, in the first subunit of the Fc domain additionally the threonine residue at position 366 is replaced with a serine residue (T366S) and the leucine residue at position 368 is replaced with an alanine residue (L368A) (numberings according to Kabat EU index).

In certain aspects, in the first subunit of the Fc domain additionally the tyrosine residue at position 349 is replaced by a cysteine residue (Y349C) (numberings according to Kabat EU index), and in the second subunit of the Fc domain additionally the serine residue at position 354 is replaced with a cysteine residue (S354C) or the glutamic acid residue at position 356 is replaced with a cysteine residue (E356C) (particularly the serine residue at position 354 is replaced with a cysteine residue). Introduction of these two cysteine residues results in formation of a disulfide bridge between the two subunits of the Fc domain, further stabilizing the dimer (Carter, J Immunol Methods 248, 7-15 (2001)).

In certain aspects, the first subunit of the Fc domain comprises the amino acid substitutions Y349C, T366S, L368A and Y407V (numbering according to Kabat EU index), and the second subunit of the Fc domain comprises the amino acid substitutions S354C and T366W. In certain aspects, the second antigen binding domain that is capable of binding to human PD-L1 and to human IFN-α2 is fused (either directly or via a peptidic linker) to the second subunit of the Fc domain comprising the “knob” modification.

Other techniques of CH3-modification for enforcing the heterodimerization are contemplated as alternatives according to the invention and are described e.g. in WO 96/27011, WO 98/050431, EP 1870459, WO 2007/110205, WO 2007/147901, WO 2009/089004, WO 2010/129304, WO 2011/90754, WO 2011/143545, WO 2012/058768, WO 2013/157954, WO 2013/096291.

In one aspect, the heterodimerization approach described in EP 1870459, is used. This approach is based on the introduction of charged amino acids with opposite charges at specific amino acid positions in the CH3/CH3 domain interface between the two subunits of the Fc domain. One particular aspect of the fusion protein or antibody variant described herein are amino acid mutations R409D; K370E in one of the two CH3 domains (of the Fc domain) and amino acid mutations D399K; E357K in the other one of the CH3 domains of the Fc domain (numbering according to Kabat EU index).

In another aspect, the fusion protein or antibody variant described herein comprises the amino acid mutations T366S, L368A, Y407V in the CH3 domain of the first subunit of the Fc domain and the amino acid mutation T366W in the CH3 domain of the second subunit of the Fc domain, and additionally amino acid mutations D399K; E357K in the CH3 domain of the first subunit of the Fc domain and amino acid mutations R409D; K370E in the CH3 domain of the second subunit of the Fc domain (numberings according to Kabat EU index).

In certain aspects, the fusion protein or antibody variant described herein comprises amino acid mutations Y349C, T366S, L368A, Y407V in the CH3 domain of the first subunit of the Fc domain and amino acid mutations S354C, T366W in the CH3 domain of the second subunit of the Fc domain, or said fusion protein or antibody variant described herein comprises amino acid mutations S354C, T366S, L368A, Y407V in the CH3 domain of the first subunit of the Fc domain and amino acid mutations Y349C, T366W in the CH3 domains of the second subunit of the Fc domain and additionally amino acid mutations D399K; E357K in the CH3 domain of the first subunit of the Fc domain and amino acid mutations R409D; K370E in the CH3 domain of the second subunit of the Fc domain (all numberings according to Kabat EU index).

In one aspect, the heterodimerization approach described in WO 2013/157953 is used alternatively. In one aspect, one CH3 domain comprises amino acid mutation T366K and the other CH3 domain comprises amino acid mutation L351D (numberings according to Kabat EU index). In a further aspect, the former CH3 domain comprises further amino acid mutation L351K, and/or the latter CH3 domain comprises further an amino acid mutation selected from Y349E, Y349D and L368E (particularly L368E) (numberings according to Kabat EU index).

In one aspect, the heterodimerization approach described in WO 2012/058768 is used alternatively. In one aspect, a first CH3 domain comprises amino acid mutations L351Y, Y407A and a second CH3 domain comprises amino acid mutations T366A, K409F. In a further aspect the second CH3 domain comprises a further amino acid mutation at position T411, D399, S400, F405, N390, or K392, e.g. selected from a) T41IN, T41IR, T411Q, T411K, T411D, T411E or T411W, b) D399R, D399W, D399Y or D399K, c) S400E, S400D, S400R, or S400K, d) F4051, F405M, F405T, F405S, F405V or F405W, c) N390R, N390K or N390D, f) K392V, K392M, K392R, K392L, K392F or K392E (numberings according to Kabat EU index). In a further aspect, a first CH3 domain comprises amino acid mutations L351Y, Y407A and a second CH3 domain comprises amino acid mutations T366V, K409F. In a further aspect, a first CH3 domain comprises amino acid mutation Y407A and a second CH3 domain comprises amino acid mutations T366A, K409F. In a further aspect, the second CH3 domain further comprises amino acid mutations K392E, T411E, D399R and S400R (numberings according to Kabat EU index).

In one aspect, the heterodimerization approach described in WO 2011/143545 is used alternatively, e.g. with the amino acid modification at a position selected from the group consisting of 368 and 409 (numbering according to Kabat EU index).

In one aspect, the heterodimerization approach described in WO 2011/090762, which also uses the knobs-into-holes technology described above, is used alternatively. In one aspect, a first CH3 domain comprises amino acid mutation T366W and a second CH3 domain comprises amino acid mutation Y407A. In one aspect, a first CH3 domain comprises amino acid mutation T366Y and a second CH3 domain comprises amino acid mutation Y407T (numberings according to Kabat EU index).

In one aspect, the fusion protein or antibody variant described herein or its Fc domain is of IgG2 subclass and the heterodimerization approach described in WO 2010/129304 is used alternatively.

In an alternative aspect, a modification promoting association of the first and the second subunit of the Fc domain comprises a modification mediating electrostatic steering effects, e.g. as described in PCT publication WO 2009/089004. Generally, this method involves replacement of one or more amino acid residues at the interface of the two Fc domain subunits by charged amino acid residues so that homodimer formation becomes electrostatically unfavorable but heterodimerization electrostatically favorable. In one such aspect, a first CH3 domain comprises amino acid substitution of K392 or N392 with a negatively charged amino acid (e.g. glutamic acid (E), or aspartic acid (D), particularly K392D or N392D) and a second CH3 domain comprises amino acid substitution of D399, E356, D356, or E357 with a positively charged amino acid (e.g. lysine (K) or arginine (R), particularly D399K, E356K, D356K, or E357K, and more particularly D399K and E356K). In a further aspect, the first CH3 domain further comprises amino acid substitution of K409 or R409 with a negatively charged amino acid (e.g. glutamic acid (E), or aspartic acid (D), particularly K409D or R409D). In a further aspect the first CH3 domain further or alternatively comprises amino acid substitution of K439 and/or K370 with a negatively charged amino acid (e.g. glutamic acid (E), or aspartic acid (D)) (all numberings according to Kabat EU index).

In yet a further aspect, the heterodimerization approach described in WO 2007/147901 is used alternatively. In one aspect, a first CH3 domain comprises amino acid mutations K253E, D282K, and K322D and a second CH3 domain comprises amino acid mutations D239K, E240K, and K292D (numberings according to Kabat EU index).

In still another aspect, the heterodimerization approach described in WO 2007/110205 can be used alternatively.

In one aspect, the first subunit of the Fc domain comprises amino acid substitutions K392D and K409D, and the second subunit of the Fc domain comprises amino acid substitutions D356K and D399K (numbering according to Kabat EU index).

The C-terminus of the heavy chain of the antibody or fusion protein as reported herein can be a complete C-terminus ending with the amino acid residues PGK. The C-terminus of the heavy chain can be a shortened C-terminus in which one or two of the C terminal amino acid residues have been removed. In one preferred aspect, the C-terminus of the heavy chain is a shortened C-terminus ending PG. In one aspect of all aspects as reported herein, an antibody or fusion protein comprising a heavy chain including a C-terminal CH3 domain as specified herein, comprises the C-terminal glycine-lysine dipeptide (G446 and K447, EU index numbering of amino acid positions). In one aspect of all aspects as reported herein, an antibody comprising a heavy chain including a C-terminal CH3 domain, as specified herein, comprises a C-terminal glycine residue (G446, EU index numbering of amino acid positions).

### d) Cysteine Engineered Antibody Variants

In certain aspects, it may be desirable to create cysteine-engineered antibodies, e.g., THIOMAB™ antibodies, in which one or more residues of an antibody are substituted with cysteine residues. In particular aspects, the substituted residues occur at accessible sites of the antibody. By substituting those residues with cysteine, reactive thiol groups are thereby positioned at accessible sites of the antibody and may be used to conjugate the antibody to other moieties, such as drug moieties or linker-drug moieties, to create an immunoconjugate, as described further herein. Cysteine engineered antibodies may be generated as described, e.g., in U.S. Pat. Nos. 7,521,541, 8,30,930, 7,855,275, 9,000,130, or WO 2016040856.

### e) Antibody Derivatives

In certain aspects, a fusion protein or antibody provided herein may be further modified to contain additional nonproteinaceous moieties that are known in the art and readily available. The moieties suitable for derivatization of the antibody include but are not limited to water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1, 3-dioxolane, poly-1,3,6-trioxane, ethylene/maleic anhydride copolymer, polyaminoacids (either homopolymers or random copolymers), and dextran or poly(n-vinyl pyrrolidone) polyethylene glycol, propropylene glycol homopolymers, prolypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight, and may be branched or unbranched. The number of polymers attached to the antibody may vary, and if more than one polymer are attached, they can be the same or different molecules. In general, the number and/or type of polymers used for derivatization can be determined based on considerations including, but not limited to, the particular properties or functions of the antibody to be improved, whether the antibody derivative will be used in a therapy under defined conditions, etc.

### B. Recombinant Methods and Compositions

Antibodies or fusion proteins as described herein may be produced using recombinant methods and compositions, e.g., as described in U.S. Pat. No. 4,816,567. For these methods, one or more isolated nucleic acid(s) encoding an antibody or a fusion protein are provided.

In case of a native antibody or native antibody fragment two nucleic acids are required, one for the light chain or a fragment thereof and one for the heavy chain or a fragment thereof. Such nucleic acid(s) encode an amino acid sequence comprising the VL and/or an amino acid sequence comprising the VH of the antibody (e.g., the light and/or heavy chain(s) of the antibody). These nucleic acids can be on the same expression vector or on different expression vectors.

In case of a bispecific antibody with heterodimeric heavy chains four nucleic acids are required, one for the first light chain, one for the first heavy chain comprising the first heteromonomeric Fc-region polypeptide, one for the second light chain, and one for the second heavy chain comprising the second heteromonomeric Fc-region polypeptide. In case of a fusion protein as described herein, most commonly four nucleic acids are required, one for the first light chain, one for the first heavy chain comprising the first heteromonomeric Fc-region polypeptide, one for the second light chain comprising the IFN-α2, and one for the second heavy chain comprising the second heteromonomeric Fc-region polypeptide. The four nucleic acids can be comprised in one or more nucleic acid molecules or expression vectors. Such nucleic acid(s) encode an amino acid sequence comprising the first VL and/or an amino acid sequence comprising the first VH including the first heteromonomeric Fc-region and/or an amino acid sequence comprising the second VL and/or an amino acid sequence comprising the second VH including the second heteromonomeric Fc-region of the antibody (e.g., the first and/or second light and/or the first and/or second heavy chains of the antibody). These nucleic acids can be on the same expression vector or on different expression vectors, normally these nucleic acids are located on two or three expression vectors, i.e. one vector can comprise more than one of these nucleic acids. Examples of these bispecific antibodies are CrossMabs (see, e.g., Schaefer, W. et al, PNAS, 108 (2011) 11187-1191). For example, one of the heteromonomeric heavy chain comprises the so-called “knob mutations” (T366W and optionally one of S354C or Y349C) and the other comprises the so-called “hole mutations” (T366S, L368A and Y407V and optionally Y349C or S354C) (see, e.g., Carter, P. et al., Immunotechnol. 2 (1996) 73) according to EU index numbering.

In one aspect, isolated nucleic acids encoding an antibody or a fusion protein as used in the methods as reported herein are provided.

In one aspect, a method of making a PD-L1-targeted huIFN-α2a fusion protein as described herein is provided, wherein the method comprises culturing a host cell comprising nucleic acid(s) encoding the fusion protein, as provided above, under conditions suitable for expression of the fusion protein, and optionally recovering the fusion protein from the host cell (or host cell culture medium). In another aspect, a method of making an anti-huPD-L1 antibody is provided, wherein the method comprises culturing a host cell comprising nucleic acid(s) encoding the antibody, as provided above, under conditions suitable for expression of the antibody, and optionally recovering the antibody from the host cell (or host cell culture medium).

For recombinant production of a PD-L1-targeted huIFN-α2a fusion protein and/or an anti-PD-L1 antibody, nucleic acids encoding the fusion protein or antibody, respectively, e.g., as described above, are isolated and inserted into one or more vectors for further cloning and/or expression in a host cell. Such nucleic acids may be readily isolated and sequenced using conventional procedures (e.g., by using oligonucleotide probes that are capable of binding specifically to genes encoding the heavy and light chains of the fusion protein or antibody) or produced by recombinant methods or obtained by chemical synthesis.

Suitable host cells for cloning or expression of fusion protein- or antibody-encoding vectors include prokaryotic or eukaryotic cells described herein. For example, fusion proteins or antibodies may be produced in bacteria, in particular when glycosylation and Fc effector function are not needed. For expression of antibody fragments and polypeptides in bacteria, see, e.g., U.S. Pat. Nos. 5,648,237, 5,789,199, and 5,840,523. (See also Charlton, K. A., In: Methods in Molecular Biology, Vol. 248, Lo, B. K. C. (ed.), Humana Press, Totowa, NJ (2003), pp. 245-254, describing expression of antibody fragments in E. coli.) After expression, the fusion protein or antibody may be isolated from the bacterial cell paste in a soluble fraction and can be further purified.

In addition to prokaryotes, eukaryotic microbes such as filamentous fungi or yeast are suitable cloning or expression hosts for antibody-encoding vectors, including fungi and yeast strains whose glycosylation pathways have been “humanized”, resulting in the production of an antibody with a partially or fully human glycosylation pattern. Sec Gerngross, T. U., Nat. Biotech. 22 (2004) 1409-1414; and Li, H. et al., Nat. Biotech. 24 (2006) 210-215.

Suitable host cells for the expression of (glycosylated) antibody and/or fusion proteins are also derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells.

Plant cell cultures can also be utilized as hosts. See, e.g., U.S. Pat. Nos. 5,959,177, 6,040,498, 6,420,548, 7,125,978, and 6,417,429 (describing PLANTIBODIES™ technology for producing antibodies in transgenic plants).

Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293T cells as described, e.g., in Graham, F. L. et al., J. Gen Virol. 36 (1977) 59-74); baby hamster kidney cells (BHK); mouse sertoli cells (TM4 cells as described, e.g., in Mather, J. P., Biol. Reprod. 23 (1980) 243-252); monkey kidney cells (CV1); African green monkey kidney cells (VERO-76); human cervical carcinoma cells (HELA); canine kidney cells (MDCK; buffalo rat liver cells (BRL 3A); human lung cells (W138); human liver cells (Hep G2); mouse mammary tumor (MMT 060562); TRI cells (as described, e.g., in Mather, J. P. et al., Annals N.Y. Acad. Sci. 383 (1982) 44-68); MRC 5 cells; and FS4 cells. Other useful mammalian host cell lines include Chinese hamster ovary (CHO) cells, including DHFR-CHO cells (Urlaub, G. et al., Proc. Natl. Acad. Sci. USA 77 (1980) 4216-4220); and myeloma cell lines such as Y0, NS0 and Sp2/0. For a review of certain mammalian host cell lines suitable for antibody production, see, e.g., Yazaki, P. and Wu, A. M., Methods in Molecular Biology, Vol. 248, Lo, B. K. C. (ed.), Humana Press, Totowa, NJ (2004), pp. 255-268.

In one aspect, the host cell is eukaryotic, e.g., a Chinese Hamster Ovary (CHO) cell or lymphoid cell (e.g., Y0, NS0, Sp20 cell).

### C. Assays

Fusion proteins provided herein, antigen-binding domains for use in fusion proteins provided herein and anti-huPD-L1 antibodies provided herein may be identified, screened for, or characterized for their physical/chemical properties and/or biological activities by various assays known in the art.

### 1. Binding Assays and Other Assays

In one aspect, an antibody of the invention is tested for its antigen binding activity, e.g., by known methods such as ELISA, Western blot, etc.

In another aspect, competition assays may be used to identify an antibody that competes with Atezolizumab, Durvalumab, and Avelumab for binding to PD-L1. In certain aspects, such a competing antibody binds to the epitope (e.g., a linear or a conformational epitope) that is bound by Atezolizumab, Durvalumab and/or Avelumab. In another aspect, competition assays may be used to identify an antibody that does not compete with Atezolizumab, Durvalumab, and/or Avelumab for binding to PD-L1. In certain aspects, such a non-competing antibody binds to another epitope (e.g., a linear or a conformational epitope) than the one that is bound by Atezolizumab, Durvalumab, and Avelumab. Detailed exemplary methods for mapping an epitope to which an antibody binds are provided in Morris (1996) “Epitope Mapping Protocols”, in Methods in Molecular Biology vol. 66 (Humana Press, Totowa, NJ).

In an exemplary competition assay, immobilized PD-L1 is incubated in a solution comprising a first labeled antibody that binds to PD-L1 (e.g., Atezolizumab, BMS-936559, Avelumab, Durvalumab) and a second unlabeled antibody that is being tested for its ability to compete with the first antibody for binding to PD-L1. The second antibody may be present in a hybridoma supernatant. As a control, immobilized PD-L1 is incubated in a solution comprising the first labeled antibody but not the second unlabeled antibody. After incubation under conditions permissive for binding of the first antibody to PD-L1, excess unbound antibody is removed, and the amount of label associated with immobilized PD-L1 is measured. If the amount of label associated with immobilized PD-L1 is substantially reduced in the test sample relative to the control sample, then that indicates that the second antibody is competing with the first antibody for binding to PD-L1. See Harlow and Lane (1988) Antibodies: A Laboratory Manual ch.14 (Cold Spring Harbor Laboratory, Cold Spring Harbor, NY). Alternatively, competition can also be examined using an SPR assay as described in Example 7 c.

### 2. Activity Assays

In one aspect, assays are provided for identifying anti-huPD-L1 antibodies thereof having biological activity. Biological activity may include, e.g., the blocking of′PD-1/PD-L1 interaction, or internalization (into a cell) upon PD-L1 binding. Antibodies having such biological activity in vivo and/or in vitro are also provided.

In certain aspects, an antibody of the invention is tested for such biological activity. An exemplary assay to identify whether an antibody blocks the interaction PD-1 and PD-L1 may be the PD1/PD-L1 blockade bioassay (Promega).

In an exemplary assay to determine internalization of an anti-huPD-L1 antibody, live cell imaging can be used. For this, PD-L1 expressing cells are contacted with the anti-huPD-L1 antibodies and a reagent suitable for labeling the antibody with a detectable label, e.g. a fluorescence label. Internalization is then detected using live cell imaging.

In one aspect, assays are provided for identifying PD-L1-targeted huIFN-α2a fusion proteins thereof having biological activity. Biological activity may include, e.g., IFNα activity, tumor growth inhibition, induction of IFNα-mediated release of immunomodulating cytokines, PD-L1 and or MHCI upregulation, and/or secretion, such as IFNγ IL6, CXCL10 etc., and/or the ability to enhance the activation and/or proliferation of different immune cells, such as NK cells, DC cells or T cells. Antibodies having such biological activity in vivo and/or in vitro are also provided.

Such an activity assay may be an in vitro HEK-blue human IFNAR 1/2 reporter cell assay, as described in Examples 12 and 13. For this assay, a huPD-11 high-expressing HEKBlue IFNα/β reporter cell line is generated by transfecting HEK-Blue IFNα/β (Invivogen, Catalog number hkb-IFNα/B) cells with a vector carrying full-length cDNA encoding human PD-L1 and selecting cells with high cell surface expression of PD-L1 to establish stable cell clones. Quantification of the cell-surface PD-L1 is possible e.g. using a bead based Quantification kit (BD, Catalog number 340495). As second reporter cell line, the HekBlue-IFNα/β wildtype cells are used as second reporter cell line, which have low cell surface expression of PD-L1 (≈300 PD-L1 molecules/cell on cell surface). Self-regulated activity of the fusion proteins provided herein can be shown by contacting the two reporter cell lines independently with a fusion protein to be tested in a titration series of different concentrations. Fusion proteins can only activate IFNAR1/2 receptors when huPD-L1 is present in sufficient amount on the cell surface so that the PD-L1-binding side of the Dutaflip arm binds to the huPD-L1 on the cell surface, thus releasing IFN-α2a and setting it free to bind to its receptor on the cell surface. Each concentration of the fusion protein is contacted with an aliquot of the reporter cells in a suitable medium and incubated for a defined time span at 37° C. Upon binding of IFN-α2a to IFNAR1/2 receptors, the JAK/STAT/ISGF3 pathway is triggered, leading finally to the expression of the reporter gene which is under the control of the ISG54 promoter containing an IFN-stimulated response element (ISRE). The reporter gene SEAP (secreted alkaline phosphatase) is produced by the cell and secreted into the medium. Its amount correlates with the extent of IFNAR 1/2 receptor activation. The SEAP levels in the medium can be measured by using a SEAP detection reagent like QUANTI-Blue™ and the color change of the detection reagent by the SEAP activity can be measured with a spectrophotometer at the optical density (OD) at 640 nm.

In certain embodiments, a fusion protein or an antibody of the invention is tested for such biological activity as described e.g. in the examples below.

### D. Methods and Compositions for Diagnostics and Detection

In certain aspects, any of the anti-huPD-L1 antibodies provided herein is useful for detecting the presence of huPD-L1 in a biological sample. The term “detecting” as used herein encompasses quantitative or qualitative detection. In certain aspects, a biological sample comprises a cell or tissue, such as tumor tissue or tumor cells.

In one aspect, an anti-huPD-L1 antibody for use in a method of diagnosis or detection is provided. In a further aspect, a method of detecting the presence of huPD-L1 in a biological sample is provided. In certain aspects, the method comprises contacting the biological sample with an anti-huPD-L1 antibody as described herein under conditions permissive for binding of the anti-huPD-L1 antibody to human PD-L1, and detecting whether a complex is formed between the anti-huPD-L1 antibody and human PD-L1. Such method may be an in vitro or in vivo method. In one aspect, an anti-huPD-L1 antibody is used to select subjects eligible for therapy with an anti-huPD-L1 antibody, e.g., where human PD-L1 is a biomarker for selection of patients.

In certain aspects, labeled anti-huPD-L1 antibodies are provided. Labels include, but are not limited to, labels or moieties that are detected directly (such as fluorescent, chromophoric, electron-dense, chemiluminescent, and radioactive labels), as well as moieties, such as enzymes or ligands, that are detected indirectly, e.g., through an enzymatic reaction or molecular interaction. Exemplary labels include, but are not limited to, the radioisotopes ³²P, ¹⁴C, ¹²⁵I, ³H, and ¹³¹I, fluorophores such as rare earth chelates or fluorescein and its derivatives, rhodamine and its derivatives, dansyl, umbelliferone, luceriferases, e.g., firefly luciferase and bacterial luciferase (U.S. Pat. No. 4,737,456), luciferin, 2,3-dihydrophthalazinediones, horseradish peroxidase (HRP), alkaline phosphatase, β-galactosidase, glucoamylase, lysozyme, saccharide oxidases, e.g., glucose oxidase, galactose oxidase, and glucose-6-phosphate dehydrogenase, heterocyclic oxidases such as uricase and xanthine oxidase, coupled with an enzyme that employs hydrogen peroxide to oxidize a dye precursor such as HRP, lactoperoxidase, or microperoxidase, biotin/avidin, spin labels, bacteriophage labels, stable free radicals, and the like.

### E. Pharmaceutical Compositions

In a further aspect, provided are pharmaceutical compositions comprising any of the antibodies provided herein, e.g., for use in any of the below therapeutic methods. In one aspect, a pharmaceutical composition comprises any of the antibodies provided herein and a pharmaceutically acceptable carrier. In another aspect, a pharmaceutical composition comprises any of the antibodies provided herein and at least one additional therapeutic agent, e.g., as described below.

Pharmaceutical compositions of a fusion protein provided herein or an anti-huPD-L1 antibody provided herein are prepared by mixing such antibody having the desired degree of purity with one or more optional pharmaceutically acceptable carriers (Remington's Pharmaceutical Sciences 16th edition, Osol, A. Ed. (1980)), in the form of lyophilized compositions or aqueous solutions. Pharmaceutically acceptable carriers are generally nontoxic to recipients at the dosages and concentrations employed, and include, but are not limited to: buffers such as histidine, phosphate, citrate, acetate, and other organic acids; 20 antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as polyethylene glycol (PEG). Exemplary pharmaceutically acceptable carriers herein further include interstitial drug dispersion agents such as soluble neutral-active hyaluronidase glycoproteins (sHASEGP), for example, human soluble PH-20 hyaluronidase glycoproteins, such as rHuPH20 (HYLENEX®, Halozyme, Inc.). Certain exemplary sHASEGPs and methods of use, including rHuPH20, are described in US Patent Publication Nos. 2005/0260186 and 2006/0104968. In one aspect, a sHASEGP is combined with one or more additional glycosaminoglycanases such as chondroitinases.

Exemplary lyophilized antibody compositions are described in U.S. Pat. No. 6,267,958. Aqueous antibody compositions include those described in U.S. Pat. No. 6,171,586 and WO 2006/044908, the latter compositions including a histidine-acetate buffer.

The pharmaceutical composition herein may also contain more than one active ingredients as necessary for the particular indication being treated, preferably those with complementary activities that do not adversely affect each other. Such active ingredients are suitably present in combination in amounts that are effective for the purpose intended.

Active ingredients may be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

Pharmaceutical compositions for sustained-release may be prepared. Suitable examples of sustained-release preparations include semipermeable matrices of solid hydrophobic polymers containing the antibody, which matrices are in the form of shaped articles, e.g., films, or microcapsules.

The pharmaceutical compositions to be used for in vivo administration are generally sterile. Sterility may be readily accomplished, e.g., by filtration through sterile filtration membranes.

### F. Therapeutic Methods and Routes of Administration

Any of the fusion proteins or anti-huPD-L1 antibodies provided herein may be used in therapeutic methods.

In one aspect, a fusion protein or an anti-huPD-L1 antibody for use as a medicament is provided. In further aspects, a fusion protein or an anti-huPD-L1 antibody for use in treating cancer is provided. In certain aspects, a fusion protein or an anti-huPD-L1 antibody for use in a method of treatment is provided. In certain aspects, the invention provides a fusion protein or an anti-huPD-L1 antibody for use in a method of treating an individual having cancer comprising administering to the individual an effective amount of the fusion protein or the anti-huPD-L1 antibody. In one such aspect, the method further comprises administering to the individual an effective amount of at least one additional therapeutic agent (e.g., one, two, three, four, five, or six additional therapeutic agents), e.g., as described below. In further aspects, the invention provides a fusion protein or an anti-huPD-L1 antibody for use in inhibiting cell proliferation and/or tumor growth, and for use as immunomodulatory agent to directly or indirectly induce proliferation and/or activation of immune cells (like T cells, B cells and myeloid cells including monocytes, macrophages, dendritic cells, plasmacytoid dendritic cells) e.g. by secretion of immunostimulatory cytokines like IFNgamma (IFNγ) or further recruitment of immune cells. In certain aspects, the invention provides a fusion protein or an anti-huPD-L1 antibody for use in a method of inhibiting cell proliferation and/or tumor growth, and for use as immunomodulatory agent to directly or indirectly induce proliferation and/or activation of immune cells (like T cells, B cells and myeloid cells including monocytes, macrophages, dendritic cells, plasmacytoid dendritic cells) e.g. by secretion of immunostimulatory cytokines like IFNgamma (IFNγ) or further recruitment of immune cells in an individual comprising administering to the individual an effective amount of the fusion protein or the anti-huPD-L1 antibody to inhibit cell proliferation and/or tumor growth, and/or to directly or indirectly induce proliferation and/or activation of immune cells (like T cells, B cells and myeloid cells including monocytes, macrophages, dendritic cells, plasmacytoid dendritic cells) e.g. by secretion of immunostimulatory cytokines like IFNgamma (IFNγ) or further recruitment of immune cells. An “individual” according to any of the above aspects is preferably a human.

In a further aspect, the invention provides for the use of a fusion protein or an anti-huPD-L1 antibody in the manufacture or preparation of a medicament. In one aspect, the medicament is for treatment of cancer. In a further aspect, the medicament is for use in a method of treating cancer comprising administering to an individual having cancer an effective amount of the medicament. In one such aspect, the method further comprises administering to the individual an effective amount of at least one additional therapeutic agent, e.g., as described below. In a further aspect, the medicament is for inhibiting cell proliferation and/or tumor growth, and/or for modulating the immune system by directly or indirectly inducing proliferation and/or activation of immune cells (like T cells, B cells and myeloid cells including monocytes, macrophages, dendritic cells, plasmacytoid dendritic cells) e.g. by secretion of immunostimulatory cytokines like TNFalpha (TNFα) and IFNgamma (IFNγ) or further recruitment of immune cells. In a further aspect, the medicament is for use in a method of inhibiting cell proliferation and/or tumor growth, and/or for modulating the immune system by directly or indirectly inducing proliferation and/or activation of immune cells (like T cells, B cells and myeloid cells including monocytes, macrophages, dendritic cells, plasmacytoid dendritic cells) e.g. by secretion of immunostimulatory cytokines like TNFalpha (TNFα) and IFNgamma (IFNγ) or further recruitment of immune cells in an individual comprising administering to the individual an effective amount of the medicament to inhibit cell proliferation and/or tumor growth, and/or to modulate the immune system by directly or indirectly inducing proliferation and/or activation of immune cells (like T cells, B cells and myeloid cells including monocytes, macrophages, dendritic cells, plasmacytoid dendritic cells) e.g. by secretion of immunostimulatory cytokines like TNFalpha (TNFα) and IFNgamma (IFNγ) or further recruitment of immune cells. An “individual” according to any of the above aspects may be a human.

In a further aspect, the invention provides a method for treating a cancer. In one aspect, the method comprises administering to an individual having such cancer an effective amount of a fusion protein or an anti-huPD-L1 antibody. In one such aspect, the method further comprises administering to the individual an effective amount of at least one additional therapeutic agent, as described below.

The term “cancer” as used herein may be, for example, lung cancer, non-small cell lung (NSCL) cancer, bronchioloalviolar cell lung cancer, bone cancer, pancreatic cancer, skin cancer, cancer of the head or neck, cutaneous or intraocular melanoma, uterine cancer, ovarian cancer, rectal cancer, cancer of the anal region, stomach cancer, gastric cancer, colon cancer, breast cancer, uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, cancer of the bladder, cancer of the kidney or ureter, renal cell carcinoma, carcinoma of the renal pelvis, mesothelioma, hepatocellular cancer, biliary cancer, neoplasms of the central nervous system (CNS), spinal axis tumors, brain stem glioma, glioblastoma multiforme, astrocytomas, schwanomas, ependymonas, medulloblastomas, meningiomas, squamous cell carcinomas, pituitary adenoma, lymphoma, lymphocytic leukemia, including refractory versions of any of the above cancers, or a combination of one or more of the above cancers.

An “individual” according to any of the above aspects may be a human.

In a further aspect, the invention provides a method for inhibiting cell proliferation and/or tumor growth, and/or for modulating the immune system by directly or indirectly inducing proliferation and/or activation of immune cells (like T cells, B cells and myeloid cells including monocytes, macrophages, dendritic cells, plasmacytoid dendritic cells) e.g. by secretion of immunostimulatory cytokines like TNFalpha (TNFα) and IFNgamma (IFNγ) or further recruitment of immune cells, in an individual. In one aspect, the method comprises administering to the individual an effective amount of a fusion protein or an anti-huPD-L1 antibody to inhibit cell proliferation and/or tumor growth, and/or to modulate the immune system by directly or indirectly inducing proliferation and/or activation of immune cells (like T cells, B cells and myeloid cells including monocytes, macrophages, dendritic cells, plasmacytoid dendritic cells) e.g. by secretion of immunostimulatory cytokines like TNFalpha (TNFα) and IFNgamma (IFNγ) or further recruitment of immune cells. In one aspect, an “individual” is a human.

In a further aspect, the invention provides pharmaceutical compositions comprising any of the fusion proteins or the anti-huPD-L1 antibodies provided herein, e.g., for use in any of the above therapeutic methods. In one aspect, a pharmaceutical composition comprises any of the fusion proteins or the anti-huPD-L1 antibodies provided herein and a pharmaceutically acceptable carrier. In another aspect, a pharmaceutical composition comprises any of the fusion proteins or the anti-huPD-L1 antibodies provided herein and at least one additional therapeutic agent, e.g., as described below.

Antibodies of the invention can be administered alone or used in a combination therapy. For instance, the combination therapy includes administering an antibody of the invention and administering at least one additional therapeutic agent (e.g. one, two, three, four, five, or six additional therapeutic agents). In certain aspects, the combination therapy comprises administering an antibody of the invention and administering at least one additional therapeutic agent, such as a PD-L1 inhibitor, e.g. Atezolizumab, Durvalumab or Avelumab.

Such combination therapies noted above encompass combined administration (where two or more therapeutic agents are included in the same or separate pharmaceutical compositions), and separate administration, in which case, administration of the antibody of the invention can occur prior to, simultaneously, and/or following, administration of the additional therapeutic agent or agents. In one aspect, administration of the fusion protein or the anti-huPD-L1 antibody and administration of an additional therapeutic agent occur within about one month, or within about one, two or three weeks, or within about one, two, three, four, five, or six days, of each other. In one aspect, the antibody and additional therapeutic agent are administered to the patient on Day 1 of the treatment. Antibodies of the invention can also be used in combination with radiation therapy.

An antibody of the invention (and any additional therapeutic agent) can be administered by any suitable means, including parenteral, intrapulmonary, and intranasal, and, if desired for local treatment, intralesional administration. Parenteral infusions include intramuscular, intravenous, intraarterial, intraperitoneal, or subcutaneous administration. Dosing can be by any suitable route, e.g., by injections, such as intravenous or subcutaneous injections, depending in part on whether the administration is brief or chronic. Various dosing schedules including but not limited to single or multiple administrations over various time-points, bolus administration, and pulse infusion are contemplated herein.

Antibodies of the invention would be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The antibody need not be, but is optionally formulated with one or more agents currently used to prevent or treat the disorder in question. The effective amount of such other agents depends on the amount of antibody present in the pharmaceutical composition, the type of disorder or treatment, and other factors discussed above. These are generally used in the same dosages and with administration routes as described herein, or about from 1 to 99% of the dosages described herein, or in any dosage and by any route that is empirically/clinically determined to be appropriate.

For the prevention or treatment of disease, the appropriate dosage of an antibody of the invention (when used alone or in combination with one or more other additional therapeutic agents) will depend on the type of disease to be treated, the type of antibody, the severity and course of the disease, whether the antibody is administered for preventive or therapeutic purposes, previous therapy, the patient's clinical history and response to the antibody, and the discretion of the attending physician. The antibody is suitably administered to the patient at one time or over a series of treatments. Depending on the type and severity of the disease, about 1 μg/kg to 15 mg/kg (e.g., 0.1 mg/kg-10 mg/kg) of antibody can be an initial candidate dosage for administration to the patient, whether, for example, by one or more separate administrations, or by continuous infusion. One typical daily dosage might range from about 1 μg/kg to 100 mg/kg or more, depending on the factors mentioned above. For repeated administrations over several days or longer, depending on the condition, the treatment would generally be sustained until a desired suppression of disease symptoms occurs. One exemplary dosage of the antibody would be in the range from about 0.05 mg/kg to about 10 mg/kg. Thus, one or more doses of about 0.5 mg/kg, 2.0 mg/kg, 4.0 mg/kg or 10 mg/kg (or any combination thereof) may be administered to the patient. Such doses may be administered intermittently, e.g., every week or every three weeks (e.g., such that the patient receives from about two to about twenty, or, e.g., about six doses of the antibody). An initial higher loading dose, followed by one or more lower doses may be administered. An exemplary dosing regimen comprises administering an initial loading dose of about 4 mg/kg, followed by a weekly maintenance dose of about 2 mg/kg of the fusion protein. However, other dosage regimens may be useful. The progress of this therapy is easily monitored by conventional techniques and assays.

### G. Articles of Manufacture

In another aspect of the invention, an article of manufacture containing materials useful for the treatment, prevention and/or diagnosis of the disorders described above is provided. The article of manufacture comprises a container and a label or package insert on or associated with the container. Suitable containers include, for example, bottles, vials, syringes, IV solution bags, etc. The containers may be formed from a variety of materials such as glass or plastic. The container holds a composition which is by itself or combined with another composition effective for treating, preventing and/or diagnosing the condition and may have a sterile access port (for example the container may be an intravenous solution bag or a vial having a stopper pierceable by a hypodermic injection needle). At least one active agent in the composition is an antibody of the invention. The label or package insert indicates that the composition is used for treating the condition of choice. Moreover, the article of manufacture may comprise (a) a first container with a composition contained therein, wherein the composition comprises an antibody of the invention; and (b) a second container with a composition contained therein, wherein the composition comprises a further cytotoxic or otherwise therapeutic agent. The article of manufacture in this aspect of the invention may further comprise a package insert indicating that the compositions can be used to treat a particular condition. Alternatively, or additionally, the article of manufacture may further comprise a second (or third) container comprising a pharmaceutically acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

### Embodiments of the Invention

In the following specific embodiments of the invention are listed:

1. A fusion protein that comprises
a. a first antigen-binding domain capable of binding to human PD-L1;
b. a second antigen-binding domain capable of binding to human PD-L1 and to human IFN-α2; and
c. a human IFN-α2;
wherein
iv. the second antigen-binding domain is a bispecific Fab; and
v. the human IFN-α2 is fused at its C-terminus to the N-terminus of the heavy chain or the light chain of the second antigen-binding domain.

2. The fusion protein of embodiment 1, wherein the first antigen-binding domain is monospecific for human PD-L1.

3. The fusion protein of embodiment 1 or 2, wherein the second antigen-binding domain is capable of blocking the human IFN-α2 from binding to IFNAR.

4. The fusion protein of embodiment 1 to 3, wherein
a. the bispecific Fab is blocked from binding human PD-L1 when it is bound to the human IFN-α2 and is blocked from binding the human IFN-α2 when it is bound to human PD-L1, and/or
b. the human IFN-α2 is blocked from binding to IFNAR when it is bound to the bispecific Fab and is able to bind to IFNAR when the bispecific Fab is bound to PD-L1.

5. The fusion protein of any one of embodiments 1 to 4, wherein the first antigen-binding domain capable of binding to human PD-L1 is a Fab.

6. The fusion protein of any one of embodiments 1 to 5, wherein the first antigen-binding domain or the second antigen-binding domain, in particular the first antigen-binding domain, is a crossover Fab molecule wherein either the variable or the constant regions of the Fab light chain and the Fab heavy chain are exchanged.

7. The fusion protein of any one of embodiments 5 or 6, wherein in the constant domain CL of one Fab the amino acid at position 124 is substituted independently by lysine (K), arginine (R) or histidine (H) (numbering according to Kabat EU Index), and in the constant domain CH1 the amino acids at positions 147 and 213 are substituted independently by glutamic acid (E) or aspartic acid (D) (numbering according to Kabat EU index).

8. The fusion protein of any one of embodiments 5 to 7, wherein in the constant domain CL of one of the Fab fragments the amino acid at position 123 (EU numbering) has been replaced by arginine (R) and the amino acid at position 124 (EU numbering) has been substituted by lysine (K) and wherein in one of the CH1 domains the amino acids at position 147 (EU numbering) and at position 213 (EU numbering) have been substituted by glutamic acid (E).

9. The fusion protein of any one of embodiments 5 to 8, wherein in the constant domain CL of the Fab fragment of the second antigen binding domain the amino acid at position 124 is substituted independently by lysine (K), arginine (R) or histidine (H) (numbering according to Kabat EU Index), and in the constant domain CH1 the amino acids at positions 147 and 213 are substituted independently by glutamic acid (E) or aspartic acid (D) (numbering according to Kabat EU index).

10. The fusion protein of any one of embodiments 5 to 9, wherein in the constant domain CL of the Fab fragment of the second antigen binding domain the amino acid at position 123 (EU numbering) has been replaced by arginine (R) and the amino acid at position 124 (EU numbering) has been substituted by lysine (K) and wherein in one of the CH1 domains the amino acids at position 147 (EU numbering) and at position 213 (EU numbering) have been substituted by glutamic acid (E).

11. The fusion protein of any one of embodiments 1 to 10, wherein the fusion protein comprises not more than one human IFN-α2.

12. The fusion protein of any one of embodiments 1 to 11, wherein the human IFN-α2 is selected from human IFN-α2a (SEQ ID NO:79), human IFN-α2b (SEQ ID NO:80), or functional variants thereof.

13. The fusion protein of any one of embodiments 1 to 12, wherein the human IFN-α2 comprises an amino acid sequence selected from SEQ ID NO:79, SEQ ID NO:80, SEQ ID NO: 81, SEQ ID NO:82, preferably selected from SEQ ID NO:79 and SEQ ID NO:80.

14. The fusion protein of any one of embodiments 1 to 13, wherein the human IFN-α2 comprises an amino acid sequence of SEQ ID NO:79.

15. The fusion protein of any one of embodiments 1 to 14 wherein the human IFN-α2 comprises one or more mutations which modify the binding of the human IFN-α2 to the IFNAR1/2 receptor.

16. The fusion protein of any one of embodiments 1 to 15 wherein the human IFN-α2 is fused at its N-terminus or its C-terminus to the second-antigen binding domain via a peptidic linker.

17. The fusion protein of any one of embodiments 1 to 16, wherein the human IFN-α2 is fused at its C-terminus to the N-terminus of the light chain of the second antigen-binding domain.

18. The fusion protein of embodiment 16 or 17, wherein the peptidic linker has a length of 16 to 24 amino acids, particularly of 20 amino acids.

19. The fusion protein of any one of embodiments 16 to 18, wherein the peptidic linker is a glycine serine (GS) linker comprising an amino acid sequence selected from the group consisting of (GS) n, (GSGGS), (SEQ ID NO:96), (GGGS) n (SEQ ID NO:97), (GSGGG) n (SEQ ID NO:98), (GGGSG), (SEQ ID NO:99), (GSSSG) n (SEQ ID NO:100), (GGGGS) n (SEQ ID NO:101), (GGSGG) n (SEQ ID NO:102), where n represents an integer of at least 1, preferably from 4 to 6.

20. The fusion protein of any one of embodiments 16 to 19, wherein the peptidic linker comprises an amino acid sequence selected from GGSGGGS GGGSGGGSGGGSG (SEQ ID NO: 103), GSGSGGSGSGGSGSGGSGSGGSGSG (SEQ ID NO:104), GSGGGGSGGGGSGGGGSGGG (SEQ ID NO:105), GGGSGGGG SGGGGSGGGGSGGGGSG (SEQ ID NO:106), GSSS GGSSSGGSSSGGSSSG (SEQ ID NO: 107), GGGGSGGGGSGGGGSGGGGS (SEQ ID NO:108), GGGGSGG GGSGGGGSGGGGS (SEQ ID NO:109), and GGSGGGGGGGGSGGGGSGG (SEQ ID NO: 110).

21. The fusion protein of any one of embodiments 1 to 20 wherein the bispecific Fab is a DutaFab.

22 The fusion protein of any one of embodiments 1 to 21, wherein the affinity of the first antigen-binding domain to human PD-L1 is characterized by a K_{D} of 1.1 nM or lower as measured using a BIACORE® surface plasmon resonance assay at 25° C.

23. The fusion protein of any one of embodiments 1 to 22, wherein the affinity of the bispecific Fab to human PD-L1 is from 1-fold to 10-fold, in particular from 2-fold to 5-fold, of the affinity to human IFN-α2.

24. The fusion protein of any one of embodiments 1 to 23, wherein the affinity of the bispecific Fab to human PD-L1 is characterized by a K_{D} from 1 nM to 10 nM and the affinity to human IFN-α2 is characterized by a K_{D} from 10 nM to 20 nM, as measured using a BIACORE® surface plasmon resonance assay at 25° C.

25. The fusion protein of any one of embodiments 1 to 24, wherein the second antigen-binding domain capable of binding to human PD-L1 and to human IFN-α2 comprises a human PD-L1 paratope and a human IFN-α2 paratope within one cognate pair of a variable light chain domain (VL domain) and a variable heavy chain domain (VH domain), wherein the human IFN-α2 paratope comprises amino acid residues from CDR-H2, CDR-L1 and CDR-L3 of the antigen-binding domain, and wherein the human PD-L1 paratope comprises amino acid residues from the CDR-H1, CDR-H3 and CDR-L2 of the antigen-binding domain.

26. The fusion protein of any one of embodiments 1 to 25, wherein the fusion protein further comprises an Fc domain composed of a first and a second subunit.

27. The fusion protein of embodiment 26, wherein the Fc domain is an IgG Fc domain, particularly an IgG1 Fc domain or an IgG4 Fc domain.

28. The fusion protein of embodiment 26 or 27, wherein the Fc domain comprises one or more amino acid substitutions that reduce binding towards an Fc receptor, in particular towards Fcγ receptor.

29. The fusion protein of any one of embodiments 26 to 28, wherein the Fc domain is an Fc domain of human IgG1 subclass with the amino acid mutations L234A, L235A and P329G (numbering according to Kabat EU index).

30. The fusion protein of any one of embodiments 26 to 29, wherein the first subunit of the Fc domain comprises the amino acid substitutions Y349C, T366S, L368A and Y407V (numbering according to Kabat EU index) and the second subunit of the Fc domain comprises the amino acid substitutions S354C and T366W (numbering according to Kabat EU index).

31. The fusion protein of any one of embodiments 26 to 30, wherein the first antigen-binding domain is a Fab and is fused at the C-terminus of its Fab heavy chain to the N-terminus of the first subunit of the Fc domain, and the second antigen-binding domain is a bispecific Fab, in particular a DutaFab, and is fused at the C-terminus of its Fab heavy chain to the N-terminus of the second subunit of the Fc domain, and wherein the human IFN-α2 is fused at its C-terminus to the N-terminus of the light chain of the second antigen-binding domain.

32. The fusion protein of any one of embodiments 1 to 31, wherein the second antigen-binding domain capable of binding to human PD-L1 and to human IFN-α2 comprises
i. a heavy chain variable domain (VH) comprising (a) CDR-H1 comprising the amino acid sequence of SEQ ID NO:1, (b) CDR-H2 comprising the amino acid sequence of SEQ ID NO:2, and (c) CDR-H3 comprising the amino acid sequence of SEQ ID NO:3, and a light chain variable domain (VL) comprising (d) CDR-L1 comprising the amino acid sequence of SEQ ID NO:4, (e) CDR-L2 comprising the amino acid sequence of SEQ ID NO:5, and (f) CDR-L3 comprising the amino acid sequence of SEQ ID NO:6;
ii. a heavy chain variable domain (VH) comprising (a) CDR-H1 comprising the amino acid sequence of SEQ ID NO:1, (b) CDR-H2 comprising the amino acid sequence of SEQ ID NO:2, and (c) CDR-H3 comprising the amino acid sequence of SEQ ID NO:3, and a light chain variable domain (VL) comprising (d) CDR-L1 comprising the amino acid sequence of SEQ ID NO:9, (c) CDR-L2 comprising the amino acid sequence of SEQ ID NO:5, and (f) CDR-L3 comprising the amino acid sequence of SEQ ID NO:6;
iii. a heavy chain variable domain (VH) comprising (a) CDR-H1 comprising the amino acid sequence of SEQ ID NO:1, (b) CDR-H2 comprising the amino acid sequence of SEQ ID NO:2, and (c) CDR-H3 comprising the amino acid sequence of SEQ ID NO:3, and a light chain variable domain (VL) comprising (d) CDR-L1 comprising the amino acid sequence of SEQ ID NO:4, (c) CDR-L2 comprising the amino acid sequence of SEQ ID NO:11, and (f) CDR-L3 comprising the amino acid sequence of SEQ ID NO:6; or
iv. a heavy chain variable domain (VH) comprising (a) CDR-H1 comprising the amino acid sequence of SEQ ID NO:13, (b) CDR-H2 comprising the amino acid sequence of SEQ ID NO:14, and (c) CDR-H3 comprising the amino acid sequence of SEQ ID NO: 15, and a light chain variable domain (VL) comprising (d) CDR-L1 comprising the amino acid sequence of SEQ ID NO:16, (e) CDR-L2 comprising the amino acid sequence of SEQ ID NO:17, and (f) CDR-L3 comprising the amino acid sequence of SEQ ID NO:18.

33. The fusion protein of any one of embodiments 1 to 32, wherein the second antigen-binding domain capable of binding to human PD-L1 and to human IFN-α2 comprises a heavy chain variable domain (VH) comprising (a) CDR-H1 comprising the amino acid sequence of SEQ ID NO:1, (b) CDR-H2 comprising the amino acid sequence of SEQ ID NO: 2, and (c) CDR-H3 comprising the amino acid sequence of SEQ ID NO:3, and a light chain variable domain (VL) comprising (d) CDR-L1 comprising the amino acid sequence of SEQ ID NO:4, (e) CDR-L2 comprising the amino acid sequence of SEQ ID NO:5, and (f) CDR-L3 comprising the amino acid sequence of SEQ ID NO:6.

34 The fusion protein of any one of embodiments 1 to 33, wherein the second antigen-binding domain capable of binding to human PD-L1 and to human IFN-α2 comprises
i. a VH domain comprising the amino acid sequence of SEQ ID NO:7 and a VL domain comprising the amino acid sequence of SEQ ID NO:8;
ii. a VH domain comprising the amino acid sequence of SEQ ID NO:7 and a VL domain comprising the amino acid sequence of SEQ ID NO:10;
iii. a VH domain comprising the amino acid sequence of SEQ ID NO:7 and a VL domain comprising the amino acid sequence of SEQ ID NO:12; or
iv. a VH domain comprising the amino acid sequence of SEQ ID NO: 19 and a VL domain comprising the amino acid sequence of SEQ ID NO:20.

35 The fusion protein of any one of embodiments 1 to 34, wherein the second antigen-binding domain capable of binding to human PD-L1 and to human IFN-α2 comprises a VH domain comprising the amino acid sequence of SEQ ID NO:7 and a VL domain comprising the amino acid sequence of SEQ ID NO:8.

36. The fusion protein of any one of embodiments 1 to 35, wherein the first antigen binding domain capable of binding to human PD-L1 comprises
i. a heavy chain variable domain (VH) comprising (a) CDR-H1 comprising the amino acid sequence of SEQ ID NO:21, (b) CDR-H2 comprising the amino acid sequence of SEQ ID NO:22, and (c) CDR-H3 comprising the amino acid sequence of SEQ ID NO:23, and a light chain variable domain (VL) comprising (d) CDR-L1 comprising the amino acid sequence of SEQ ID NO:24, (e) CDR-L2 comprising the amino acid sequence of SEQ ID NO:25, and (f) CDR-L3 comprising the amino acid sequence of SEQ ID NO:26; or
ii. a heavy chain variable domain (VH) comprising (a) CDR-H1 comprising the amino acid sequence of SEQ ID NO:29, (b) CDR-H2 comprising the amino acid sequence of SEQ ID NO:30, and (c) CDR-H3 comprising the amino acid sequence of SEQ ID NO:31, and a light chain variable domain (VL) comprising (d) CDR-L1 comprising the amino acid sequence of SEQ ID NO:32, (e) CDR-L2 comprising the amino acid sequence of SEQ ID NO:33 and (f) CDR-L3 comprising the amino acid sequence of SEQ ID NO:34.

37. The fusion protein of any one of embodiments 1 to 36, wherein the first antigen binding domain capable of binding to human PD-L1 comprises
i. a VH domain comprising the amino acid sequence of SEQ ID NO:27 and a VL domain comprising the amino acid sequence of SEQ ID NO:28; or
ii. a VH domain comprising the amino acid sequence of SEQ ID NO:35 and a VL domain comprising the amino acid sequence of SEQ ID NO:36.

38. The fusion protein of any one of embodiments 1 to 37, wherein
a. the first antigen-binding domain capable of binding to human PD-L1 comprises
   i. a heavy chain variable domain (VH) comprising (a) CDR-H1 comprising the amino acid sequence of SEQ ID NO:21, (b) CDR-H2 comprising the amino acid sequence of SEQ ID NO:22, and (c) CDR-H3 comprising the amino acid sequence of SEQ ID NO:23, and a light chain variable domain (VL) comprising (d) CDR-L1 comprising the amino acid sequence of SEQ ID NO:24, (e) CDR-L2 comprising the amino acid sequence of SEQ ID NO:25, and (f) CDR-L3 comprising the amino acid sequence of SEQ ID NO:26; or
   ii. a heavy chain variable domain (VH) comprising (a) CDR-H1 comprising the amino acid sequence of SEQ ID NO:29, (b) CDR-H2 comprising the amino acid sequence of SEQ ID NO:30, and (c) CDR-H3 comprising the amino acid sequence of SEQ ID NO:31, and a light chain variable domain (VL) comprising (d) CDR-L1 comprising the amino acid sequence of SEQ ID NO:32, (e) CDR-L2 comprising the amino acid sequence of SEQ ID NO:33, and (f) CDR-L3 comprising the amino acid sequence of SEQ ID NO:34; and
b. the second antigen-binding domain capable of binding to human PD-L1 and to human IFN-α2 comprises
   i. a heavy chain variable domain (VH) comprising (a) CDR-H1 comprising the amino acid sequence of SEQ ID NO:1, (b) CDR-H2 comprising the amino acid sequence of SEQ ID NO:2, and (c) CDR-H3 comprising the amino acid sequence of SEQ ID NO:3, and a light chain variable domain (VL) comprising (d) CDR-L1 comprising the amino acid sequence of SEQ ID NO:4, (e) CDR-L2 comprising the amino acid sequence of SEQ ID NO: 5, and (f) CDR-L3 comprising the amino acid sequence of SEQ ID NO: 6;
   ii. a heavy chain variable domain (VH) comprising (a) CDR-H1 comprising the amino acid sequence of SEQ ID NO:1, (b) CDR-H2 comprising the amino acid sequence of SEQ ID NO:2, and (c) CDR-H3 comprising the amino acid sequence of SEQ ID NO:3, and a light chain variable domain (VL) comprising (d) CDR-L1 comprising the amino acid sequence of SEQ ID NO:9, (e) CDR-L2 comprising the amino acid sequence of SEQ ID NO: 5, and (f) CDR-L3 comprising the amino acid sequence of SEQ ID NO: 6;
   iii. a heavy chain variable domain (VH) comprising (a) CDR-H1 comprising the amino acid sequence of SEQ ID NO:1, (b) CDR-H2 comprising the amino acid sequence of SEQ ID NO:2, and (c) CDR-H3 comprising the amino acid sequence of SEQ ID NO:3, and a light chain variable domain (VL) comprising (d) CDR-L1 comprising the amino acid sequence of SEQ ID NO:4, (e) CDR-L2 comprising the amino acid sequence of SEQ ID NO: 11, and (f) CDR-L3 comprising the amino acid sequence of SEQ ID NO: 6; or
   iv. a heavy chain variable domain (VH) comprising (a) CDR-H1 comprising the amino acid sequence of SEQ ID NO:13, (b) CDR-H2 comprising the amino acid sequence of SEQ ID NO: 14, and (c) CDR-H3 comprising the amino acid sequence of SEQ ID NO:15, and a light chain variable domain (VL) comprising (d) CDR-L1 comprising the amino acid sequence of SEQ ID NO:16, (e) CDR-L2 comprising the amino acid sequence of SEQ ID NO:17, and (f) CDR-L3 comprising the amino acid sequence of SEQ ID NO:18.

39. The fusion protein of any one of embodiments 1 to 38, wherein
a. the first antigen-binding domain capable of binding to human PD-L1 comprises a heavy chain variable domain (VH) comprising (a) CDR-H1 comprising the amino acid sequence of SEQ ID NO:21, (b) CDR-H2 comprising the amino acid sequence of SEQ ID NO:22, and (c) CDR-H3 comprising the amino acid sequence of SEQ ID NO:23, and a light chain variable domain (VL) comprising (d) CDR-L1 comprising the amino acid sequence of SEQ ID NO:24, (e) CDR-L2 comprising the amino acid sequence of SEQ ID NO:25, and (f) CDR-L3 comprising the amino acid sequence of SEQ ID NO:26; and
b. the second antigen-binding domain capable of binding to human PD-L1 and to human IFN-α2 comprises a heavy chain variable domain (VH) comprising (a) CDR-H1 comprising the amino acid sequence of SEQ ID NO:1, (b) CDR-H2 comprising the amino acid sequence of SEQ ID NO:2, and (c) CDR-H3 comprising the amino acid sequence of SEQ ID NO:3, and a light chain variable domain (VL) comprising (d) CDR-L1 comprising the amino acid sequence of SEQ ID NO:4, (e) CDR-L2 comprising the amino acid sequence of SEQ ID NO:5, and (f) CDR-L3 comprising the amino acid sequence of SEQ ID NO: 6.

40. The fusion protein of any one of embodiments 1 to 39, wherein
a. the first antigen-binding domain capable of binding to human PD-L1 is a Fab comprising
   i. a heavy chain variable domain (VH) comprising (a) CDR-H1 comprising the amino acid sequence of SEQ ID NO:21, (b) CDR-H2 comprising the amino acid sequence of SEQ ID NO:22, and (c) CDR-H3 comprising the amino acid sequence of SEQ ID NO:23, and a light chain variable domain (VL) comprising (d) CDR-L1 comprising the amino acid sequence of SEQ ID NO:24, (e) CDR-L2 comprising the amino acid sequence of SEQ ID NO:25, and (f) CDR-L3 comprising the amino acid sequence of SEQ ID NO:26; or
   ii. a heavy chain variable domain (VH) comprising (a) CDR-H1 comprising the amino acid sequence of SEQ ID NO:29, (b) CDR-H2 comprising the amino acid sequence of SEQ ID NO:30, and (c) CDR-H3 comprising the amino acid sequence of SEQ ID NO:31, and a light chain variable domain (VL) comprising (d) CDR-L1 comprising the amino acid sequence of SEQ ID NO:32, (e) CDR-L2 comprising the amino acid sequence of SEQ ID NO:33, and (f) CDR-L3 comprising the amino acid sequence of SEQ ID NO:34, wherein the first antigen-binding moiety is a crossover Fab molecule wherein either the variable or the constant regions of the Fab light chain and the Fab heavy chain are exchanged; and
b. the second antigen-binding domain capable of binding to human PD-L1 and to human IFN-α2 is a bispecific Fab, preferably a DutaFab, and comprises
   i. a heavy chain variable domain (VH) comprising (a) CDR-H1 comprising the amino acid sequence of SEQ ID NO:1, (b) CDR-H2 comprising the amino acid sequence of SEQ ID NO:2, and (c) CDR-H3 comprising the amino acid sequence of SEQ ID NO:3, and a light chain variable domain (VL) comprising (d) CDR-L1 comprising the amino acid sequence of SEQ ID NO:4, (e) CDR-L2 comprising the amino acid sequence of SEQ ID NO: 5, and (f) CDR-L3 comprising the amino acid sequence of SEQ ID NO: 6;
   ii. a heavy chain variable domain (VH) comprising (a) CDR-H1 comprising the amino acid sequence of SEQ ID NO:1, (b) CDR-H2 comprising the amino acid sequence of SEQ ID NO:2, and (c) CDR-H3 comprising the amino acid sequence of SEQ ID NO:3, and a light chain variable domain (VL) comprising (d) CDR-L1 comprising the amino acid sequence of SEQ ID NO:9, (e) CDR-L2 comprising the amino acid sequence of SEQ ID NO: 5, and (f) CDR-L3 comprising the amino acid sequence of SEQ ID NO: 6;
   iii. a heavy chain variable domain (VH) comprising (a) CDR-H1 comprising the amino acid sequence of SEQ ID NO:1, (b) CDR-H2 comprising the amino acid sequence of SEQ ID NO:2, and (c) CDR-H3 comprising the amino acid sequence of SEQ ID NO:3, and a light chain variable domain (VL) comprising (d) CDR-L1 comprising the amino acid sequence of SEQ ID NO:4, (e) CDR-L2 comprising the amino acid sequence of SEQ ID NO: 11, and (f) CDR-L3 comprising the amino acid sequence of SEQ ID NO: 6; or
   iv. a heavy chain variable domain (VH) comprising (a) CDR-H1 comprising the amino acid sequence of SEQ ID NO:13, (b) CDR-H2 comprising the amino acid sequence of SEQ ID NO:14, and (c) CDR-H3 comprising the amino acid sequence of SEQ ID NO:15, and a light chain variable domain (VL) comprising (d) CDR-L1 comprising the amino acid sequence of SEQ ID NO:16, (e) CDR-L2 comprising the amino acid sequence of SEQ ID NO:17, and (f) CDR-L3 comprising the amino acid sequence of SEQ ID NO:18; and
c. an Fc domain composed of a first and a second subunit;
   wherein the first antigen-binding domain is fused at the C-terminus of its Fab heavy chain to the N-terminus of the first subunit of the Fc domain, and the second antigen-binding domain is fused at the C-terminus of its Fab heavy chain to the N-terminus of the second subunit of the Fc domain, and wherein the IFN-α2 or variant thereof is fused at its C-terminus to the N-terminus of the Fab light chain of the second antigen-binding domain.

41. The fusion protein of any one of embodiments 1 to 40, wherein
a. the first antigen-binding domain capable of binding to human PD-L1 comprises
   i. a VH domain comprising the amino acid sequence of SEQ ID NO:27 and a VL domain comprising the amino acid sequence of SEQ ID NO:28; or
   ii. a VH domain comprising the amino acid sequence of SEQ ID NO:35 and a VL domain comprising the amino acid sequence of SEQ ID NO:36. and
b. the second antigen-binding domain capable of binding to human PD-L1 and to human IFN-α2 comprises
   i. a VH domain comprising the amino acid sequence of SEQ ID NO:7 and a VL domain comprising the amino acid sequence of SEQ ID NO:8;
   ii. a VH domain comprising the amino acid sequence of SEQ ID NO:7 and a VL domain comprising the amino acid sequence of SEQ ID NO:10;
   iii. a VH domain comprising the amino acid sequence of SEQ ID NO:7 and a VL domain comprising the amino acid sequence of SEQ ID NO:12; or
   iv. a VH domain comprising the amino acid sequence of SEQ ID NO: 19 and a VL domain comprising the amino acid sequence of SEQ ID NO:20.

42. The fusion protein of any one of embodiments 1 to 41, wherein
a. the first antigen-binding domain capable of binding to human PD-L1 comprises
   i. a VH domain comprising the amino acid sequence of SEQ ID NO:27 and a VL domain comprising the amino acid sequence of SEQ ID NO:28; or
   ii. a VH domain comprising the amino acid sequence of SEQ ID NO:35 and a VL domain comprising the amino acid sequence of SEQ ID NO:36; and
b. the second antigen-binding domain capable of binding to human PD-L1 and to human IFN-α2 comprises a VH domain comprising the amino acid sequence of SEQ ID NO:7 and a VL domain comprising the amino acid sequence of SEQ ID NO:8.

43. The fusion protein of any one of embodiments 1 to 42, wherein the first antigen-binding domain capable of binding to human PD-L1 comprises a VH domain comprising the amino acid sequence of SEQ ID NO:27 and a VL domain comprising the amino acid sequence of SEQ ID NO:28 and the second antigen-binding domain capable of binding to human PD-L1 and to human IFN-α2 comprises a VH domain comprising the amino acid sequence of SEQ ID NO:7 and a VL domain comprising the amino acid sequence of SEQ ID NO: 8.

44. The fusion protein of any one of embodiments 26 to 43, wherein the first antigen-binding domain is a Fab, and wherein the fusion protein is composed of
(a) a first polypeptide, comprising (a1) the heavy chain of the first antigen binding domain, fused at its C-terminus to the N-terminus of one of the subunits (e.g. the first subunit) of the Fc domain, and (a2) one of the subunits (e.g. the first subunit) of the Fc domain;
(b) a second polypeptide, comprising the light chain of the first antigen-binding domain;
(c) a third polypeptide, comprising (c1) the heavy chain of the second antigen-binding domain, fused at its C-terminus to the N-terminus of the other one of the subunits (e.g. the second subunit) of the Fc domain, and (c2) the other one of the subunits (e.g. the second subunit) of the Fc domain; and
(d) a fourth polypeptide, comprising (d1) the human IFN-α2, fused at its C-terminus to the N-terminus of the light chain of the second antigen-binding domain, and (d2) the light chain of the second antigen-binding domain.

45. A fusion protein comprising
a) a first antigen-binding domain capable of binding to human PD-L1;
b) a second antigen-binding domain capable of binding to human PD-L1 and to human IFN-α2;
c) a human IFN-α2; and
d) an Fc domain composed of a first and a second subunit;
   wherein the first antigen-binding domain is a Fab, preferably a monospecific Fab, and the second antigen-binding domain is a bispecific Fab, wherein the first and the second antigen-binding domain each comprise a heavy chain and a light chain, and
   wherein the fusion protein is composed of
(a) a first polypeptide, comprising (a1) the heavy chain of the first antigen binding domain, fused at its C-terminus to the N-terminus of one of the subunits (e.g. the first subunit) of the Fc domain, and (a2) one of the subunits (e.g. the first subunit) of the Fc domain;
(b) a second polypeptide, comprising the light chain of the first antigen-binding domain;
(c) a third polypeptide, comprising (c1) the heavy chain of the second antigen-binding domain, fused at its C-terminus to the N-terminus of the other one of the subunits (e.g. the second subunit) of the Fc domain, and (c2) the other one of the subunits (e.g. the second subunit) of the Fc domain; and
(d) a fourth polypeptide, comprising (d1) the human IFN-α2, fused at its C-terminus to the N-terminus of the light chain of the second antigen-binding domain, and (d2) the light chain of the second antigen-binding domain.

46. The fusion protein of embodiment 44 or 45, wherein
the first polypeptide comprises an amino acid sequence that is at least about 95%, 96%, 97%, 98%, 99% or 100% identical to the amino acid sequence of SEQ ID NO:37, the second polypeptide comprises an amino acid sequence that is at least about 95%, 96%, 97%, 98%, 99% or 100% identical to the amino acid sequence of SEQ ID NO:38, the third polypeptide comprises an amino acid sequence that is at least about 95%, 96%, 97%, 98%, 99% or 100% identical to the amino acid sequence of SEQ ID NO:39, and the fourth polypeptide comprises an amino acid sequence that is at least about 95%, 96%, 97%, 98%, 99% or 100% identical to the amino acid sequence of SEQ ID NO:40; or
wherein the first polypeptide comprises an amino acid sequence that is at least about 95%, 96%, 97%, 98%, 99% or 100% identical to the amino acid sequence of SEQ ID NO:41, the second polypeptide comprises an amino acid sequence that is at least about 95%, 96%, 97%, 98%, 99% or 100% identical to the amino acid sequence of SEQ ID NO:42, the third polypeptide comprises an amino acid sequence that is at least about 95%, 96%, 97%, 98%, 99% or 100% identical to the amino acid sequence of SEQ ID NO:43, and the fourth polypeptide comprises an amino acid sequence that is at least about 95%, 96%, 97%, 98%, 99% or 100% identical to the amino acid sequence of SEQ ID NO:44; or
wherein the first polypeptide comprises an amino acid sequence that is at least about 95%, 96%, 97%, 98%, 99% or 100% identical to the amino acid sequence of SEQ ID NO:45, the second polypeptide comprises an amino acid sequence that is at least about 95%, 96%, 97%, 98%, 99% or 100% identical to the amino acid sequence of SEQ ID NO:46, the third polypeptide comprises an amino acid sequence that is at least about 95%, 96%, 97%, 98%, 99% or 100% identical to the amino acid sequence of SEQ ID NO:47, and the fourth polypeptide comprises an amino acid sequence that is at least about 95%, 96%, 97%, 98%, 99% or 100% identical to the amino acid sequence of SEQ ID NO:48; or
wherein the first polypeptide comprises an amino acid sequence that is at least about 95%, 96%, 97%, 98%, 99% or 100% identical to the amino acid sequence of SEQ ID NO:49, the second polypeptide comprises an amino acid sequence that is at least about 95%, 96%, 97%, 98%, 99% or 100% identical to the amino acid sequence of SEQ ID NO:50, the third polypeptide comprises an amino acid sequence that is at least about 95%, 96%, 97%, 98%, 99% or 100% identical to the amino acid sequence of SEQ ID NO:51, and the fourth polypeptide comprises an amino acid sequence that is at least about 95%, 96%, 97%, 98%, 99% or 100% identical to the amino acid sequence of SEQ ID NO:52; or
wherein the first polypeptide comprises an amino acid sequence that is at least about 95%, 96%, 97%, 98%, 99% or 100% identical to the amino acid sequence of SEQ ID NO:53, the second polypeptide comprises an amino acid sequence that is at least about 95%, 96%, 97%, 98%, 99% or 100% identical to the amino acid sequence of SEQ ID NO:54, the third polypeptide comprises an amino acid sequence that is at least about 95%, 96%, 97%, 98%, 99% or 100% identical to the amino acid sequence of SEQ ID NO:55, and the fourth polypeptide comprises an amino acid sequence that is at least about 95%, 96%, 97%, 98%, 99% or 100% identical to the amino acid sequence of SEQ ID NO:56; or
wherein the first polypeptide comprises an amino acid sequence that is at least about 95%, 96%, 97%, 98%, 99% or 100% identical to the amino acid sequence of SEQ ID NO:57, the second polypeptide comprises an amino acid sequence that is at least about 95%, 96%, 97%, 98%, 99% or 100% identical to the amino acid sequence of SEQ ID NO:58, the third polypeptide comprises an amino acid sequence that is at least about 95%, 96%, 97%, 98%, 99% or 100% identical to the amino acid sequence of SEQ ID NO:59, and the fourth polypeptide comprises an amino acid sequence that is at least about 95%, 96%, 97%, 98%, 99% or 100% identical to the amino acid sequence of SEQ ID NO:60.

47. The fusion protein of any one of embodiments 44 to 46, wherein the first polypeptide comprises an amino acid sequence that is at least about 95%, 96%, 97%, 98%, 99% or 100% identical to the amino acid sequence of SEQ ID NO:37, the second polypeptide comprises an amino acid sequence that is at least about 95%, 96%, 97%, 98%, 99% or 100% identical to the amino acid sequence of SEQ ID NO:38, the third polypeptide comprises an amino acid sequence that is at least about 95%, 96%, 97%, 98%, 99% or 100% identical to the amino acid sequence of SEQ ID NO:39, and the fourth polypeptide comprises an amino acid sequence that is at least about 95%, 96%, 97%, 98%, 99% or 100% identical to the amino acid sequence of SEQ ID NO:40.

48. A fusion protein comprising four polypeptides wherein the first polypeptide comprises an amino acid sequence that is at least about 95%, 96%, 97%, 98%, 99% or 100% identical to the amino acid sequence of SEQ ID NO:37, the second polypeptide comprises an amino acid sequence that is at least about 95%, 96%, 97%, 98%, 99% or 100% identical to the amino acid sequence of SEQ ID NO:38, the third polypeptide comprises an amino acid sequence that is at least about 95%, 96%, 97%, 98%, 99% or 100% identical to the amino acid sequence of SEQ ID NO:39, and the fourth polypeptide comprises an amino acid sequence that is at least about 95%, 96%, 97%, 98%, 99% or 100% identical to the amino acid sequence of SEQ ID NO:40.

49. A fusion protein comprising four polypeptides wherein the first polypeptide comprises an amino acid sequence that is at least about 95%, 96%, 97%, 98%, 99% or 100% identical to the amino acid sequence of SEQ ID NO:41, the second polypeptide comprises an amino acid sequence that is at least about 95%, 96%, 97%, 98%, 99% or 100% identical to the amino acid sequence of SEQ ID NO:42, the third polypeptide comprises an amino acid sequence that is at least about 95%, 96%, 97%, 98%, 99% or 100% identical to the amino acid sequence of SEQ ID NO:43, and the fourth polypeptide comprises an amino acid sequence that is at least about 95%, 96%, 97%, 98%, 99% or 100% identical to the amino acid sequence of SEQ ID NO:44.

50. A fusion protein comprising four polypeptides wherein the first polypeptide comprises an amino acid sequence that is at least about 95%, 96%, 97%, 98%, 99% or 100% identical to the amino acid sequence of SEQ ID NO:45, the second polypeptide comprises an amino acid sequence that is at least about 95%, 96%, 97%, 98%, 99% or 100% identical to the amino acid sequence of SEQ ID NO:46, the third polypeptide comprises an amino acid sequence that is at least about 95%, 96%, 97%, 98%, 99% or 100% identical to the amino acid sequence of SEQ ID NO:47, and the fourth polypeptide comprises an amino acid sequence that is at least about 95%, 96%, 97%, 98%, 99% or 100% identical to the amino acid sequence of SEQ ID NO:48.

51. A fusion protein comprising four polypeptides wherein the first polypeptide comprises an amino acid sequence that is at least about 95%, 96%, 97%, 98%, 99% or 100% identical to the amino acid sequence of SEQ ID NO:49, the second polypeptide comprises an amino acid sequence that is at least about 95%, 96%, 97%, 98%, 99% or 100% identical to the amino acid sequence of SEQ ID NO:50, the third polypeptide comprises an amino acid sequence that is at least about 95%, 96%, 97%, 98%, 99% or 100% identical to the amino acid sequence of SEQ ID NO:51, and the fourth polypeptide comprises an amino acid sequence that is at least about 95%, 96%, 97%, 98%, 99% or 100% identical to the amino acid sequence of SEQ ID NO:52.

52. A fusion protein comprising four polypeptides wherein the first polypeptide comprises an amino acid sequence that is at least about 95%, 96%, 97%, 98%, 99% or 100% identical to the amino acid sequence of SEQ ID NO:53, the second polypeptide comprises an amino acid sequence that is at least about 95%, 96%, 97%, 98%, 99% or 100% identical to the amino acid sequence of SEQ ID NO:54, the third polypeptide comprises an amino acid sequence that is at least about 95%, 96%, 97%, 98%, 99% or 100% identical to the amino acid sequence of SEQ ID NO:55, and the fourth polypeptide comprises an amino acid sequence that is at least about 95%, 96%, 97%, 98%, 99% or 100% identical to the amino acid sequence of SEQ ID NO:56.

53. A fusion protein comprising four polypeptides wherein the first polypeptide comprises an amino acid sequence that is at least about 95%, 96%, 97%, 98%, 99% or 100% identical to the amino acid sequence of SEQ ID NO:57, the second polypeptide comprises an amino acid sequence that is at least about 95%, 96%, 97%, 98%, 99% or 100% identical to the amino acid sequence of SEQ ID NO:58, the third polypeptide comprises an amino acid sequence that is at least about 95%, 96%, 97%, 98%, 99% or 100% identical to the amino acid sequence of SEQ ID NO:59, and the fourth polypeptide comprises an amino acid sequence that is at least about 95%, 96%, 97%, 98%, 99% or 100% identical to the amino acid sequence of SEQ ID NO:60.

54. A fusion protein of any of embodiments 1 to 53, wherein
a) the first antigen-binding domain is a crossover Fab and is fused at the C-terminus of its heavy chain to the N-terminus of one of the two subunits of the Fc domain;
b) the second antigen-binding domain is a DutaFab and is fused at the C-terminus of its heavy chain to the N-terminus of the other of the two subunits of the Fc domain; and
c) the human IFN-α2 is wildtype human IFN-α2 of SEQ ID NO: 79 and is fused at its C-terminus via a peptidic linker to the N-terminus of the light chain of the second antigen-binding domain.

55. An isolated nucleic acid encoding the fusion protein of any of embodiments 1 to 54.

56. A host cell comprising the nucleic acid of embodiment 55.

57. A method, preferably an in vitro method, of producing a fusion protein of any of embodiments 1 to 54 comprising culturing the host cell of embodiment 56 under conditions suitable for the expression of the antibody.

58. The method of embodiment 57, further comprising recovering the fusion protein from the host cell.

59. A fusion protein produced by the method of embodiment 57 or 58.

60. A pharmaceutical composition comprising the fusion protein of any of embodiments 1 to 54 or 59 and a pharmaceutically acceptable carrier.

61. The fusion protein of any one of embodiments 1 to 54 or 59 or the pharmaceutical composition of embodiment 60 for use as a medicament.

62. The fusion protein of any one of embodiments 1 to 54 or 59 or the pharmaceutical composition of embodiment 60 for use in treating cancer.

63. Use of the fusion protein of any one of embodiments 1 to 54 or 59 or the pharmaceutical composition of embodiment 60 in the manufacture of a medicament, particularly a medicament for treatment of cancer.

64. Use of the fusion protein of any one of embodiments 1 to 54 or 59 or the pharmaceutical composition of embodiment 60 in the manufacture of a medicament for inhibiting cell proliferation and/or tumor growth.

65. Use of the fusion protein of any one of embodiments 1 to 54 or 59 or the pharmaceutical composition of embodiment 60 in the manufacture of a medicament for modulating the immune system by directly or indirectly inducing proliferation and/or activation of immune cells.

66. A method of treating an individual having cancer comprising administering to the individual an effective amount of the fusion protein of any one of embodiments 1 to 54 or 59 or the pharmaceutical composition of embodiment 60.

67. A method of inhibiting cell proliferation and/or tumor growth in an individual comprising administering to the individual an effective amount of the fusion protein of any one of embodiments 1 to 54 or 59 or the pharmaceutical composition of embodiment 60 to inhibit cell proliferation and/or tumor growth.

68. A method of modulating the immune system by directly or indirectly inducing proliferation and/or activation of immune cells in an individual comprising administering to the individual an effective amount of the fusion protein of any one of embodiments 1 to 54 or 59 or the pharmaceutical composition of embodiment 60 to modulate the immune system by directly or indirectly inducing proliferation and/or activation of immune cells.

69. An antibody that binds to human PD-L1, wherein the antibody comprises a heavy chain variable domain (VH) comprising (a) CDR-H1 comprising the amino acid sequence of SEQ ID NO:21, (b) CDR-H2 comprising the amino acid sequence of SEQ ID NO: 22, and (c) CDR-H3 comprising the amino acid sequence of SEQ ID NO:23, and a light chain variable domain (VL) comprising (d) CDR-L1 comprising the amino acid sequence of SEQ ID NO:24, (e) CDR-L2 comprising the amino acid sequence of SEQ ID NO: 25, and (f) CDR-L3 comprising the amino acid sequence of SEQ ID NO:26.

70. The antibody of embodiment 69, comprising a sequence selected from the group consisting of
(a) a VH sequence having at least 95% sequence identity to the amino acid sequence of SEQ ID NO:27;
(b) a VL sequence having at least 95% sequence identity to the amino acid sequence of SEQ ID NO:28; and
(c) a VH sequence as defined in (a) and a VL sequence as defined in (b).

71. The antibody of embodiment 69 or 70, comprising a VH sequence of SEQ ID NO: 27 and a VL sequence of SEQ ID NO:28.

72. An antibody that specifically binds to human PD-L1 comprising a VH sequence of SEQ ID NO:27 and a VL sequence of SEQ ID NO:28.

73. The antibody of any of embodiments 69 to 72 comprising a heavy chain of SEQ ID NO: 142 and a light chain of SEQ ID NO:143.

74. An antibody that specifically binds to human PD-L1 comprising a heavy chain of SEQ ID NO:142 and a light chain of SEQ ID NO:143.

75 The antibody of any of embodiments 69 to 74, which is a monoclonal antibody.

76. The antibody of any of embodiments 66 to 75, which is a humanized or chimeric antibody.

77. The antibody of any of embodiments 69 to 76, which is an antibody fragment that binds to PD-L1.

78. The antibody of any of embodiments 69 to 77, which is a (monospecific) DutaFab that binds to PD-L1.

79. The antibody of any of embodiments 69 to 78, which is a full-length IgG₁ antibody.

80. The antibody of any of embodiments 69 to 79, wherein the antibody binds human PD-L1 with an affinity of 1.1 nM or lower, in particular an affinity of 0.8 nM or lower, as measured using a BIACORE® surface plasmon resonance assay at 25° C.

81. The antibody of any of embodiments 69 to 80, wherein the antibody binds to the C-terminal domain of human PD-L1.

82. The antibody of any of embodiments 69 to 81, wherein the binding of the antibody to human PD-L1 blocks the interaction of human PD-L1 and human PD1 as measured using a BIACORE® surface plasmon resonance assay at 25° C.

83. The antibody of any of embodiments 69 to 82, wherein the antibody is a multispecific antibody.

84. An isolated nucleic acid encoding the antibody of any of embodiments 69 to 83.

85. A host cell comprising the nucleic acid of embodiment 84.

86. A method of producing an antibody that binds to human PD-L1 comprising culturing the host cell of embodiment 85 under conditions suitable for the expression of the antibody.

87. The method of embodiment 86, further comprising recovering the antibody from the host cell.

88. An antibody produced by the method of any one of embodiments 86 or 87.

89. A pharmaceutical composition comprising the antibody of any of embodiments 69 to 83 or 88 and a pharmaceutically acceptable carrier.

90. The antibody of any one of embodiments 69 to 83 or 88 or the pharmaceutical composition of embodiment 89 for use as a medicament.

91. The antibody of any one of embodiments 69 to 83 or 88 or the pharmaceutical composition of embodiment 89 for use in treating cancer.

92. Use of the antibody of any one of embodiments 69 to 83 or 88 or the pharmaceutical composition of embodiment 89 in the manufacture of a medicament for treatment of cancer.

93. Use of the antibody of any one of embodiments 69 to 83 or 88 or the pharmaceutical composition of embodiment 89 in the manufacture of a medicament for targeting a therapeutically active agent to a tumor cell and/or an immune cell in the tumor micro environment.

94. A method of treating an individual having cancer comprising administering to the individual an effective amount of the antibody of any one of embodiments 69 to 83 or 88 or the pharmaceutical composition of embodiment 89.

95. A method of inhibiting cell proliferation and/or tumor growth in an individual comprising administering to the individual an effective amount of the antibody of any of embodiments 69 to 83 or 88 or the pharmaceutical composition of embodiment 89.

96. A method of modulating the immune system by directly or indirectly inducing proliferation and/or activation of immune cells in an individual comprising administering to the individual an effective amount of the antibody of any of embodiments 69 to 83 or 88 or the pharmaceutical composition of embodiment 89 to modulate the immune system by directly or indirectly inducing proliferation and/or activation of immune cells.

TABLE 2
List of Amino Acid Sequences

| SEQ | | |
|---|---|---|
| ID | | |
| NO: | Name | Sequence |
| 1 | Heavy chain CDR-H1, anti-huPD- | NAMS |
| | L1/anti-huIFN-α2 DutaFab | |
| | M14HH17L | |
| 2 | Heavy chain CDR-H2, anti-huPD- | SISGSGGSISLARSVKG |
| | L1/anti-huIFN-α2 DutaFab | |
| | M14HH17L | |
| 3 | Heavy chain CDR-H3, anti-huPD- | DLGFMDV |
| | L1/anti-huIFN-α2 DutaFab | |
| | M14HH17L | |
| 4 | Light chain CDR-L1, anti-huPD- | DAVITISQELA |
| | L1/anti-huIFN-α2 DutaFab | |
| | M14HH17L | |
| 5 | Light chain CDR-L2, anti-huPD- | DASYHES |
| | L1/anti-huIFN-α2 DutaFab | |
| | M14HH17L | |
| 6 | Light chain CDR-L3, anti-huPD- | QQYGNWPHT |
| | L1/anti-huIFN-α2 DutaFab | |
| | M14HH17L | |
| 7 | Heavy chain variable domain VH, | MTQLVESGGGLVQPGGSLRLSCAADHTTEY |
| | anti-huPD-L1/anti-huIFN-α2 | NAMSWVRQAPGKGLEWVGSISGSGGSTSLA |
| | DutaFab M14HH17L | RSVKGRFTISRDNSKNTLYLQMNSLRAEDT |
| | | AVYYCARDLGFMDVWGQGTLVTVSS |
| 8 | Light chain variable domain VL, | DIQMTQSPSSLSASVGDRVTITCDAVITIS |
| | anti-huPD-L1/anti-huIFN-α2 | QELAWYQQKPGKAPKLLIYDASYHESGVPS |
| | DutaFab M14HH17L | RFSGSGSGTDFTLTISSLQPEDFATYYCQQ |
| | | YGNWPHTFGQGTKVEIK |
| 9 | Light chain CDR-L1 anti-huPD- | DALITISQELA |
| | L1/anti-huIFN-α2 (P1AI4748, | |
| | P1AI4797) | |
| 10 | Light chain variable domain anti- | DIQMTQSPSSLSASVGDRVTITCDALITIS |
| | huPD-L1/anti-huIFN-α2 VL | QELAWYQQKPGKAPKLLIYDASYHESGVPS |
| | (P1AI4748, P1AI4797) | RFSGSGSGTDFTLTISSLQPEDFATYYCQQ |
| | | YGNWPHTFGQGTKVEIK |
| 11 | Light chain CDR-L2 anti-huPD- | DASYHDT |
| | L1/anti-huIFN-α2 (P1AI4298) | |
| 12 | Light chain variable domain anti- | DIQMTQSPSSLSASVGDRVTITCDAVITIS |
| | huPD-L1/anti-huIFN-α2 VL | QELAWYQQKPGKAPKLLIYDASYHDTGVPS |
| | (P1AI4298) | RFSGSGSGTDFTLTISSLQPEDFATYYCQQ |
| | | YGNWPHTFGQGTKVEIK |
| 13 | Heavy chain CDR-H1, anti-huPD- | EQAMS |
| | L1/anti-huIFN-α2 DutaFab | |
| | E06P07 | |
| 14 | Heavy chain CDR-H2, anti-huPD- | SISPKGGSKSLNRNFNG |
| | L1/anti-huIFN-α2 DutaFab | |
| | E06P07 | |
| 15 | Heavy chain CDR-H3, anti-huPD- | DIGFMDG |
| | L1/anti-huIFN-α2 DutaFab | |
| | E06P07 | |
| 16 | Light chain CDR-L1, anti-huPD- | HGHVFISNYLA |
| | L1/anti-huIFN-α2 DutaFab | |
| | E06P07 | |
| 17 | Light chain CDR-L2, anti-huPD- | DGEWRDF |
| | L1/anti-huIFN-α2 DutaFab | |
| | E06P07 | |
| 18 | Light chain CDR-L3, anti-huPD- | QQYSNWPHT |
| | L1/anti-huIFN-α2 DutaFab | |
| | E06P07 | |
| 19 | Heavy chain variable domain VH, | ARQLVESGGGLVQPGGSLRLSCAASGFHFE |
| | anti-huPD-L1/anti-huIFN-α2 | EQAMSWVRQAPGKGLEWVGSISPKGGSKSL |
| | DutaFab E06P07 | NRNFNGRFTISRDNSKNTLYLQMNSLRAED |
| | | TAVYYCAKDIGFMDGWGQGTLVTVSS |
| 20 | Light chain variable domain VL, | YQMTQSPSSLSASVGDRVTITCHGHVFISN |
| | anti-huPD-L1/anti-huIFN-α2 | YLAWYQQKPGKAPKLLIYDGEWRDFGVPSR |
| | DutaFab E06P07 | FSGSGSDDSFTLTISSLQPEDFATYYCQQY |
| | | SNWPHTFGQGTKVEIK |
| 21 | Heavy chain CDR-H1, anti-huPD- | NAMS |
| | L1 M14 (P1AI3438/P1AI4475) | |
| 22 | Heavy chain CDR-H2, anti-huPD- | AISGSGGSTYYADSVKG |
| | L1 M14 (P1AI3438/P1AI4475) | |
| 23 | Heavy chain CDR-H3, anti-huPD- | DLGFMDV |
| | L1 M14 (P1AI3438/P1AI4475) | |
| 24 | Light chain CDR-L1, anti-huPD- | RASQSISSYLN |
| | L1 M14 (P1AI3438/P1AI4475) | |
| 25 | Light chain CDR-L2, anti-huPD- | DASYHEF |
| | L1 M14 (P1AI3438/P1AI4475) | |
| 26 | Light chain CDR-L3, anti-huPD- | QQSYSTPWT |
| | L1 M14 (P1AI3438/P1AI4475) | |
| 27 | Heavy chain variable domain VH, | MTQLVESGGGLVQPGGSLRLSCAADHTTEY |
| | anti-huPD-L1 M14 | NAMSWVRQAPGKGLEWVSAISGSGGSTYYA |
| | (P1AI3438/P1AI4475) | DSVKGRFTISRDNSKNTLYLQMNSLRAEDT |
| | | AVYYCARDLGFMDVWGQGTLVTVSS |
| 28 | Light chain variable domain VL, | DIQMTQSPSSLSASVGDRVTITCRASQSIS |
| | anti-huPD-L1 M14 | SYLNWYQQKPGKAPKLLIYDASYHEFGVPS |
| | (P1AI3438/P1AI4475) | RFSGSGSGTDFTLTISSLQPEDFATYYCQQ |
| | | SYSTPWTFGQGTKVEIK |
| 29 | Heavy chain CDR-H1, anti-huPD- | DSWIH |
| | L1 Atezolizumab | |
| 30 | Heavy chain CDR-H2, anti-huPD- | WISPYGGSTYYADSVKG |
| | L1 Atezolizumab | |
| 31 | Heavy chain CDR-H3, | RHWPGGFDY |
| | Atezolizumab | |
| 32 | Light chain CDR-L1, anti-huPD- | RASQDVSTAVA |
| | L1 Atezolizumab | |
| 33 | Light chain CDR-L2, anti-huPD- | SASFLYS |
| | L1 Atezolizumab | |
| 34 | Light chain CDR-L3, anti-huPD- | QQYLYHPAT |
| | L1 Atezolizumab | |
| 35 | Heavy chain variable domain VH, | EVQLVESGGGLVQPGGSLRLSCAASGFTFS |
| | anti-huPD-L1 Atezolizumab | DSWIHWVRQAPGKGLEWVAWISPYGGSTYY |
| | | ADSVKGRFTISADTSKNTAYLQMNSLRAED |
| | | TAVYYCARRHWPGGFDYWGQGTLVTVSS |
| 36 | Light chain variable domain VL, | DIQMTQSPSSLSASVGDRVTITCRASQDVS |
| | anti-huPD-L1 Atezolizumab | TAVAWYQQKPGKAPKLLIYSASFLYSGVPS |
| | | RFSGSGSGTDFTLTISSLQPEDFATYYCQQ |
| | | YLYHPATFGQGTKVEIK |
| 37 | heavy chain 1 of targeted IFN-α2a | DIQMTQSPSSLSASVGDRVTITCRASQSIS |
| | based on Dutaflip M14HH17L | SYLNWYQQKPGKAPKLLIYDASYHEFGVPS |
| | and anti-PD-L1 M14 (P1AI4295) | RFSGSGSGTDFTLTISSLQPEDFATYYCQQ |
| | H-Chain | SYSTPWTFGQGTKVEIKSSASTKGPSVFPL |
| | | APSSKSTSGGTAALGCLVKDYFPEPVTVSW |
| | | NSGALTSGVHTFPAVLQSSGLYSLSSVVTV |
| | | PSSSLGTQTYICNVNHKPSNTKVDKKVEPK |
| | | SCDKTHTCPPCPAPEAAGGPSVFLFPPKPK |
| | | DTLMISRTPEVTCVVVDVSHEDPEVKFNWY |
| | | VDGVEVHNAKTKPREEQYNSTYRVVSVLTV |
| | | LHQDWLNGKEYKCKVSNKALGAPIEKTISK |
| | | AKGQPREPQVCTLPPSRDELTKNQVSLSCA |
| | | VKGFYPSDIAVEWESNGQPENNYKTTPPVL |
| | | DSDGSFFLVSKLTVDKSRWQQGNVFSCSVM |
| | | HEALHNHYTQKSLSLSP |
| 38 | light chain 1 of targeted IFN-α2a | MTQLVESGGGLVQPGGSLRLSCAADHTTEY |
| | based on Dutaflip M14HH17L | NAMSWVRQAPGKGLEWVSAISGSGGSTYYA |
| | and anti-PD-L1 M14 (P1AI4295) | DSVKGRFTISRDNSKNTLYLQMNSLRAEDT |
| | B Chain | AVYYCARDLGFMDVWGQGTLVTVSSASVAA |
| | | PSVFIFPPSDEQLKSGTASVVCLLNNFYPR |
| | | EAKVQWKVDNALQSGNSQESVTEQDSKDST |
| | | YSLSSTLTLSKADYEKHKVYACEVTHQGLS |
| | | SPVTKSFNRGEC |
| 39 | heavy chain 2 of targeted IFN-α2a | MTQLVESGGGLVQPGGSLRLSCAADHTTEY |
| | based on Dutaflip M14HH17L | NAMSWVRQAPGKGLEWVGSISGSGGSTSLA |
| | and anti-PD-L1 M14 (P1AI4295) | RSVKGRFTISRDNSKNTLYLQMNSLRAEDT |
| | K Chain | AVYYCARDLGFMDVWGQGTLVTVSSASTKG |
| | | PSVFPLAPSSKSTSGGTAALGCLVEDYFPE |
| | | PVTVSWNSGALTSGVHTFPAVLQSSGLYSL |
| | | SSVVTVPSSSLGTQTYICNVNHKPSNTKVD |
| | | EKVEPKSCDKTHTCPPCPAPEAAGGPSVFL |
| | | FPPKPKDTLMISRTPEVTCVVVDVSHEDPE |
| | | VKFNWYVDGVEVHNAKTKPREEQYNSTYRV |
| | | VSVLTVLHQDWLNGKEYKCKVSNKALGAPI |
| | | EKTISKAKGQPREPQVYTLPPCRDELTKNQ |
| | | VSLWCLVKGFYPSDIAVEWESNGQPENNYK |
| | | TTPPVLDSDGSFFLYSKLTVDKSRWQQGNV |
| | | FSCSVMHEALHNHYTQKSLSLSP |
| 40 | light chain 2 of targeted IFN-α2a | CDLPQTHSLGSRRTLMLLAQMRKISLFSCL |
| | based on Dutaflip M14HH17L | KDRHDFGFPQEEFGNQFQKAETIPVLHEMI |
| | and anti-PD-L1 M14 (P1AI4295) | QQIFNLFSTKDSSAAWDETLLDKFYTELYQ |
| | A chain | QLNDLEACVIQGVGVTETPLMKEDSILAVR |
| | | KYFQRITLYLKEKKYSPCAWEVVRAEIMRS |
| | | FSLSTNLQESLRSKEGGSGGGGSGGGGSGG |
| | | GGSGGDIQMTQSPSSLSASVGDRVTITCDA |
| | | VITISQELAWYQQKPGKAPKLLIYDASYHE |
| | | SGVPSRFSGSGSGTDFTLTISSLQPEDFAT |
| | | YYCQQYGNWPHTFGQGTKVEIKRTVAAPSV |
| | | FIFPPSDRKLKSGTASVVCLLNNFYPREAK |
| | | VQWKVDNALQSGNSQESVTEQDSKDSTYSL |
| | | SSTLILSKADYEKHKVYACEVTHQGLSSPV |
| | | TKSFNRGEC |
| 41 | heavy chain 1 of targeted IFN-α2a | DIQMTQSPSSLSASVGDRVTITCRASQSIS |
| | based on Dutaflip 24664 and anti- | SYLNWYQQKPGKAPKLLIYDASYHEFGVPS |
| | PD-L1 M14 (P1AI4748) | RFSGSGSGTDFTLTISSLQPEDFATYYCQQ |
| | H Chain | SYSTPWTFGQGTKVEIKSSASTKGPSVFPL |
| | | APSSKSTSGGTAALGCLVKDYFPEPVTVSW |
| | | NSGALTSGVHTFPAVLQSSGLYSLSSVVTV |
| | | PSSSLGTQTYICNVNHKPSNTKVDKKVEPK |
| | | SCDKTHTCPPCPAPEAAGGPSVFLFPPKPK |
| | | DTLMISRTPEVTCVVVDVSHEDPEVKFNWY |
| | | VDGVEVHNAKTKPREEQYNSTYRVVSVLTV |
| | | LHQDWLNGKEYKCKVSNKALGAPIEKTISK |
| | | AKGQPREPQVCTLPPSRDELTKNQVSLSCA |
| | | VKGFYPSDIAVEWESNGQPENNYKTTPPVL |
| | | DSDGSFFLVSKLTVDKSRWQQGNVFSCSVM |
| | | HEALHNHYTQKSLSLSP |
| 42 | light chain 1 of targeted IFN-α2a | MTQLVESGGGLVQPGGSLRLSCAADHTTEY |
| | based on Dutaflip 24664 and anti- | NAMSWVRQAPGKGLEWVSAISGSGGSTYYA |
| | PD-L1 M14 (P1AI4748) | DSVKGRFTISRDNSKNTLYLQMNSLRAEDT |
| | B Chain | AVYYCARDLGFMDVWGQGTLVTVSSASVAA |
| | | PSVFIFPPSDEQLKSGTASVVCLLNNFYPR |
| | | EAKVQWKVDNALQSGNSQESVTEQDSKDST |
| | | YSLSSTLTLSKADYEKHKVYACEVTHQGLS |
| | | SPVTKSFNRGEC |
| 43 | heavy chain 2 of targeted IFN-α2a | MTQLVESGGGLVQPGGSLRLSCAADHTTEY |
| | based on Dutaflip 24664 and anti- | NAMSWVRQAPGKGLEWVGSISGSGGSTSLA |
| | PD-L1 M14 (P1AI4748) | RSVKGRFTISRDNSKNTLYLQMNSLRAEDT |
| | K Chain | AVYYCARDLGFMDVWGQGTLVTVSSASTKG |
| | | PSVFPLAPSSKSTSGGTAALGCLVEDYFPE |
| | | PVTVSWNSGALTSGVHTFPAVLQSSGLYSL |
| | | SSVVTVPSSSLGTQTYICNVNHKPSNTKVD |
| | | EKVEPKSCDKTHTCPPCPAPEAAGGPSVFL |
| | | FPPKPKDTLMISRTPEVTCVVVDVSHEDPE |
| | | VKFNWYVDGVEVHNAKTKPREEQYNSTYRV |
| | | VSVLTVLHQDWLNGKEYKCKVSNKALGAPI |
| | | EKTISKAKGQPREPQVYTLPPCRDELTKNQ |
| | | VSLWCLVKGFYPSDIAVEWESNGQPENNYK |
| | | TTPPVLDSDGSFFLYSKLTVDKSRWQQGNV |
| | | FSCSVMHEALHNHYTQKSLSLSP |
| 44 | light chain 2 of targeted IFN-α2a | CDLPQTHSLGSRRTLMLLAQMRKISLFSCL |
| | based on Dutaflip 24664 and anti- | KDRHDFGFPQEEFGNQFQKAETIPVLHEMI |
| | PD-L1 M14 (P1AI4748) | QQIFNLFSTKDSSAAWDETLLDKFYTELYQ |
| | A Chain | QLNDLEACVIQGVGVTETPLMKEDSILAVR |
| | | KYFQRITLYLKEKKYSPCAWEVVRAEIMRS |
| | | FSLSTNLQESLRSKEGGSGGGGSGGGGSGG |
| | | GGSGGDIQMTQSPSSLSASVGDRVTITCDA |
| | | LITISQELAWYQQKPGKAPKLLIYDASYHE |
| | | SGVPSRFSGSGSGTDFTLTISSLQPEDFAT |
| | | YYCQQYGNWPHTFGQGTKVEIKRTVAAPSV |
| | | FIFPPSDRKLKSGTASVVCLLNNFYPREAK |
| | | VQWKVDNALQSGNSQESVTEQDSKDSTYSL |
| | | SSTLTLSKADYEKHKVYACEVTHQGLSSPV |
| | | TKSFNRGEC |
| 45 | heavy chain 1 of targeted IFN-α2a | DIQMTQSPSSLSASVGDRVTITCRASQSIS |
| | based on Dutaflip 23932 and anti- | SYLNWYQQKPGKAPKLLIYDASYHEFGVPS |
| | PD-L1 M14 (P1AI4298) | RFSGSGSGTDFTLTISSLQPEDFATYYCQQ |
| | H Chain | SYSTPWTFGQGTKVEIKSSASTKGPSVFPL |
| | | APSSKSTSGGTAALGCLVKDYFPEPVTVSW |
| | | NSGALTSGVHTFPAVLQSSGLYSLSSVVTV |
| | | PSSSLGTQTYICNVNHKPSNTKVDKKVEPK |
| | | SCDKTHTCPPCPAPEAAGGPSVFLFPPKPK |
| | | DTLMISRTPEVTCVVVDVSHEDPEVKFNWY |
| | | VDGVEVHNAKTKPREEQYNSTYRVVSVLTV |
| | | LHQDWLNGKEYKCKVSNKALGAPIEKTISK |
| | | AKGQPREPQVCTLPPSRDELTKNQVSLSCA |
| | | VKGFYPSDIAVEWESNGQPENNYKTTPPVL |
| | | DSDGSFFLVSKLTVDKSRWQQGNVFSCSVM |
| | | HEALHNHYTQKSLSLSP |
| 46 | light chain 1 of targeted IFN-α2a | MTQLVESGGGLVQPGGSLRLSCAADHTTEY |
| | based on Dutaflip 23932 and anti- | NAMSWVRQAPGKGLEWVSAISGSGGSTYYA |
| | PD-L1 M14 (P1AI4298) | DSVKGRFTISRDNSKNTLYLQMNSLRAEDT |
| | B Chain | AVYYCARDLGFMDVWGQGTLVTVSSASVAA |
| | | PSVFIFPPSDEQLKSGTASVVCLLNNFYPR |
| | | EAKVQWKVDNALQSGNSQESVTEQDSKDST |
| | | YSLSSTLTLSKADYEKHKVYACEVTHQGLS |
| | | SPVIKSFNRGEC |
| 47 | heavy chain 2 of targeted IFN-α2a | MTQLVESGGGLVQPGGSLRLSCAADHTTEY |
| | based on Dutaflip 23932 and anti- | NAMSWVRQAPGKGLEWVGSISGSGGSTSLA |
| | PD-L1 M14 (P1AI4298) | RSVKGRFTISRDNSKNTLYLQMNSLRAEDT |
| | K Chain | AVYYCARDLGFMDVWGQGTLVTVSSASTKG |
| | | PSVFPLAPSSKSTSGGTAALGCLVEDYFPE |
| | | PVTVSWNSGALTSGVHTFPAVLQSSGLYSL |
| | | SSVVTVPSSSLGTQTYICNVNHKPSNTKVD |
| | | EKVEPKSCDKTHICPPCPAPEAAGGPSVFL |
| | | FPPKPKDTLMISRTPEVTCVVVDVSHEDPE |
| | | VKFNWYVDGVEVHNAKTKPREEQYNSTYRV |
| | | VSVLTVLHQDWLNGKEYKCKVSNKALGAPI |
| | | EKTISKAKGQPREPQVYTLPPCRDELTKNQ |
| | | VSLWCLVKGFYPSDIAVEWESNGQPENNYK |
| | | TTPPVLDSDGSFFLYSKLTVDKSRWQQGNV |
| | | FSCSVMHEALHNHYTQKSLSLSP |
| 48 | light chain 2 of targeted IFN-α2a | CDLPQTHSLGSRRTLMLLAQMRKISLFSCL |
| | based on Dutaflip 23932 and anti- | KDRHDFGFPQEEFGNQFQKAETIPVLHEMI |
| | PD-L1 M14 (P1AI4298) | QQIFNLFSTKDSSAAWDETLLDKFYTELYQ |
| | A Chain | QLNDLEACVIQGVGVTETPLMKEDSILAVR |
| | | KYFQRITLYLKEKKYSPCAWEVVRAEIMRS |
| | | FSLSTNLQESLRSKEGGSGGGGSGGGGSGG |
| | | GGSGGDIQMTQSPSSLSASVGDRVTITCDA |
| | | VITISQELAWYQQKPGKAPKLLIYDASYHD |
| | | TGVPSRFSGSGSGTDFTLTISSLQPEDFAT |
| | | YYCQQYGNWPHTFGQGTKVEIKRTVAAPSV |
| | | FIFPPSDRKLKSGTASVVCLLNNFYPREAK |
| | | VQWKVDNALQSGNSQESVTEQDSKDSTYSL |
| | | SSTLTLSKADYEKHKVYACEVTHQGLSSPV |
| | | TKSFNRGEC |
| | | DIQMTQSPSSLSASVGDRVTITCRASQDVS |
| 49 | heavy chain 1 of targeted IFN-α2a | TAVAWYQQKPGKAPKLLIYSASFLYSGVPS |
| | based on Dutaflip 24664 and anti- | RFSGSGSGTDFTLTISSLQPEDFATYYCQQ |
| | PD-L1 Atezolizumab (P1AI4797) | YLYHPATFGQGTKVEIKSSASTKGPSVFPL |
| | H Chain | APSSKSTSGGTAALGCLVKDYFPEPVTVSW |
| | | NSGALTSGVHTFPAVLQSSGLYSLSSVVTV |
| | | PSSSLGTQTYICNVNHKPSNTKVDKKVEPK |
| | | SCDKTHTCPPCPAPEAAGGPSVFLFPPKPK |
| | | DTLMISRTPEVTCVVVDVSHEDPEVKFNWY |
| | | VDGVEVHNAKTKPREEQYNSTYRVVSVLTV |
| | | LHQDWLNGKEYKCKVSNKALGAPIEKTISK |
| | | AKGQPREPQVCTLPPSRDELTKNQVSLSCA |
| | | VKGFYPSDIAVEWESNGQPENNYKTTPPVL |
| | | DSDGSFFLVSKLTVDKSRWQQGNVFSCSVM |
| | | HEALHNHYTQKSLSLSP |
| 50 | light chain 1 of targeted IFN-α2a | EVQLVESGGGLVQPGGSLRLSCAASGFTFS |
| | based on Dutaflip 24664 and anti- | DSWIHWVRQAPGKGLEWVAWISPYGGSTYY |
| | PD-L1 Atezolizumab (P1AI4797) | ADSVKGRFTISADTSKNTAYLQMNSLRAED |
| | B Chain | TAVYYCARRHWPGGFDYWGQGTLVTVSSAS |
| | | VAAPSVFIFPPSDEQLKSGTASVVCLLNNF |
| | | YPREAKVQWKVDNALQSGNSQESVTEQDSK |
| | | DSTYSLSSTLTLSKADYEKHKVYACEVTHQ |
| | | GLSSPVTKSFNRGEC |
| 51 | heavy chain 2 of targeted IFN-α2a | MTQLVESGGGLVQPGGSLRLSCAADHTTEY |
| | based on Dutaflip 24664 and anti- | NAMSWVRQAPGKGLEWVGSISGSGGSTSLA |
| | PD-L1 Atezolizumab (P1AI4797) | RSVKGRFTISRDNSKNTLYLQMNSLRAEDT |
| | K Chain | AVYYCARDLGFMDVWGQGTLVTVSSASTKG |
| | | PSVFPLAPSSKSTSGGTAALGCLVEDYFPE |
| | | PVTVSWNSGALTSGVHTFPAVLQSSGLYSL |
| | | SSVVTVPSSSLGTQTYICNVNHKPSNTKVD |
| | | EKVEPKSCDKTHTCPPCPAPEAAGGPSVFL |
| | | FPPKPKDTLMISRTPEVTCVVVDVSHEDPE |
| | | VKFNWYVDGVEVHNAKTKPREEQYNSTYRV |
| | | VSVLTVLHQDWLNGKEYKCKVSNKALGAPI |
| | | EKTISKAKGQPREPQVYTLPPCRDELTKNQ |
| | | VSLWCLVKGFYPSDIAVEWESNGQPENNYK |
| | | TTPPVLDSDGSFFLYSKLTVDKSRWQQGNV |
| | | FSCSVMHEALHNHYTQKSLSLSP |
| 52 | light chain 2 of targeted IFN-α2a | CDLPQTHSLGSRRTLMLLAQMRKISLFSCL |
| | based on Dutaflip 24664 and anti- | KDRHDFGFPQEEFGNQFQKAETIPVLHEMI |
| | PD-L1 Atezolizumab (P1AI4797) | QQIFNLFSTKDSSAAWDETLLDKFYTELYQ |
| | A Chain | QLNDLEACVIQGVGVTETPLMKEDSILAVR |
| | | KYFQRITLYLKEKKYSPCAWEVVRAEIMRS |
| | | FSLSTNLQESLRSKEGGSGGGGSGGGGSGG |
| | | GGSGGDIQMTQSPSSLSASVGDRVTITCDA |
| | | LITISQELAWYQQKPGKAPKLLIYDASYHE |
| | | SGVPSRFSGSGSGTDFTLTISSLQPEDFAT |
| | | YYCQQYGNWPHTFGQGTKVEIKRTVAAPSV |
| | | FIFPPSDRKLKSGTASVVCLLNNFYPREAK |
| | | VQWKVDNALQSGNSQESVTEQDSKDSTYSL |
| | | SSTLTLSKADYEKHKVYACEVTHQGLSSPV |
| | | TKSFNRGEC |
| 53 | heavy chain 1 of targeted IFN-α2a | DIQMTQSPSSLSASVGDRVTITCRASQDVS |
| | based on Dutaflip 23932 and anti- | TAVAWYQQKPGKAPKLLIYSASFLYSGVPS |
| | PD-L1 Atezolizumab (P1AI4798) | RFSGSGSGTDFTLTISSLQPEDFATYYCQQ |
| | H Chain | YLYHPATFGQGTKVEIKSSASTKGPSVFPL |
| | | APSSKSTSGGTAALGCLVKDYFPEPVTVSW |
| | | NSGALTSGVHTFPAVLQSSGLYSLSSVVTV |
| | | PSSSLGTQTYICNVNHKPSNTKVDKKVEPK |
| | | SCDKTHTCPPCPAPEAAGGPSVFLFPPKPK |
| | | DTLMISRTPEVTCVVVDVSHEDPEVKFNWY |
| | | VDGVEVHNAKTKPREEQYNSTYRVVSVLTV |
| | | LHQDWLNGKEYKCKVSNKALGAPIEKTISK |
| | | AKGQPREPQVCTLPPSRDELTKNQVSLSCA |
| | | VKGFYPSDIAVEWESNGQPENNYKTTPPVL |
| | | DSDGSFFLVSKLTVDKSRWQQGNVFSCSVM |
| | | HEALHNHYTQKSLSLSP |
| 54 | light chain 1 of targeted IFN-α2a | EVQLVESGGGLVQPGGSLRLSCAASGFTFS |
| | based on Dutaflip 23932 and anti- | DSWIHWVRQAPGKGLEWVAWISPYGGSTYY |
| | PD-L1 Atezolizumab (P1AI4798) | ADSVKGRFTISADTSKNTAYLQMNSLRAED |
| | B Chain | TAVYYCARRHWPGGFDYWGQGTLVTVSSAS |
| | | VAAPSVFIFPPSDEQLKSGTASVVCLLNNF |
| | | YPREAKVQWKVDNALQSGNSQESVTEQDSK |
| | | DSTYSLSSTLILSKADYEKHKVYACEVTHQ |
| | | GLSSPVIKSFNRGEC |
| 55 | heavy chain 2 of targeted IFN-α2a | MTQLVESGGGLVQPGGSLRLSCAADHTTEY |
| | based on Dutaflip 23932 and anti- | NAMSWVRQAPGKGLEWVGSISGSGGSTSLA |
| | PD-L1 Atezolizumab (P1AI4798) | RSVKGRFTISRDNSKNTLYLQMNSLRAEDT |
| | K Chain | AVYYCARDLGFMDVWGQGTLVTVSSASTKG |
| | | PSVFPLAPSSKSTSGGTAALGCLVEDYFPE |
| | | PVTVSWNSGALTSGVHTFPAVLQSSGLYSL |
| | | SSVVTVPSSSLGTQTYICNVNHKPSNTKVD |
| | | EKVEPKSCDKTHTCPPCPAPEAAGGPSVFL |
| | | FPPKPKDTLMISRTPEVTCVVVDVSHEDPE |
| | | VKFNWYVDGVEVHNAKTKPREEQYNSTYRV |
| | | VSVLTVLHQDWLNGKEYKCKVSNKALGAPI |
| | | EKTISKAKGQPREPQVYTLPPCRDELTKNQ |
| | | VSLWCLVKGFYPSDIAVEWESNGQPENNYK |
| | | TTPPVLDSDGSFFLYSKLTVDKSRWQQGNV |
| | | FSCSVMHEALHNHYTQKSLSLSP |
| 56 | light chain 2 of targeted IFN-α2a | CDLPQTHSLGSRRTLMLLAQMRKISLFSCL |
| | based on Dutaflip 23932 and anti- | KDRHDFGFPQEEFGNQFQKAETIPVLHEMI |
| | PD-L1 Atezolizumab (P1AI4798) | QQIFNLFSTKDSSAAWDETLLDKFYTELYQ |
| | A Chain | QLNDLEACVIQGVGVTETPLMKEDSILAVR |
| | | KYFQRITLYLKEKKYSPCAWEVVRAEIMRS |
| | | FSLSTNLQESLRSKEGGSGGGGSGGGGSGG |
| | | GGSGGDIQMTQSPSSLSASVGDRVTITCDA |
| | | VITISQELAWYQQKPGKAPKLLIYDASYHD |
| | | TGVPSRFSGSGSGTDFTLTISSLQPEDFAT |
| | | YYCQQYGNWPHTFGQGTKVEIKRTVAAPSV |
| | | FIFPPSDRKLKSGTASVVCLLNNFYPREAK |
| | | VQWKVDNALQSGNSQESVTEQDSKDSTYSL |
| | | SSTLTLSKADYEKHKVYACEVTHQGLSSPV |
| | | TKSFNRGEC |
| 57 | heavy chain 1 of targeted IFN-α2a | DIQMTQSPSSLSASVGDRVTITCRASQDVS |
| | based on Dutaflip E06P07and | TAVAWYQQKPGKAPKLLIYSASFLYSGVPS |
| | anti-PD-L1 Atezolizumab | RFSGSGSGTDFTLTISSLQPEDFATYYCQQ |
| | (P1AH1111) | YLYHPATFGQGTKVEIKSSASTKGPSVFPL |
| | K Chain | APSSKSTSGGTAALGCLVKDYFPEPVTVSW |
| | | NSGALTSGVHTFPAVLQSSGLYSLSSVVTV |
| | | PSSSLGTQTYICNVNHKPSNTKVDKKVEPK |
| | | SCDKTHTCPPCPAPEAAGGPSVFLFPPKPK |
| | | DTLMISRTPEVTCVVVDVSHEDPEVKFNWY |
| | | VDGVEVHNAKTKPREEQYNSTYRVVSVLTV |
| | | LHQDWLNGKEYKCKVSNKALGAPIEKTISK |
| | | AKGQPREPQVYTLPPCRDELTKNQVSLWCL |
| | | VKGFYPSDIAVEWESNGQPENNYKTTPPVL |
| | | DSDGSFFLYSKLTVDKSRWQQGNVFSCSVM |
| | | HEALHNHYTQKSLSLSP |
| 58 | light chain 1 of targeted IFN-α2a | EVQLVESGGGLVQPGGSLRLSCAASGFTFS |
| | based on Dutaflip E06P07and | DSWIHWVRQAPGKGLEWVAWISPYGGSTYY |
| | anti-PD-L1 Atezolizumab | ADSVKGRFTISADISKNTAYLQMNSLRAED |
| | (P1AH1111) | TAVYYCARRHWPGGFDYWGQGTLVTVSSAS |
| | A Chain | VAAPSVFIFPPSDEQLKSGTASVVCLLNNF |
| | | YPREAKVQWKVDNALQSGNSQESVTEQDSK |
| | | DSTYSLSSTLTLSKADYEKHKVYACEVTHQ |
| | | GLSSPVTKSFNRGEC |
| 59 | heavy chain 2 of targeted IFN-α2a | ARQLVESGGGLVQPGGSLRLSCAASGFHFE |
| | based on Dutaflip E06P07and | EQAMSWVRQAPGKGLEWVGSISPKGGSKSL |
| | anti-PD-L1 Atezolizumab | NRNFNGRFTISRDNSKNTLYLQMNSLRAED |
| | (P1AH1111) | TAVYYCAKDIGFMDGWGQGTLVTVSSASTK |
| | H Chain | GPSVFPLAPSSKSTSGGTAALGCLVEDYFP |
| | | EPVTVSWNSGALTSGVHIFPAVLQSSGLYS |
| | | LSSVVTVPSSSLGTQTYICNVNHKPSNTKV |
| | | DEKVEPKSCDKTHTCPPCPAPEAAGGPSVF |
| | | LFPPKPKDTLMISRTPEVTCVVVDVSHEDP |
| | | EVKFNWYVDGVEVHNAKTKPREEQYNSTYR |
| | | VVSVLTVLHQDWLNGKEYKCKVSNKALGAP |
| | | IEKTISKAKGQPREPQVCTLPPSRDELTKN |
| | | QVSLSCAVKGFYPSDIAVEWESNGQPENNY |
| | | KTTPPVLDSDGSFFLVSKLTVDKSRWQQGN |
| | | VFSCSVMHEALHNHYTQKSLSLSP |
| 60 | light chain 2 of targeted IFN-α2a | CDLPQTHSLGSRRTLMLLAQMRKISLFSCL |
| | based on Dutaflip E06P07and | KDRHDFGFPQEEFGNQFQKAETIPVLHEMI |
| | anti-PD-L1 Atezolizumab | QQIFNLFSTKDSSAAWDETLLDKFYTELYQ |
| | (P1AH1111) | QLNDLEACVIQGVGVTETPLMKEDSILAVR |
| | K Chain | KYFQRITLYLKEKKYSPCAWEVVRAEIMRS |
| | | FSLSTNLQESLRSKEGGSGGGGSGGGGSGG |
| | | GGSGGYQMTQSPSSLSASVGDRVTITCHGH |
| | | VFISNYLAWYQQKPGKAPKLLIYDGEWRDF |
| | | GVPSRFSGSGSDDSFTLTISSLQPEDFATY |
| | | YCQQYSNWPHTFGQGTKVEIKRTVAAPSVF |
| | | IFPPSDRKLKSGTASVVCLLNNFYPREAKV |
| | | QWKVDNALQSGNSQESVTEQDSKDSTYSLS |
| | | STLTLSKADYEKHKVYACEVTHQGLSSPVT |
| | | KSFNRGEC |
| 61 | heavy chain 1 of reference | CDLPQTHSLGSRRTLMLLAQMRKISLFSCL |
| | molecule DBA_N-term_IFNα | KDRHDFGFPQEEFGNQFQKAETIPVLHEMI |
| | C08 (P1AI0336) | QQIFNLFSTKDSSAAWDETLLDKFYTELYQ |
| | | QLNDLEACVIQGVGVTETPLMKEDSILAVR |
| | | KYFQRITLYLKEKKYSPCAWEVVRAEIMRS |
| | | FSLSTNLQESLRSKEGGSGGGGSGGGGSGG |
| | | GGSGGQVQLVQSGAEVKKPGASVKVSCKAS |
| | | GGTFSSYAISWVRQAPGQGLEWMGIIDPSV |
| | | TYTRYAQKFQGRVTMTRDISTSTVYMELSS |
| | | LRSEDTAVYYCARSLFPTIFGVEVAFDIWG |
| | | QGTLVTVSSASTKGPSVFPLAPSSKSTSGG |
| | | TAALGCLVEDYFPEPVTVSWNSGALTSGVH |
| | | TFPAVLQSSGLYSLSSVVTVPSSSLGTQTY |
| | | ICNVNHKPSNTKVDEKVEPKSCDKTHTCPP |
| | | CPAPEAAGGPSVFLFPPKPKDTLMISRTPE |
| | | VTCVVVDVSHEDPEVKFNWYVDGVEVHNAK |
| | | TKPREEQYNSTYRVVSVLTVLHQDWLNGKE |
| | | YKCKVSNKALGAPIEKTISKAKGQPREPQV |
| | | CTLPPSRDELTKNQVSLSCAVKGFYPSDIA |
| | | VEWESNGQPENNYKTTPPVLDSDGSFFLVS |
| | | KLTVDKSRWQQGNVFSCSVMHEALHNHYTQ |
| | | KSLSLSPG |
| 62 | heavy chain 2 (Fc fragment only) | DKTHTCPPCPAPEAAGGPSVFLFPPKPKDT |
| | of reference molecule DBA_N- | LMISRTPEVTCVVVDVSHEDPEVKFNWYVD |
| | term_IFNα C08 (P1AI0336) | GVEVHNAKTKPREEQYNSTYRVVSVLTVLH |
| | | QDWLNGKEYKCKVSNKALGAPIEKTISKAK |
| | | GQPREPQVYTLPPCRDELTKNQVSLWCLVK |
| | | GFYPSDIAVEWESNGQPENNYKTTPPVLDS |
| | | DGSFFLYSKLTVDKSRWQQGNVFSCSVMHE |
| | | ALHNHYTQKSLSLSPG |
| 63 | light chain of reference molecule | DIQMTQSPSSLSASVGDRVTITCQASQDIS |
| | DBA_N-term_IFNα C08 | NYLNWYQQKPGKAPKLLIYGASTLQSGVPS |
| | (P1AI0336) | RFSGSGSGTDFTLTISSLQPEDFATYYCQQ |
| | | TYSTPITFGQGTKVEIKRIVAAPSVFIFPP |
| | | SDRKLKSGTASVVCLLNNFYPREAKVQWKV |
| | | DNALQSGNSQESVTEQDSKDSTYSLSSTLT |
| | | LSKADYEKHKVYACEVTHQGLSSPVTKSFN |
| | | RGEC |
| 64 | heavy chain 1 of reference | CDLPQTHSLGSRRTLMLLAQMRKISLFSCL |
| | molecule DBA_N- | KDRHDFGFPQEEFGNQFQKAETIPVLHEMI |
| | term_IFNα_B09 (P1AI0337) | QQIFNLFSTKDSSAAWDETLLDKFYTELYQ |
| | | QLNDLEACVIQGVGVTETPLMKEDSILAVR |
| | | KYFQRITLYLKEKKYSPCAWEVVRAEIMRS |
| | | FSLSTNLQESLRSKEGGSGGGGSGGGGSGG |
| | | GGSGGQVQLVQSGAEVKKPGASVKVSCKAS |
| | | GGTFTGYYMHWVRQAPGQGLEWMGWVNPNS |
| | | GNTGYAQKFQGRVTMTRDTSTSTVYMELSS |
| | | LRSEDTAVYYCARSLEPTIFGVEVAFDIWG |
| | | QGTLVTVSSASTKGPSVFPLAPSSKSTSGG |
| | | TAALGCLVEDYFPEPVTVSWNSGALTSGVH |
| | | TFPAVLQSSGLYSLSSVVTVPSSSLGTQTY |
| | | ICNVNHKPSNTKVDEKVEPKSCDKTHTCPP |
| | | CPAPEAAGGPSVFLFPPKPKDTLMISRTPE |
| | | VTCVVVDVSHEDPEVKFNWYVDGVEVHNAK |
| | | TKPREEQYNSTYRVVSVLTVLHQDWLNGKE |
| | | YKCKVSNKALGAPIEKTISKAKGQPREPQV |
| | | CTLPPSRDELTKNQVSLSCAVKGFYPSDIA |
| | | VEWESNGQPENNYKITPPVLDSDGSFFLVS |
| | | KLTVDKSRWQQGNVFSCSVMHEALHNHYTQ |
| | | KSLSLSPG |
| 65 | heavy chain 2 (Fc fragment only) | DKTHTCPPCPAPEAAGGPSVFLFPPKPKDT |
| | of reference molecule DBA_N- | LMISRTPEVTCVVVDVSHEDPEVKFNWYVD |
| | term_IFNα_B09 (P1AI0337) | GVEVHNAKTKPREEQYNSTYRVVSVLTVLH |
| | | QDWLNGKEYKCKVSNKALGAPIEKTISKAK |
| | | GQPREPQVYTLPPCRDELTKNQVSLWCLVK |
| | | GFYPSDIAVEWESNGQPENNYKITPPVLDS |
| | | DGSFFLYSKLTVDKSRWQQGNVFSCSVMHE |
| | | ALHNHYTQKSLSLSPG |
| 66 | light chain of reference molecule | DIQMTQSPSSLSASVGDRVTITCQASQDIS |
| | DBA_N-term_IFNα_B09 | NYLNWYQQKPGKAPKLLIYAASSLQSGVPS |
| | (P1AI0337) | RFSGSGSGTDFTLTISSLQPEDFATYYCQQ |
| | | SYSPPPTFGQGTKLEIKRTVAAPSVFIFPP |
| | | SDRKLKSGTASVVCLLNNFYPREAKVQWKV |
| | | DNALQSGNSQESVTEQDSKDSTYSLSSTLT |
| | | LSKADYEKHKVYACEVTHQGLSSPVTKSFN |
| | | RGEC |
| 67 | heavy chain 1 of reference | CDLPQTHSLGSRRTLMLLAQMRKISLFSCL |
| | molecule DBA_N-term_IFNα | KDRHDFGFPQEEFGNQFQKAETIPVLHEMI |
| | C08 (P1AI0338) | QQIFNLFSTKDSSAAWDETLLDKFYTELYQ |
| | | QLNDLEACVIQGVGVTETPLMKEDSILAVR |
| | | KYFQRITLYLKEKKYSPCAWEVVRAEIMRS |
| | | FSLSTNLQESLRSKEGGSGGGGSGGGGSGG |
| | | GGSGGQVQLVQSGAEVKKPGASVKVSCKAS |
| | | GYTFSNYYVHWVRQAPGQGLEWMGWMDPNS |
| | | GGTGYAHQFQGRVTMTRDTSTSTVYMELSS |
| | | LRSEDTAVYYCAKEVFSGWYDYWGQGTLVT |
| | | VSSASTKGPSVFPLAPSSKSTSGGTAALGC |
| | | LVEDYFPEPVTVSWNSGALTSGVHTFPAVL |
| | | QSSGLYSLSSVVTVPSSSLGTQTYICNVNH |
| | | KPSNTKVDEKVEPKSCDKTHTCPPCPAPEA |
| | | AGGPSVFLFPPKPKDTLMISRTPEVTCVVV |
| | | DVSHEDPEVKFNWYVDGVEVHNAKTKPREE |
| | | QYNSTYRVVSVLTVLHQDWLNGKEYKCKVS |
| | | NKALGAPIEKTISKAKGQPREPQVCTLPPS |
| | | RDELTKNQVSLSCAVKGFYPSDIAVEWESN |
| | | GQPENNYKTTPPVLDSDGSFFLVSKLTVDK |
| | | SRWQQGNVFSCSVMHEALHNHYTQKSLSLS |
| | | PG |
| 68 | heavy chain 2 (Fc fragment only) | DKTHTCPPCPAPEAAGGPSVFLFPPKPKDT |
| | of reference molecule DBA_N- | LMISRTPEVTCVVVDVSHEDPEVKFNWYVD |
| | term_IFNα C08 (P1AI0338) | GVEVHNAKTKPREEQYNSTYRVVSVLTVLH |
| | | QDWLNGKEYKCKVSNKALGAPIEKTISKAK |
| | | GQPREPQVYTLPPCRDELTKNQVSLWCLVK |
| | | GFYPSDIAVEWESNGQPENNYKTTPPVLDS |
| | | DGSFFLYSKLTVDKSRWQQGNVFSCSVMHE |
| | | ALHNHYTQKSLSLSPG |
| 69 | light chain of reference molecule | DIQMTQSPSSLSASVGDRVTITCRASQSIS |
| | DBA_N-term_IFNα_SeqID41 | SYLNWYQQKPGKAPKLLIYAASSLQSGVPS |
| | (P1AI0338) | RFSGSGSGTDFTLTISSLQPEDFATYYCQQ |
| | | SYSTPYTFGQGTKVEIKRTVAAPSVFIFPP |
| | | SDRKLKSGTASVVCLLNNFYPREAKVQWKV |
| | | DNALQSGNSQESVTEQDSKDSTYSLSSTLT |
| | | LSKADYEKHKVYACEVTHQGLSSPVTKSFN |
| | | RGEC |
| 70 | heavy chain 1 of reference | QVQLVQSGAEVKKPGASVKVSCKASGYTFT |
| | molecule | KNYMHWVRQAPGQGLEWLGWVSPDSGYTGY |
| | DBA_Fig9a_SeqID92_109_126 | AQKFQGRVTMTRDTSTSTVYMELSSLRSED |
| | (P1AI1297) | TAVYYCTTDLLSLELDDAFDIWGQGTMVTV |
| | | SSASGGGGSGGGGSGGGGSHASDIQMTQSP |
| | | SSLSASVGDRVTITCRASQSISSWLAWYQQ |
| | | KPGKAPKLLIYAASTLQSGVPSRFSGSGSG |
| | | TDFTLTISSLQPEDFATYYCQQSYSTPLTF |
| | | GGGTKLEIKPRGPTIKPCPPCKCPAPNAAG |
| | | GPSVFIFPPKTKDVLMISLSPIVTCVVVDV |
| | | SEDDPDVQISWFVNNVEVHTAQTQTHREDY |
| | | NSTLRVVSALPIQHQDWMSGKEFKCKVNNK |
| | | DLGAPIERTISKPKGSVRAPQVYVLPPCEE |
| | | EMTKKQVTLSCAVTDFMPEDIYVEWTNNGK |
| | | TELNYKNTEPVLDSDGSYFMVSKLRVEKKN |
| | | WVERNSYSCSVVHEGLHNHHTTKSFSRTPG |
| 71 | heavy chain 2 of reference | CDLPQTHSLGSRRTLMLLAQMRRISLFSCL |
| | molecule | KDRHDFGFPQEEFGNQFQKAETIPVLHEMI |
| | DBA_Fig9a_SeqID92_109_126 | QQIFNLFSTKDSSAAWDETLLDKFCTELYQ |
| | (P1AI1297) | QLNDLEACVMQEERVGETPLMNADSILAVK |
| | | KYFRRITLYLTEKKYSPCAWEVVRAEIVRS |
| | | LSLSTNLQERLRRKEGGGGSSGGGGGGGGS |
| | | GGGGSQVQLVQSGAEVKKPGASVKVSCKAS |
| | | GYTFSNYYIHWVRQAPGQGLEWMGWMDSNS |
| | | GGTGYAQKFQGRVTMTRDTSTSTVYMELSS |
| | | LRSEDTAVYYCAKEVFSGWYDYWGQGTLVT |
| | | VSSAKTTAPSVYPLAPVCGDTTGSSVTLGC |
| | | LVKGYFPEPVILTWNSGSLSSGVHTFPAVL |
| | | QSDLYTLSSSVTVISSTWPSQSITCNVAHP |
| | | ASSTKVDKKIEPRGPTIKPCPPCKCPAPNA |
| | | AGGPSVFIFPPKTKDVLMISLSPIVTCVVV |
| | | DVSEDDPDVQISWFVNNVEVHTAQTQTHRE |
| | | DYNSTLRVVSALPIQHQDWMSGKEFKCKVN |
| | | NKDLGAPIERTISKPKGSVRAPQVCVLPPP |
| | | EEEMTKKQVTLWCMVTDFMPEDIYVEWTNN |
| | | GKTELNYKNTEPVLDSDGSYFMYSKLRVEK |
| | | KNWVERNSYSCSVVHEGLHNHHTTKSFSRT |
| | | PG |
| 72 | light chain of reference molecule | DIQMTQSPSSLSASVGDRVTITCRASQSIS |
| | DBA_Fig9a_SeqID92_109_126 | SYLNWYQQKPGKAPKLLIYAASSLESGVPS |
| | (P1AI1297) | RFSGSGSGTDFTLTISSLQPEDFATYYCQQ |
| | | SYSTPYTFGQGTKVDIKRADAAPTVSIFPP |
| | | SSEQLTSGGASVVCFLNNFYPKDINVKWKI |
| | | DGSERQNGVLNSWTDQDSKDSTYSMSSTLT |
| | | LIKDEYERHNSYTCEATHKTSTSPIVKSFN |
| | | RNEC |
| 73 | heavy chain 1 of reference | QVQLVQSGAEVKKPGASVKVSCKASGYTFT |
| | molecule DBA_Fig9a_SeqID42- | KNYMHWVRQAPGQGLEWLGWVSPDSGYTGY |
| | 44 (P1AI1298) | AQKFQGRVTMTRDTSTSTVYMELSSLRSED |
| | | TAVYYCTTDLLSLELDDAFDIWGQGTMVTV |
| | | SSASGGGGSGGGGSGGGGSHASDIQMTQSP |
| | | SSLSASVGDRVTITCRASQSISSWLAWYQQ |
| | | KPGKAPKLLIYAASTLQSGVPSRFSGSGSG |
| | | TDFTLTISSLQPEDFATYYCQQSYSTPLTF |
| | | GGGTKLEIKPRGPTIKPCPPCKCPAPNAAG |
| | | GPSVFIFPPKTKDVLMISLSPIVTCVVVDV |
| | | SEDDPDVQISWFVNNVEVHTAQTQTHREDY |
| | | NSTLRVVSALPIQHQDWMSGKEFKCKVNNK |
| | | DLGAPIERTISKPKGSVRAPQVYVLPPCEE |
| | | EMTKKQVTLSCAVTDFMPEDIYVEWTNNGK |
| | | TELNYKNTEPVLDSDGSYFMVSKLRVEKKN |
| | | WVERNSYSCSVVHEGLHNHHTTKSFSRTPG |
| 74 | heavy chain 2 of reference | CDLPQTHSLGSRRTLMLLAQMRRISLFSCL |
| | molecule DBA_Fig9a_SeqID42- | KDRHDFGFPQEEFGNQFQKAETIPVLHEMI |
| | 44 (P1AI1298) | QQIFNLFSTKDSSAAWDETLLDKFCTELYQ |
| | | QLNDLEACVMQEERVGETPLMNADSILAVK |
| | | KYFRRITLYLTEKKYSPCAWEVVRAEIVRS |
| | | LSLSTNLQERLRRKEGGGGSGGGGSGGGGS |
| | | GGGGSQVQLVQSGAEVKKPGASVKVSCKAS |
| | | GYTFSGYYIHWVRQAPGQGLEWMGWMDSNS |
| | | GGTGYAQKFQGRVTMTRDTSTSTVYMELSS |
| | | LRSEDTAVYYCAKEVFSGWYDYWGQGTLVT |
| | | VSSAKTTAPSVYPLAPVCGDTTGSSVTLGC |
| | | LVKGYFPEPVTLTWNSGSLSSGVHTFPAVL |
| | | QSDLYTLSSSVTVISSTWPSQSITCNVAHP |
| | | ASSTKVDKKIEPRGPTIKPCPPCKCPAPNA |
| | | AGGPSVFIFPPKTKDVLMISLSPIVTCVVV |
| | | DVSEDDPDVQISWFVNNVEVHTAQTQTHRE |
| | | DYNSTLRVVSALPIQHQDWMSGKEFKCKVN |
| | | NKDLGAPIERTISKPKGSVRAPQVCVLPPP |
| | | EEEMTKKQVTLWCMVTDFMPEDIYVEWTNN |
| | | GKTELNYKNTEPVLDSDGSYFMYSKLRVEK |
| | | KNWVERNSYSCSVVHEGLHNHHTTKSFSRT |
| | | PG |
| 75 | light chain of reference molecule | DIQMTQSPSSLSASVGDRVTITCRASQSIS |
| | DBA_Fig9a_SeqID42-44 | SYLNWYQQKPGKAPKLLIYAASSLQSGVPS |
| | (P1AI1298) | RFSGSGSGTDFTLTISSLQPEDFATYYCQQ |
| | | SYSTPYTFGQGTKVEIKRADAAPTVSIFPP |
| | | SSEQLTSGGASVVCFLNNFYPKDINVKWKI |
| | | DGSERQNGVLNSWIDQDSKDSTYSMSSTLT |
| | | LTKDEYERHNSYTCEATHKTSTSPIVKSFN |
| | | RNEC |
| 76 | heavy chain 1 of reference | CDLPQTHSLGSRRTLMLLAQMRRISLFSCL |
| | molecule | KDRHDFGFPQEEFGNQFQKAETIPVLHEMI |
| | DBA_Fig9a_SeqID49_44_48 | QQIFNLFSTKDSSAAWDETLLDKFCTELYQ |
| | (P1AI1326) | QLNDLEACVMQEERVGETPLMNADSILAVK |
| | | KYFRRITLYLTEKKYSPCAWEVVRAEIVRS |
| | | LSLSTNLQERLRRKEGGGGSGGGGSGGGGS |
| | | GGGGSQVQLVQSGAEVKKPGASVKVSCKAS |
| | | GGTFSSYAISWVRQAPGQGLEWMGIIDPSV |
| | | TYTRYAQKFQGRVTMTRDISTSTVYMELSS |
| | | LRSEDTAVYYCARSLFPTIFGVEVAFDIWG |
| | | QGTLVTVSSASGGGGSGGGGSGGGGSHASD |
| | | IQMTQSPSSLSASVGDRVTITCQASQDISN |
| | | YLNWYQQKPGKAPKLLIYGASTLQSGVPSR |
| | | FSGSGSGTDFTLTISSLQPEDFATYYCQQT |
| | | YSTPITFGQGTKVEIKAKTTAPSVYPLAPV |
| | | CGDTTGSSVTLGCLVKGYFPEPVILTWNSG |
| | | SLSSGVHTFPAVLQSDLYTLSSSVTVISST |
| | | WPSQSITCNVAHPASSTKVDKKIEPRGPTI |
| | | KPCPPCKCPAPNAAGGPSVFIFPPKTKDVL |
| | | MISLSPIVTCVVVDVSEDDPDVQISWFVNN |
| | | VEVHTAQTQTHREDYNSTLRVVSALPIQHQ |
| | | DWMSGKEFKCKVNNKDLGAPIERTISKPKG |
| | | SVRAPQVYVLPPCEEEMTKKQVTLSCAVTD |
| | | FMPEDIYVEWTNNGKTELNYKNTEPVLDSD |
| | | GSYFMVSKLRVEKKNWVERNSYSCSVVHEG |
| | | LHNHHTTKSFSRTPG |
| 77 | heavy chain 2 of reference | QVQLVQSGAEVKKPGASVKVSCKASGYTFS |
| | molecule | GYYIHWVRQAPGQGLEWMGWMDSNSGGTGY |
| | DBA_Fig9a_SeqID49_44_48 | AQKFQGRVTMTRDTSTSTVYMELSSLRSED |
| | (P1AI1326) | TAVYYCAKEVFSGWYDYWGQGTLVTVSSAK |
| | | TTAPSVYPLAPVCGDTTGSSVTLGCLVKGY |
| | | FPEPVTLTWNSGSLSSGVHTFPAVLQSDLY |
| | | TLSSSVTVISSTWPSQSITCNVAHPASSTK |
| | | VDKKIEPRGPTIKPCPPCKCPAPNAAGGPS |
| | | VFIFPPKTKDVLMISLSPIVTCVVVDVSED |
| | | DPDVQISWFVNNVEVHTAQTQTHREDYNST |
| | | LRVVSALPIQHQDWMSGKEFKCKVNNKDLG |
| | | APIERTISKPKGSVRAPQVCVLPPPEEEMT |
| | | KKQVTLWCMVTDFMPEDIYVEWTNNGKTEL |
| | | NYKNTEPVLDSDGSYFMYSKLRVEKKNWVE |
| | | RNSYSCSVVHEGLHNHHTTKSFSRTPG |
| 78 | light chain of reference molecule | DIQMTQSPSSLSASVGDRVTITCRASQSIS |
| | DBA_Fig9a_SeqID49_44_48 | SYLNWYQQKPGKAPKLLIYAASSLQSGVPS |
| | (P1AI1326) | RFSGSGSGTDFTLTISSLQPEDFATYYCQQ |
| | | SYSTPYTFGQGTKVEIKRADAAPTVSIFPP |
| | | SSEQLTSGGASVVCFLNNFYPKDINVKWKI |
| | | DGSERQNGVLNSWTDQDSKDSTYSMSSTLT |
| | | LTKDEYERHNSYTCEATHKISTSPIVKSFN |
| | | RNEC |
| 79 | human Interferon-alpha 2a | CDLPQTHSLGSRRTLMLLAQMRKISLFSCL |
| | (huIFN-α2a) | KDRHDFGFPQEEFGNQFQKAETIPVLHEMI |
| | | QQIFNLFSTKDSSAAWDETLLDKFYTELYQ |
| | | QLNDLEACVIQGVGVTETPLMKEDSILAVR |
| | | KYFQRITLYLKEKKYSPCAWEVVRAEIMRS |
| | | FSLSTNLQESLRSKE |
| 80 | human Interferon-alpha 2b | CDLPQTHSLGSRRTLMLLAQMRRISLFSCL |
| | (huIFN-α2b) | KDRHDFGFPQEEFGNQFQKAETIPVLHEMI |
| | | QQIFNLFSTKDSSAAWDETLLDKFYTELYQ |
| | | QLNDLEACVIQGVGVTETPLMKEDSILAVR |
| | | KYFQRITLYLKEKKYSPCAWEVVRAEIMRS |
| | | FSLSTNLQESLRSKE |
| 81 | human Interferon-alpha 2a | MALTFALLVALLVLSCKSSCSVGCDLPQTH |
| | (huIFN-α2a) precursor with N- | SLGSRRTLMLLAQMRKISLFSCLKDRHDFG |
| | terminal signal peptide | FPQEEFGNQFQKAETIPVLHEMIQQIFNLF |
| | | STKDSSAAWDETLLDKFYTELYQQLNDLEA |
| | | CVIQGVGVTETPLMKEDSILAVRKYFQRIT |
| | | LYLKEKKYSPCAWEVVRAEIMRSFSLSTNL |
| | | QESLRSKE |
| 82 | human Interferon-alpha 2b | MALTFALLVALLVLSCKSSCSVGCDLPQTH |
| | (huIFN-α2b) precursor with N- | SLGSRRTLMLLAQMRRISLFSCLKDRHDFG |
| | terminal signal peptide | FPQEEFGNQFQKAETIPVLHEMIQQIFNLF |
| | | STKDSSAAWDETLLDKFYTELYQQLNDLEA |
| | | CVIQGVGVTETPLMKEDSILAVRKYFQRIT |
| | | LYLKEKKYSPCAWEVVRAEIMRSFSLSTNL |
| | | QESLRSKE |
| 83 | exemplary human PD-L1 | MRIFAVFIFMTYWHLLNAFTVTVPKDLYVV |
| | | EYGSNMTIECKFPVEKQLDLAALIVYWEME |
| | | DKNIIQFVHGEEDLKVQHSSYRQRARLLKD |
| | | QLSLGNAALQITDVKLQDAGVYRCMISYGG |
| | | ADYKRITVKVNAPYNKINQRILVVDPVTSE |
| | | HELTCQAEGYPKAEVIWTSSDHQVLSGKTT |
| | | TTNSKREEKLFNVTSTLRINTTTNEIFYCT |
| | | FRRLDPEENHTAELVIPELPLAHPPNERTH |
| | | LVILGAILLCLGVALIFIFRLRKGRMMDVK |
| | | KCGIQDTNSKKQSDTHLEET |
| 84 | human IgG1 Fc region | DKTHTCPPCPAPELLGGPSVFLFPPKPKDT |
| | polypeptide | LMISRTPEVTCVVVDVSHEDPEVKFNWYVD |
| | | GVEVHNAKTKPREEQYNSTYRVVSVLTVLH |
| | | QDWLNGKEYKCKVSNKALPAPIEKTISKAK |
| | | GQPREPQVYTLPPSRDELTKNQVSLTCLVK |
| | | GFYPSDIAVEWESNGQPENNYKITPPVLDS |
| | | DGSFFLYSKLTVDKSRWQQGNVFSCSVMHE |
| | | ALHNHYTQKSLSLSP |
| 85 | human IgG4 Fc region | ESKYGPPCPSCPAPEFLGGPSVFLFPPKPK |
| | polypeptide | DTLMISRTPEVTCVVVDVSQEDPEVQFNWY |
| | | VDGVEVHNAKTKPREEQFNSTYRVVSVLTV |
| | | LHQDWLNGKEYKCKVSNKGLPSSIEKTISK |
| | | AKGQPREPQVYTLPPSQEEMTKNQVSLTCL |
| | | VKGFYPSDIAVEWESNGQPENNYKITPPVL |
| | | DSDGSFFLYSRLTVDKSRWQEGNVFSCSVM |
| | | HEALHNHYTQKSLSLSL |
| 86 | human IgG1 Fc-region with | DKTHTCPPCPAPEAAGGPSVFLFPPKPKDT |
| | L234A, L235A and P329G | LMISRTPEVTCVVVDVSHEDPEVKFNWYVD |
| | mutations | GVEVHNAKTKPREEQYNSTYRVVSVLTVLH |
| | | QDWLNGKEYKCKVSNKALGAPIEKTISKAK |
| | | GQPREPQVYTLPPSRDELTKNQVSLTCLVK |
| | | GFYPSDIAVEWESNGQPENNYKTTPPVLDS |
| | | DGSFFLYSKLTVDKSRWQQGNVFSCSVMHE |
| | | ALHNHYTQKSLSLSP |
| 87 | human IgG1 Fc-region with | DKTHTCPPCPAPEAAGGPSVFLFPPKPKDT |
| | L234A, L235A and P329G | LMISRTPEVTCVVVDVSHEDPEVKFNWYVD |
| | mutations and with S354C, | GVEVHNAKTKPREEQYNSTYRVVSVLTVLH |
| | T366W (Knob chain) | QDWLNGKEYKCKVSNKALGAPIEKTISKAK |
| | | GQPREPQVYTLPPCRDELTKNQVSLWCLVK |
| | | GFYPSDIAVEWESNGQPENNYKTTPPVLDS |
| | | DGSFFLYSKLTVDKSRWQQGNVFSCSVMHE |
| | | ALHNHYTQKSLSLSP |
| 88 | human IgG1 Fc-region with | DKTHTCPPCPAPEAAGGPSVFLFPPKPKDT |
| | L234A, L235A and P329G | LMISRTPEVTCVVVDVSHEDPEVKFNWYVD |
| | mutations and with Y349C, | GVEVHNAKTKPREEQYNSTYRVVSVLTVLH |
| | T366S, L368A, Y407V (Hole | QDWLNGKEYKCKVSNKALGAPIEKTISKAK |
| | chain) | GQPREPQVCTLPPSRDELTKNQVSLSCAVK |
| | | GFYPSDIAVEWESNGQPENNYKITPPVLDS |
| | | DGSFFLVSKLTVDKSRWQQGNVFSCSVMHE |
| | | ALHNHYTQKSLSLSP |
| 89 | human IgG4 Fc-region with | ESKYGPPCPPCPAPEFEGGPSVFLFPPKPK |
| | S228P, L235E and P329G | DTLMISRTPEVTCVVVDVSQEDPEVQFNWY |
| | mutations and with S354C and | VDGVEVHNAKTKPREEQFNSTYRVVSVLTV |
| | T366W mutations (knob chain) | LHQDWLNGKEYKCKVSNKGLPSSIEKTISK |
| | | AKGQPREPQVYTLPPCQEEMTKNQVSLWCL |
| | | VKGFYPSDIAVEWESNGQPENNYKTTPPVL |
| | | DSDGSFFLYSRLTVDKSRWQEGNVFSCSVM |
| | | HEALHNHYTQKSLSLSL |
| 90 | human IgG4 Fc-region with | ESKYGPPCPPCPAPEFEGGPSVFLFPPKPK |
| | S228P, L235E and P329G | DTLMISRTPEVTCVVVDVSQEDPEVQFNWY |
| | mutations and with Y349C, | VDGVEVHNAKTKPREEQFNSTYRVVSVLTV |
| | T366S, L368A and Y407 | LHQDWLNGKEYKCKVSNKGLGSSIEKTISK |
| | mutations (hole chain) | AKGQPREPQVYTLPPSQEEMTKNQVSLTCL |
| | | VKGFYPSDIAVEWESNGQPENNYKTTPPVL |
| | | DSDGSFFLYSRLTVDKSRWQEGNVFSCSVM |
| | | HEALHNHYTQKSLSLSL |
| 91 | human kappa light chain constant | RTVAAPSVFIFPPSDEQLKSGTASVVCLLN |
| | region | NFYPREAKVQWKVDNALQSGNSQESVTEQD |
| | | SKDSTYSLSSTLTLSKADYEKHKVYACEVT |
| | | HQGLSSPVTKSFNRGEC |
| 92 | human lambda light chain | QPKAAPSVTLFPPSSEELQANKATLVCLIS |
| | constant region | DFYPGAVTVAWKADSSPVKAGVETTTPSKQ |
| | | SNNKYAASSYLSLTPEQWKSHRSYSCQVTH |
| | | EGSTVEKTVAPTECS |
| 93 | human heavy chain constant | ASTKGPSVFPLAPSSKSTSGGTAALGCLVK |
| | region derived from IgG1 | DYFPEPVTVSWNSGALTSGVHTFPAVLQSS |
| | | GLYSLSSVVTVPSSSLGTQTYICNVNHKPS |
| | | NTKVDKKVEPKSCDKTHTCPPCPAPELLGG |
| | | PSVFLFPPKPKDTLMISRTPEVTCVVVDVS |
| | | HEDPEVKFNWYVDGVEVHNAKTKPREEQYN |
| | | STYRVVSVLTVLHQDWLNGKEYKCKVSNKA |
| | | LPAPIEKTISKAKGQPREPQVYTLPPSRDE |
| | | LTKNQVSLTCLVKGFYPSDIAVEWESNGQP |
| | | ENNYKTTPPVLDSDGSFFLYSKLTVDKSRW |
| | | QQGNVFSCSVMHEALHNHYTQKSLSLSP |
| 94 | human heavy chain constant | ASTKGPSVFPLAPSSKSTSGGTAALGCLVK |
| | region derived from IgG1 with | DYFPEPVTVSWNSGALISGVHIFPAVLQSS |
| | mutations L234A, L235A and | GLYSLSSVVTVPSSSLGTQTYICNVNHKPS |
| | P329G | NTKVDKKVEPKSCDKTHTCPPCPAPEAAGG |
| | | PSVFLFPPKPKDTLMISRTPEVTCVVVDVS |
| | | HEDPEVKFNWYVDGVEVHNAKTKPREEQYN |
| | | STYRVVSVLTVLHQDWLNGKEYKCKVSNKA |
| | | LGAPIEKTISKAKGQPREPQVYTLPPSRDE |
| | | LTKNQVSLTCLVKGFYPSDIAVEWESNGQP |
| | | ENNYKITPPVLDSDGSFFLYSKLTVDKSRW |
| | | QQGNVFSCSVMHEALHNHYTQKSLSLSP |
| 95 | human heavy chain constant | ASTKGPSVFPLAPCSRSTSESTAALGCLVK |
| | region derived from IgG4 | DYFPEPVTVSWNSGALTSGVHTFPAVLQSS |
| | | GLYSLSSVVTVPSSSLGTKTYTCNVDHKPS |
| | | NTKVDKRVESKYGPPCPSCPAPEFLGGPSV |
| | | FLFPPKPKDTLMISRTPEVTCVVVDVSQED |
| | | PEVQFNWYVDGVEVHNAKTKPREEQFNSTY |
| | | RVVSVLTVLHQDWLNGKEYKCKVSNKGLPS |
| | | SIEKTISKAKGQPREPQVYTLPPSQEEMTK |
| | | NQVSLTCLVKGFYPSDIAVEWESNGQPENN |
| | | YKTTPPVLDSDGSFFLYSRLTVDKSRWQEG |
| | | NVFSCSVMHEALHNHYTQKSLSLSL |
| 96 | peptidic linker | (GSGGS)ₙ |
| 97 | peptidic linker | (GGGS)ₙ |
| 98 | peptidic linker | (GSGGG)ₙ |
| 99 | peptidic linker | (GGGSG)ₙ |
| 100 | peptidic linker | (GSSSG)ₙ |
| 101 | peptidic linker | (GGGGS)ₙ |
| 102 | peptidic linker | (GGSGG)ₙ |
| 103 | peptidic linker | GGSGGGSGGGSGGGSGGGSG |
| 104 | peptidic linker | GSGSGGSGSGGSGSGGSGSGGSGSG |
| 105 | peptidic linker | GSGGGGSGGGGSGGGGSGGG |
| 106 | peptidic linker | GGGSGGGGSGGGGSGGGGSGGGGSG |
| 107 | peptidic linker | GSSSGGSSSGGSSSGGSSSG |
| 108 | peptidic linker | GGGGSGGGGSGGGGSGGGGS |
| 109 | peptidic linker | GGGGSGGGGSGGGGSGGGGS |
| 110 | peptidic linker | GGSGGGGSGGGGSGGGGSGG |
| 111 | Parental (not affinity-matured) | MQQLVESGGGLVQPGGSLRLSCAAKETWSP |
| | bispecific DutaFab M14HH17L | NAMSWVRQAPGKGLEWVGSISPKGGSKSIH |
| | (P1AG2938) heavy chain | RKFQGRFTISRDNSKNTLYLQMNSLRAEDT |
| | | AVYYCAKDLGFMDEWGQGTLVTVSSASTKG |
| | | PSVFPLAPSSKSTSGGTAALGCLVKDYFPE |
| | | PVTVSWNSGALTSGVHTFPAVLQSSGLYSL |
| | | SSVVTVPSSSLGTQTYICNVNHKPSNTKVD |
| | | KKVEPKSCDKTHISPPGSGMASMTGGQQMG |
| 112 | Parental (not affinity-matured) | KTQMTQSPSSLSASVGDRVTITCDGTYGIS |
| | bispecific DutaFab M14HH17L | NELAWYQQKPGKAPKLLIYDGEYHDFDVPS |
| | (P1AG2938) light chain | RFSGSGSGSDFTLTISSLQPEDFATYYCQQ |
| | | YGNWPHTFGQGTKVEIKRTVAAPSVFIFPP |
| | | SDEQLKSGTASVVCLLNNFYPREAKVQWKV |
| | | DNALQSGNSQESVTEQDSKDSTYSLSSTLT |
| | | LSKADYEKHKVYACEVTHQGLSSPVTKSFN |
| | | RGEC |
| 113 | Parental (not affinity-matured) | ADQLVESGGGLVQPGGSLRLSCAASGFDFS |
| | bispecific DutaFab E06HP7L | VQAMSWVRQAPGKGLEWVGSISPKGGSKSL |
| | (P1AG2926) heavy chain | NRNFNGRFTISRDNSKNTLYLQMNSLRAED |
| | | TAVYYCAKDLGFMDNWGQGTLVTVSSASTK |
| | | GPSVFPLAPSSKSTSGGTAALGCLVKDYFP |
| | | EPVTVSWNSGALTSGVHTFPAVLQSSGLYS |
| | | LSSVVTVPSSSLGTQTYICNVNHKPSNTKV |
| | | DKKVEPKSCDKTHISPPGSGMASMTGGQQM |
| | | G |
| 114 | Parental (not affinity-matured) | YQMTQSPSSLSASVGDRVTITCHGHVFISN |
| | bispecific DutaFab E06HP7L | YLAWYQQKPGKAPKLLIYDGEWRDFGVPSR |
| | (P1AG2926) light chain | FSGSGSDDSFTLTISSLQPEDFATYYCQQY |
| | | SNWPHTFGQGTKVEIKRIVAAPSVFIFPPS |
| | | DEQLKSGTASVVCLLNNFYPREAKVQWKVD |
| | | NALQSGNSQESVTEQDSKDSTYSLSSTLTL |
| | | SKADYEKHKVYACEVTHQGLSSPVIKSFNR |
| | | GEC |
| 115 | anti-PD-L1 one-armed Fab Fc | MSQLVESGGGLVQPGGSLRLSCAANNDIEF |
| | fusion heavy chain (P1AH0179) | NAMSWVRQAPGKGLEWVGSISPKGGSKSIH |
| | | RKFQGRFTISRDNSKNTLYLQMNSLRAEDT |
| | | AVYYCARDLGFMDIWGQGTLVTVSSASTKG |
| | | PSVFPLAPSSKSTSGGTAALGCLVEDYFPE |
| | | PVTVSWNSGALTSGVHTFPAVLQSSGLYSL |
| | | SSVVTVPSSSLGTQTYICNVNHKPSNTKVD |
| | | EKVEPKSCDKTHTCPPCPAPEAAGGPSVFL |
| | | FPPKPKDTLMISRTPEVTCVVVDVSHEDPE |
| | | VKFNWYVDGVEVHNAKTKPREEQYNSTYRV |
| | | VSVLTVLHQDWLNGKEYKCKVSNKALGAPI |
| | | EKTISKAKGQPREPQVCTLPPSRDELTKNQ |
| | | VSLSCAVKGFYPSDIAVEWESNGQPENNYK |
| | | TTPPVLDSDGSFFLVSKLTVDKSRWQQGNV |
| | | FSCSVMHEALHNHYTQKSLSLSPG |
| 116 | anti-PD-L1 one-armed Fab Fc | KTQMTQSPSSLSASVGDRVTITCDGTYGIS |
| | fusion light chain (P1AH0179) | NELAWYQQKPGKAPKLLIYDGEYHDFDVPS |
| | | RFSGSGSGSDFTLTISSLQPEDFATYYCQQ |
| | | YGNWPHTFGQGTKVEIKRTVAAPSVFIFPP |
| | | SDRKLKSGTASVVCLLNNFYPREAKVQWKV |
| | | DNALQSGNSQESVTEQDSKDSTYSLSSTLT |
| | | LSKADYEKHKVYACEVTHQGLSSPVTKSFN |
| | | RGEC |
| 117 | anti-PD-L1 one-armed Fab Fc | MTQLVESGGGLVQPGGSLRLSCAADHTTEY |
| | fusion heavy chain (P1AI4392) | NAMSWVRQAPGKGLEWVSAISGSGGSTYYA |
| | | DSVKGRFTISRDNSKNTLYLQMNSLRAEDT |
| | | AVYYCARDLGFMDVWGQGTLVTVSSASTKG |
| | | PSVFPLAPSSKSTSGGTAALGCLVKDYFPE |
| | | PVTVSWNSGALTSGVHTFPAVLQSSGLYSL |
| | | SSVVTVPSSSLGTQTYICNVNHKPSNTKVD |
| | | KKVEPKSCDKTHTCPPCPAPEAAGGPSVFL |
| | | FPPKPKDTLMISRTPEVTCVVVDVSHEDPE |
| | | VKFNWYVDGVEVHNAKTKPREEQYNSTYRV |
| | | VSVLTVLHQDWLNGKEYKCKVSNKALGAPI |
| | | EKTISKAKGQPREPQVCTLPPSRDELTKNQ |
| | | VSLSCAVKGFYPSDIAVEWESNGQPENNYK |
| | | TTPPVLDSDGSFFLVSKLTVDKSRWQQGNV |
| | | FSCSVMHEALHNHYTQKSLSLSPG |
| 118 | anti-PD-L1 one-armed Fab Fc | DIQMTQSPSSLSASVGDRVTITCRASQSIS |
| | fusion light chain (P1AI4392) | SYLNWYQQKPGKAPKLLIYDASYHESGVPS |
| | | RFSGSGSGIDFTLTISSLQPEDFATYYCQQ |
| | | SYSTPWTFGQGTKVEIKRTVAAPSVFIFPP |
| | | SDEQLKSGTASVVCLLNNFYPREAKVQWKV |
| | | DNALQSGNSQESVTEQDSKDSTYSLSSTLT |
| | | LSKADYEKHKVYACEVTHQGLSSPVTKSFN |
| | | RGEC |
| 119 | anti-PD-L1 one-armed Fab Fc | MTQLVESGGGLVQPGGSLRLSCAADHTTEY |
| | fusion heavy chain (P1AI4397) | NAMSWVRQAPGKGLEWVSAISGSGGSTYYA |
| | | DSVKGRFTISRDNSKNTLYLQMNSLRAEDT |
| | | AVYYCARDLGFMDVWGQGTLVTVSSASTKG |
| | | PSVFPLAPSSKSTSGGTAALGCLVKDYFPE |
| | | PVTVSWNSGALTSGVHTFPAVLQSSGLYSL |
| | | SSVVTVPSSSLGTQTYICNVNHKPSNTKVD |
| | | KKVEPKSCDKTHTCPPCPAPEAAGGPSVFL |
| | | FPPKPKDTLMISRTPEVTCVVVDVSHEDPE |
| | | VKFNWYVDGVEVHNAKTKPREEQYNSTYRV |
| | | VSVLTVLHQDWLNGKEYKCKVSNKALGAPI |
| | | EKTISKAKGQPREPQVCTLPPSRDELTKNQ |
| | | VSLSCAVKGFYPSDIAVEWESNGQPENNYK |
| | | TTPPVLDSDGSFFLVSKLTVDKSRWQQGNV |
| | | FSCSVMHEALHNHYTQKSLSLSPG |
| 120 | anti-PD-L1 one-armed Fab Fc | DIQMTQSPSSLSASVGDRVTITCRASQSIS |
| | fusion light chain (P1AI4397) | SYLNWYQQKPGKAPKLLIYDASSHEFGVPS |
| | | RFSGSGSGTDFTLTISSLQPEDFATYYCQQ |
| | | SYSTPWTFGQGTKVEIKRIVAAPSVFIFPP |
| | | SDEQLKSGTASVVCLLNNFYPREAKVQWKV |
| | | DNALQSGNSQESVTEQDSKDSTYSLSSTLT |
| | | LSKADYEKHKVYACEVTHQGLSSPVTKSFN |
| | | RGEC |
| 121 | anti-PD-L1 one-armed Fab Fc | DIQMTQSPSSLSASVGDRVTITCRASQSIS |
| | fusion heavy chain (P1AI4475) | SYLNWYQQKPGKAPKLLIYDASYHEFGVPS |
| | | RFSGSGSGTDFTLTISSLQPEDFATYYCQQ |
| | | SYSTPWTFGQGTKVEIKSSASTKGPSVFPL |
| | | APSSKSTSGGTAALGCLVKDYFPEPVTVSW |
| | | NSGALTSGVHTFPAVLQSSGLYSLSSVVTV |
| | | PSSSLGTQTYICNVNHKPSNTKVDKKVEPK |
| | | SCDKTHTCPPCPAPEAAGGPSVFLFPPKPK |
| | | DTLMISRTPEVTCVVVDVSHEDPEVKFNWY |
| | | VDGVEVHNAKTKPREEQYNSTYRVVSVLTV |
| | | LHQDWLNGKEYKCKVSNKALGAPIEKTISK |
| | | AKGQPREPQVCTLPPSRDELTKNQVSLSCA |
| | | VKGFYPSDIAVEWESNGQPENNYKTTPPVL |
| | | DSDGSFFLVSKLTVDKSRWQQGNVFSCSVM |
| | | HEALHNHYTQKSLSLSPG |
| 122 | anti-PD-L1 one-armed Fab Fc | MTQLVESGGGLVQPGGSLRLSCAADHTTEY |
| | fusion light chain (P1AI4475) | NAMSWVRQAPGKGLEWVSAISGSGGSTYYA |
| | | DSVKGRFTISRDNSKNTLYLQMNSLRAEDT |
| | | AVYYCARDLGFMDVWGQGTLVTVSSASVAA |
| | | PSVFIFPPSDEQLKSGTASVVCLLNNFYPR |
| | | EAKVQWKVDNALQSGNSQESVTEQDSKDST |
| | | YSLSSTLTLSKADYEKHKVYACEVTHQGLS |
| | | SPVTKSFNRGEC |
| 123 | anti-PD-L1 one-armed Fab Fc | DIQMTQSPSSLSASVGDRVTITCRASQSIS |
| | fusion heavy chain (P1AI4476) | SYLNWYQQKPGKAPKLLIYDAEYHDFGVPS |
| | | RFSGSGSGTDFTLTISSLQPEDFATYYCQQ |
| | | SYSTPWTFGQGTKVEIKSSASTKGPSVFPL |
| | | APSSKSTSGGTAALGCLVKDYFPEPVTVSW |
| | | NSGALTSGVHTFPAVLQSSGLYSLSSVVTV |
| | | PSSSLGTQTYICNVNHKPSNTKVDKKVEPK |
| | | SCDKTHTCPPCPAPEAAGGPSVFLFPPKPK |
| | | DTLMISRTPEVTCVVVDVSHEDPEVKFNWY |
| | | VDGVEVHNAKTKPREEQYNSTYRVVSVLTV |
| | | LHQDWLNGKEYKCKVSNKALGAPIEKTISK |
| | | AKGQPREPQVCTLPPSRDELTKNQVSLSCA |
| | | VKGFYPSDIAVEWESNGQPENNYKTTPPVL |
| | | DSDGSFFLVSKLTVDKSRWQQGNVFSCSVM |
| | | HEALHNHYTQKSLSLSPG |
| 124 | anti-PD-L1 one-armed Fab Fc | MTQLVESGGGLVQPGGSLRLSCAADHTTEY |
| | fusion light chain (P1AI4476) | NAMSWVRQAPGKGLEWVSAISGSGGSTYYA |
| | | DSVKGRFTISRDNSKNTLYLQMNSLRAEDT |
| | | AVYYCARDLGFMDVWGQGTLVTVSSASVAA |
| | | PSVFIFPPSDEQLKSGTASVVCLLNNFYPR |
| | | EAKVQWKVDNALQSGNSQESVTEQDSKDST |
| | | YSLSSTLTLSKADYEKHKVYACEVTHQGLS |
| | | SPVTKSFNRGEC |
| 125 | anti-PD-L1 one-armed Fab Fc | MTQLVESGGGLVQPGGSLRLSCAADHTTEY |
| | fusion heavy chain (P1AI4478) | NAMSWVRQAPGKGLEWVGSISGSGGSTSLA |
| | | RSVKGRFTISRDNSKNTLYLQMNSLRAEDT |
| | | AVYYCARDLGFMDVWGQGTLVTVSSASTKG |
| | | PSVFPLAPSSKSTSGGTAALGCLVEDYFPE |
| | | PVTVSWNSGALTSGVHTFPAVLQSSGLYSL |
| | | SSVVTVPSSSLGTQTYICNVNHKPSNTKVD |
| | | EKVEPKSCDKTHTCPPCPAPEAAGGPSVFL |
| | | FPPKPKDTLMISRTPEVTCVVVDVSHEDPE |
| | | VKFNWYVDGVEVHNAKTKPREEQYNSTYRV |
| | | VSVLTVLHQDWLNGKEYKCKVSNKALGAPI |
| | | EKTISKAKGQPREPQVYTLPPCRDELTKNQ |
| | | VSLWCLVKGFYPSDIAVEWESNGQPENNYK |
| | | TTPPVLDSDGSFFLYSKLTVDKSRWQQGNV |
| | | FSCSVMHEALHNHYTQKSLSLSPG |
| 126 | anti-PD-L1 one-armed Fab Fc | DIQMTQSPSSLSASVGDRVTITCDAVITIS |
| | fusion light chain (P1AI4478) | QELAWYQQKPGKAPKLLIYDAEYHDTGVPS |
| | | RFSGSGSGTDFTLTISSLQPEDFATYYCQQ |
| | | YGNWPHTFGQGTKVEIKRTVAAPSVFIFPP |
| | | SDRKLKSGTASVVCLLNNFYPREAKVQWKV |
| | | DNALQSGNSQESVTEQDSKDSTYSLSSTLT |
| | | LSKADYEKHKVYACEVTHQGLSSPVTKSFN |
| | | RGEC |
| 127 | anti-PD-L1 one-armed Fab Fc | MTQLVESGGGLVQPGGSLRLSCAADHTTEY |
| | fusion heavy chain (P1AI4479) | NAMSWVRQAPGKGLEWVGSISGSGGSTSLA |
| | | RSVKGRFTISRDNSKNTLYLQMNSLRAEDT |
| | | AVYYCARDLGFMDVWGQGTLVTVSSASTKG |
| | | PSVFPLAPSSKSTSGGTAALGCLVEDYFPE |
| | | PVTVSWNSGALTSGVHTFPAVLQSSGLYSL |
| | | SSVVTVPSSSLGTQTYICNVNHKPSNTKVD |
| | | EKVEPKSCDKTHTCPPCPAPEAAGGPSVFL |
| | | FPPKPKDTLMISRTPEVTCVVVDVSHEDPE |
| | | VKFNWYVDGVEVHNAKTKPREEQYNSTYRV |
| | | VSVLTVLHQDWLNGKEYKCKVSNKALGAPI |
| | | EKTISKAKGQPREPQVYTLPPCRDELTKNQ |
| | | VSLWCLVKGFYPSDIAVEWESNGQPENNYK |
| | | TTPPVLDSDGSFFLYSKLTVDKSRWQQGNV |
| | | FSCSVMHEALHNHYTQKSLSLSPG |
| 128 | anti-PD-L1 one-armed Fab Fc | DIQMTQSPSSLSASVGDRVTITCDAVITIS |
| | fusion light chain (P1AI4479) | QELAWYQQKPGKAPKLLIYDASYHESGVPS |
| | | RFSGSGSGTDFTLTISSLQPEDFATYYCQQ |
| | | YGNWPHTFGQGTKVEIKRTVAAPSVFIFPP |
| | | SDRKLKSGTASVVCLLNNFYPREAKVQWKV |
| | | DNALQSGNSQESVTEQDSKDSTYSLSSTLT |
| | | LSKADYEKHKVYACEVTHQGLSSPVTKSFN |
| | | RGEC |
| 129 | anti-PD-L1 one-armed Fab Fc | MTQLVESGGGLVQPGGSLRLSCAADHTTEY |
| | fusion heavy chain (P1AI4480) | NAMSWVRQAPGKGLEWVGSISGSGGSTSLA |
| | | RSVKGRFTISRDNSKNTLYLQMNSLRAEDT |
| | | AVYYCARDLGFMDVWGQGTLVTVSSASTKG |
| | | PSVFPLAPSSKSTSGGTAALGCLVEDYFPE |
| | | PVTVSWNSGALTSGVHTFPAVLQSSGLYSL |
| | | SSVVTVPSSSLGTQTYICNVNHKPSNTKVD |
| | | EKVEPKSCDKTHTCPPCPAPEAAGGPSVFL |
| | | FPPKPKDTLMISRTPEVTCVVVDVSHEDPE |
| | | VKFNWYVDGVEVHNAKTKPREEQYNSTYRV |
| | | VSVLTVLHQDWLNGKEYKCKVSNKALGAPI |
| | | EKTISKAKGQPREPQVYTLPPCRDELTKNQ |
| | | VSLWCLVKGFYPSDIAVEWESNGQPENNYK |
| | | TTPPVLDSDGSFFLYSKLTVDKSRWQQGNV |
| | | FSCSVMHEALHNHYTQKSLSLSPG |
| 130 | anti-PD-L1 one-armed Fab Fc | DIQMTQSPSSLSASVGDRVTITCDAVIVIS |
| | fusion light chain (P1AI4480) | QELAWYQQKPGKAPKLLIYDASYHDTGVPS |
| | | RFSGSGSGTDFTLTISSLQPEDFATYYCQQ |
| | | YGNWPHTFGQGTKVEIKRTVAAPSVFIFPP |
| | | SDRKLKSGTASVVCLLNNFYPREAKVQWKV |
| | | DNALQSGNSQESVTEQDSKDSTYSLSSTLT |
| | | LSKADYEKHKVYACEVTHQGLSSPVTKSFN |
| | | RGEC |
| 131 | anti-PD-L1 one-armed Fab Fc | EVQLVESGGGLVQPGGSLRLSCAADHTTEY |
| | fusion light chain (P1AI4481) | NAMSWVRQAPGKGLEWVGSISGSGGSTSLA |
| | | RSVKGRFTISRDNSKNTLYLQMNSLRAEDT |
| | | AVYYCARDLGFMDVWGQGTLVTVSSASTKG |
| | | PSVFPLAPSSKSTSGGTAALGCLVEDYFPE |
| | | PVTVSWNSGALTSGVHTFPAVLQSSGLYSL |
| | | SSVVTVPSSSLGTQTYICNVNHKPSNTKVD |
| | | EKVEPKSCDKTHTCPPCPAPEAAGGPSVFL |
| | | FPPKPKDTLMISRTPEVTCVVVDVSHEDPE |
| | | VKFNWYVDGVEVHNAKTKPREEQYNSTYRV |
| | | VSVLTVLHQDWLNGKEYKCKVSNKALGAPI |
| | | EKTISKAKGQPREPQVYTLPPCRDELTKNQ |
| | | VSLWCLVKGFYPSDIAVEWESNGQPENNYK |
| | | TTPPVLDSDGSFFLYSKLIVDKSRWQQGNV |
| | | FSCSVMHEALHNHYTQKSLSLSPG |
| 132 | anti-PD-L1 one-armed Fab Fc | DIQMTQSPSSLSASVGDRVTITCDAVITIS |
| | fusion light chain (P1AI4481) | QELAWYQQKPGKAPKLLIYDASYHEFGVPS |
| | | RFSGSGSGTDFTLTISSLQPEDFATYYCQQ |
| | | YGNWPHTFGQGTKVEIKRTVAAPSVFIFPP |
| | | SDRKLKSGTASVVCLLNNFYPREAKVQWKV |
| | | DNALQSGNSQESVTEQDSKDSTYSLSSTLT |
| | | LSKADYEKHKVYACEVTHQGLSSPVTKSFN |
| | | RGEC |
| 133 | anti-PD-L1 one-armed Fab Fc | MTQLVESGGGLVQPGGSLRLSCAADHTTEY |
| | fusion heavy chain (P1AI4483) | NAMSWVRQAPGKGLEWVGSISGSGGSISLA |
| | | RSVKGRFTISRDNSKNTLYLQMNSLRAEDT |
| | | AVYYCARDLGFMDVWGQGTLVTVSSASTKG |
| | | PSVFPLAPSSKSTSGGTAALGCLVEDYFPE |
| | | PVTVSWNSGALTSGVHTFPAVLQSSGLYSL |
| | | SSVVTVPSSSLGTQTYICNVNHKPSNTKVD |
| | | EKVEPKSCDKTHTCPPCPAPEAAGGPSVFL |
| | | FPPKPKDTLMISRTPEVTCVVVDVSHEDPE |
| | | VKFNWYVDGVEVHNAKTKPREEQYNSTYRV |
| | | VSVLTVLHQDWLNGKEYKCKVSNKALGAPI |
| | | EKTISKAKGQPREPQVYTLPPCRDELTKNQ |
| | | VSLWCLVKGFYPSDIAVEWESNGQPENNYK |
| | | TTPPVLDSDGSFFLYSKLTVDKSRWQQGNV |
| | | FSCSVMHEALHNHYTQKSLSLSPG |
| 134 | anti-PD-L1 one-armed Fab Fc | DIQMTQSPSSLSASVGDRVTITCDAVITIS |
| | fusion light chain (P1AI4483) | QELAWYQQKPGKAPKLLIYDASYHDTGVPS |
| | | RFSGSGSGTDFTLTISSLQPEDFATYYCQQ |
| | | YGNWPHTFGQGTKVEIKRIVAAPSVFIFPP |
| | | SDRKLKSGTASVVCLLNNFYPREAKVQWKV |
| | | DNALQSGNSQESVTEQDSKDSTYSLSSTLT |
| | | LSKADYEKHKVYACEVTHQGLSSPVTKSFN |
| | | RGEC |
| 135 | anti-PD-L1 one-armed Fab Fc | MTQLVESGGGLVQPGGSLRLSCAADHTTEY |
| | fusion heavy chain (P1AI4485) | NAMSWVRQAPGKGLEWVGSISGSGGSTSLA |
| | | RSVKGRFTISRDNSKNTLYLQMNSLRAEDT |
| | | AVYYCARDLGFMDVWGQGTLVTVSSASTKG |
| | | PSVFPLAPSSKSTSGGTAALGCLVEDYFPE |
| | | PVTVSWNSGALTSGVHTFPAVLQSSGLYSL |
| | | SSVVTVPSSSLGTQTYICNVNHKPSNTKVD |
| | | EKVEPKSCDKTHTCPPCPAPEAAGGPSVFL |
| | | FPPKPKDTLMISRTPEVTCVVVDVSHEDPE |
| | | VKFNWYVDGVEVHNAKTKPREEQYNSTYRV |
| | | VSVLTVLHQDWLNGKEYKCKVSNKALGAPI |
| | | EKTISKAKGQPREPQVYTLPPCRDELTKNQ |
| | | VSLWCLVKGFYPSDIAVEWESNGQPENNYK |
| | | TTPPVLDSDGSFFLYSKLTVDKSRWQQGNV |
| | | FSCSVMHEALHNHYTQKSLSLSPG |
| 136 | anti-PD-L1 one-armed Fab Fc | DIQMTQSPSSLSASVGDRVTITCDAVITIS |
| | fusion light chain (P1AI4485) | QELAWYQQKPGKAPKLLIYDASSHEFGVPS |
| | | RFSGSGSGTDFTLTISSLQPEDFATYYCQQ |
| | | YGNWPHTFGQGTKVEIKRTVAAPSVFIFPP |
| | | SDRKLKSGTASVVCLLNNFYPREAKVQWKV |
| | | DNALQSGNSQESVTEQDSKDSTYSLSSTLT |
| | | LSKADYEKHKVYACEVTHQGLSSPVTKSFN |
| | | RGEC |
| 137 | anti-PD-L1 one-armed Fab Fc | MTQLVESGGGLVQPGGSLRLSCAADHTTEY |
| | fusion heavy chain (P1AI4903) | NAMSWVRQAPGKGLEWVGSISGSGGSTSLA |
| | | RSVKGRFTISRDNSKNTLYLQMNSLRAEDT |
| | | AVYYCARDLGFMDVWGQGTLVTVSSASTKG |
| | | PSVFPLAPSSKSTSGGTAALGCLVEDYFPE |
| | | PVTVSWNSGALTSGVHTFPAVLQSSGLYSL |
| | | SSVVTVPSSSLGTQTYICNVNHKPSNTKVD |
| | | EKVEPKSCDKTHTCPPCPAPEAAGGPSVFL |
| | | FPPKPKDTLMISRTPEVTCVVVDVSHEDPE |
| | | VKFNWYVDGVEVHNAKTKPREEQYNSTYRV |
| | | VSVLTVLHQDWLNGKEYKCKVSNKALGAPI |
| | | EKTISKAKGQPREPQVYTLPPCRDELTKNQ |
| | | VSLWCLVKGFYPSDIAVEWESNGQPENNYK |
| | | TTPPVLDSDGSFFLYSKLTVDKSRWQQGNV |
| | | FSCSVMHEALHNHYTQKSLSLSPG |
| 138 | anti-PD-L1 one-armed Fab Fc | DIQMTQSPSSLSASVGDRVTITCDALITIS |
| | fusion light chain (P1AI4903) | QELAWYQQKPGKAPKLLIYDASYHESGVPS |
| | | RFSGSGSGTDFTLTISSLQPEDFATYYCQQ |
| | | YGNWPHTFGQGTKVEIKRTVAAPSVFIFPP |
| | | SDRKLKSGTASVVCLLNNFYPREAKVQWKV |
| | | DNALQSGNSQESVTEQDSKDSTYSLSSTLT |
| | | LSKADYEKHKVYACEVTHQGLSSPVTKSFN |
| | | RGEC |
| 139 | Fc domain heavy chain with Avi- | DKTHTCPPCPAPEAAGGPSVFLFPPKPKDT |
| | Tag used for one-armed Fab Fc | LMISRTPEVTCVVVDVSHEDPEVKFNWYVD |
| | fusion proteins P1AI4478, | GVEVHNAKTKPREEQYNSTYRVVSVLTVLH |
| | P1AI4479, P1AI4480, P1AI4481, | QDWLNGKEYKCKVSNKALGAPIEKTISKAK |
| | P1AI4483, P1AI4485, and | GQPREPQVCTLPPSRDELTKNQVSLSCAVK |
| | P1AI4903 | GFYPSDIAVEWESNGQPENNYKTTPPVLDS |
| | | DGSFFLVSKLTVDKSRWQQGNVFSCSVMHE |
| | | ALHNHYTQKSLSLSPGGGSGGGLNDIFEAQ |
| | | KIEWHE |
| 140 | Fc domain heavy chain with Avi- | DKTHTCPPCPAPEAAGGPSVFLFPPKPKDT |
| | Tag used for one-armed Fab Fc | LMISRTPEVTCVVVDVSHEDPEVKFNWYVD |
| | fusion proteins P1AI4392, | GVEVHNAKTKPREEQYNSTYRVVSVLTVLH |
| | P1AI4397, P1AI4475 and | QDWLNGKEYKCKVSNKALGAPIEKTISKAK |
| | P1AI4476 | GQPREPQVYTLPPCRDELTKNQVSLWCLVK |
| | | GFYPSDIAVEWESNGQPENNYKTTPPVLDS |
| | | DGSFFLYSKLTVDKSRWQQGNVFSCSVMHE |
| | | ALHNHYTQKSLSLSPGGGSGGGLNDIFEAQ |
| | | KIEWHE |
| 141 | Fc domain heavy chain used for | DKTHTCPPCPAPEAAGGPSVFLFPPKPKDT |
| | one-armed Fab Fc fusion protein | LMISRTPEVTCVVVDVSHEDPEVKFNWYVD |
| | P1AH0179 | GVEVHNAKTKPREEQYNSTYRVVSVLTVLH |
| | | QDWLNGKEYKCKVSNKALGAPIEKTISKAK |
| | | GQPREPQVYTLPPCRDELTKNQVSLWCLVK |
| | | GFYPSDIAVEWESNGQPENNYKTTPPVLDS |
| | | DGSFFLYSKLTVDKSRWQQGNVFSCSVMHE |
| | | ALHNHYTQKSLSLSPG |
| 142 | Full length heavy chain of M14 | MTQLVESGGGLVQPGGSLRLSCAADHTTEY |
| | anti-PD-L1 antibody (PGLALA) | NAMSWVRQAPGKGLEWVSAISGSGGSTYYA |
| | | DSVKGRFTISRDNSKNTLYLQMNSLRAEDT |
| | | AVYYCARDLGFMDVWGQGTLVTVSSASTKG |
| | | PSVFPLAPSSKSTSGGTAALGCLVKDYFPE |
| | | PVTVSWNSGALTSGVHTFPAVLQSSGLYSL |
| | | SSVVTVPSSSLGTQTYICNVNHKPSNTKVD |
| | | KKVEPKSCDKTHTCPPCPAPEAAGGPSVFL |
| | | FPPKPKDTLMISRTPEVTCVVVDVSHEDPE |
| | | VKFNWYVDGVEVHNAKTKPREEQYNSTYRV |
| | | VSVLTVLHQDWLNGKEYKCKVSNKALGAPI |
| | | EKTISKAKGQPREPQVYTLPPSRDELTKNQ |
| | | VSLTCLVKGFYPSDIAVEWESNGQPENNYK |
| | | TTPPVLDSDGSFFLYSKLTVDKSRWQQGNV |
| | | FSCSVMHEALHNHYTQKSLSLSP |
| 143 | Full length light chain of M14 | DIQMTQSPSSLSASVGDRVTITCRASQSIS |
| | anti-PD-L1 antibody (PGLALA) | SYLNWYQQKPGKAPKLLIYDASYHEFGVPS |
| | | RFSGSGSGTDFTLTISSLQPEDFATYYCQQ |
| | | SYSTPWTFGQGTKVEIKRTVAAPSVFIFPP |
| | | SDEQLKSGTASVVCLLNNFYPREAKVQWKV |
| | | DNALQSGNSQESVTEQDSKDSTYSLSSTLT |
| | | LSKADYEKHKVYACEVTHQGLSSPVIKSFN |
| | | RGEC |
| 144 | Human PD-L1 ECD (CD274) | FTVTVPKDLYVVEYGSNMTIECKFPVEKQL |
| | (P1AD9695) (generated via | DLAALIVYWEMEDKNIIQFVHGEEDLKVQH |
| | papaine digestion of P1AD8569) | SSYRQRARLLKDQLSLGNAALQITDVKLQD |
| | | AGVYRCMISYGGADYKRITVKVNAPYNKIN |
| | | QRILVVDPVTSEHELTCQAEGYPKAEVIWT |
| | | SSDHQVLSGKTTTTNSKREEKLFNVTSTLR |
| | | INTTTNEIFYCTFRRLDPEENHTAELVIPE |
| | | LPLA |
| 145 | Cynomolgus PD-L1 ECD (amino | FTVTVPKDLYVVEYGSNMTIECKFPVEKQL |
| | acid 18-238) | DLISLIVYWEMEDKNIIQFVHGEEDLKVQH |
| | | SNYRQRAQLLKDQLSLGNAALRITDVKLQD |
| | | AGVYRCMISYGGADYKRITVKVNAPYNKIN |
| | | QRILVVDPVTSEHELTCQAEGYPKAEVIWT |
| | | SSDHQVLSGKTTTINSKREEKLLNVTSTLR |
| | | INTTANEIFYCIFRRLDPEENHTAELVIPE |
| | | LPLALPPNER |
| 146 | IFN-α2a R149A | CDLPQTHSLGSRRTLMLLAQMRKISLESCL |
| | | KDRHDFGFPQEEFGNQFQKAETIPVLHEMI |
| | | QQIFNLFSTKDSSAAWDETLLDKFYTELYQ |
| | | QLNDLEACVIQGVGVTETPLMKEDSILAVR |
| | | KYFQRITLYLKEKKYSPCAWEVVRAEIMAS |
| | | FSLSTNLQESLRSKE |
| 147 | huIFNAR2 ECD (extracellular | ISYDSPDYTDESCTFKISLRNFRSILSWEL |
| | domain) | KNHSIVPTHYTLLYTIMSKPEDLKVVKNCA |
| | | NTTRSFCDLTDEWRSTHEAYVTVLEGFSGN |
| | | TTLFSCSHNFWLAIDMSFEPPEFEIVGFTN |
| | | HINVMVKFPSIVEEELQFDLSLVIEEQSEG |
| | | IVKKHKPEIKGNMSGNFTYIIDKLIPNTNY |
| | | CVSVYLEHSDEQAVIKSPLKCTLLPPGQES |
| | | ESAESAK |
| 148 | Human CD79B ECD | ARSEDRYRNPKGSACSRIWQSPRFIARKRG |
| | (extracellular domain) (amino acid | FTVKMHCYMNSASGNVSWLWKQEMDENPQQ |
| | 29-159, uniprot ID P40259) | LKLEKGRMEESQNESLATLTIQGIRFEDNG |
| | | IYFCQQKCNNTSEVYQGSGTELRVMGFSTL |
| | | AQLKQRNTLKDGGNSVT |
| 149 | avi_his Tag | GLNDI FEAQKIEWHE ARAHHHHHH |
| 150 | Fc_LALAPG Avi-His Tag (knob | DKTHTCPPCPAPEAAGGPSVFLFPPKPKDT |
| | chain) | LMISRTPEVTCVVVDVSHEDPEVKFNWYVD |
| | | GVEVHNAKTKPREEQYNSTYRVVSVLTVLH |
| | | QDWLNGKEYKCKVSNKALGAPIEKTISKAK |
| | | GQPREPQVYTLPPCRDELTKNQVSLWCLVK |
| | | GFYPSDIAVEWESNGQPENNYKTTPPVLDS |
| | | DGSFFLYSKLTVDKSRWQQGNVFSCSVMHE |
| | | ALHNHYTQKSLSLSP |
| 151 | Fc_LALAPG Avi-His Tag (hole | DKTHTCPPCPAPEAAGGPSVFLFPPKPKDT |
| | chain) with Avi-His | LMISRTPEVTCVVVDVSHEDPEVKFNWYVD |
| | | GVEVHNAKTKPREEQYNSTYRVVSVLTVLH |
| | | QDWLNGKEYKCKVSNKALGAPIEKTISKAK |
| | | GQPREPQVCTLPPSRDELTKNQVSLSCAVK |
| | | GFYPSDIAVEWESNGQPENNYKTTPPVLDS |
| | | DGSFFLVSKLTVDKSRWQQGNVFSCSVMHE |
| | | ALHNRFTQKSLSLSPGGSGLNDIFEAQKIE |
| | | WHEARAHHHHHH |
| 152 | N-terminal Fc-AviTag ™ (knob | GLNDIFEAQKIEWHEDKTHTCPPCPAPEAA |
| | chain) | GGPSVFLFPPKPKDTLMISRTPEVTCVVVD |
| | | VSHEDPEVKFNWYVDGVEVHNAKTKPREEQ |
| | | YNSTYRVVSVLTVLHQDWLNGKEYKCKVSN |
| | | KALGAPIEKTISKAKGQPREPQVYTLPPCR |
| | | DELTKNQVSLWCLVKGFYPSDIAVEWESNG |
| | | QPENNYKTTPPVLDSDGSFFLYSKLTVDKS |
| | | RWQQGNVFSCSVMHEALHNHYTQKSLSLSP |
| 153 | N-terminal Fc-AviTag ™ (hole | DKTHTCPPCPAPEAAGGPSVFLFPPKPKDT |
| | chain) | AMASRTPEVTCVVVDVSHEDPEVKFNWYVD |
| | | GVEVHNAKTKPREEQYNSTYRVVSVLTVLH |
| | | QDWANGKEYKCKVSNKALGAPIEKTISKAK |
| | | GQPREPQVCTLPPSRDELTKNQVSLSCAVK |
| | | GFYPSDIAVEWESNGQPENNYKTTPPVLDS |
| | | DGSFFLVSKLTVDKSRWQQGNVFSCSVMHE |
| | | ALHNHYTQKSLSLSP |
| 154 | C-terminal Fc-AviHisTag ™ (hole | DKTHTCPPCPAPEAAGGPSVFLFPPKPKDT |
| | chain) | LMISRTPEVTCVVVDVSHEDPEVKFNWYVD |
| | | GVEVHNAKTKPREEQYNSTYRVVSVLTVLH |
| | | QDWLNGKEYKCKVSNKALGAPIEKTISKAK |
| | | GQPREPQVCTLPPSRDELTKNQVSLSCAVK |
| | | GFYPSDIAVEWESNGQPENNYKTTPPVLDS |
| | | DGSFFLVSKLTVDKSRWQQGNVFSCSVMHE |
| | | ALHNRFTQKSLSLSPGGSGLNDIFEAQKIE |
| | | WHEARAHHHHHH |
| 155 | C-terminal Fc-AviHisTag ™ (knob | DKTHTCPPCPAPEAAGGPSVFLFPPKPKDT |
| | chain) | LMISRTPEVTCVVVDVSHEDPEVKFNWYVD |
| | | GVEVHNAKTKPREEQYNSTYRVVSVLTVLH |
| | | QDWLNGKEYKCKVSNKALGAPIEKTISKAK |
| | | GQPREPQVYTLPPCRDELTKNQVSLWCLVK |
| | | GFYPSDIAVEWESNGQPENNYKTTPPVLDS |
| | | DGSFFLYSKLTVDKSRWQQGNVFSCSVMHE |
| | | ALHNHYTQKSLSLSPGK |
| 156 | His-AviTag ™ | HHHHHHGS GLNDIFEAQK IEWHE |
| 157 | C-terminal Fc-tag | DKTHTCPPCPAPELLGGPSVFLFPPKPKDT |
| | | LMISRTPEVTCVVVDVSHEDPEVKFNWYVD |
| | | GVEVHNAKTKPREEQYNSTYRVVSVLTVLH |
| | | QDWLNGKEYKCKVSNKALPAPIEKTISKAK |
| | | GQPREPQVYTLPPSRDELTKNQVSLTCLVK |
| | | GFYPSDIAVEWESNGQPENNYKITPPVLDS |
| | | DGSFFLYSKLTVDKSRWQQGNVFSCSVMHE |
| | | ALHNHYTQKSLSLSP |
| 158 | C-terminal Fc-AviTag ™ (hole | DKTHTCPPCPAPEAAGGPSVFLFPPKPKDT |
| | chain) | LMISRTPEVTCVVVDVSHEDPEVKFNWYVD |
| | | GVEVHNAKTKPREEQYNSTYRVVSVLTVLH |
| | | QDWLNGKEYKCKVSNKALGAPIEKTISKAK |
| | | GQPREPQVCTLPPSRDELTKNQVSLSCAVK |
| | | GFYPSDIAVEWESNGQPENNYKITPPVLDS |
| | | DGSFFLVSKLTVDKSRWQQGNVFSCSVMHE |
| | | ALHNHYTQKSLSLSPG |
| 159 | C-terminal Fc-AviTag ™ (knob | DKTHTCPPCPAPEAAGGPSVFLFPPKPKDT |
| | chain) | LMISRTPEVTCVVVDVSHEDPEVKFNWYVD |
| | | GVEVHNAKTKPREEQYNSTYRVVSVLTVLH |
| | | QDWLNGKEYKCKVSNKALGAPIEKTISKAK |
| | | GQPREPQVYTLPPCRDELTKNQVSLWCLVK |
| | | GFYPSDIAVEWESNGQPENNYKITPPVLDS |
| | | DGSFFLYSKLTVDKSRWQQGNVFSCSVMHE |
| | | ALHNHYTQKSLSLSPGGGSGGGLNDIFEAQ |
| | | KIEWHE |
| 160 | Heavy chain 1 of P1AI4848 (knob | MTQLVESGGGLVQPGGSLRLSCAADHTTEY |
| | chain) | NAMSWVRQAPGKGLEWVGSISGSGGSTSLA |
| | | RSVKGRFTISRDNSKNTLYLQMNSLRAEDT |
| | | AVYYCARDLGFMDVWGQGTLVTVSSASTKG |
| | | PSVFPLAPSSKSTSGGTAALGCLVEDYFPE |
| | | PVTVSWNSGALTSGVHIFPAVLQSSGLYSL |
| | | SSVVTVPSSSLGTQTYICNVNHKPSNTKVD |
| | | EKVEPKSCDKTHTCPPCPAPEAAGGPSVFL |
| | | FPPKPKDTLMISRTPEVTCVVVDVSHEDPE |
| | | VKFNWYVDGVEVHNAKTKPREEQYNSTYRV |
| | | VSVLTVLHQDWLNGKEYKCKVSNKALGAPI |
| | | EKTISKAKGQPREPQVYTLPPCRDELTKNQ |
| | | VSLWCLVKGFYPSDIAVEWESNGQPENNYK |
| | | TTPPVLDSDGSFFLYSKLTVDKSRWQQGNV |
| | | FSCSVMHEALHNHYTQKSLSLSPG |
| 161 | Heavy chain 2 of P1AI4848 (hole | DIQMTQSPSSLSASVGDRVTITCRASQDVS |
| | chain) | TAVAWYQQKPGKAPKLLIYSASFLYSGVPS |
| | | RFSGSGSGTDFTLTISSLQPEDFATYYCQQ |
| | | YLYHPATFGQGTKVEIKSSASTKGPSVFPL |
| | | APSSKSTSGGTAALGCLVKDYFPEPVTVSW |
| | | NSGALTSGVHTFPAVLQSSGLYSLSSVVTV |
| | | PSSSLGTQTYICNVNHKPSNTKVDKKVEPK |
| | | SCGGSGGGGSGGGGSGGGGSGGDIQMTQSP |
| | | SSLSASVGDRVTITCRASQDVSTAVAWYQQ |
| | | KPGKAPKLLIYSASFLYSGVPSRFSGSGSG |
| | | TDFTLTISSLQPEDFATYYCQQYLYHPAIF |
| | | GQGTKVEIKSSASTKGPSVFPLAPSSKSTS |
| | | GGTAALGCLVKDYFPEPVTVSWNSGALTSG |
| | | VHTFPAVLQSSGLYSLSSVVTVPSSSLGTQ |
| | | TYICNVNHKPSNTKVDKKVEPKSCDKTHTC |
| | | PPCPAPEAAGGPSVFLFPPKPKDTLMISRT |
| | | PEVTCVVVDVSHEDPEVKFNWYVDGVEVHN |
| | | AKTKPREEQYNSTYRVVSVLTVLHQDWLNG |
| | | KEYKCKVSNKALGAPIEKTISKAKGQPREP |
| | | QVCTLPPSRDELTKNQVSLSCAVKGFYPSD |
| | | IAVEWESNGQPENNYKTTPPVLDSDGSFFL |
| | | VSKLTVDKSRWQQGNVFSCSVMHEALHNHY |
| | | TQKSLSLSPG |
| 162 | Light chain 1 of P1AI4848 | CDLPQTHSLGSRRTLMLLAQMRKISLFSCL |
| | including IFNα2a (A chain) | KDRHDFGFPQEEFGNQFQKAETIPVLHEMI |
| | | QQIFNLFSTKDSSAAWDETLLDKFYTELYQ |
| | | QLNDLEACVIQGVGVTETPLMKEDSILAVR |
| | | KYFQRITLYLKEKKYSPCAWEVVRAEIMRS |
| | | FSLSTNLQESLRSKEGGSGGGGSGGGGSGG |
| | | GGSGGDIQMTQSPSSLSASVGDRVTITCDA |
| | | VITISQELAWYQQKPGKAPKLLIYDASYHE |
| | | SGVPSRFSGSGSGTDFTLTISSLQPEDFAT |
| | | YYCQQYGNWPHTFGQGTKVEIKRTVAAPSV |
| | | FIFPPSDRKLKSGTASVVCLLNNFYPREAK |
| | | VQWKVDNALQSGNSQESVTEQDSKDSTYSL |
| | | SSTLILSKADYEKHKVYACEVTHQGLSSPV |
| | | TKSFNRGEC |
| 163 | Light chain 2 of P1AI4848 (B | EVQLVESGGGLVQPGGSLRLSCAASGFTFS |
| | chain) | DSWIHWVRQAPGKGLEWVAWISPYGGSTYY |
| | | ADSVKGRFTISADTSKNTAYLQMNSLRAED |
| | | TAVYYCARRHWPGGFDYWGQGTLVTVSSAS |
| | | VAAPSVFIFPPSDEQLKSGTASVVCLLNNF |
| | | YPREAKVQWKVDNALQSGNSQESVTEQDSK |
| | | DSTYSLSSTLTLSKADYEKHKVYACEVTHQ |
| | | GLSSPVTKSFNRGEC |
| 164 | Heavy chain 1 of P1AI4853 (knob | MTQLVESGGGLVQPGGSLRLSCAADHTTEY |
| | chain) | NAMSWVRQAPGKGLEWVGSISGSGGSTSLA |
| | | RSVKGRFTISRDNSKNTLYLQMNSLRAEDT |
| | | AVYYCARDLGFMDVWGQGTLVTVSSASTKG |
| | | PSVFPLAPSSKSTSGGTAALGCLVEDYFPE |
| | | PVTVSWNSGALTSGVHTFPAVLQSSGLYSL |
| | | SSVVTVPSSSLGTQTYICNVNHKPSNTKVD |
| | | EKVEPKSCGGSGGGGSGGGGSGGGGSGGDI |
| | | QMTQSPSSLSASVGDRVTITCRASQDVSTA |
| | | VAWYQQKPGKAPKLLIYSASFLYSGVPSRF |
| | | SGSGSGTDFILTISSLQPEDFATYYCQQYL |
| | | YHPATFGQGTKVEIKSSASTKGPSVFPLAP |
| | | SSKSTSGGTAALGCLVKDYFPEPVTVSWNS |
| | | GALTSGVHTFPAVLQSSGLYSLSSVVTVPS |
| | | SSLGTQTYICNVNHKPSNTKVDKKVEPKSC |
| | | DKTHTCPPCPAPEAAGGPSVFLFPPKPKDT |
| | | LMISRTPEVTCVVVDVSHEDPEVKFNWYVD |
| | | GVEVHNAKTKPREEQYNSTYRVVSVLTVLH |
| | | QDWLNGKEYKCKVSNKALGAPIEKTISKAK |
| | | GQPREPQVYTLPPCRDELTKNQVSLWCLVK |
| | | GFYPSDIAVEWESNGQPENNYKTTPPVLDS |
| | | DGSFFLYSKLTVDKSRWQQGNVFSCSVMHE |
| | | ALHNHYTQKSLSLSPG |
| 165 | Heavy chain 2 of P1AI4853 (hole | DIQMTQSPSSLSASVGDRVTITCRASQDVS |
| | chain) | TAVAWYQQKPGKAPKLLIYSASFLYSGVPS |
| | | RFSGSGSGIDFTLTISSLQPEDFATYYCQQ |
| | | YLYHPATFGQGTKVEIKSSASTKGPSVFPL |
| | | APSSKSTSGGTAALGCLVKDYFPEPVTVSW |
| | | NSGALTSGVHTFPAVLQSSGLYSLSSVVTV |
| | | PSSSLGTQTYICNVNHKPSNTKVDKKVEPK |
| | | SCDKTHTCPPCPAPEAAGGPSVFLFPPKPK |
| | | DTLMISRTPEVTCVVVDVSHEDPEVKFNWY |
| | | VDGVEVHNAKTKPREEQYNSTYRVVSVLTV |
| | | LHQDWLNGKEYKCKVSNKALGAPIEKTISK |
| | | AKGQPREPQVCTLPPSRDELTKNQVSLSCA |
| | | VKGFYPSDIAVEWESNGQPENNYKTTPPVL |
| | | DSDGSFFLVSKLTVDKSRWQQGNVFSCSVM |
| | | HEALHNHYTQKSLSLSPG |
| 166 | Light chain 1 of P1AI4853 (A | EVQLVESGGGLVQPGGSLRLSCAASGFTFS |
| | chain) | DSWIHWVRQAPGKGLEWVAWISPYGGSTYY |
| | | ADSVKGRFTISADTSKNTAYLQMNSLRAED |
| | | TAVYYCARRHWPGGFDYWGQGTLVTVSSAS |
| | | VAAPSVFIFPPSDEQLKSGTASVVCLLNNF |
| | | YPREAKVQWKVDNALQSGNSQESVTEQDSK |
| | | DSTYSLSSTLTLSKADYEKHKVYACEVTHQ |
| | | GLSSPVTKSFNRGEC |
| 167 | Light chain 2 of P1AI4853 | CDLPQTHSLGSRRTLMLLAQMRKISLFSCL |
| | including IFNα2a (B chain) | KDRHDFGFPQEEFGNQFQKAETIPVLHEMI |
| | | QQIFNLFSTKDSSAAWDETLLDKFYTELYQ |
| | | QLNDLEACVIQGVGVTETPLMKEDSILAVR |
| | | KYFQRITLYLKEKKYSPCAWEVVRAEIMRS |
| | | FSLSTNLQESLRSKEGGSGGGGSGGGGSGG |
| | | GGSGGDIQMTQSPSSLSASVGDRVTITCDA |
| | | VITISQELAWYQQKPGKAPKLLIYDASYHE |
| | | SGVPSRFSGSGSGIDFTLTISSLQPEDFAT |
| | | YYCQQYGNWPHTFGQGTKVEIKRTVAAPSV |
| | | FIFPPSDRKLKSGTASVVCLLNNFYPREAK |
| | | VQWKVDNALQSGNSQESVTEQDSKDSTYSL |
| | | SSTLTLSKADYEKHKVYACEVTHQGLSSPV |
| | | TKSFNRGEC |
| 168 | Heavy chain 1 of P1AH1108 | DIQMTQSPSSLSASVGDRVTITCRASQDVS |
| | (knob chain) | TAVAWYQQKPGKAPKLLIYSASFLYSGVPS |
| | | RFSGSGSGTDFTLTISSLQPEDFATYYCQQ |
| | | YLYHPATFGQGTKVEIKSSASTKGPSVFPL |
| | | APSSKSTSGGTAALGCLVKDYFPEPVTVSW |
| | | NSGALTSGVHTFPAVLQSSGLYSLSSVVTV |
| | | PSSSLGTQTYICNVNHKPSNTKVDKKVEPK |
| | | SCDKTHTCPPCPAPEAAGGPSVFLFPPKPK |
| | | DTLMISRTPEVTCVVVDVSHEDPEVKFNWY |
| | | VDGVEVHNAKTKPREEQYNSTYRVVSVLTV |
| | | LHQDWLNGKEYKCKVSNKALGAPIEKTISK |
| | | AKGQPREPQVYTLPPCRDELTKNQVSLWCL |
| | | VKGFYPSDIAVEWESNGQPENNYKITPPVL |
| | | DSDGSFFLYSKLTVDKSRWQQGNVFSCSVM |
| | | HEALHNHYTQKSLSLSPG |
| 169 | Heavy chain 2 of P1AH1108 (hole | MSQLVESGGGLVQPGGSLRLSCAANNDIEF |
| | chain) | NAMSWVRQAPGKGLEWVGSISPKGGSKSIH |
| | | RKFQGRFTISRDNSKNTLYLQMNSLRAEDT |
| | | AVYYCARDLGFMDIWGQGTLVTVSSASTKG |
| | | PSVFPLAPSSKSTSGGTAALGCLVEDYFPE |
| | | PVTVSWNSGALTSGVHTFPAVLQSSGLYSL |
| | | SSVVTVPSSSLGTQTYICNVNHKPSNTKVD |
| | | EKVEPKSCDKTHTCPPCPAPEAAGGPSVFL |
| | | FPPKPKDTLMISRTPEVTCVVVDVSHEDPE |
| | | VKFNWYVDGVEVHNAKTKPREEQYNSTYRV |
| | | VSVLTVLHQDWLNGKEYKCKVSNKALGAPI |
| | | EKTISKAKGQPREPQVCTLPPSRDELTKNQ |
| | | VSLSCAVKGFYPSDIAVEWESNGQPENNYK |
| | | TTPPVLDSDGSFFLVSKLTVDKSRWQQGNV |
| | | FSCSVMHEALHNHYTQKSLSLSPG |
| 170 | Light chain 1 of P1AH1108 | CDLPQTHSLGSRRTLMLLAQMRKISLFSCL |
| | including IFNα2a (A chain) | KDRHDFGFPQEEFGNQFQKAETIPVLHEMI |
| | | QQIFNLFSTKDSSAAWDETLLDKFYTELYQ |
| | | QLNDLEACVIQGVGVTETPLMKEDSILAVR |
| | | KYFQRITLYLKEKKYSPCAWEVVRAEIMRS |
| | | FSLSTNLQESLRSKEGGSGGGGSGGGGSGG |
| | | GGSGGKTQMTQSPSSLSASVGDRVTITCDG |
| | | TYGISNELAWYQQKPGKAPKLLIYDGEYHD |
| | | FDVPSRFSGSGSGSDFTLTISSLQPEDFAT |
| | | YYCQQYGNWPHTFGQGTKVEIKRTVAAPSV |
| | | FIFPPSDRKLKSGTASVVCLLNNFYPREAK |
| | | VQWKVDNALQSGNSQESVTEQDSKDSTYSL |
| | | SSTLILSKADYEKHKVYACEVTHQGLSSPV |
| | | TKSFNRGEC |
| 171 | Light chain 2 of P1AH1108 (B | EVQLVESGGGLVQPGGSLRLSCAASGFTFS |
| | chain) | DSWIHWVRQAPGKGLEWVAWISPYGGSTYY |
| | | ADSVKGRFTISADTSKNTAYLQMNSLRAED |
| | | TAVYYCARRHWPGGFDYWGQGTLVTVSSAS |
| | | VAAPSVFIFPPSDEQLKSGTASVVCLLNNF |
| | | YPREAKVQWKVDNALQSGNSQESVTEQDSK |
| | | DSTYSLSSTLTLSKADYEKHKVYACEVTHQ |
| | | GLSSPVTKSFNRGEC |

### III. EXAMPLES

The following are examples of methods and compositions of the invention. It is understood that various other embodiments may be practiced, given the general description provided above.

Although the foregoing invention has been described in some detail by way of illustration and example for purposes of clarity of understanding, the descriptions and examples should not be construed as limiting the scope of the invention. The disclosures of all patent and scientific literature cited herein are expressly incorporated in their entirety by reference.

### Recombinant DNA Techniques

Standard methods were used to manipulate DNA as described in Sambrook, J. et al., Molecular cloning: A laboratory manual; Cold Spring Harbor Laboratory Press, Cold Spring Harbor, New York, 1989. The molecular biological reagents were used according to the manufacturer's instructions.

### Gene and Oligonucleotide Synthesis

Desired gene segments were prepared by chemical synthesis at Geneart GmbH (Regensburg, Germany). The synthesized gene fragments were cloned into an E. coli plasmid for propagation/amplification. The DNA sequences of subcloned gene fragments were verified by DNA sequencing. Alternatively, short synthetic DNA fragments were assembled by annealing chemically synthesized oligonucleotides or via PCR. The respective oligonucleotides were prepared by metabion GmbH (Planegg-Martinsried, Germany)

### Description of the Basic/Standard Mammalian Expression Plasmid

For the expression of a desired gene/protein (e.g. full length antibody or fusion protein heavy chain, full length antibody or fusion protein light chain, or an antigen used in the assays herein, e.g. PD-L1, IFNα-2 or others) a transcription unit comprising the following functional elements is used:
the immediate early enhancer and promoter from the human cytomegalovirus (P-CMV) including intron A,
a human heavy chain immunoglobulin 5′-untranslated region (5′UTR),
a murine immunoglobulin heavy chain signal sequence,
a gene/protein to be expressed (e.g. full length antibody or fusion protein heavy chain), and
the bovine growth hormone polyadenylation sequence (BGH pA).

Beside the expression unit/cassette including the desired gene to be expressed the basic/standard mammalian expression plasmid contains
an origin of replication from the vector pUC18 which allows replication of this plasmid in E. coli, and
a beta-lactamase gene which confers ampicillin resistance in E. coli.

### Protein Determination

The protein concentration of purified polypeptides was determined by determining the optical density (OD) at 280 nm, using the molar extinction coefficient calculated on the basis of the amino acid sequence of the polypeptide.

### Example 1: Generation of Recombinant Proteins Used in Antibody and Fusion Protein Generation and Characterization

Table 3 lists the recombinant proteins used during generation and characterization of the bispecific DutaFabs, Fabs, IgGs and fusion proteins of the invention, such as recombinant human and cynomolgus PD-L1 extracellular domain protein and human interferon alpha 2a protein.

TABLE 3
Description of recombinant proteins used during generation
and characterization of molecules of the invention

| Molecule ID | Name | Description |
|---|---|---|
| P1AD8569 | Human PD-L1 (CD274) | human PD-L1 ECD (amino acid 19-239) fused |
| | ECD with C-terminal Fc- | at its C-terminus to the N-terminus of C- |
| | Tag | terminal Fc-Tag (SEQ ID NO: 157) |
| P1AF6937 | OneArmed-huPD- | human PD-L1 ECD (amino acid 18-238) fused |
| | L1_ECD_Fc_LALAPG | at its C-terminus to the knob chain of |
| | | Fc_LALAPG Avi-His (and SEQ ID NO: 151) |
| P1AF6938 | OneArmed-cyPD- | Cynomolgus PD-L1 ECD (amino acid 18-238) |
| | L1_ECD_Fc_LALAPG_avi_his | fused at its C-terminus to Fc_LALAPG Avi-His |
| | | (SEQ ID NO: 150 and SEQ ID NO: 151) and |
| P1AF7352 | OneArmed_PD-L1 cell- | Human PD-L1 C-terminal domain (amino acid |
| | surface proximal domain, | 133-238) fused at its N-terminus via a peptide |
| | Fc-LALAPG_avi_his | linker to the C-terminus of the knob chain of the |
| | | Fc-PGLALA avi-his Tag (SEQ ID NO: 150 and |
| | | SEQ ID NO: 151) |
| P1AF6939 | huPD-L1-avi_his | Human PD-L1 ECD (amino acid 18-238) fused |
| | | at its C-terminus via a peptide linker to the N- |
| | | terminus of the avi-his Tag (SEQ ID NO: 149) |
| P1AD8636 | huIFNα-2a (amino acid | Human IFNα-2a (mature protein without signal |
| | 24-188) | peptide) |
| P1AE9514 | huIFNα-2a (amino acid | Human IFNα-2a linked at its N-terminus via a |
| | 24-188) with N-terminal | peptide linker to the C-terminus of the knob |
| | Fc-AviTag ™ | chain of an N-terminal Fc-AviTag ™ (SEQ ID |
| | | NO: 152 and SEQ ID NO: 153) |
| P1AF6940 | huIFNα-2a (amino acid | Human IFNα-2a linked at its C-terminus via a |
| | 24-188) with C-terminal | peptide linker to the N-terminus of the knob |
| | Fc-AviTag ™ | chain of an C-terminal Fc-AviHisTag ™ (SEQ |
| | | ID NO: 154 and SEQ ID NO: 155) |
| P1AF6942 | huIFNα-2a (amino acid | Human IFNα-2a linked at its C-terminus via a |
| | 24-188) with C-terminal | peptide linker to the N-terminus of the Avi-His- |
| | Avi His-Tag ™ | Tag ™ (SEQ ID NO: 149) |
| P1AD9385 | huIFNAR2 ECD with C- | Human IFNAR2 extracellular domain linked at |
| | terminal His-AviTag ™ | its C-terminus to the N-terminus of the His-Avi- |
| | | Tag ™ (SEQ ID NO: 156) |
| P1AI4841 | OneArmed anti-PD-L1 | anti-PD-L1 antibody Atezolizumab CrossFab |
| | antibody Atezolizumab C- | linked at the C-terminus of the Fab heavy chain |
| | terminal Avi-Tag | to the N-terminus of the knob chain of a C- |
| | | terminal Fc-AviTag ™ (SEQ ID NO: 158 and |
| | | SEQ ID NO: 159) |
| P1AE1979 | Human CD79B | Human CD79B extracellular domain fused at its |
| | extracellular domain | C-terminus via a peptide linker to the N- |
| | (amino acid 29-159, | terminus of each polypeptide of an Fc domain |
| | Uniprot ID P40259) with | (SEQ ID NO: 157) |
| | C-terminal Fc-tag | |
| P1AG2011 | OneArmed_Atezolizumab_{—} | anti-PD-L1 antibody Atezolizumab Fab linked |
| | Fab_hu-IFNA2-R149A | at the C-terminus of the Fab heavy chain to the |
| | | N-terminus of the knob chain of a Fc domain |
| | | (PGLALA), with an IFN-α2a R149A variant |

### Expression and Purification

All proteins used for the experiments, including tool proteins for antibody generation and binding analyses, if not stated otherwise, were generated by gene synthesis (Twist Bioscience, USA) and cloned into Roche's in-house expression vector using standard cloning procedures. All antigens were expressed under the control of the CMV-promoter. For the generation of expression plasmids for antibodies and antibody-based formats, such as recombinant monoclonal antibodies and one-armed antibody constructs, the recombinant monoclonal antibody genes encoding the respective immunoglobulin heavy and light chains were cloned into the expression plasmids for the transient expression which comprised besides the heavy or light chain expression cassette an origin of replication from the vector pUC18, which allows replication of this plasmid in E. coli, and a beta-lactamase gene which confers ampicillin resistance in E. coli.

The transcription unit of a respective heavy or light chain comprised the following functional elements:
the immediate early enhancer and promoter from the human cytomegalovirus (P-CMV) including intron A,
a human heavy chain immunoglobulin 5′-untranslated region (5′UTR),
a murine immunoglobulin heavy chain signal sequence, and
the bovine growth hormone polyadenylation sequence (BGH pA).

For transient expression of the protein constructs, Expi293F™ cells (Thermo Fisher Scientific, USA) or HEK293F cells (Thermo Fisher Scientific, USA) were transfected with the respective plasmids (in the case of antibodies and antibody-based formats, co-transfected with plasmids containing the respective immunoglobulin heavy- and light chain) and expressed as described for IgG-like proteins, except for untagged human IFN-α2a which was expressed in E. coli cells according to standard methods. The cells were subsequently harvested by centrifugation and the protein-containing supernatant was filtered using a 0.22 μm vacuum filtration system (Millipore). The His-AviTag™ and Avi-HisTag™ tagged proteins were purified by IMAC affinity chromatography using complete-His-Tag resin (Roche Diagnostics). After washing with 50 mM Na₂PO₄, 300 mM NaCl, pH 8.0, His-AviTag™ fusion proteins were eluted using a washing buffer supplemented with 500 mM imidazole at pH 7.0. Fc-tagged proteins were captured from cell culture supernatants by affinity chromatography using MabSelectSure-Sepharosc™ (Cytiva Life Sciences, USA) using PBS buffer (10 mM sodium phosphate, 1 mM potassium phosphate, 137 mM sodium chloride and 2.7 mM potassium chloride, pH 7.4) as equilibration and washing buffer and 100 mM sodium acetate, pH 3.0 as elution buffer. Aggregated protein was separated from monomeric fusion proteins by size exclusion chromatography (Superdex® 200, Cytiva Life Sciences, USA) in 20 mM Histidine, 140 mM NaCl, pH 6.0. Monomeric protein fractions were pooled, concentrated, if required, using e.g. a MILLIPORE Amicon Ultra (5 or 10 kDa MWCO) centrifugal concentrator and stored at −80° C. General information regarding the recombinant expression of human immunoglobulins in e.g. HEK293 cells is given in: Meissner, P. et al., Biotechnol. Bioeng. 75 (2001) 197-203. Sample aliquots were used for subsequent analytical characterization e.g. by CE-SDS, size exclusion chromatography and mass spectrometry.

Untagged human PD-L1 extracellular domain (aa 19-232, P1AD9695) was produced by papain digestion of P1AD8569 (human PD-L1 (CD274) ECD with C-terminal Fc) according standard procedure. Briefly, human PD-L1 ECD with C-terminal Fc was incubated with papain (Sigma, 4 mU/mg substrate) in the presence of 0.2 mM cysteine at 37° C. for about 2 hours and stopped by addition of 2-iodoacetamide to a final concentration of 5 mM 2-iodoacetamaide. The ECD fragment was purified in flow-through mode via MabSelectSure (Cytiva life sciences, USA) and subsequent size exclusion chromatography using 20 mM Tris, 150 mM NaCl pH 7.5 as buffer.

### Biotinylation of Tool Proteins

Enzymatic site-specific biotinylation of human or cynomolgus PD-L1 extracellular domain constructs or human IFN-α2 containing an AviTag™ was performed by using the BirA biotin-protein ligase kit (Avidity LLC, USA) according to manufacturer's instruction. Briefly, 1/10 volume of BiomixA (10× concentration: 0.5 M bicine buffer, pH 8.3) and BiomixB (10× concentration: 100 mM ATP, 100 mM MgOAc, 500 μM d-biotin) was added to AviTag™ containing protein followed by addition of 2.5 μg BirA ligase per 10 nmol protein. The reaction mixture was incubated at 30° C. for 1 hour and purified by size exclusion chromatography on a Superdex75 or Superdex 200 prep grade pre-packed HiLoad column (Cytiva Life Sciences, USA).

Recombinant IFN-α2a protein was chemically biotinylated by using a 2.5 molar excess of EZ-Link™ Sulfo-NHS-SS-Biotin (ThermoFischer Scientific, #A39258). The reaction was carried out for 1 hour at room temperature and the excess of biotin was removed by two steps of dialysis to PBS 1×, pH 7.4. Biotinylation was assessed via pull-down assay, showing that approximately 80% of the protein was biotinylated.

The PD-L1 ECD (extracellular domain) generated by digestion of P1AD8569 with papain was chemically biotinylated using a 7-fold molar excess of EZ-Link™ Sulfo-NHS—SS-Biotin (ThermoFischer Scientific, #A39258). Human and cynomolgus PD-L1 Fc were similarly biotinylated using a 3-fold molar excess of the same reagent. The reactions were carried out for 1 hour at room temperature and the excess of biotin was removed by two steps of dialysis to PBS 1×, pH 7.4. Successful biotinylation was assessed via pull-down assay, showing that approximately ˜90% of human PD-L1 extracellular domain, ˜70% of human PD-L1-Fc fusion and ˜50% of cynomolgus PD-L1-Fc fusion were biotinylated.

### Example 2: Generation of a Mutually Exclusive Anti-huPD-L1/Anti-huIFN-α2a Binding DutaFab

The fusion proteins as described herein comprise DutaFabs, engineered therapeutic Fab fragments that are bispecific, i.e. they can bind two distinct antigens. DutaFabs are dual-targeting Fab molecules which comprise two binding sites with different specificities through the assignment of residues into two sides: the so-called H-side paratope encompassing CDR-H1, CDR-H3 and CDR-L2, and the L-side paratope encompassing CDR-L1, CDR-L3 and CDR-H2. Generally, DutaFabs are designed to bind two target molecules simultaneously at the same Fv region comprising a VH-VL heterodimer and can be used as bispecific binding agents. In the present invention, the DutaFab platform is modified to generate a DutaFab capable of binding to human PD-L1 and to human Interferon alpha 2a which-instead of binding those targets simultaneously-binds to them in a mutually exclusive manner (“Dutaflip”).

### a) Selection of PD-L1 Binding DutaFabs

In an initial step to generate mutually exclusively binding DutaFabs, DutaFabs binding to human PD-L1 were generated by phage display. For this, two sets of Roche in-house phage display libraries of synthetic Fab fragments were utilized. In the first set, the residues within the CDR-H1, CDR-H3, VH N-terminus and CDR-L2 regions of the Fab fragments (corresponding to the so-called H-side paratope) were diversified, whereas in the second set the residues within the CDR-L1, CDR-L3, VL N-terminus and CDR-H2 regions of the Fab fragments (corresponding to the so-called L-side paratope) were diversified. In each library set, the other three CDR regions were kept non-diversified as either invariant dummy sequence or in some contexts as CDRs specific for an irrelevant epitope. In both libraries, the CH1 domains of the Fab fragments were fused via a linker to truncated gene-III protein to facilitate phage display.

Phage library panning was performed in four rounds, wherein the first round was performed with 250 nM in 1 ml of randomly S—S linker biotinylated PD-L1 ECD (expressed and biotinylated as described in Example 1) pre-immobilized on Dynabcads™ M-280 Streptavidin magnetic beads (Thermo Fisher catalog number 11206D), and rounds 2 to 4 were performed with 250 nM, 100 nM, and 100 nM in 1 ml of randomly S—S linker biotinylated PD-L1 ECD in solution, followed by capture of Fab-on-phage/target complexes on the beads. Only in round two, the Streptavidin beads were replaced by Neutravidin-coupled magnetic beads (Sera-Mag SpeedBead Neutravidin-Coated Magnetic Particles, Cytiva: 78152104010350). In round one, captured phage clones bearing target-specific DutaFabs in combination with the beads were directly infected into log-phase TG1 E. coli cells, and rescued using M13KO7 helper phage, according to standard protocols. In rounds 2 to 4, the DutaFab-bearing phages were eluted from the magnetic beads using cither 100 mM DTT or 2.5 μM Atezolizumab prior to being used for infection.

For screening of selection outputs, a polyclonal plasmid miniprep of the respective selection round was prepared from the infected TG1 E. coli cells. Plasmids were digested using BamHI restriction endonuclease, which cuts the phagemid pDuta4 upstream and downstream of the phage Fd gene 3 domain. Plasmids were recircularized by ligation, generating an in-frame fusion of a T7 tag at the C-terminus of the DutaFab CH1 domain. The ligated polyclonal plasmids encoding T7-tagged DutaFabs were transformed into TG1 E. coli cells, and single colonies were picked into microtiter plates. Soluble DutaFabs were expressed in microtiter plates and supernatants were clarified by centrifugation.

Human PD-L1 specific binders were identified by ELISA as follows: 20 μL mixture of biotinylated antigen huPD-L1(ECD)-huFc fusion (P1AF6937; produced as described in Example 1) (0.5 μg/mL final concentration in assay) and detection antibody anti-human Ig kappa chain specific antibody POD (peroxidase) (Millipore AP502, 1:6000 final concentration in assay) were mixed with 5 μL DutaFab-containing bacterial supernatant and added to streptavidin coated microtiter plates (Microcoat 384 SA, 11974998001). After an incubation for 60 min at RT, the plates were washed 6× with PBST, 0.1% Tween20. The binding of DutaFabs to huPD-L1 (ECD)-huFc (P1AF6937) was detected by adding 30 μL TMB substrate to the wells. After an incubation of 5 min at RT, the absorbance was measured at 370 nm with an EnVision Reader (PerkinElmer).

Cynomolgus PD-L1 specific binders were identified by ELISA as follows: 20 u L mixture of biotinylated antigen cynoPD-L1 (ECD)-huFc (P1AF6938, produced as described in Example 1) (0.5 μg/mL final in assay) and detection antibody anti-human Ig kappa chain specific antibody POD (Millipore AP502P, 1:6000 final in assay) were mixed with 5 μL DutaFab-containing bacterial supernatant and added to streptavidin coated microtiter plates (Microcoat 384 SA, 11974998001). After an incubation for 60 min at room temperature, the plates were washed 6 times with PBST, 0.1% Tween20. The binding of DutaFabs to cynoPD-L1 (ECD)-huFc (P1AF6938) was detected by adding 30 μL TMB substrate to the wells. After an incubation of 5 min at RT, the absorbance was measured at OD 370 nm with an EnVision Reader (PerkinElmer). ELISA-positive clones were bacterially expressed as soluble DutaFab fragments in 96-well format. Clones expressing PD-L1-specific DutaFabs were identified, and the corresponding phagemids were sequenced and 115 unique sequences were discovered.

A selection of these unique clones was thereafter expressed in E. coli and purified via a one-step affinity capture using CaptureSelect™ IgG-CH1 resin (Thermo Fisher; Catalog number: 19432001L) in order to be characterized. Binding to PD-L1-positive cells was assessed via FACS. DutaFabs were examined using analytical size exclusion chromatography. SPR (Biacore 8K or 8K+) was used to determine binding kinetics to huPD-L1-Fc (P1AF6937), human PD-L1 (CD274) C-terminal extracellular domain (P1AF7352), cynomolgus PD-L1-Fc (P1AF6938) and CD79B ECD dimer (P1AE1979) (negative control). Briefly, anti-human Fab antibody (Cytiva; Catalog number 28958325) was immobilized on a CM5 chip according to the manufacturer's instructions.

TABLE 4
PD-L1 affinities of selected DutaFabs from phage display

| | | Affinity | Epitope | | |
|---|---|---|---|---|---|
| | | to PD- | [N- or | Competitive | Blocking |
| | | L1; | C-terminal | PD-L1 | PD-L1/ |
| | | kDa | domain of | binding with | PD1 |
| Sample | ID | [nM] | PD-L1) | Atezolizumab | interaction |
| C16 | P1AG5366 | 23 | N-terminal | yes | no |
| E24 | P1AG2233 | 20 | N-terminal | yes | yes |
| J12 | P1AG4949 | 56 | C-terminal | no | n.d. |
| O06 | P1AG4948 | 75 | C-terminal | no | no |
| H13 | P1AG2349 | 41 | N-terminal | yes | yes |
| D10 | P1AG2248 | 1.2 | N-terminal | yes | yes |
| M14 | P1AG2310 | 4.4 | C-terminal | no | yes |
| (par.) | | | | | |
| E06 | P1AG2356 | 34 | C-terminal | no | no |

100 nM DutaFabs were captured 10 μl/min, 60 sec) and 0 nM, 10 nM, 50 nM and 150 nM of antigen was flown at 30 μl/min for 120 sec followed by a 240 second dissociation window at a flow rate of 30 μl/min. The surface was regenerated by injecting 10 mM glycine, pH 2, for 60s at a flow rate of 30 μl/min. Additionally, competitive binding to PD-L1 with Atezolizumab was investigated by SPR in a competitive binding assay set-up. Table 4 shows affinity to PD-L1, epitope binding, competitive binding with Atezolizumab and blocking of PD-L1/PD1 binding (measured in an cellular assay as described in Example 6 e) for several of the generated anti-PD-L1 DutaFabs. Notably, M14 (parental), a DutaFab with high affinity to PD-L1 bound to the C-terminus of PD-L1, thus not competing with Atezolizumab (which binds to the N-terminus of PD-L1) for PD-L1 binding, but it surprisingly blocked the interaction between PD-L1 and PD1. This was unexpected as usually only antibodies binding to the N-terminus of PD-L1 are able to block PD-L1/PD1 interaction.

14 huPD-L1-binding DutaFabs covering a range of affinities and including DutaFabs binding to either PD-L1 N-terminus or C-terminus were selected to create matrices to generate and screen mutually exclusive DutaFabs binding to huPD-L1 and huIFN-α2a (see Example 2 c).

### b) Selection and Characterization of IFN-α2a Binding DutaFabs

Generation of DutaFabs binding specifically to human Interferon α-2a was carried out by phage display. For this, recombinant biotinylated IFN-α2a was used (biotinylation was performed as described in Example 1 above).

Two distinct sets of Roche in-house phage display libraries of synthetic Fab fragments were utilized. In the first phage display library set (“H-side libraries”) residues within the VH N-terminus, CDR-H1, CDR-H3, VH outer loop and CDR-L2 regions of the Fab fragments were diversified, whereas in the second phage display library set (“L-side libraries”) residues within the Vκ N-terminus, CDR-L1, CDR-L3, Vκ outer loop and CDR-H2 regions of the Fab fragments were diversified. In each library, the other three CDR regions, the N-terminus and the outer loop were kept non-diversified as invariant dummy sequence or as CDRs specific for an irrelevant target in some contexts. In both sets of libraries, the CH1 domain of the Fab fragments was fused via a linker to a truncated gene-III protein to facilitate phage display.

Phage library panning was performed in four rounds, wherein the first round was performed with 250 nM of biotinylated mature full-length IFN-α2a (without signal peptide) pre-immobilized on Dynabeads™ M-280 Streptavidin magnetic beads (Thermo Fisher catalog number 11206D), and rounds 2-4 were performed with 250 nM, 100 nM, and 100 nM of biotinylated target in solution, followed by capture of Fab-on-phage/target complexes on the beads. In round 2 and 4, Neutravidin-coupled magnetic beads (Sera-Mag SpeedBeads Neutravidin-Coated Magnetic Particles, Cytiva: 78152104010350) were used.

In round one, captured phage clones bearing target-specific DutaFabs in combination with the beads were directly infected into log-phase TG1 E. coli cells, and rescued using M13KO7 helper phage, according to standard protocols. In rounds 2-4, the DutaFab bearing phages were eluted from the magnetic beads using either 100 mM DTT or 2 μM Rontalizumab prior to being used for infection.

For screening of selection outputs, a polyclonal plasmid miniprep of the respective selection round was prepared from the infected TG1 E. coli cells. Plasmids were digested using BamHI restriction endonuclease, which cuts the phagemid pDuta4 upstream and downstream of the phage Fd gene 3 domain. Plasmids were recircularized by ligation, generating an in-frame fusion of a T7 tag at the C-terminus of the DutaFab CH1 domain. The ligated polyclonal plasmids encoding T7-tagged DutaFabs were transformed into TG1 E. coli cells, and single colonies were picked into microtiter plates. Soluble DutaFabs were expressed in microtiter plates and supernatants were clarified by centrifugation.

The DutaFab culture supernatants were screened and specific binders were identified by ELISA as follows: 20 μL mixture of biotinylated antigen human IFN-α2a-hFc with N-terminal Fc-AviTag™ (monomer; P1AE9514; produced and biotinylated as described in Example 1a) (1 μg/mL final concentration in assay) and detection antibody anti-human Ig kappa chain specific antibody POD (Millipore AP502P, 1:6000 final in assay) were mixed with 5 μL DutaFab containing bacterial supernatant and added to streptavidin coated microtiter plates (MicroCoat 384 SA, 11974998001). After an incubation for 60 min at room temperature, the plates were washed 6 times with PBST, 0.1% Tween20. The binding of DutaFabs to human IFN-α2a was detected by adding 30 μL TMB substrate to the wells. After an incubation of 5 min at RT, the absorbance was measured at OD 370 nm with an EnVision Reader (PerkinElmer). Clones expressing IFN-α2a specific DutaFabs were identified and the corresponding phagemids were sequenced.

685 unique sequences were discovered and subsequently grouped into families. 157 clones were chosen according to the ELISA results, sequence frequency and family coverage and expressed in E. coli. DutaFabs were purified from the E. coli supernatant via a one-step affinity chromatography purification on CaptureSelect™ IgG-CH1 resin (Thermo Fisher; Catalog number: 19432001L). The quality of the purified DutaFabs was analyzed via size exclusion chromatography. The binding affinity of the DutaFabs was assessed by SPR-analysis using a Biacore 8K or 8K+ instrument (Cytiva) as described above. Binding kinetics of binding to human IFN-α2a, to cynomolgus IFN-α2a and to CD79B dimer (control) were analyzed.

Competitive binding to IFNAR2 was investigated as follows. A sensor chip Biacore™ Sensor Chip NTA (Cytiva, Catalog number 28994951) was loaded with biotinylated IFNAR2 (P1AD9385) by flowing 100 nM of the protein for 120 seconds at a flow rate of 10 μl/min followed by 100 nM IFN-α2a for 120 seconds. 100 nM DutaFabs were subsequently flown for 120 seconds. Competitive binding was assessed by individual evaluation of the sensorgrams. The chip was finally regenerated by injecting 350 mM EDTA for 60 seconds at a flow rate of 30 μl/min. 15 hits showing high binding to human IFN-α2a and competition with IFNAR2 were tested in a HEK blue assay for IFN-α2a pathway inhibition (assay as described in Example 13) and IFNAR blocking DutaFabs were selected for further screening. Overall, nine human IFN-α2a binding DutaFabs were selected to create matrices to generate mutually exclusive DutaFabs binding to PD-L1 and IFN-α2a (see Example 2 c).

### c) Creation and Characterization of Mutually Exclusive Bispecific DutaFabs (“DutaFlips”)

A bispecific design matrix was generated for the identification of mutually exclusively binding DutaFabs, combining H-sides of six anti-PD-L1 DutaFabs with the L-side of two anti-IFN-α2 DutaFabs, as well as seven H-sides of anti-IFN-α2 DutaFabs with the L-sides of eight anti-PD-L1 DutaFabs, resulting in 68 molecules in total.

In each of those molecules, one pair of an H-side monospecific paratope against the first target was combined with an L-side monospecific paratope against the second target to obtain the bispecific candidate. The bispecific protein sequence was then generated in silico by making an alignment of the germline-like scaffold sequence with the H-side binder and the L-side binder, and substituting all potential paratope residues from both binders into the scaffold sequence. The bispecific DutaFab genes were synthesized (Twist Bioscience, USA) and cloned into an E. coli expression vector. The vector was transformed into E. coli TG1 cells, and individual colonies were cultured for soluble expression of the bispecific DutaFab fragments. The bispecific DutaFab fragments were purified from the E. coli culture supernatant by affinity chromatography via a one-step affinity chromatography purification on CaptureSelect™ IgG-CH1 resin (Thermo Fisher; Catalog number: 19432001L), and specific binding to human IFN-α2a, to human PD-L1 and cynomolgus PD-L1 was verified by SPR-analysis, essentially as described above in sections a) and b), respectively, of this example. Binding to CD79B was used as a negative control. Out of 68 bispecific DutaFabs, 25 were not expressed well and could not be characterized. Of the remaining DutaFabs, nine showed good/excellent, 16 medium/low and 18 no binding for IFN-α2a; and 24 showed good/excellent, 11 medium/low and 8 no binding for PD-L1. In summary, 13 had good binding for both targets, 11 had low binding for at least one target and 19 had no binding for at least one target.

In order to determine whether the selected DutaFabs bind their targets simultaneously or mutually exclusively, bridging experiments were performed. In a first experiment, a Human Antibody capture kit (Cytiva, Catalog number BR 100839) was used to immobilize 10 μg/ml of IgG capture antibody (applied for 500 seconds at a flow rate of 10 μl/min) to a Series S CM5 Sensor Chip (Cytiva, Catalog number 29149603) using standard amine coupling chemistry. As running and dilution buffer, HBS-EP+ (0.1 M HEPES, 1.5 M NaCl, 0.03 M EDTA and 0.5% v/v Surfactant P20, Cytiva BR100669) was used. To assess non-simultaneous binding, a mixture of 75 nM Fc-IFN-α2a (P1AF9514) and 75 nM IFN-α2a-Fc (P1AF6940) were captured to the surface of the chip by flowing them at 10 μl/min for 120 seconds. DutaFabs (150 nM) and PD-L1 Avi-His (P1AF6939) (150 nM) were flown subsequently for 120 seconds each at 30 μl/min. In a second experiment, 150 nM PD-L1 Fc (P1AF6939) was captured to the surface of the chip by flowing them at 10 μl/min for 120 seconds at 30 μl/min. DutaFabs (150 nM) and IFN-α2a (150 nM) were flown subsequently for 120 seconds each.

In both experiments, mutually exclusive binding was assessed by individual evaluation of the sensorgrams. Finally, the chip surface was regenerated by injecting 3 M MgCl₂, for 60 seconds at a flow rate of 30 μl/min.

In a third experiment, a Human Antibody capture kit (Cytiva, Catalog number BR100839) was used to immobilize 10 μg/ml of IgG capture antibody (applied for 500 seconds at a flow rate of 10 μl/min) to a Series S CM5 Sensor Chip (Cytiva, Catalog number 29149603) using standard amine coupling chemistry. As running and dilution buffer, HBS-EP+ (0.1 M HEPES, 1.5 M NaCl, 0.03 M EDTA and 0.5% v/v Surfactant P20, Cytiva BR100669) was used. To assess mutually exclusive binding, a mixture of 75 nM Fc-IFN-α2a (P1AE9514) and 75 nM IFN-α2a-Fc (P1AF6940) were captured to the surface of the chip by flowing them at 10 μl/min for 60 seconds. DutaFabs mixed with 0 nM, 300 nM and 1000 nM of PD-L1 Avi His were flown subsequently for 120 seconds at 30 μl/min. Mutually exclusive binding was evaluated comparing the sensorgrams in the presence of PD-L1 versus in the absence of PD-L1 (0 nM). Finally, the chip surface was regenerated by injecting 3M MgCl₂ for 60 seconds at a flow rate of 30 μl/min.

The two clones M14HH17L (parental) and E06HP7L (parental) were shown to bind PD-L1 and IFN-α2a in a mutually exclusive manner and were chosen for affinity maturation.

### Example 3: Characterization of Bispecific Anti-IFN-α2a/Anti-PD-L1 DutaFabs During Maturation and Sequence Polishing

During the maturation of the PD-L1 and IFN-α2a paratopes (described below in Examples 4 and 5), candidate antibodies derived from DutaFab M14HH17L and E06HP7L, expressed as Fab fragments in E. coli, were screened and selected based on their desired properties inter alia with respect to antigen binding, hydrophilicity, negative and positive charge patch volume and thermal stability. Binding affinity was assessed in vitro essentially as described in section a) of Example 2. Monomeric content, hydrophilicity, thermal stability and charge patches of DutaFabs M14HH17L- and E06HP7L-derived variants were assessed as follows:

### a) Size Exclusion Chromatography (SEC)

Monomeric Fab content was determined by injecting 5-25 μg (100 μL) of the respective DutaFab onto a Superdex 75 10/300 GL column (Cytiva) equilibrated with 1×PBS pH 7.4. Elution was performed using 1×PBS pH 7.4 within 35 minutes at 0.75 ml/min. Retention time was compared to commercial protein standards.

### b) Hydrophobic Interaction Chromatography (HIC)

Apparent hydrophobicity was determined by injecting 20 μg of the bispecific anti-IFNα2a/anti-PD-L1 DutaFab onto a HIC-Ether-5 PW column (Tosoh) equilibrated with 25 mM Na-phosphate, 1.5 M ammonium sulfate, pH 7.0. Elution was performed with a linear gradient from 0 to 100% buffer B (25 mM Na-phosphate, pH 7.0) within 60 minutes. Retention times were compared to protein standards with known hydrophobicity.

### c) Differential Scanning Fluorimetry (DSF)

4 μL sample (at a concentration of at least 2 μM) was mixed with 1 μL Sypro® Orange and transferred to a LightCycler® 480 Multiwell Plate 384 sealed with a LightCycler 480 Sealing Foil. Prior to the measurement, the instrument was blanked with deionized water. The measurement was carried out on a LightCycler 480-II Serie 5004 increasing the temperature from 20° C. to 95° C. at a scan rate of 0.06° C./s. (3.6° C./min, 10 acquisitions per° C.). The thermodynamic properties were determined using the LightCycler 480 software 1.5.1. The Tm calculation and data evaluation were done via Genedata Screener®.

### d) In Silico Analysis of Positive and Negative Charge Patches

Candidates with a) predicted CDR Asn/Asp chemical degradation hotspots, b) predicted CDR Trp/Met oxidation hotspots, c) predicted T-cell epitopes, and d) large charge patch volumes were deselected in silico. For a), using an Fv 3D homology model, a decision tree algorithm (Sydow et al., 2014. PLOS ONE 9 (6): e100736. https://doi.org/10.1371/journal.pone.0100736) was used to predict the likelihood of Asp isomerization and Asn deamidation, based on structural descriptors. For b), Trp and Met oxidation is predicted based on solvent accessibility of the respective side chains using a homology model, essentially as described by Sharma et al., 2014 (PNAS 111 (52): p.18601-18606). Side chain solvent exposure higher than a threshold value is indicative for oxidation susceptibility in the presence of an oxidizing agent. For c), the NetMHCII pan algorithm was applied to make MHCII presentation predictions by breaking down chain sequences into all possible peptides and predict the probability of presentation on different MHC class II allotypes. For d), charge patches are calculated by a method developed at the University of Innsbruck. Using a MoFvAb 3D homology model of the Fv with capped C-terminus, protonation states at the requested pH value(s) are assigned with PROPKA, 3D charge distribution is calculated with APBS at 150 mM ionic strength and the volume integral of all positive and negative voxels is reported as the “score”.

### Example 4: Paratope Maturation of PD-L1 Paratope in M14HH17L and E06HP7L Clones in Bispecific Format

For clinical application, the parental clones M14HH17L and E06HP7L were further improved with respect to PD-L1 binding and other properties. Three phage display libraries were created using each of the clones as template for the purposes of clone enhancement. In each library, a selection of residues within the regions of the clone that are assigned to the H-side were partially randomized. Phage display panning was performed using each of these six libraries in three conditions whereby each condition differs by the concentration of antigen used in panning rounds 2 to 4. Phage library panning was performed in four rounds, wherein the first round was performed with 60 nM of biotinylated PD-L1 pre-immobilized on Dynabeads™ M-280 Streptavidin magnetic beads (ThermoFischer, catalog number 11206D), and rounds 2 to 4 were performed with 0.35 to 50 nM, 0.35 to 50 nM, and 0.35 to 50 nM of biotinylated target in solution (1 ml total volume of antigen and phage), followed by capture of Fab-on-phage/target complexes on the beads. Randomly S—S biotinylated human (P1AF6937) and cynomolgus monkey (P1AF6938) PD-L1 ECD in fusion with an Fc were used in an alternating fashion. In round one, captured phage clones bearing target-specific DutaFabs in combination with the beads were directly infected into log-phase TG1 E. coli cells, and rescued using M13KO7 helper phage, according to standard protocols. In rounds 2 to 4, the DutaFab bearing phages were eluted from the magnetic beads using 100 mM DTT prior to being used for infection. 30 Specific binders with improved affinity compared to the parental clone were identified by ELISA as follows: 25 μl of capture antibody hulgG F(ab′)₂ (Jackson Immuno Research; Catalog number 109-005-097, diluted in PBS to 300 ng/ml), were coated to microtiter plates (384-well Nunc Maxisorp, Catalog number 464718) and incubated for 1 hour at room temperature. Plates were washed 3× with Wash Buffer (PBST, 0.1% Tween20), blocked with 80 μl Blocking Buffer (PBS with 2% BSA and 0,2% Tween) and incubated for 1 hour at room temperature. After incubation, the wells were washed three times with Wash Buffer and incubated with 25 μl DutaFab containing bacterial supernatant, 1:5 diluted in Assay Buffer (PBS with 0.5% BSA and 0.05% Tween) and incubated for 1 hour at room temperature. After washing three times with Wash Buffer, 25 μl of the biotinylated antigen human PD-L1 (ECD) (P1AD9695) in final concentrations of 0.6 nM, 6.0 nM and 60 nM were added and incubated for 1 hour at room temperature. After washing three times with Wash Buffer, the wells were incubated with 25 μl detection agent Streptavidin-POD (Roche 11089153001, 1:3000 diluted in Assay Buffer) for 1 hour at room temperature. The plates were washed 6 times with Wash Buffer and 30 μl of TMB substrate was added. After an incubation of 5 minutes at room temperature, the absorbance was measured at OD 370 nm with an EnVision Reader (PerkinElmer).

Clones expressing DutaFabs with a range of PD-L1 ELISA absorbance values were selected but those with higher values were more likely to be included. The DutaFabs of unique clones were recombinantly expressed and SPR was performed to evaluate binding to human and cynomolgus PD-L1 in addition to IFNα.

Panning was followed by multiple rounds of clone optimization using a combination of single site mutagenesis, whole CDR or other sequence stretch exchange and CDR shuffling mediated by Gibson Assembly® (Gibson, D. G., et al. (2009) Nat. Methods 6, 343-345; Gibson, D. G., et al. (2010) Science 329, 52-56). During these maturations, clones derived from M14HH17L (parental) and E06HP7L (parental) were evaluated based on their desired properties including yield, affinity for human and cynomolgus PD-L1, affinity for IFNα, hydrophilicity, thermal stability, potential immunogenicity and other parameters. These processes of iterative clone creation were used to generate variants of the binders with various binding affinities for PD-L1. Tuning the affinity of the PD-L1 binders modulated the optimal conditions for switching and was therefore important to the overall function of the molecule. The Fabs of these clones were then assessed following E. coli expression using SPR and SEC. Since E06HP7L (parental) delivered comparatively poor affinity matured PD-L1 binding, only M14HH17L (parental) underwent a full maturation of the IFN-α2a paratope, and maturation of E06HP7L was stopped.

### Example 5: Paratope Maturation of IFN-α2a Paratope in M14HH17L (Parental)-Derived Clones in Bispecific Format

For affinity maturation of the IFN-α2a paratope, DutaFab genes were re-diversified by overlap PCR amplification, using combinations of degenerate oligonucleotides. In a first affinity maturation library, the re-diversification was directed at Vκ N-terminus, CDR-L1, CDR-L3 and CDR-H2. In a second library, the re-diversification was directed at CDR-L1, CDR-L3 and CDR-H2, while Vκ N terminus was fixed germline for immunogenicity reasons. Affinity maturation libraries were cloned into the pDuta4 vector, transformed into TG1 E. coli cells, and rescued using M13KO7 helper phage according to standard molecular biology methods. Both libraries reached a diversity of approximately 2E10 independent transformants.

Generation of affinity-matured DutaFabs binding specifically to human IFN-α2a was carried out by phage display. Phage library panning was performed as follows. In a first experiment, rounds 1 to 2 and 3 to 4 were performed with 4 nM and 0.8 nM of biotinylated target, huIFNα-2a (amino acid 24-188; P1AD8636), respectively, in solution. In a second experiment, rounds 1 to 2 and 3 to 4 were performed with 0.8 nM and 0.16 nM of biotinylated target, respectively, in solution. In a third experiment, round 1 was performed using 4 nM biotinylated IFN-α2a, round 2 with 100 nM biotinylated Fc-PD-L1 (OneArmed-huPD-L1_ECD_Fc_LALAPG, P1AF6937), round 3 with 0.4 nM biotinylated IFN-α2a and round 4 with 100 nM biotinylated Fc-PD-L1. In each round, Fab-on-phage/target complexes were afterwards captured on magnetic beads. Dynabeads™ M-280 Streptavidin magnetic beads (Thermo Fisher; catalog number 11206D) were used in round 1 and 3 and Neutravidin coupled magnetic beads (SpeedBeads Magnetic Neutravidin Coated particles) were used in round 2 and 4. DutaFab-bearing phages were eluted from the magnetic beads using 100 mM DTT prior to being used to infect log-phase TG1 E. coli cells, and rescued using M13KO7 helper phage, according to standard protocols.

For screening of selection outputs, a polyclonal plasmid miniprep of the respective selection round was prepared from the infected TG1 E. coli cells. Plasmids were digested using BamHI restriction endonuclease, which cuts the phagemid pDuta4 upstream and downstream of the phage Fd gene 3 domain. Plasmids were recircularized by ligation, generating an in-frame fusion of a T7 tag at the C-terminus of the DutaFab CH1 domain. The ligated polyclonal plasmids encoding T7-tagged DutaFabs were transformed into TG1 E. coli cells, and single colonies were picked into microtiter plates. Soluble DutaFabs were expressed in microtiter plates and supernatants were clarified by centrifugation.

The DutaFab culture supernatants were screened for improved affinity compared to the parental clones by ELISA as follows: 25 μl of capture antibody hulgG F(ab′)2 (Jackson Immuno Research, Catalog number 109-005-097, diluted in PBS to 300 ng/ml), were coated to microtiter plates (384-well Nunc Maxisorp, Catalog number 464718) and incubated for 1 hour at room temperature. Plates were washed three times with Wash Buffer (PBST, 0.1% Tween20), blocked with 80 μl Blocking Buffer (PBS with 2% BSA and 0,2% Tween) and incubated for one hour at room temperature. After incubation, the wells were washed three times with Wash Buffer, incubated with 25 μl DutaFab containing bacterial supernatant, 1:5 diluted in Assay Buffer (PBS with 0.5% BSA and 0.05% Tween) and incubated for 1 hour at room temperature. After washing three times with Wash Buffer, 25 μl of the biotinylated antigen human IFN-α2a-hFc (monomer) in final concentrations of 0.5 nM and 50 nM were added and incubated for one hour at room temperature. After washing 3 times with Wash Buffer, the wells were incubated with 25 μl detection agent Streptavidin-POD (Roche, Catalog number 11089153001, 1:3000 diluted in Assay Buffer) for 1 hour at room temperature. The plates were washed 6 times with Wash Buffer and 30 μl of TMB substrate was added. After an incubation of 5 minutes at room temperature, the absorbance was measured at OD 370 nm with an EnVision Reader (PerkinElmer).

Clones showing improved IFN-α2a binding compared to the parent were identified and the corresponding phagemids were sequenced. 78 unique sequences were discovered and expressed in E. coli. Fabs were purified from the E. coli supernatant via a one-step affinity chromatography purification on Capture Select IgG-CH1 resin (19432001L). The quality of the purified Fabs was analyzed via size exclusion chromatography. Fabs were subjected to a kinetic screening experiment binding affinity was assessed by SPR-analysis using a Biacore 8K or 8K+ instrument (Cytiva). Binding kinetics of binding to human IFN-α2a, human PD-L1 and to CD79B dimer (control) were analyzed essentially as described in section a) of Example 2A.

The maturation was followed by seven rounds of sequence polishing during which the VH and VH sequences were germlined, hydrophobicity and positive charges decreased, and the IFNα2a binding affinity fine-tuned.

### Example 6: Affinity Matrix for Identification of Optimal Ratio of Affinities

The functioning of the bispecific anti-huIFN-α2a/anti-huPD-L1 DutaFab as a mutually exclusive binder and as a molecular switch in the fusion proteins according to the invention is influenced by the affinities of the human PD-L1 and human IFN-α2a paratopes in the DutaFab and by the epitope and its affinity to it of the huPD-L1-targeting arm. In order to evaluate the impact of huPD-L1 and huIFN-α2 affinities of the Dutaflip arm on huPD-L1-dependent IFNα activation in a cell based assay, various molecules of the format shown in FIG. 1A-1B were generated, consisting of the same anti-PD-L1-targeting binder M14, but differing in the <PD-L1> and <IFN-α2> affinities of the Dutaflip domains.

### a) Impact of PD-L1 and IFN-α2 Affinities of the Dutaflip Arm on PD-L1 Dependent IFNα Activation

For a systematic comparison, affinity of one paratope was kept constant while varying the affinity of the other. To analyze PD-L1-controlled IFN-α2 activation, the HEK blue IFNα/β reporter gene assay was performed as described in Example 13 below, using the parental (Invivogen) HEK cell line with low PD-L1 expression on the cell surface (about 300 PD-L1 molecules/cell on the cell surface) and a modified cell line expressing recombinant PD-L1 on the cell surface (“clone 45”, about 25,000 PD-L1 molecules/cell on the cell surface), generated as described in Example 12.

The EC50 values were normalized to a reference molecule (an attenuated IFN-α2a R149A variant fused via its N-terminus to the C-terminus of a one-armed anti-PD-L1 antibody (P1AG2011)), by dividing the EC50 of the sample though the EC50 value of the reference molecule.

In this experiment, the PD-L1 affinities of the Dutaflip domain varied from high affinity (0.5 nM) to medium affinity (2 nM and 20 nM) while IFN-α2 affinity was kept constant (ca. 14 nM). As can been seen in Table 5, the experiments demonstrate that PD-L1 affinities of the Dutaflip arm affected PD-L1-dependent IFNα activation.

TABLE 5
Optimization of affinity ratio of bispecific mutually exclusive
anti-IFN-α2a/anti-PD-L1 DutaFab by varying PD-L1 affinity

| | | | Experiment 1: EC50 | | Experiment 2: EC50 | |
|---|---|---|---|---|---|---|
| | | | values | | values | |
| | | | HEK Blue | HEK Blue | HEK Blue | HEK Blue |
| | K_{D} Dutaflip | | IFNα/β | IFNα/β | IFNα/β | IFNα/β |
| | huPD-L1 | IFN-α2 | PD-L1 low | PD-L1 high- | PD-L1 low | PD-L1 high- |
| | [nM] | [nM] | expressing | expressing | expressing | expressing |
| P1AH4372 | 0.5 | 14 | 14.7 | 1.0 | 24.5 | 0.8 |
| P1AH4361 | 2 | 14 | 45.0 | 4.3 | 72.5 | 1.7 |
| P1AH4369 | 20 | 15 | 102.9 | 1.2 | 176.0 | 2.3 |

Higher PD-L1 affinities (lower KD values) led to stronger PD-L1-dependent IFN-α2 activation (lower EC50 values) and less affine binding resulted in lower PD-L1-dependent IFN-α2 activation (higher EC50 values). The effect was more pronounced at low PD-L1 cell surface levels.

In the next experiment, different IFN-α2 affinities of the Dutaflip domain (5.5 nM and 15 nM) were analyzed while keeping the affinity to PD-L1 constant (20 nM). Lower IFN-α2 affinity (higher K_{D} value) led to less masking of IFN-α2 and stronger PD-L1 dependent IFN-α2 activation. As expected, masking of the IFN-α2 moiety depended on the IFN-α2 affinity of the Dutaflip arm.

As shown in Table 6, higher IFN-α2 affinities (lower K_{D} values) led to lower IFN-α2 activation (higher EC50 values) and less affine binding resulted in higher IFN-α2 activation (lower EC50 values), demonstrating that the activation can be modulated by optimizing the affinities of the Dutaflip arm towards the paratope. The effect was similar at low and at high PD-L1 cell surface levels. The results clearly demonstrate that PD-L1 and IFN-α2 affinities of the Dutaflip are important parameters for adjusting PD-L1 dependent IFN-α2 activation.

TABLE 6
Optimization of affinity ratio of bispecific mutually exclusive anti-
IFN-α2a/anti-PD-L1 DutaFabs by varying IFN-α2 affinity

| | | | Experiment 1: EC50 | | Experiment 2: EC50 | |
|---|---|---|---|---|---|---|
| | | | values | | values | |
| | | | | HEK Blue | | HEK Blue |
| | K_{D} Dutaflip | | HEK Blue | IFNα/b | HEK Blue | IFNα/b |
| | huP | | IFNα/b | PD-L1 | IFNα/b | PD-L1 |
| Molecule | D-L1 | IFN-α2 | PD-L1 low | high- | PD-L1 low | high- |
| ID | [nM] | [nM] | expressing | expressing | expressing | expressing |
| P1AH4359 | 20 | 5.5 | 126.1 | 6.2 | 241.0 | 4.3 |
| P1AH4369 | 20 | 15 | 102.9 | 1.2 | 176 | 2.3 |

### b) Affinity Matrix for Identification of Optimal Ratio of Affinities

The PD-L1-dependent IFN-α2 activation based on the mutually exclusive binding approach can be controlled by the affinities of the Dutaflip to PD-L1 and IFN-α2. In order to identify the optimal affinity ranges for our purpose and their affinity relation to each other, we generated a full matrix of molecules of the format shown in FIG. 1A-1B, varying in the affinities of the Dutaflip module from around 0.4 nM to 40 nM for PD-L1 and from around 1 nM to 40 nM for IFN-α2.

In addition, we used two different affinities on the monospecific targeting arm. The molecules were tested in the HEK blue assay as described in example 6 a).

We looked for optimal affinity compositions that show superior relation of low IFNα pathway activation at low huPD-L1 surface level (HEK blue IFNα/β parental) and high IFNα pathway activation at high huPD-L1 surface level (HEK blue IFNα/β clone 45). The two columns on the right of Table 7 show the subsequent confirmatory determination of K_{D} values of selected candidates.

For affinity determination, Dutaflip Fab-fragments without IFN-α2 fusion were recombinantly expressed in E. coli and affinities were analyzed as described in Example 2 a) and b) (with P1AF6937 and P1AD8636 as analytes). In the cell-based assay, an affinity of the Dutaflip domain towards human PD-L1 in the range of 1-10 nM and an affinity towards huIFN-α2 that is about >3-fold weaker compared to the affinity towards huPD-L1 was identified as desirable.

TABLE 7
Affinity matrix for identification of optimal ratio of affinities

| | EC50 (nM) | | | | |
|---|---|---|---|---|---|
| | HEK | HEK Blue | | K_{D}_{_{—}} | K_{D}_{_{—}} |
| | Blue | PD-L1 | | PDL1 | IFN-α |
| | parental | Cl045 | targeting arm | (nM) | (nM) |
| P1AH4374 | 4.12 | 0.0246 | M14 | — | — |
| P1AH4230 | 6.10 | 0.0061 | M14 | — | — |
| P1AH4362 | 15.74 | 0.0025 | M14 | — | — |
| P1AH4367 | 0.45 | 0.0010 | M14 | — | — |
| P1AH4372 | 0.49 | 0.0014 | M14 | — | — |
| P1AH4378 | 2.56 | 0.0055 | M14 | — | — |
| P1AH4361 | 1.45 | 0.0030 | M14 | — | — |
| P1AH4366 | 0.85 | 0.0014 | M14 | — | — |
| P1AH1111 | — | 0.0014 | Atezolizumab | 37 | 4.3 |
| P1AH1108 | — | 0.0004 | Atezolizumab | 6 | 15 |
| P1AH4357 | 5.52 | 0.0175 | M14 | 2 | 14 |
| P1AH4377 | 2.78 | 0.0060 | M14 | — | — |
| P1AH4359 | 4.82 | 0.0078 | M14 | 20 | 5.5 |
| P1AH4360 | 1.67 | 0.0045 | M14 | 1.1 | 10 |
| P1AH4365 | 0.42 | 0.0011 | M14 | — | — |
| P1AH4369 | 3.52 | 0.0042 | M14 | 20 | 15 |
| P1AH4370 | 0.29 | 0.0009 | M14 | — | — |
| P1AH3929 | — | 0.0019 | Atezolizumab | — | — |
| rec. IFN-α2a | 0.02 | 0.0151 | | — | — |

### Example 7: Generation and Characterization of the Anti-PD-L1 Antibody M14 Used for Targeting

a) Generation of the Anti-PD-L1 Fab M14 Derived from the H-Side of the Bispecific DutaFab M14HH17L

The M14HH17L bispecific DutaFab clone was used as a basis to generate a PD-L1 specific targeting arm. In order to remove IFN-α2a binding and reduce potential immunogenicity, the L-side CDRs on the heavy and light chains that were specific for IFN-α2a (CDR-L1, CDR-L3 and CDR-H2) were replaced with amino acid stretches closer to those found in human antibody germlines.

As with the mutually exclusive bispecific DutaFab, when engineering the PD-L1-targeting Fab we sought to generate molecules that were optimized with respect to yield, affinity for human and cynomolgus PD-L1, hydrophilicity, thermal stability, potential immunogenicity and other parameters. Generated molecules were variably analyzed using combinations of charge, hydrophilicity and immunogenicity predicting algorithms, SPR, analytical SEC, analytical HIC, and Differential Scanning Fluorimetry (DSF).

Engineering of the PD-L1-targeting Fab was carried out alongside switch maturations of the M14HH17L (parental) clone and therefore the H-side residues also varied, as germlining of the L-side was ongoing. Initially, the CDRs of the M14HH17L (parental) variant were replaced with near germline Dummy DutaFab CDRs by gene synthesis (1 clone, Twist Bioscience, USA). Thereafter, 12 further germline L-side variants within an M14HH17L (parental)-derived clone which were generated by gene synthesis (Twist Bioscience, USA) were tested. Next, further germline CDR variants of CDRs L1 and L3 were tested individually for function (24 variants) and promising CDRs were taken forwards and formed a matrix of L1 and L3 CDRs (16 variants) using Gibson Assembly® cloning in each case. Finally the effects of 5 germline Kappa J segments on 3 promising M14HH17L (parental) germlined clones were assessed.

The H side of the M14HH17L (parental)-derived anti-huPD-L1 Fab was also modified. In order to limit sequence diversity and associated immunogenic risk, a single residue within the H side was identified that could be altered to deliver the desired affinities to either the switch or targeting PD-L1 binders. Accordingly, by this “H-side” diversity was reduced to a single residue within the CDR-L2.

### b) Affinity Determination

Affinities for human and cynomolgus PD-L1 of selected PD-L1 monospecific binders which were derived from M14HH17L (parental) as described in Example 7 a) were analyzed. For this, Fab-fragments of corresponding binders were analyzed by SPR using Fc-tagged human and cynomolgus PD-L1 and His-tagged IFNα, essentially as described in section a) of Example 2. Affinity to human IFN-α2a was tested to ensure that any binding to IFN-α2a had been eradicated entirely by replacing the original L-side CDRs responsible for binding to IFN-α2a (CDR-L1, CDR-L3 and CDR-H2).

TABLE 8
Affinities of M14HH17L-derived anti-huPD-L1
Fabs for human or cynomolgus PD-L1

| | Human PD-L1 | | | Cynomolgus PD-L1 | | | Human |
|---|---|---|---|---|---|---|---|
| Molecule | Kₒₙ | K_{off} | K_{D} | Kₒₙ | K_{off} | K_{D} | IFNα |
| ID | [1/Ms] | [1/s] | [nM] | [1/Ms] | [1/s] | [nM] | K_{D} [nM] |
| P1AI3443 | 1.6E+05 | 9.1E−04 | 5.8 | 1.6E+05 | 3.6E−03 | 22.6 | no binding |
| P1AI3444 | 1.5E+05 | 7.7E−04 | 5.0 | 1.4E+05 | 2.7E−03 | 19.1 | no binding |
| P1AI3445 | 1.7E+05 | 7.8E−04 | 4.7 | 1.7E+05 | 2.8E−03 | 16.5 | no binding |
| P1AI3436 | 1.6E+05 | 1.2E−04 | 0.7 | 1.4E+05 | 1.3E−04 | 0.9 | no binding |
| P1AI3438 | 2.0E+05 | 1.2E−04 | 0.6 | 1.6E+05 | 1.5E−04 | 0.9 | no binding |
| P1AI3433 | 1.5E+05 | 5.0E−04 | 3.3 | 1.3E+05 | 2.3E−03 | 18.3 | no binding |
| P1AI3434 | 1.7E+05 | 4.4E−04 | 2.6 | 1.4E+05 | 2.6E−03 | 18.3 | no binding |
| P1AI4260 | 1.1E+05 | 4.5E−04 | 4.0 | 8.6E+04 | 3.6E−03 | 30 | no binding |

As can be seen from the data shown in Table 8, all tested binders show binding to human as well as to cynomolgus PD-L1. P1AI3436 and P1AI3438 show particularly high affinity, both to human and to cynomolgus PD-L1. None of the tested Fabs showed binding to IFN-α2a. As high affinity to PD-L1 had been shown in cellular assays to perform best, P1AI3438 (M14) was selected for creating targeted IFN-α2a fusion proteins.

In order to describe the binding site of selected M14HH17L (parental)-derived anti-PD-L1 Fabs to PD-L1 in more detail, it was tested whether the M14HH17L (parental)-derived anti-PD-L1 Fabs can (1) bind to the C-terminal domain of PD-L1, (2) compete for binding with Atezolizumab (which bind to the N-terminal domain of PD-L1) and (3) inhibit binding of PD1 to PD-L1 by SPR experiments.

### c) Binding of M14HH17L (Parental)-Derived Anti-PD-L1 Fabs to the C-Terminal Domain of PD-L1

The SPR experiments were performed on a Biacore T200 at 25° C. with PBS-P+ as running and sample dilution buffer (0.2 M phosphate buffer with 27 mM KCl, 1.37 M NaCl and 0.5% Surfactant P20 (Tween 20), Cytiva).

Anti-Fab antibody (ThermoFischer) was directly immobilized on the second flow cell of a CM3 as described above. For the assay, the different anti-huPD-L1 Fabs had been expressed recombinantly as one-armed Fab-Fc fusion proteins. The Fab-Fc-fusion proteins were captured by injecting for 120 seconds at a flow rate of 10 μl/min. As a second step PD-L1 (P1AF6939) or C-terminal domain of PD-L1 (P1AF7352) was injected for 120 sec at 30 μl/min. Binding curves were evaluated using Biacore T200 evaluation software 3.1 (Cytiva). Anti-huPD-L1 binding antibody Atezolizumab was run in parallel as a negative control, as it is known to bind to the extracellular domain of huPD-L1, but not to its C-terminal domain.

TABLE 9
Affinities of selected M14HH17L(parental)-derived
anti-huPD-L1 Fabs to the extracellular domain
or the C-terminal domain of huPD-L1

| Ligand | Analyte | kₐ (1/Ms) | k_{d} (1/s) | K_{D}[nM] |
|---|---|---|---|---|
| Atezolizumab | P1AF6939 | 2.23E+05 | 3.20E−05 | 0.1 |
| P1AI4475 | (PD-L1_ECD) | 1.41E+05 | 5.63E−05 | 0.4 |
| P1AI4392 | | 1.28E+05 | 4.69E−04 | 3.7 |
| Atezolizumab | | no binding | | n.d. |
| P1AI4475 | P1AF7352 | 5.79E+04 | 4.44E−04 | 7.7 |
| P1AI4392 | (C-terminal | 2.84E+04 | 1.23E−03 | 43.3 |
| | domain of PD- | | | |
| | L1) | | | |

As shown in Table 9, the results of the assay confirm experimentally that the selected binder M14 (P1AI4475) and the binder P1AI4392 (like the parental binder M14HH17L they are both derived from) bind to the C-terminal domain of PD-L1. Atezolizumab, which is known to bind to the N-terminal domain of PD-L1, was used as a control.

d) SPR Assays Demonstrating Lack of Competitive Binding with Atezolizumab

The SPR experiments were performed on a Biacore T200 at 25° C. with PBS-P+ (0.2 M phosphate buffer with 27 mM KCl, 1.37 M NaCl and 0.5% Surfactant P20 (Tween 20), Cytiva) as running and sample dilution buffer.

One-armed anti-PD-L1 Fab-Fc fusion (P1AI4841; Atezolizumab) was directly immobilized on the second flow cell of a CM3 chip at pH 5.0 using the standard amine coupling kit (Cytiva) aiming for a sensor density of 500 RU. After activation of the sensor surface with a 1:1 mixture of 0.4 M 1-ethyl-3-(3-dimethylaminopropyl)-carbodiimide (EDC) and 0.1 M N-hydroxysuccinimide (NHS), 50 nM P1AI4841 (diluted in 10 mM acetate pH 5.0) was injected with a flow rate of 10 μl/min. After blocking with 1 M ethanolamine-HCl pH 8.5, the coupling procedure led to a sensor density of 500 RU.

Recombinant one-armed human PD-L1-Fc fusion (P1AF6937) at a concentration of 25 nM was captured by injecting for 120 seconds at a flow rate of 10 μl/min reaching a signal of approximately 30 RU. As a second step, the anti-PD-L1 Fab derived from M14HH17L (parental) or a reference anti-PD-L1 or a control molecule was injected at a concentration of 75 nM for 120 sec at 30 μl/min. An increasing signal during the second injection phase indicates a different epitope because the binding epitope was not blocked by the capture PD-L1 binder on the surface. No additional binding indicates the same epitope region. The chip surface was regenerated after every cycle by injection of 0.85% H₃PO₄ for 60 seconds at a flow of 10 μl/min. Bulk refractive index differences were corrected by subtracting the response obtained on the reference flow cell. Binding curves were evaluated using Biacore T200 evaluation software 3.1 (Cytiva).

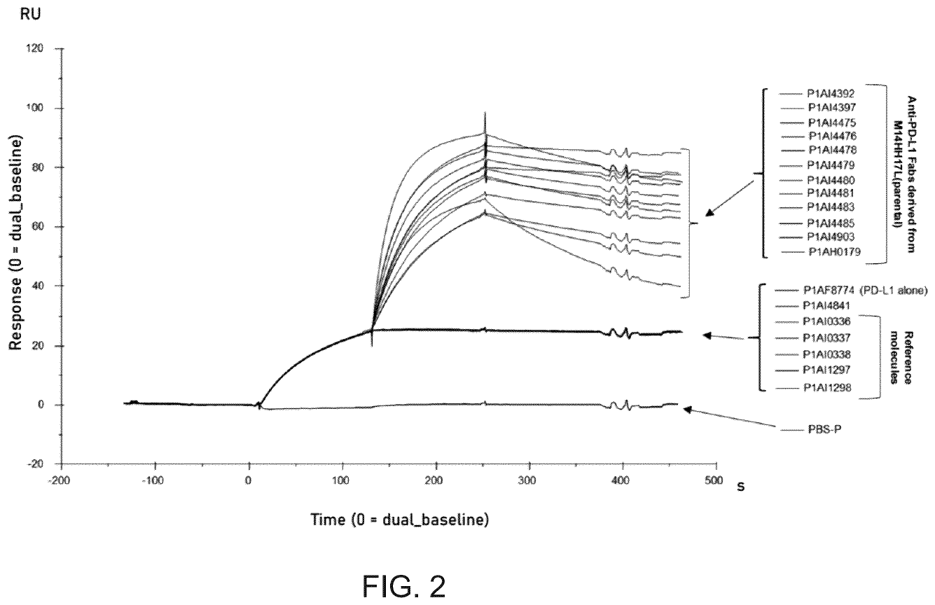

The results of the experiment are shown in the sensorgram of FIG. 2. The SPR experiments clearly demonstrated that M14HH17L (parental)-derived anti-PD-L1 Fabs (P1AI4392, P1AI4397, P1AI4475, P1AI4476, P1AI4478, P1AI4479, P1AI4480, P1AI4481, P1AI4483, P1AI4485, P1AI4903 and PIAH0179) did not compete with Atezolizumab binding to PD-L1. In contrast, the reference molecules P1AI0336, PAI0337, P1AI0338, P1AI1297 and P1AI1298 all compete with Atezolizumab binding to PD-L1. PD-L1-Fc (P1AF6937) and anti-PD-L1 Fab based on Atezolizumab (P1AI4841) were used as negative controls.

### c) Blocking the Interaction of PD1 and PD-L1 by PD-L1 Binding of M14HH17L (Parental)-Derived Fab Fragments

Antibodies that block interaction of human PD-L1 and human PD1 (e.g. Atezolizumab, BMS-936559, Avelumab, Durvalumab) usually bind to the N-terminal domain of PD-L1, which overlaps with the PD1-binding site of PD-L1. In contrast, the M14HH17L (parental)-derived anti-huPD-L1 monospecific Fabs and anti-PD-L1/anti-IFN-α2a bispecific DutaFabs are directed against the C-terminal domain of PD-L1 but surprisingly are still capable to block the interaction of PD-L1 and PD1 in a SPR binding assay.

To demonstrate this, SPR experiments were performed on a Biacore T200 at 25° C. with PBS-P+ as running and sample dilution buffer (0.2 M phosphate buffer with 27 mM KCl, 1.37 M NaCl and 0.5% Surfactant P20 (Tween 20), Cytiva). A CAP Chip (provided in the Biotin CAPture Kit, series S, Cytiva, 28920234) was hybridized with ssDNA-SA according manufacturer's instructions and loaded with biotinylated human PD1 extracellular domain (P1AF6937) for 180 seconds with a flow rate of 5 L/min and at a concentration of 100 nM. Premixtures containing 50 nM of human PD-L1_Fc fusion (P1AD8568) and 3-fold molar excess of M14HH17L (parental)-derived Fab-Fc were injected on both flow cells for 300 seconds at a flow speed of 5 L/min. Dissociation time is set to 300 s. An increasing signal during the injection phase indicates that the binding of PD-L1 to PD1 is not blocked by the analyzed antibody, while no signal increase indicates blocking by the antibody.

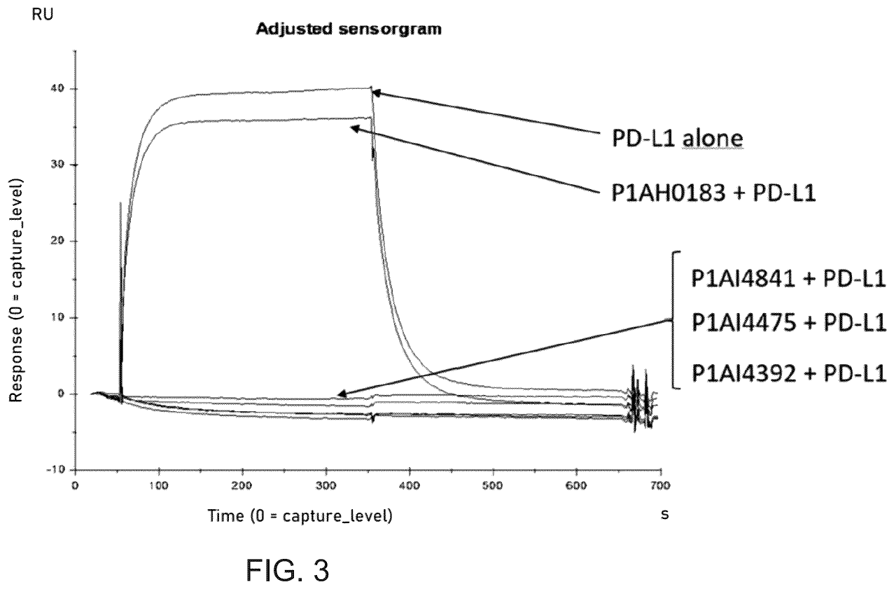

As shown in the sensorgrams in FIG. 3, the two M14HH17L (parental)-derived tested anti-PD-L1 Fabs (P1AI4475 and P1AI4392) that were derived from the anti-PD-L1 binding CDRs (H-side) of the anti-PD-L1/anti-IFN-α2a bispecific DutaFab M14HH17L (parental), as described in Example 7, block binding of PD-L1 to PD1, like the Atezolizumab-based anti-PD-L1 Fab (P1AI4841) which was used as a positive control. P1AH0183 is a PD-L1-specific antibody derived from bispecific DutaFab E06HP7L, which does not block PD-L1/PD1 interaction.

The PD1/PD-L1 blockade bioassay (Promega) was also used to evaluate if M14HH17L (parental)-derived anti-PD-L1 Fabs can also block PD1/PD-L1 interaction in a cellular context. The principle of the assay is a bioluminescent cell-based assay that is based on the use of two different cell lines: a) Jurkat T cells genetically engineered to express human PD-1 and a luciferase reporter driven by an NFAT response element (NFAT-RE) (=PD-1 Effector Cells) and b) CHO-K1 cells genetically engineered to express human PD-L1 and an engineered cell surface protein designed to activate cognate TCRs in an antigen-independent manner (=PD-L1 aAPC/CHO-K1 Cells). When the two cell types are co-cultured, the PD-1/PD-L1 interaction inhibits TCR signaling and NFAT-RE-mediated luminescence. Addition of either an anti-PD-1 or an anti-PD-L1 antibody that blocks the PD-1/PD-L1 interaction releases the inhibitory signal and results in TCR activation and NFAT-RE-mediated luminescence. The bioluminescent signal can be detected and quantified using the Bio-Glo™ Luciferase Assay System.

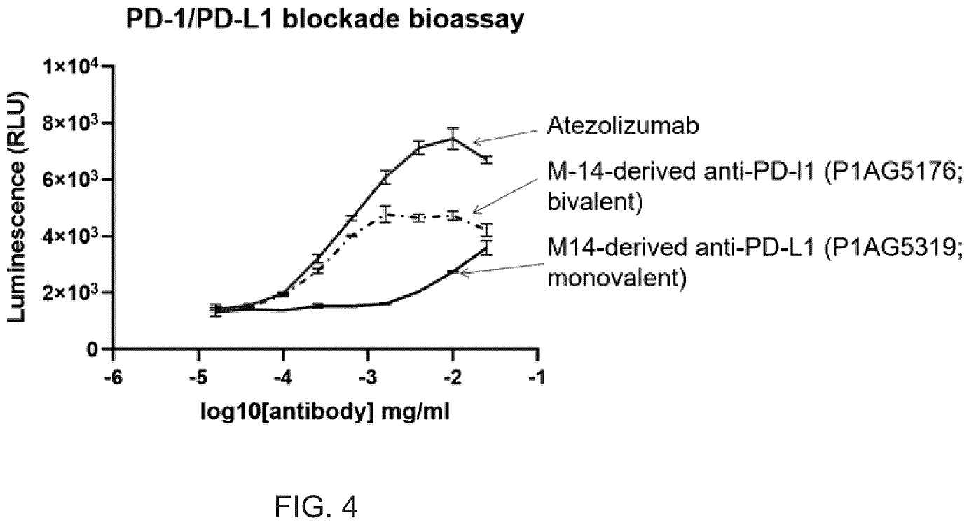

In this assay, monovalent or bivalent anti-PD-L1 binders were tested for blocking of PD1/PD-L1 interaction. The assay was performed as described according to the manufacturer's instructions. Atezolizumab which is known to block PD1/PD-L1 interaction was used as a positive control. As can be seen in the graph shown in FIG. 4, already the parental (i.e. derived from the parental M14HH17L DutaFab) anti-PD-L1 Fabs (PIAG5319 (monovalent) and PIAG5176 (bivalent)) before affinity maturation and germlining—like the Atezolizumab-based anti-PD-L1 Fab (P1AI4841)—block binding of PD-L1 to PD1, even though they—as shown in Example 7 c)—bind to the C-terminus of PD-L1 extracellular domain and therefore do not bind to the PD1/PD-L1 interface region. Another C-terminal PD-L1 binder (P1AH0183) which does not block PD-L1/PD1 interaction was used as negative control.

### Example 8: Generation of PD-L1-Targeted Interferon-α2 Fusion Proteins

### a) Manufacture of a PD-L1-Targeted Interferon Alpha Fusion Protein and Reference Molecules

A schematic illustration of a PD-L1-targeted Interferon alpha fusion protein as described herein is shown in FIG. 1A-1B. FIG. 1A shows the PD-L1-targeted Interferon alpha fusion protein in a form where the fused huIFN-α2a molecule is bound to the bispecific anti-PD-L1/anti-IFN-α2a binder. In this form, the binding to the bispecific DutaFab would prevent the huIFN-α2a molecule from binding to its natural receptor IFNAR1/2.

FIG. 1B shows the molecule in a form where the fused huIFN-α2a molecule is not bound to the bispecific anti-huPD-L1/anti-huIFN-α2a binder, as would be the case, for instance, if the bispecific anti-huPD-L1/anti-huIFN-α2a binder had bound to PD-L1, e.g. on the surface of a target cell, thus preventing the fused huIFN-α2a from binding to the bispecific DutaFab. (Black dot: modification in the Fc domain promoting heterodimerization. *: amino acids of opposite charges introduced in the CH and CL domains)

The molecule comprises a Fab arm specifically binding to PD-L1, a bispecific DutaFab that specifically and mutually exclusively binds to either PD-L1 or IFN-α2a acting as a molecular switch (Dutaflip), a wildtype IFN-α2a molecule (SEQ ID NO:79) fused to the Dutaflip via a (G2S-(G4S) 3-G2) peptide linker (SEQ ID NO:110), and an Fc domain. In the anti-PD-L1-targeting Fab arm, the light chain variable domain and the heavy chain variable domain were exchanged (“CrossFab”) in order to promote the correct assembly of the two different light chains. The anti-PD-L1 Fab arm and the Dutaflip arm were each attached to one of the N-termini of the two subunits of the Fc domain via the C-termini of their respective CH1 domain heavy chains. The PD-L1-targeted IFN-α2 fusion proteins according to the invention were engineered using the knob-into-hole mutations Y349C, T366S, L368A, Y407V (hole) and S354C, T366W (knob). Immune effector functions of the Fc domain were abolished using the LALA PG mutations (L234A, L235A and P329G; Schlothauer et al. (2016) Protein Engineering, Design & Selection, vol. 29 no. 10, pp. 457-466).

Table 10 shows the amino acid sequences of the variable domain regions of six molecules according to the invention that were used in the following examples.

TABLE 10
Amino acid sequences of variable domains

| | 1^{st} variable | | 2^{nd} variable | 2^{nd} variable |
|---|---|---|---|---|
| | domain - | 1^{st} variable | domain - | domain - |
| | heavy chain | domain - light | heavy chain | light chain |
| | (SEQ ID | chain | (SEQ ID | (SEQ ID |
| | NO:) | (SEQ ID NO:) | NO:) | NO:) |
| P1AI4295 | 7 | 8 | 27 | 28 |
| P1AI4748 | 7 | 10 | 27 | 28 |
| P1AI4298 | 7 | 12 | 27 | 28 |
| P1AI4797 | 7 | 10 | 35 | 36 |
| P1AI4798 | 7 | 12 | 35 | 36 |
| P1AH1111 | 19 | 20 | 35 | 36 |

TABLE 11
Amino acid sequences of full-length chains

| | 1^{st} heavy | 1^{st} light | 2^{nd} heavy | 2^{nd} light |
|---|---|---|---|---|
| | chain | chain | chain | chain |
| | (SEQ ID | (SEQ ID | (SEQ ID | (SEQ ID |
| | NO:) | NO:) | NO:) | NO:) |
| P1AI4295 | 37 | 38 | 39 | 40 |
| P1AI4748 | 41 | 42 | 43 | 44 |
| P1AI4298 | 45 | 46 | 47 | 48 |
| P1AI4797 | 49 | 50 | 51 | 52 |
| P1AI4798 | 53 | 54 | 55 | 56 |
| P1AH1111 | 57 | 58 | 59 | 60 |

Table 11 shows the amino acid sequences of the full-length chains of these six molecules. For the experiments described below, the proteins listed in Table 11 were recombinantly expressed with a single Glycine residue attached C-terminally to each of the two Fc domain subunits. Table 12 shows an overview of the reference molecules that were used in the experiments for comparison.

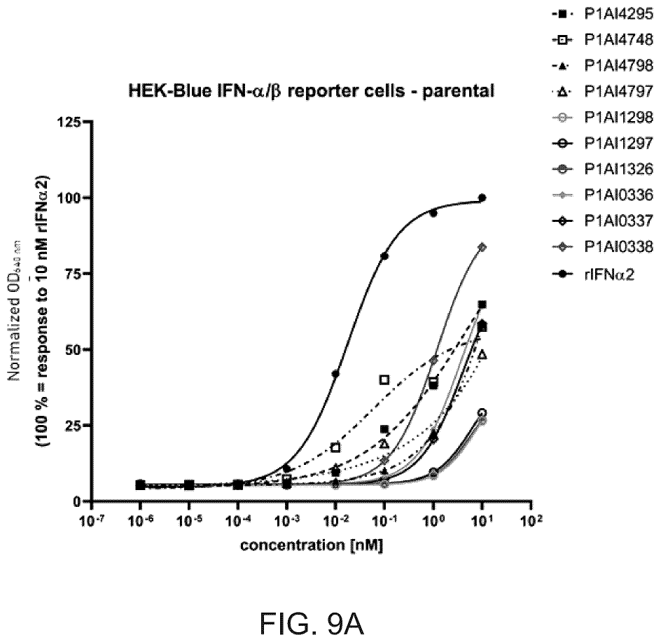

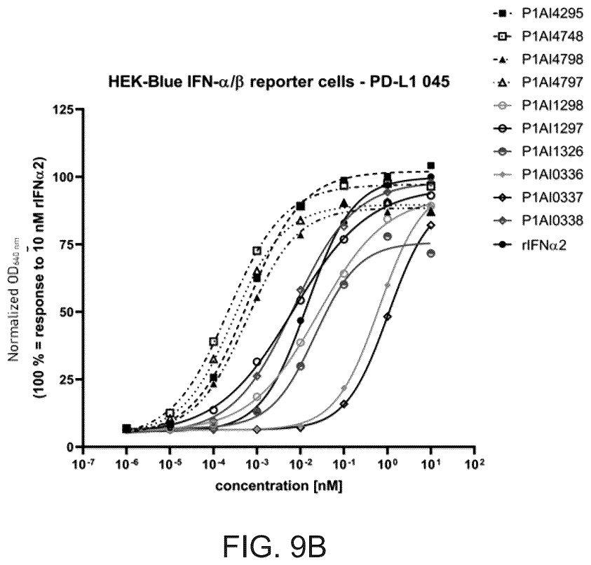

TABLE 12
Reference molecules used in the experiments described herein:

| | | | 1^{st} poly- | 2^{nd} poly- | 3^{rd} poly- |
|---|---|---|---|---|---|
| | | | peptide | peptide | peptide |
| | | | chain | chain | chain |
| Molecule | | Name in the reference | (SEQ ID | (SEQ ID | (SEQ ID |
| ID | Reference | document | NO:) | NO:) | NO:) |
| P1AI1297 | WO 2021/231773 | AF003219 | 70 | 71 | 72 |
| | (e.g. in Table | | | | |
| | 15/16) | | | | |
| P1AI1298 | WO 2021/231773 | PDL1-IFN_uIFN | 73 | 74 | 75 |
| | (e.g. in Table 1) | 2D10scFv KiH PDL1- | | | |
| | | IFN_1A05H_N36G | | | |
| | | (PD-L1/IFNα DBA | | | |
| | | cytokine complex) | | | |
| P1AI1326 | WO 2021/231773 | PD-LI/IFNα DBA | 76 | 77 | 78 |
| | (e.g. in Table 12 | cytokine complex of the | | | |
| | or in FIG. 9A | asymmetric Cytokine | | | |
| | and FIG. 9B | Complex scFv-IgG | | | |
| | | format named | | | |
| | | Exemplary Seq_C | | | |
| P1AI0336 | WO 2021/231773 | DBA_N-term_IFNα | 61 | 62 | 63 |
| | | C08 | | | |
| P1AI0337 | WO 2021/231773 | DBA_N- | 64 | 65 | 66 |
| | | term_IFNα_{——}B09 | | | |
| P1AI0338 | WO 2021/231773 | DBA_N- | 67 | 68 | 69 |
| | | term_IFNα_{——}SeqID41 | | | |

P1AI0336-P1AI0338 in the reference document are dual binding antibodies (DBAs) fused to IFNα. To express them in cell culture, they were fused via the C-terminus of the heavy chain of the DBA to an Fc domain (one-armed).

### b) Transient Production of IgG-Like Proteins in Expi293F™ Cells

The recombinant fusion protein genes encode the respective fusion protein heavy and light chains. The expression plasmids for the transient expression of the fusion proteins comprised besides the fusion protein heavy or light chain expression cassette an origin of replication from the vector pUC18, which allows replication of this plasmid in E. coli, and a beta-lactamase gene which confers ampicillin resistance in E. coli.

The transcription unit of a respective fusion protein heavy or light chain comprised the following functional elements:
the immediate early enhancer and promoter from the human cytomegalovirus (P-CMV) including intron A,
a human heavy chain immunoglobulin 5′-untranslated region (5′UTR),
a murine immunoglobulin heavy chain signal sequence, and
the bovine growth hormone polyadenylation sequence (BGH pA).

Proteins, antibodies and cytokine IgG fusion proteins were expressed by transient transfection of Expi293F™ cells (ThermoFisher scientific, USA). Cells were seeded in Expi293™ medium (Gibco, Cat. N° 1435101) at a density of 2.5×10⁶/ml. Cells were co-transfected with plasmids containing the respective fusion protein heavy- and light chain by separately mixing expression vectors and ExpiFectamine (Gibco, ExpiFectamine™ transfection kit, Catalog number 13385544) in OptiMEM™ reduced serum medium (Gibco, Catalog number 11520386). After 5 minutes, both solutions were combined, mixed by pipetting and incubated for 25 minutes at room temperature. Cells were added to the expression vector/ExpiFectamine solution and incubated for 24 hours at 37° C. in a shaking incubator with a 5% CO₂ atmosphere. One-day post transfection, supplements (Transfection Enhancers 1 and 2, ExpiFectamine™ transfection kit) were added. Cell supernatants were harvested after 4-5 days by centrifugation and subsequent filtration (0.2 μm filter), and proteins were purified from the harvested supernatant by standard methods as indicated below. General information regarding the recombinant expression of human immunoglobulins in e.g. HEK293 cells is given in: Meissner, P. et al., Biotechnol. Bioeng. 75 (2001) 197-203.

### c) Protein Purification

Recombinant immunoglobulin-like proteins were purified from cell culture supernatants by affinity chromatography using MabSelectSure-Sepharose™ (Cytiva, USA). Briefly, sterile filtered cell culture supernatants were captured on a MabSelect SuRe resin equilibrated with PBS buffer (10 mM sodium phosphate, 1 mM potassium phosphate, 137 mM sodium chloride and 2.7 mM potassium chloride, pH 7.4), washed with equilibration buffer, eluted with 100 mM sodium acetate, pH 3.0. After neutralization (about pH 5.5) with 1 M Tris pH 9.0, aggregated protein was separated from monomeric antibody species by size exclusion chromatography (Superdex 200, Cytiva) in 20 mM histidine, 140 mM NaCl, pH 6.0. Monomeric protein fractions were pooled, concentrated if required using e.g. a MILLIPORE Amicon Ultra (30 kDa MWCO) centrifugal concentrator and stored at −80° C. In some examples, an additional cation exchange chromatography (cIEX) purification step was performed before size exclusion chromatography. In this respect, the pH adjusted MabSelectSure eluate was diluted 1:1 with water and loaded on a Poros XS cation exchange resin equilibrated with 20 mM histidine pH 5.5 and eluted with 20 mM histidine, 500 mM NaCl, pH 5.5 applying a linear gradient.

### d) Protein Analytics by CE-SDS

Product purity and integrity were analyzed by CE-SDS using microfluidic Labchip technology (PerkinElmer, USA) under reducing and non-reducing conditions. For this purpose, 5 μl of sample solution was prepared using the HT Protein Express Reagent Kit according manufacturer's instructions and analyzed on LabChip GXII system using a HT Protein Express Chip. Data were analyzed using LabChip GX Software.

### e) Protein Analytics by SEC

Size exclusion chromatography (SEC) for the determination of the aggregation and oligomeric state of recombinant immunoglobulins was performed by HPLC chromatography. Briefly, purified product was applied to a TSKgel QC-PAK GFC 300 column (Tosoh Bioscience) or to a Tosoh TSKgel UP-SW3000 column in 250 mM KCl, 200 mM K₂HPO₄/KH₂PO₄ buffer (pH 6.2) on a Dionex Ultimate® HPLC system (ThermoFischer Scientific, USA). The eluted antibody was quantified by UV absorbance and integration of peak areas. BioRad Gel Filtration Standard #151-1901 served as a gel filtration calibration standard.

### f) Antibody Characterization by UPLC Coupled to UHR-ESI-QTOF Mass Spectrometry

The samples were analyzed by Ultimate 3000 UPLC (Thermo Fisher Scientific GmbH, Dreieich, Germany) on a PLRP-S 150×2.1 mm column (Agilent Technologies Deutschland GmbH, Waldbronn, Germany) using a 6.5 min gradient with acetonitrile with 0.1% formic acid (v/v) and water with 0.1% formic acid (v/v), respectively (both Thermo Fisher Scientific GmbH, Dreieich, Germany). For total mass determination, the UPLC was coupled to a UHR-ESI-QTOF maXis II ETD system (Bruker Daltonics, Bremen, Germany). Calibration was performed with sodium iodide (Honeywell, Morristown, NJ). For the human IgG₁ antibodies, data acquisition was done at 800-4000 m/z (ISCID: 85.0 eV). The raw mass spectra were evaluated and transformed into individual relative molar masses using an in-house developed software tool.

### g) PD-L1 Affinity Measurement by SPR

Affinities of different PD-L1 binders to human PD-L1 were assessed by surface plasmon resonance (SPR). The SPR experiments were performed on a Biacore T200 at 25° C. with PBS-P+ as running and sample dilution buffer (0.2 M phosphate buffer with 27 mM KCl, 1.37 M NaCl and 0.5% Surfactant P20 (Tween 20), Cytiva).

Anti-PGLALA antibody (in-house) or another appropriate capturing system was directly immobilized on a CM5 chip at pH 5.0 using the standard amine coupling kit (Cytiva). After activation of the sensor surface with a 1:1 mixture of 0.4 M 1-ethyl-3-(3-dimethylaminopropyl)-carbodiimide (EDC) and 0.1 M N-hydroxysuccinimide (NHS), 20 μg/ml anti-PGLALA (diluted in 10 mM acetate pH 5.0) was injected for 720 seconds with a flow rate of 10 μl/min. After blocking with 1 M ethanolamine-HCl pH 8.5, the coupling procedure led to more than 12000 capture surface density.

PD-L1 binders were captured for 60s at a flow rate of 10 μl/min with a concentration of 10 nM. Recombinant huPD-L1 (P1AF6939) was injected at a concentration of 50 nM and then serially diluted with running buffer in 1:3 ratio to 16.7 nM, 5.6 nM, 1.9 nM and 0.6 nM with a flow of 30 μl/min through the flow cells. Association and dissociation were monitored for 120 seconds and 600 seconds respectively. The chip surface were regenerated after every cycle by using injection of 10 mM NaOH (Cytiva) for 90 seconds at a flow of 30 μl/min. Bulk refractive index differences were corrected by subtracting the response obtained on reference flow cell. Binding curves were evaluated using Biacore T200 evaluation software 3.1 (Cytiva) and for the calculation of binding properties 1:1 Langmuir binding model was used.

### h) IFN-α2a Affinity Measurement by SPR

Affinities of different IFN-α2a binders to human IFN-α2a were assessed by surface plasmon resonance (SPR)

The SPR experiments were performed on a Biacore 8K or 8K+ instrument (Cytiva) at 25° C. Anti-human Fab antibody (Cytiva; Catalog number 28958325) was immobilized on a CM5 chip according to the manufacturer's instructions. 100 nM anti-PD-L1/anti-IFN-α2a DutaFabs were captured (10 μl/min, 60 sec) and 0 nM, 10 nM, 50 nM and 150 nM of untagged huIFN-α2a (SEQ ID NO:79) was flown at 30 μl/min for 120 sec followed by a 240 second dissociation window at a flow rate of 30 μl/min. The surface was regenerated by injecting 10 mM glycine, pH 2, for 60s at a flow rate of 30 μl/min. Binding curves were evaluated using Biacore 8K evaluation software (Cytiva) and for the calculation of binding properties 1:1 Langmuir binding model was used.

In other experiments, the SPR experiments were performed on a Biacore T200 at 25° C. with PBS-P+ as running and sample dilution buffer (0.2 M phosphate buffer with 27 mM KCl, 1.37 M NaCl and 0.5% Surfactant P20 (Tween 20), Cytiva).

Briefly, anti-His antibody (Cytiva) was directly immobilized on a CM3 chip at pH 5.0 using the standard amine coupling kit (Cytiva). After activation of the sensor surface with a 1:1 mixture of 0.4 M 1-ethyl-3-(3-dimethylaminopropyl)-carbodiimide (EDC) and 0.1 M N-hydroxysuccinimide (NHS), 10 μg/ml anti-His (diluted in 10 mM acetate pH 5.0) was injected for 720 seconds with a flow rate of 10 μl/min. After blocking with 1 M ethanolamine-HCl pH 8.5, the coupling procedure led to more than 5000 RU anti-His surface density.

Recombinant human IFN-α2a (P1AF6942) was captured on the second flow cell for 60 seconds at a flow rate of 10 μl/min with a concentration of 50 nM. IFN-α2a binders were injected at a concentration of 500 nM and then serially diluted with running buffer in 1:3 ratio to 166.7 nM, 55.6 nM, 18.5 nM and 6 nM with a flow of 30 μl/min through the flow cells. Association and dissociation were monitored for 300 seconds and 600 seconds respectively. The chip surface was regenerated after every cycle by using injection of Glycine, pH 1.5 (Cytiva), for 60 seconds at a flow of 30 μl/min. Bulk refractive index differences were corrected by subtracting the response obtained on reference flow cell. Binding curves were evaluated using Biacore 8K evaluation software (Cytiva) and for the calculation of binding properties 1:1 Langmuir binding model was used.

### i) Hydrophobic Interaction Chromatography (HIC)

Apparent hydrophobicity was determined by injecting 20 μg of the respective bispecific anti-IFN-α2a/anti-PD-L1 DutaFab onto a HIC-Ether-5 PW column (Tosoh) equilibrated with 25 mM Na-phosphate, 1.5 M ammonium sulfate, pH 7.0. Elution was performed with a linear gradient from 0 to 100% buffer B (25 mM Na-phosphate, pH 7.0) within 60 minutes. Retention times were compared to protein standards with known hydrophobicity.

### j) Thermal Stability

Samples of the respective bispecific anti-IFN-α2a/anti-PD-L1 DutaFab were prepared at a concentration of 1 mg/mL in 20 mM histidine/histidine chloride, 140 mM NaCl, pH 6.0, transferred by centrifugation into an optical 384-well plate through a 0.4 μm filter plate and covered with paraffin oil. The hydrodynamic radius was measured repeatedly by dynamic light scattering on a DynaPro Plate Reader (Wyatt) while the samples are heated with a rate of 0.05° C./min from 25° C. to 80° C. Alternatively, samples were transferred into a 10 μL micro-cuvette array and static light scattering data as well as fluorescence data upon excitation with a 266 nm laser were recorded with an Optim1000 instrument (Avacta Inc.), while they were heated at a rate of 0.1° C./min from 25° C. to 90° C.

### k) Stability Upon Thermal Stress

The PD-L1-targeted IFN-α2a fusion proteins were further assessed by subjecting them to thermal stress in the following stress set-ups, at 10 mg/ml for 2 weeks: i) stress storage under in-vivo-mimicking conditions in PBS buffer (pH 7.4) at 37° C., ii) stress storage under formulation-mimicking conditions in His NaCl buffer (pH 6) at 40° C. Reference samples that had been stored for two weeks at −80° C. were analyzed in comparison to the stressed material by SEC for occurrence of high molecular weight species (HMWs), by CE-SDS to monitor low-molecular weight species such as cleavage products and by a relative active concentration assay (RAC) by surface plasmon resonance (SPR) in order to detect loss of binding to either of the targets (huPD-L1, huIFNα-2, huIFNAR2) upon stress. Additionally, LC-MS/MS peptide mapping experiments were performed of the reference and formulation-mimicking material. All CDRs were analyzed regarding deamidation of Asn residues as well as isomerization of Asp residues including the respective reaction intermediates (succinimide). For P1AI4295, only negligible levels of HMWs, LMWs, chemical degradation of Asn and Asp as well as no loss in target binding upon stress were observed in the performed assays.

### l) Serum and Plasma Stability

The molecules P1AI4798, P1AI4748 and P1AI4295 were tested for stability in human and mouse plasma and human hypodermal tissue homogenate. Ex vivo human abdominal skin models obtained as a byproduct of cosmetic surgery were provided in 6-well plates. The explants are standardized, ready to use models with preserved tissue integrity and cell viability. The models can be kept alive for seven days. The medium is provided by the vendor. The tissue explants were dissected using a disposable scalpel to isolate the fat tissue (hypodermis), which was transferred to 7 mL Precellys tubes containing ceramic beads. 2 mL of PBS containing 0.02% Tween 20 added to hypodermal tissue and the samples were lysed using a Precellys Evolution (Bertin Instruments, Montigny-le-bretonneux, France) homogenizer by 3×15 seconds burst with 30 seconds on ice in between the bursts. The sample tubes were spun down at 1,000×g for 5 minutes at 4° C. The supernatant was collected and transferred to 2 mL Eppendorf tubes. Insoluble material was pelleted by centrifugation at 15,000×g for 15 minutes at 5° C. The supernatant was filtered using Acrodisc PES 0.2 μm, 13 mm syringe filters to separate particles from the lysate. The protein concentration of all samples was measured using a BCA protein assay (Thermo Fisher Scientific) according to manufacturer's protocol.

Test compounds were incubated either in human plasma using heparin and mouse plasma using EDTA as an anticoagulant, with an incubation time of 14 days at 37° C. and 5% CO₂, or in human skin tissue lysate for 120 hours at 37° C. and 5% CO₂. As positive control, Dulaglutide was used.

The Kingfisher duo prime magnetic processor was used for immunoaffinity extraction of the proteins. Customized protocols are provided by the vendor or can be designed individually with the associated BindIt Software. Prior to the extraction steps, a 96-well deep well plate was prepared with following reagents: A: Washing Buffer PBS containing 0.02% Tween20, C: High Capacity Magne® Streptavidin Beads, D: CaptureSelect Biotin Anti-IgG-Fc Conjugate or Anti-PG-LALA capture antibody, E: Washing Buffer PBS containing 0.02% Tween 20, F: Target matrix, G, H: Washing Buffer PBS. The Elution buffer of 5% formic acid was prepared in a separate Elution strip.

In a first step, 50 μl High Capacity Magne® streptavidin beads were washed in PBS containing 0.02% Tween 20 and were bound to a biotinylated Anti-PG-LALA or an anti-human IgG-Fc capture antibody for 30 minutes. For extraction, the lysates were incubated with the magnetic beads coupled to the capture antibody for 30 minutes. After two washing steps with PBS containing 0.02% Tween20, the purified material was eluted in 50 μL 100 mM glycine for 10 minutes. The beads were removed using a magnet block and the supernatants were analyzed by Intact liquid chromatography high-resolution mass spectrometry (LC-MS/MS).

For this, all intact measurements were performed on a maxis II HR-QTOF mass spectrometer (Bruker Daltonics, Bremen, Germany) coupled to an UltiMate 3000 UHPLC liquid chromatography system (Thermo Fisher Scientific, Waltham, MA, USA). Protein separation was achieved by reverse phase mAbPac RP (2.1×150 mm; Thermo Fisher Scientific, Waltham, MA, USA) at a flow rate of 400 μl/min and column temperature of 80° C. The mobile phase consisted of 0.1% acid and 5-95% acetonitrile running in a linear gradient. Full scan MS spectra were obtained in positive ion polarity at a spectra rate of 0.5 Hz covering an m/z range from 800-6000. The ESI source parameters were 500 V end plate offset, 4500 V capillary voltage, 2 bar nebulizer pressure, 10 l/min dry gas and 280° C. dry temperature. Funnel 1 RF and Multipole RF were set to 400 Vpp and the isCID Energy was set to 60 eV. Quadrupole ion energy was set to 5.0 eV with a low mass setting of 1000 m/z. The collision cell energy was 10 eV at a collision RF of 4000 Vpp, 200 us transfer time and 15 μs pre-pulse storage.

For data analysis, the mass spectrometry data was analyzed with Compass DataAnalysis (v4.4 SR1, Bruker Daltonics, Bremen, Germany). Mass spectra were deconvoluted using the maximum entropy algorithm with a set instrument resolving power of 8000. The mass lists for deconvoluted mass spectra were calculated through SumPeak using the vendors default parameters.

Upon 14 day incubation of the molecules P1AI4798, P1AI4748 and P1AI4295 in mouse plasma, a loss of sialic acid at IFN-α2a amino acid position Thr106 (O-glycosylation site) was observed. No instability was found after 120 hours incubation in human hypodermal tissue. Activity of the homogenate was confirmed by biotransformation of the positive control Dulaglutide.

### Example 9: huIFN-α2a-Linker Attachment Site Affects Dimer Formation Tendency

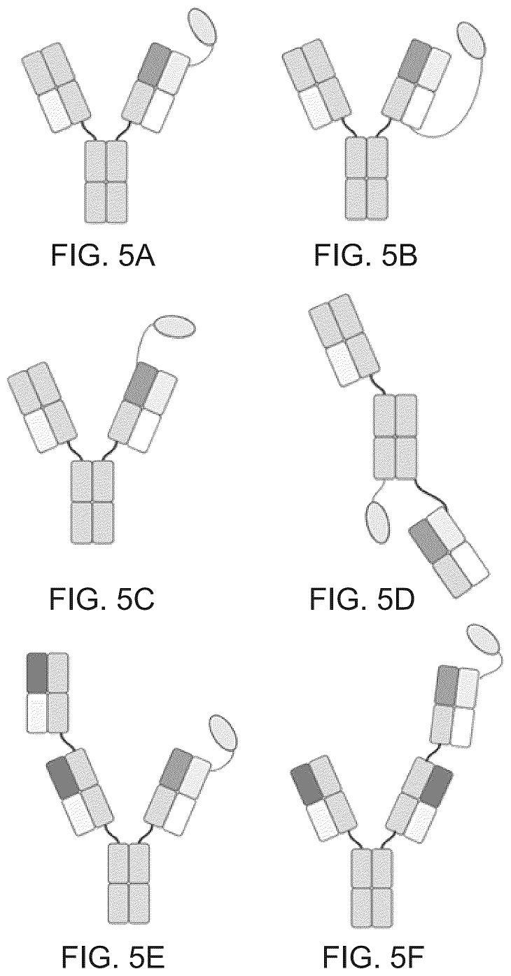

Human interferon alpha-2a (huIFN-α2a) was attached to the Dutaflip-IgG molecule at different sites in order to evaluate conjugation site dependency on functional as well as physical properties. Therefore, fusion proteins were generated in which huIFN-α2a (SEQ ID NO: 79) fused via a (GGSGG)ₙ linker of different lengths at the N- or C-terminus of the Dutaflip light chain or to the C-terminus of the targeting heavy chain (FIG. 5A-5F). Dimer formation after affinity chromatography was assessed by analytical size exclusion chromatography and mass photometry (see Table 13).

huIFN-α2a fusions to the N-terminal ends of the light chain (FIG. 5A; P1AH1108, P1AH1111) with a short (20 aa) linker showed low dimer formation after capturing. Existing dimers could be removed by a preparative size exclusion chromatography (SEC) and/or cation exchange (cIEX) chromatography purification step. The monomeric molecule did not show a tendency for dimer formation as shown by analytical size exclusion chromatography or mass photometry (see Table 13).

TABLE 13
Dependence of dimer formation on IFN-α2a attachment site

| | IFN-α2 | GGSGG | % dimer | dimers |
|---|---|---|---|---|
| Molecule | attachment | linker | after | after cIEX |
| ID | point | length | capture | or SEC |
| P1AH1108 | N-term LC | 20 | 5.0 | <1 |
| P1AH1111 | N-term LC | 20 | 1.3 | <1 |
| P1AG9500 | C-term LC | 37 | 53 | reformation |
| P1AG9456 | C-term LC | 37 | 34.1 | reformation |
| P1AH0172 | C-term LC | 37 | 52.8 | reformation |
| P1AH1122 | C-term LC | 60 | 9.9 | not removable by |
| | | | | cIEX, reformation |
| P1AH1123 | C-term LC | 60 | 6.2 | not removable by |
| | | | | cIEX, reformation |
| P1AG4740 | C-terminal HC | 18 | 7.3 | reformation |
| P1AI1297 | N-terminal HC | 20 | 19.9 | reformation |

In contrast, IFN-α2a fusions to the C-terminal ends of the light chain (FIG. 5 B; P1AG9500, P1AG9456, P1AH0172, P1AH1122, P1AH1123) with longer linker lengths (37aa and 60 aa) led to increased dimer levels (see Table 13). Removal of the dimers by preparative SEC or cIEX were unsuccessful as isolated monomers tend to dimer reformation until a certain equilibrium. Similar behavior was observed when IFN-α2a was fused to the C-terminus of the heavy chain (FIG. 5 D; P1AG4739 and P1AG4740).

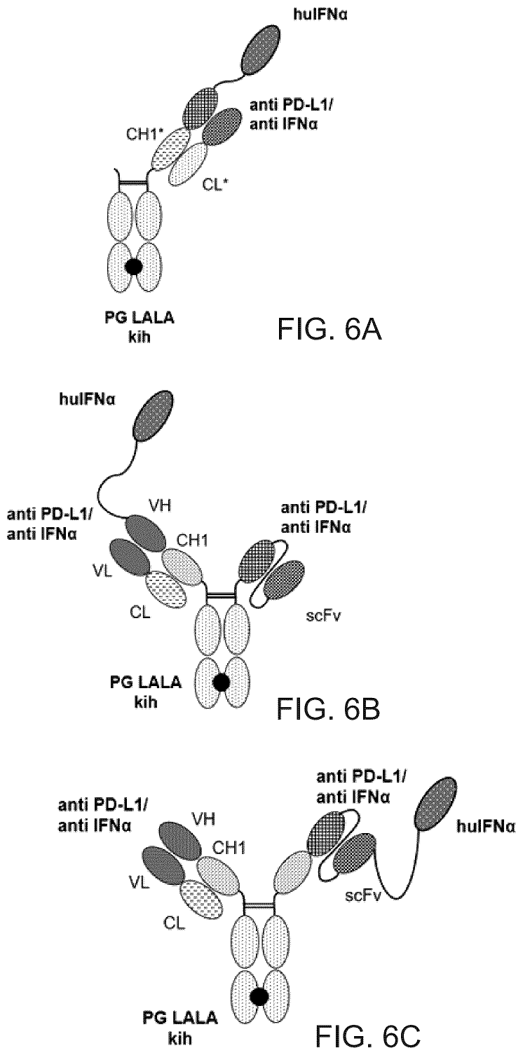

The reference molecule P1AI1297 with IFN-α2a fused to the N-terminus of the heavy chain DBA (FIG. 6 B) also showed significant dimer level after affinity capture by Protein A chromatography, although the linker used in this molecule is also 20 aa long. Although dimers could be strongly reduced by cIEX and/or SEC, the dimer reformed after some equilibration time and at a concentration of about 3 mg/ml to significant levels. This demonstrates that IFN-α2 attachment site, linker length and IFN-α2a affinities have an influence on dimer formation.

### Mass Photometry Measurements

The tendency of dimer formation of the PD-L1-targeted IFN-α2a fusion proteins was evaluated by mass photometry. The experiments were carried out on a OneMP instrument (Refeyn, UK) at room temperature. Microscope coverslips (24×50 mm, Fisher Scientific) were prepared by rinsing them consecutively with isopropanol and H₂O and drying them under a stream of clean nitrogen. All protein dilutions and measurements were prepared in filtrated (0.22 μm filter) 1×PBS pH 7.4 buffer, prepared from a 10×PBS premixed stock solution (Roche 11666789001). The samples were diluted to 3 μM, 1.5 μM and 250 nM, respectively, to check for concentration dependency. The final protein concentration in the droplet was calculated with 7.5 nM. For calibration purposes, Thermo Fisher Scientific NativeMark (LC0725) mass standard was analyzed (66 kDa, 146 kDa, 480 kDa). Data was collected from the 2.9 μm×10.8 μm instrument field of view for 60 seconds at a 1 kHz frame rate using the AcquireMP 2.4.1 software. Images were processed using the DiscoverMP 2.4.2 software. For processing, the default settings (number of averaged frames=5, threshold 1=1.5, threshold 2=0.25) were used.

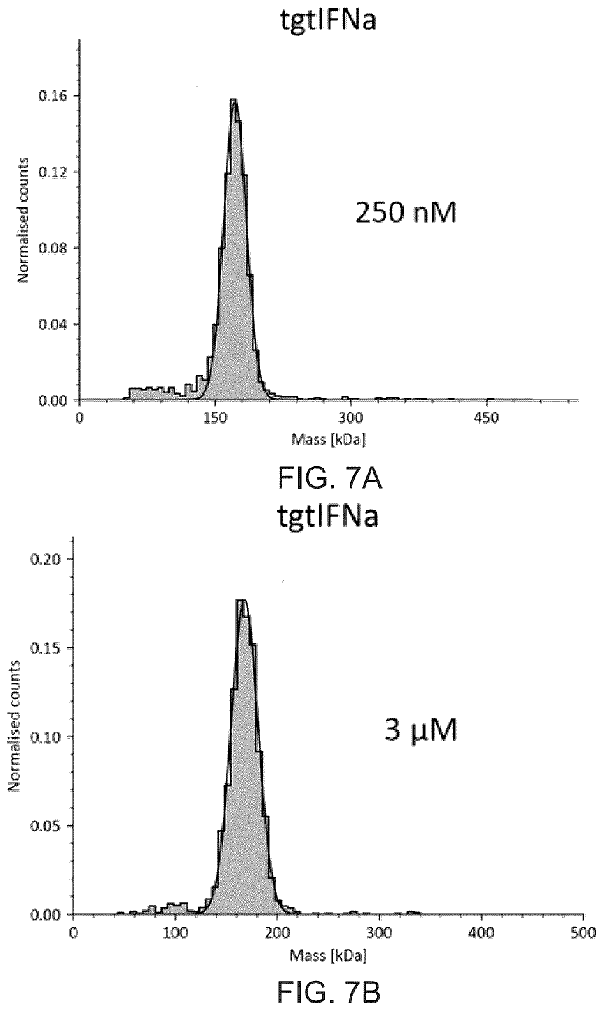

Mass photometry measurement of molecule P1AI4295 at a concentration of 250 nM (FIG. 7A) and 3 μM (FIG. 7 B) revealed a mass of approximately 172 kDa, corresponding to the monomer of the fusion protein. Even after an increase of the concentration, no tendency for dimer formation was observed. Control experiments with fusion proteins with a tendency for dimerization were performed and showed the expected results, i.e. dimer formation (data not shown).

### Example 10: Evaluation of Formats: 2× PD-L1-Targeting Vs 1+1 Format (Functional)

Various IgG formats for the PD-L1-targeted IFN-α2a Dutaflip fusion proteins were generated comprising different monovalent or bivalent PD-L1-targeting modules and the same Dutaflip IFN-α2 fusion module. The “2+1” molecules P1AI4848 and P1AI4853 (having a format as shown in FIG. 5 E or 5F, respectively) were generated by transient expression in HEK293 Expi™ and purified as described above. PD-L1 controlled IFNα activation was analyzed in HEKblue IFNα/β assay using cell lines with low and high PD-L1 levels, essentially described as in Example 13. The 2+1 formats did not show superior PD-L1 dependent IFNα activation in HEKblue assay compared to 1+1 formats (P1AI4295, P1AI4797).

### Example 11: Thermal Stability

Measuring thermal transitions is a good indicator for aggregation propensity and thermal stability of macromolecules. Melting temperatures (Tₘ) and aggregation temperatures (T_{agg}) were determined using the UNCLE system (Unchained labs) that combines tryptophan fluorescence, Static Light Scattering (SLS) and Dynamic Light Scattering (DLS) for measuring Tₘ and T_{agg}.

TABLE 14
Melting temperatures and aggregation temperatures

| | T_{agg} | Tₘ |
|---|---|---|
| P1AI4295 | 57° C. | 72° C. |
| P1AH1111 | 59° C. | 71° C. |
| P1AI1297 | 48.5° C. | 53° C. |

Samples were formulated in 20 mM histidine, 140 mM NaCl, pH 6.0 at a concentration of 1 mg/ml for analysis. A temperature ramp from 30° C. to 90° C. with a heating rate of 0.1° C./min was used. All samples were measured in triplicates. Data analysis were performed by the client software 1.0 (unchained labs). The Tₘ and T_{agg} values for molecules P1AI4295, P1AH1111 and the reference molecule P1AI1297 are shown in Table 14. The molecules according to the invention show significantly higher Tₘ and T_{agg} values, and thus thermal stability, than the reference molecule P1AI1297.

### Example 12: Generation of PD-L1 Expressing HEKBlue IFNα/β Reporter Cell Line

Full-length cDNA encoding human PD-L1 was subcloned into a mammalian expression vector. The plasmid was transfected into HEK-Blue IFNα/β (Invivogen, Catalog number hkb-IFNα/B) cells using Lipofectamine 3000 Reagent (Invitrogen, Catalog number L3000015) according to the manufacturer's protocol. HEK-Blue IFNα/β cells were maintained in DMEM media (PAN, #P04-03596) supplemented with 10% FCS (Gibco, Catalog number 10500), 2 mM L-Glutamine (PAN, Catalog number P04-80100), 30 μg/mL Blasticidin (Gibco, Catalog number A1113903) and 100 μg/mL Zeocin (Gibco, Catalog number R25001). Two days after transfection, Hygromycin (PAN, Catalog number P06-08020) was added to 200 μg/ml. After initial selection, the cells with the highest cell surface expression of PD-L1 were sorted by BD FACSAria III cell sorter (BD Biosciences) and cultured to establish stable cell clones. The expression level and stability was confirmed by FACS analysis using PE mouse anti-human CD274 (Clone MIH1) (BD Biosciences, #557924) over a period of 4 weeks. In addition, a QUANTIblue assay (Invivogen, Catalog number rep-qbs2) was performed according to the manufacturer's protocol in order to ensure that the reporter activation via the IFNα pathway was not significantly affected by stable transfection of PD-L1. Selected clone 45 (RNCB accession IDCL022702)) showed comparable IFNα stimulation to HekBlue-IFNα/β wildtype cells. Quantification of the cell-surface PD-L1 using a bead based Quantification kit (BD, Catalog number 340495) revealed low PD-L1-surface levels for HekBlue-IFNα/β wildtype cells (˜300 PD-L1 molecules/cell on cell surface; herein also referred to as “parental cells”) and high PD-L1-surface levels of clone 45 (˜25000 PD-L1 molecules/cell on cell surface).

### Example 13: HEK-Blue Human IFNAR1/2 Reporter Cell In Vitro Assay

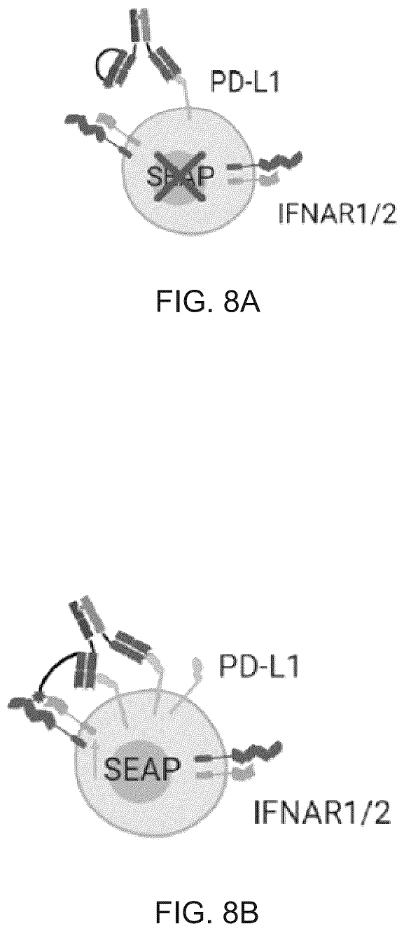

Parental and huPD-L1 transfected HEK-Blue IFNα/β cells (generated as described in Example 12) were used as reporter cell lines to analyze huPD-L1 dependent IFNAR1/2 receptor activation by huPD-L1-targeted IFN-α2a Dutaflip fusion proteins (the principle of the assay is illustrated in FIGS. 8A and 8B). Fusion proteins can only activate IFNAR 1/2 receptors when huPD-L1 is present in sufficient amount on the cell surface so that the PD-L1-binding side of the Dutaflip arm binds to the huPD-L1 on the cell surface, thus releasing IFN-α2a and setting it free to bind to its receptor on the cell surface. Upon binding of IFN-α2a to IFNAR1/2 receptors, the JAK/STAT/ISGF3 pathway is triggered, leading finally to the expression of the reporter gene which is under the control of the ISG54 promoter containing an IFN-stimulated response element (ISRE). The reporter gene SEAP (secreted alkaline phosphatase) is produced by the cell and secreted into the medium. Its amount correlates with the extent of IFNAR 1/2 receptor activation. The SEAP levels in the medium can be measured by using a SEAP detection reagent like QUANTI-Blue™ and the color change of the detection reagent by the SEAP activity can be measured with a spectrophotometer.

First, 10× titration series (8 steps) of the different PD-L1-targeted IFN-α2a Dutaflip fusion proteins were prepared, starting at 100 nM (final concentration in well between 10 nM and 0.000001 nM). 20 u L per fusion protein of each concentration were transferred to 96-well flat bottom plates in duplicates. Subsequently, 4×10⁴ reporter cells/well were seeded in 180 μL medium (DMEM high glucose (4.5 g/L glucose) (PAN, Catalog number P04-03609)+10% heat-inactivated FBS (Anprotec, #AC-SM-0014Hi)+2 mM L-glutamine (PAN, Catalog number P04-80100)). As positive control, recombinant IFN-α2a (PBL Assay Science, Catalog number 11101-2) was used in the same concentration range. After 24 hours of incubation at 37° C., assay plates were centrifuged at 300 g for 5 min. Meanwhile, QUANTI-Blue solution (Invivogen, #rep-qbs) was prepared and 180 μL/well were distributed among wells of new 96-well flat bottom plates, followed by addition of 20 μL of supernatants of treated parental or huPD-L1 transfected HEK-Blue IFNα/β cells, respectively. Substrate turnover by produced SEAP was allowed for 30 to 45 min before measuring the optical density (OD) at 640 nm. The results of the assay are shown in FIG. 9A (PD-L1 low expressing parental cells) and 9B (PD-L1 high-expressing cells).

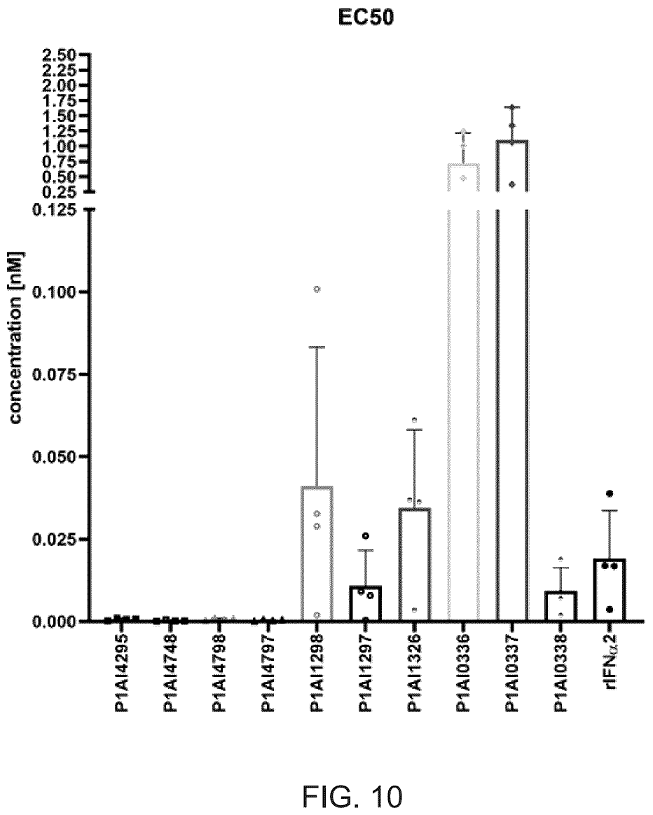

As shown in FIG. 10, the EC50 values calculated for reference molecules P1AI1297, P1AI1298, P1AI1326, P1AI0336, P1AI0337 and P1AI0338 based on the dose-response curves obtained with huPD-L1 transfected HEK-Blue IFNα/β cells were found to be between 0.006 nM and 1.1 nM, corresponding to at least 10 times higher EC50 values than the EC50 values that were observed for the molecules according to the invention (P14295, P14748, P1AI4798, P1AI4797) or even recombinant IFN-α2a. The calculated EC50 values calculated from the dose-response curves are also shown in Table 15. These results show that the molecules according to the invention are more potent on PD-L1 high expressing cells compared to the reference molecules.

EC50 values from dose-response curves obtained with parental HEK-Blue IFNα/β cells could not be determined because an upper plateau was not reached. PD-L1 expression on the cell surface of parental HEK-Blue IFNα/β cells was not sufficient to fully release the IFN-α2 moiety from the PD-L1-targeted IFN-α2a Dutaflip fusion proteins at most of the tested concentrations and activation of all tested fusion proteins was lower than that of recombinant IFN-α2a (FIG. 9 B).

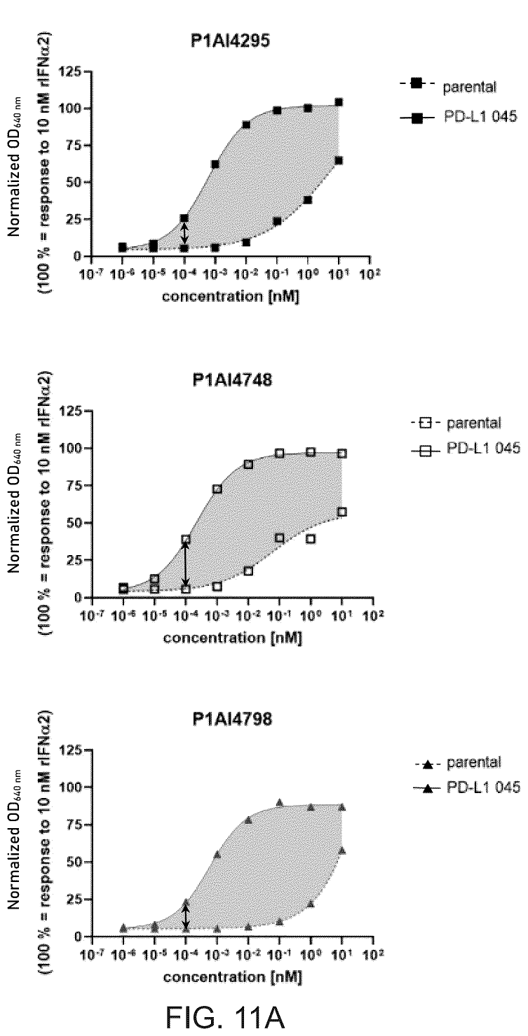

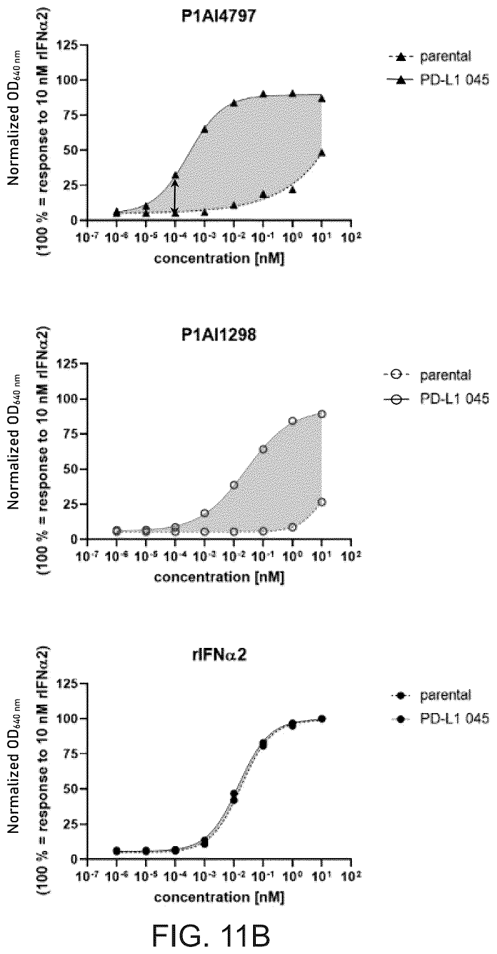

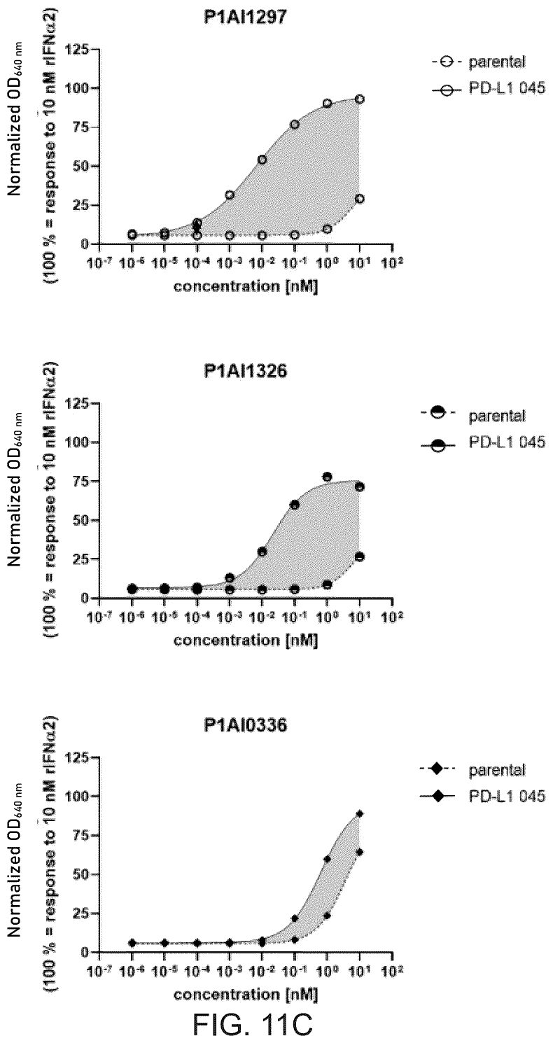

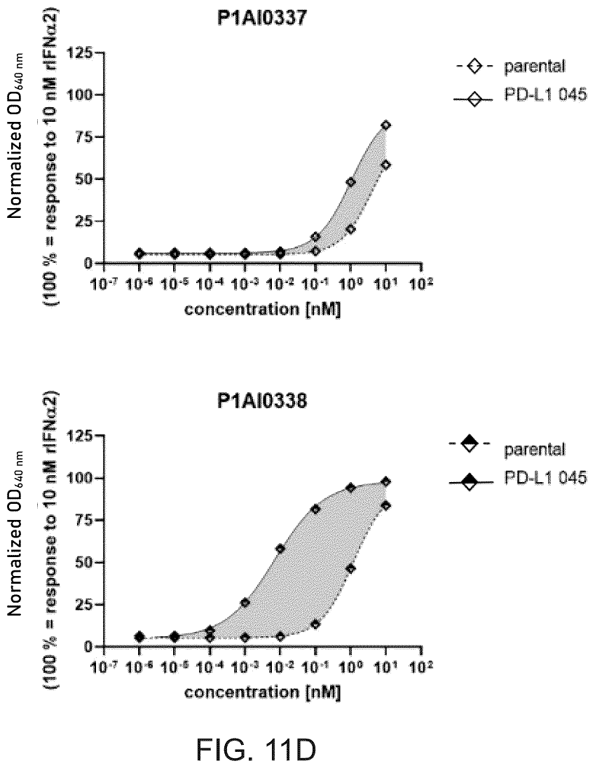

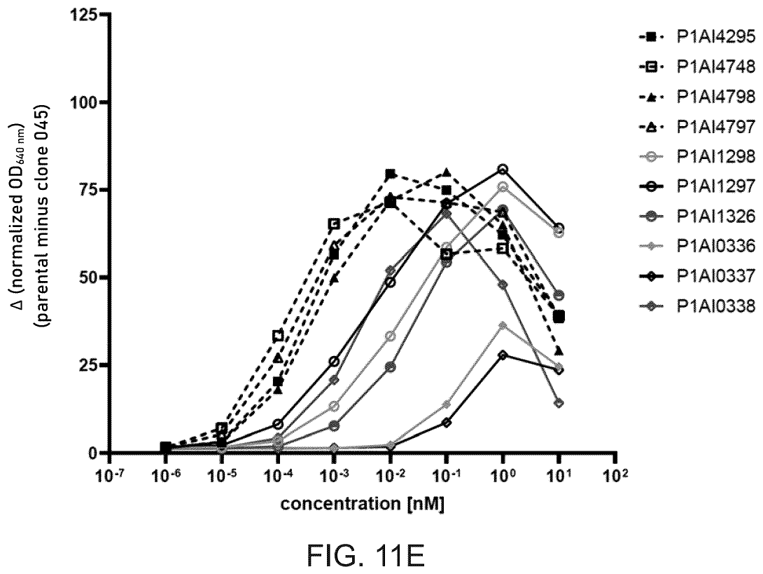

As shown in FIGS. 9A and 9B and in FIGS. 11A-11D, PD-L1-targeted IFN-α2a Dutaflip fusion proteins show low activity on PD-L1 low expressing cells (HEK-blue parental; PD-L1 per cell: ˜300) and high activity on PD-L1 high expressing cells (HEK-blue clone 04; PD-L1 per cell: ˜25,000), demonstrating a high PD-L1 dependency of IFNAR 1/2 activation for all molecules and showing that the molecules according to the invention have a particularly large activity window between PD-L1 high and low expressing cells.

TABLE 15
EC50 values calculated from dose-response curves of HEK-
Blue IFN-α/β cells transfected with PD-L1

| | EC50 value (PD-L1 high |
|---|---|
| Molecule | expressing cells) |
| P1AI4295 | 0.0006096 |
| P1AI4748 | 0.0002183 |
| P1AI4798 | 0.0005809 |
| P1AI4797 | 0.0002822 |
| P1AI1298 | 0.02544 |
| P1AI1297 | 0.006719 |
| P1AI1326 | 0.02166 |
| P1AI0336 | 0.6435 |
| P1AI0337 | 1.053 |
| P1AI0338 | 0.006973 |
| rIFN-α2 | 0.01445 |

In FIGS. 11A-11D, the values observed for each molecules have been normalized by setting the activation observed with recombinant human IFN-α2a to 100%), and the IFN-α2 activation generated with PD-L1 low and high expressing cells for tested each molecule is shown in direct comparison. The difference (Δ) in activation observed between PD-L1 high and low expressing cells, represented by the area between the two curves, is marked in gray. Remarkably, the difference in activation between high and low expressing cells is already detectable at much lower concentrations with the molecules of the invention, as compared to the reference molecules (indicated in FIG. 11A though FIG. 11C by an arrow).

In FIG. 11 E, in order to visualize this effect, the difference (Δ) between IFN-α2 activation in PD-L1 low- and high-expressing cells of each molecule (Δ_{normalized OD640 nm}) has been plotted against the concentration of the molecule used in the assay. As can been seen in FIG. 11 E, the molecules of the invention show already significant difference in activation at around 10⁻⁴ nM and generally reach their maximum window between IFNα activation in PD-L1 high-expressing cells (e.g. in tumor cells or immune cells in the tumor micro environment) and PD-L1 low-expressing cells (e.g. in healthy tissue) at a lower concentration than the reference molecules. As a consequence, lower concentrations of the molecules of the invention are required to achieve maximal therapeutic effect when used as a pharmaceutical agent, allowing the reduction of undesirable side effects.

This assay represents a model for the therapeutic window of the tested molecules in the patient. The parental HEK-Blue IFNα/β cells mimic the environment in the periphery where cells have little to no PD-L1 surface expression and where as little as possible IFNα activation is desired as it can lead to toxicity in the patient. The huPD-L1 transfected HEK-Blue IFNα/β cells on the other hand mimic the situation in the targeted tumor tissue where cells express high levels of PD-L1 on the cell surface. In the presence of high PD-L1 cell surface expression, high IFNα activity on PD-L1 expressing cells is desired in order to unfold the best possible desired therapeutic effect against the target cells, while at the same time minimizing undesirable side effects.

### Example 14: PD-L1-IFN-α2a Induced Inhibition of Tumor Cell Proliferation and CXCL10 Secretion

The effect of the PD-L1-targeted IFN-α2a Dutaflip fusion proteins on the proliferation rate of the human tumor cell line HCC1954, BT-20, SK-BR3 and COR-L105 was measured using the cell confluence application of the IncuCyte SX5 (Sartorius). Additionally, lymphoma suspension cell line HDLM-2 was measured using object count application of the IncuCyte SX5.

TABLE 16
Overview of tested molecules and cell lines

| | HCC1954 | BT-20 | SK-BR3 | COR-L105 | HDLM-2 |
|---|---|---|---|---|---|
| P1AI4295 | X | X | X | X | X |
| P1AI4797 | X | X | X | | |
| P1AI4798 | X | X | X | | |
| P1AI4748 | X | X | X | | |
| P1AI1297 | X | X | X | | |
| (reference) | | | | | |

HCC1954 cells (ATCC No. CRL-2338) were cultivated with 90% RPMI 1640, 2 mM L-Glutamine, 1 mM Sodium Pyruvate, 10 mM HEPES, 4.5 g/L Glucose, 1.5 g/L NaHCO₃(PAN Biotech, Cat.No.P04-18047) supplemented with 10% Fetal Bovine Serum (Anprotec, Cat.No. AC-SM-0014Hi)) and Penicillin/Streptomycin (Roche, Cat.no 11074440001). Cells from the human breast cancer cell line BT-20 (ATCC No. HTB-19) were cultivated in Eagles MEM+ Earles BSS (Anprotech #AC-LM-0046), 10% FCS (Anprotech #AC-SM-001-4Hi), 2 mM L-Glutamine (PAN P04-8010), 0.1 mM NEAA (PAN #P08-32100) and 1 mM Sodium Pyruvate (PAN #P04-43100) and cells from the human breast cancer cell line SK-BR3 (ATCC No. HTB-30) were cultivated in McCoys 5A (Anprotech #AC-LM-0036), 20% FCS (Anprotech #AC-SM-001-4Hi) and 1.5 mM L-Glutamine (PAN P04-8010). Cells from the non-small cell lung adenocarcinoma cell line COR-L105 (ECACC #92031918) were cultivated in RPMI1640 (PAN #P04-17500), 10% FCS (Anprotech #AC-SM-001-4Hi) and 2 mM L-Glutamine (PAN P04-8010). Suspension cells from Hodgkin's lymphoma cell line HDLM-2 (DSMZ no. ACC 17) were cultivated in RPMI1640 (PAN #P04-17500), 20% FCS (Anprotech #AC-SM-001-4Hi) and 2 mM L-Glutamine (PAN P04-8010). 7,500 HCC1954, 10,000 BT-20, 7,500 SK-BR3 and 7,500 COR-L105 tumor cells were seeded in 180 μl cultivation medium in a flat-bottom 96-well plate (Corning, Cat.No. 3585) and 20 μl of pre-diluted molecules (final conc. 10 nM) were added. For the suspension cell line HDLM-2 20,000 cells were seeded in 150 μl cultivation medium in 96-well-plates coated with Poly-L-Ornithin (Sigma #4957) and 50 μL of molecule titrations (final conc. 10 nM to 0.0001 nM, 1:10 titration) were added. The plates were transferred to the IncuCyte® and the confluence of the cells or the phase object count was measured every 4 hours for up to ten days (phase contrast, 10× objective, for suspension 20× objective). Data analysis was performed with the IncuCyte® Software 2022A (basic analysis).

The CXCL10 (IP10) secretion levels of human tumor cells treated with PD-L1-targeted IFN-α2a fusion proteins was measured by ELISA. Therefore, 100,000-200,000 cells per well (150 μl/well) were seeded in corresponding medium and 50 μl of respective fusion protein, reference molecule or recombinant human IFN-α2a (PBL Assay Science #11101-2, Lot #7191) dilutions (100 nM to 0.001 nM, 1:10 dilution) were added per well. After 24 hours incubation, plates were centrifuged and supernatants for an IP-10 ELISA (Human CXCL10/IP-10 DuoSet ELISA, R&D #DY266 and DuoSet Ancillary Reagent Kit2, R&D #DY008) collected. The IP-10 ELISA was performed as described in the protocol provided by the manufacturer. Undiluted samples or standards (2000 μg/ml diluted in Reagent Diluent 1:2 until 31.2 pg/ml) were added (100 μl per well) to the ELISA plate. The plate was scaled and incubated for two hours at room temperature. Afterwards the supernatant was aspirated, wells were washed and detection antibody (20 ng per ml diluted in Reagent Diluent) was added to each well (100 μl per well), sealed and incubated for 2 hours at room temperature. After another aspiration and wash step, streptavidin-HRP (40-fold dilution in Reagent Diluent) was added to each well (100 μl per well) and incubated for 20 minutes at room temperature. The aspiration and wash step was then repeated. Substrate Solution (equal volumes of Color Reagent A and B) was added to each well (100 μl per well) and incubated for 20 minutes in the dark at room temperature. Finally the stop solution (50 μl per well) was added to each well and the optical density using a microplate reader (set to 450 nm, wavelength correction 570 nm) was determined.

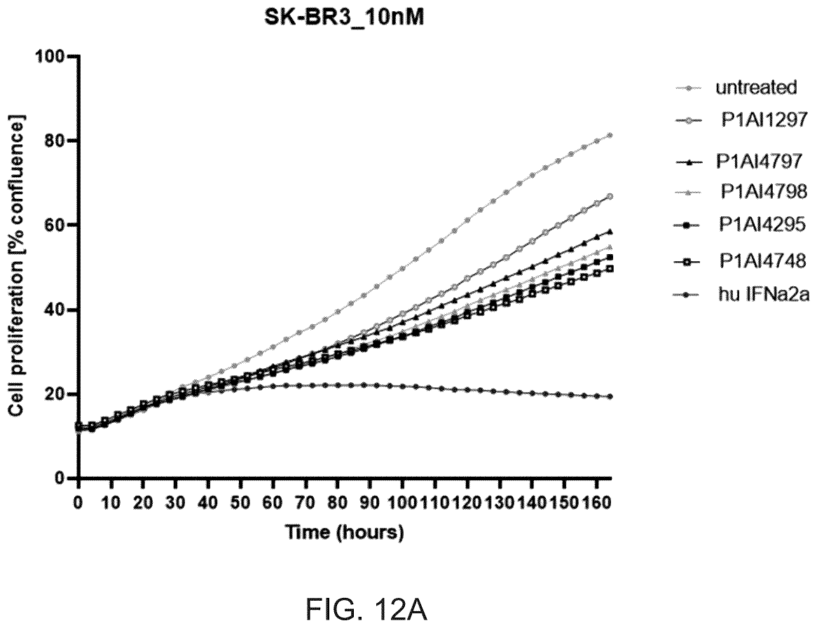

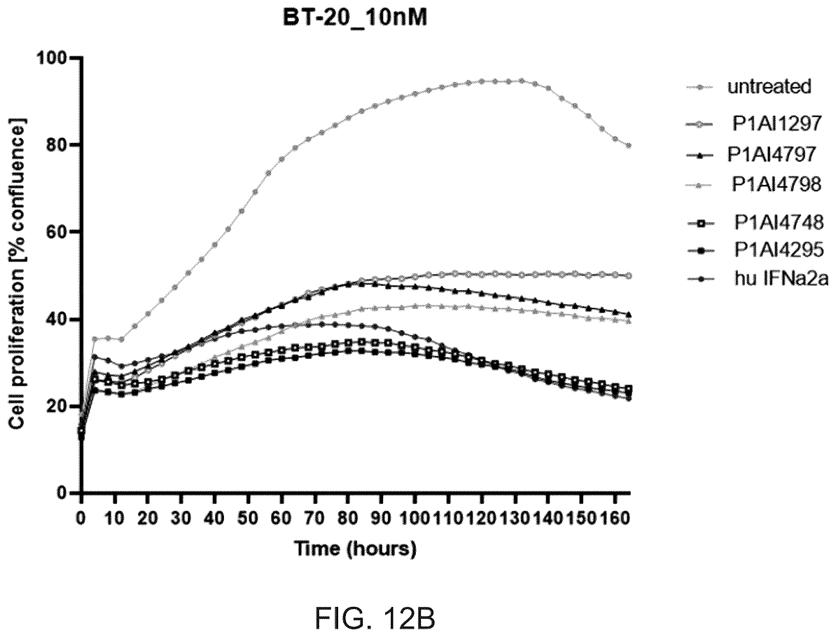

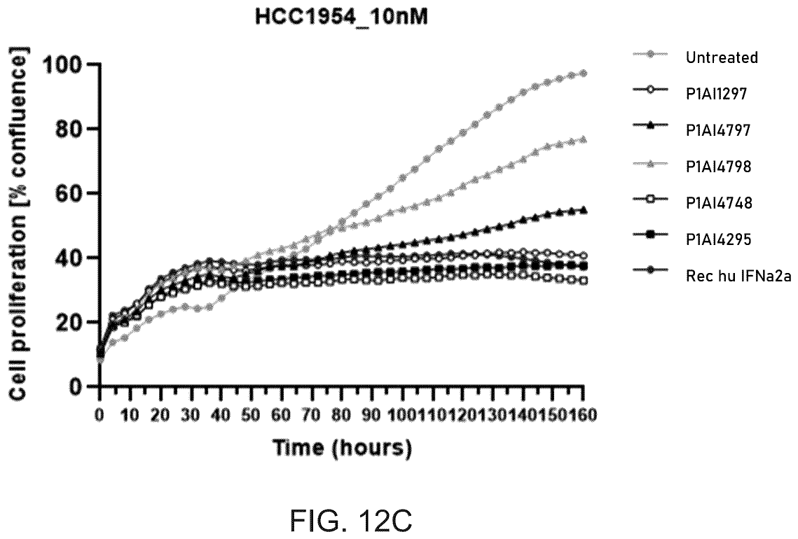

As can be seen from FIGS. 12A-12C, the PD-L1-targeted IFN-α2a Dutaflip fusion proteins P1AI4797, P1AI4798, P1AI4748 and P1AI4295 substantially decrease the proliferation rate of human SK-BR3 (7,500 cells; FIG. 12A), BT-20 (10,000 cells; FIG. 12B) and HCC1954 (7,500 cells; FIG. 12C) tumor cells and induce high CXCL10 secretion (key chemoattractant for T cells). The efficacy of the PD-L1-targeted IFN-α2a Dutaflip fusion proteins is considerably higher than that of the reference molecule P1AI1297. Wildtype IFN-α2a was used as a control.

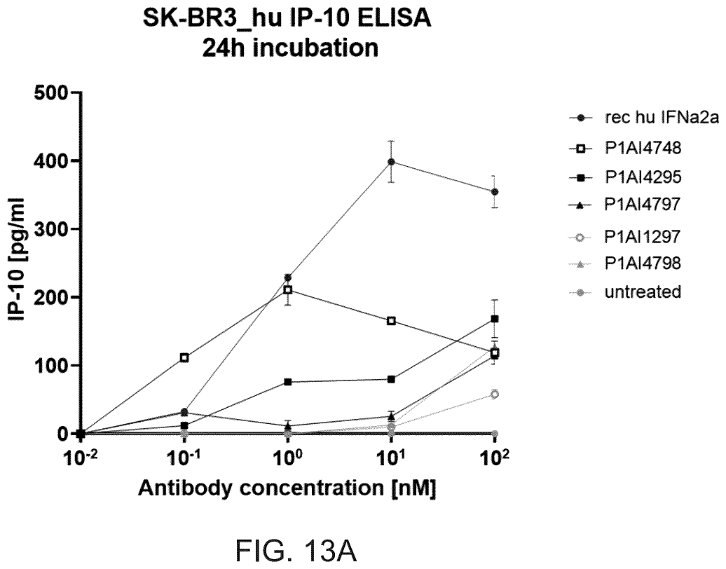

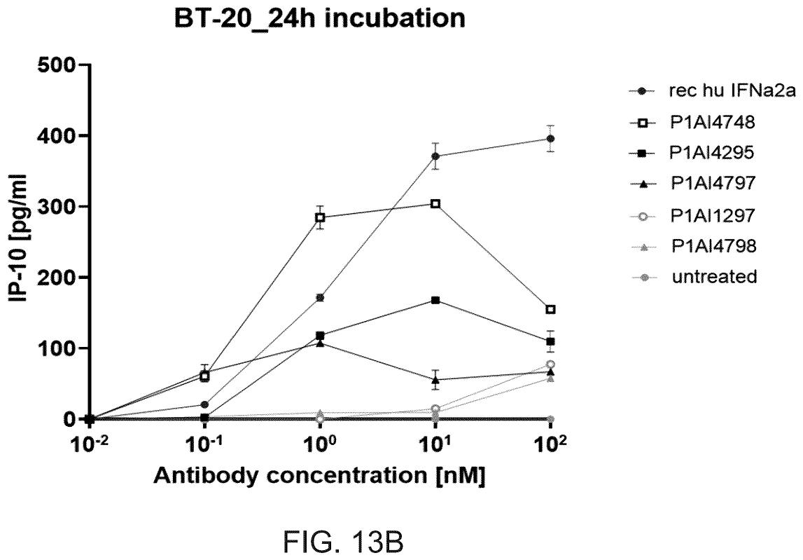

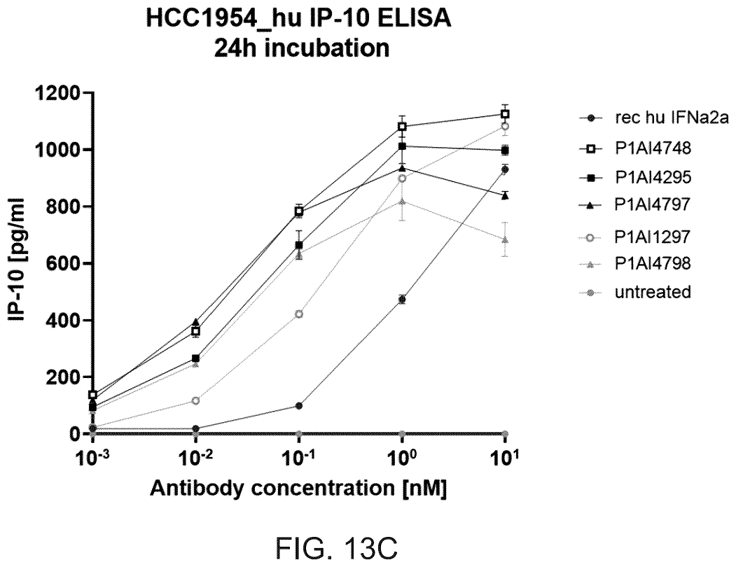

FIGS. 13A-13C (SK-BR3: 100,000 cells; BT-20:200,000 cells; HCC1954: 30.000 cells) shows the CXCL10 (labelled “IP-10 in the figures) secretion levels observed in the cells. As shown in the figures, the PD-L1-targeted IFN-α2a Dutaflip fusion proteins P1AI4797, P1AI4798, P1AI4748 and P1AI4295 induce higher CXCL10 secretion levels than the reference molecule P1AI1297. In the tumor cell line HCC1954 (FIG. 13 C), the tested molecules are able to induce IFNα activation in this cell line at even higher levels than recombinant IFN-α2a, because this cell line has a higher PD-L1 expression than the other two cell lines and IFN-α activation is dependent on the level of PD-L1 expression on the cell surface.

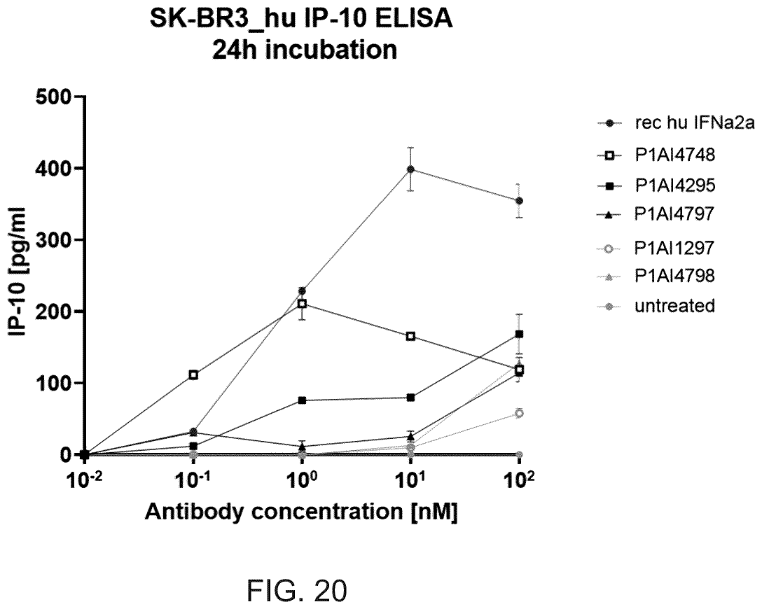

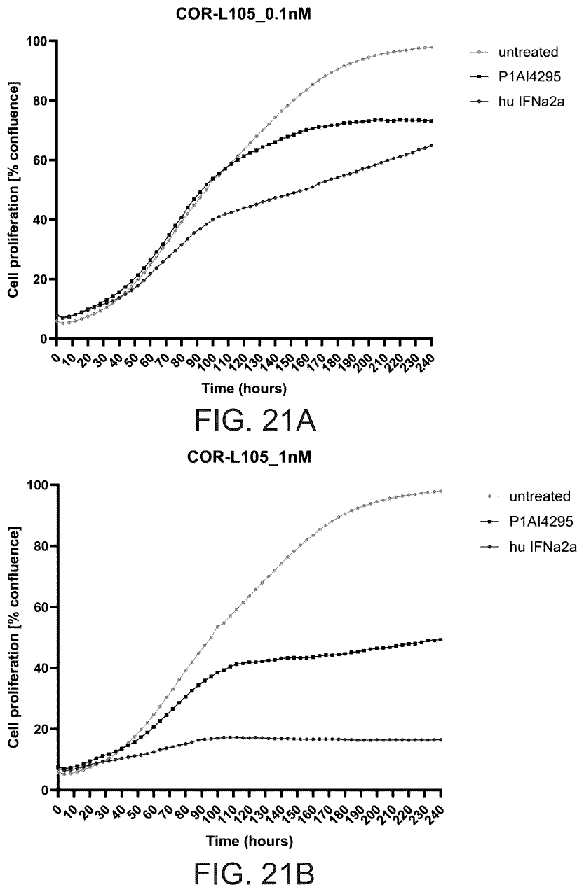

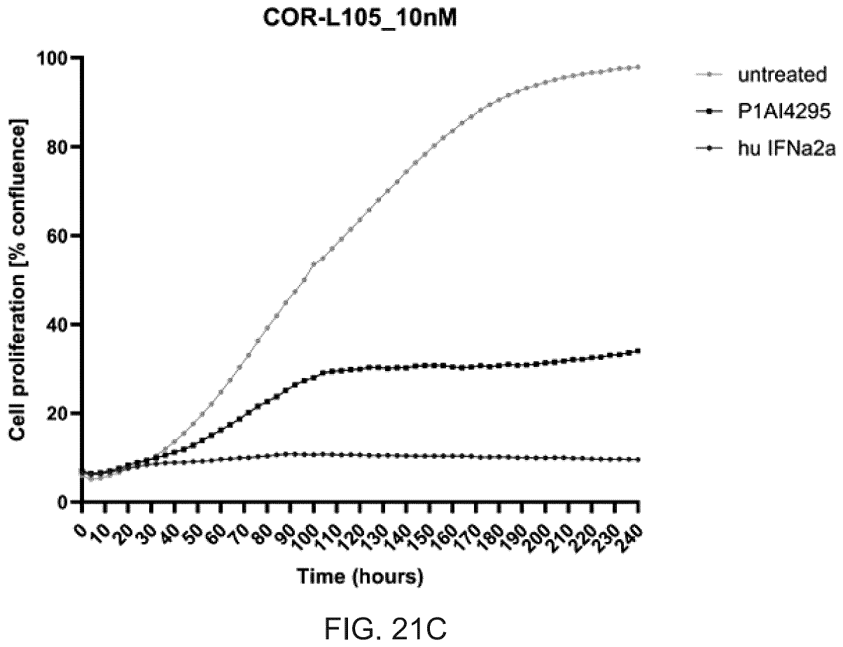

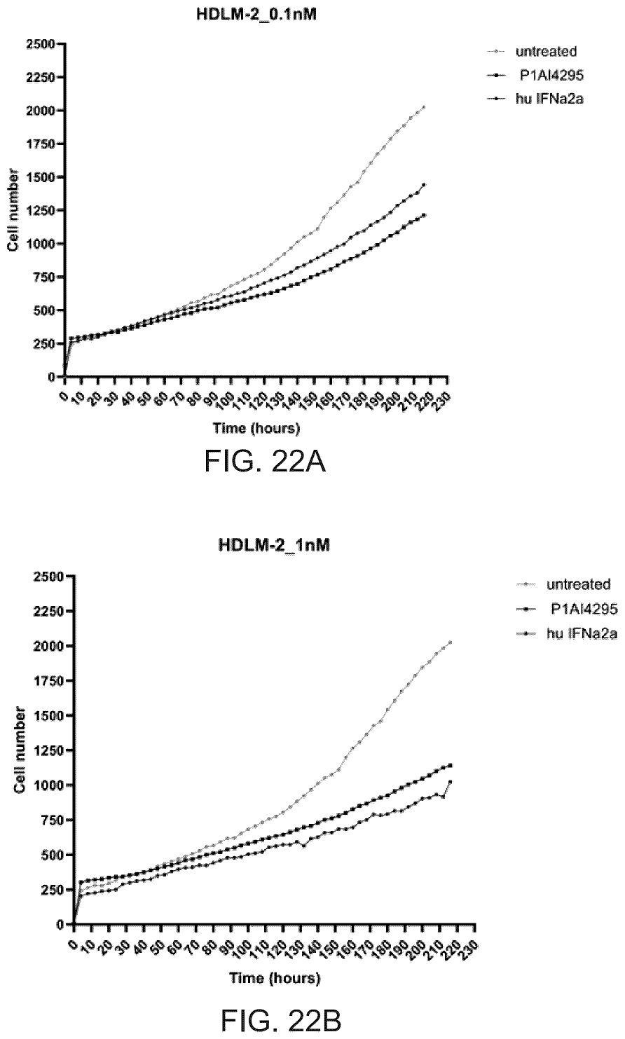

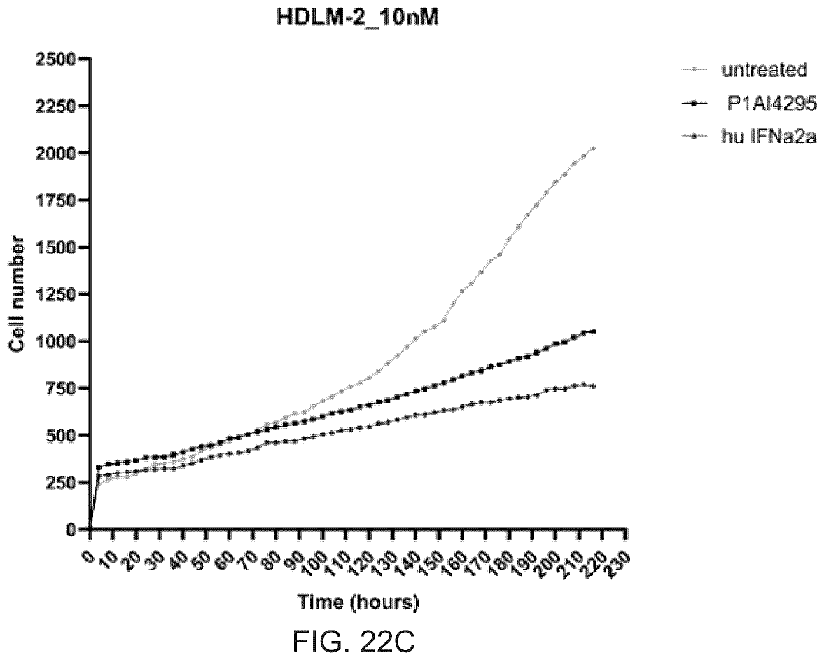

The PD-L1-targeted IFN-α2a Dutaflip fusion protein P1AI4295 was also tested in the human tumor cell lines COR-L105 and HDLM-2. Recombinant IFN-α2a was used as a positive control, and untreated cells were used as a negative control. In both cell lines, a clear dose dependent effect of P1AI4295 could be observed. In the COR-L105 cell line (FIGS. 21A-21C), P1AI4295 led to a dose-dependent decrease of tumor cell proliferation, from a reduction of 60-70% cell confluency at 0.1 nM (FIG. 21A) to 20-30% cell confluency at 10 nM (FIG. 21 C). In HDLM-2 cells (FIGS. 22A-22C), P1AI4295 demonstrated a similar reduction in tumor cell numbers compared to the control recombinant IFN-α2a.

### Example 15: PD-L1-Targeted IFN-α2a Fusion Proteins Induced MHC I and PD-L1 Expression in Human HCC1954 Tumor Cells (Breast Cancer Cell Line)

MHC1 and PD-L1 expression in HCC1954 breast cancer cell line treated with PD-L1-targeted IFN-α2a Dutaflip fusion proteins P1AI4797 and P1AI4295 was measured using the live cell imaging of the IncuCyte SX5 (Sartorius). Recombinant human IFN-α2a was used as control. Cells were cultivated with 90% RPMI 1640, 2 mM L-Glutamine, 1 mM Sodium Pyruvate, 10 mM HEPES, 4.5 g/L Glucose, 1.5 g/L NaHCO₃(PAN Biotech, Cat.No.P04-18047) supplemented with 10% Fetal Bovine Serum (Anprotec, Cat.No. AC-SM-0014Hi)) and Penicillin/Streptomycin (Roche, Cat.no 11074440001). 10,000 tumor cells were seeded in 50 μl cultivation medium in a flat-bottom 96-well plate (Corning, Cat.No. 3585). The IncuCyte Mouse IgG₁ Fabfluor-488 Antibody Labeling Dye (Sartorius, Cat.No. 4745) and the IncuCyte Mouse IgG₂a Fabfluor-594 Antibody Labeling Dye (Sartorius, Cat.No. BA-04863) were mixed with a molar ratio of 1:3 with a final concentration of 1 μg/ml Ultra-LEAF™ Purified anti-human CD274 (B7-H1, PD-L1, Isotype Mouse IgG1, Biolegend Clone M1H2, Cat.No. 393602) Antibody and Ultra-LEAF™ Purified anti-human HLA-A,B,C Antibody (Clone W6/32, Isotype Mouse IgG2a, Biolegend Cat.No. 311428) and incubated for 15 minutes to allow conjugation. 50 μl Opti-Green background suppressor (final conc. 0.5 μM) and 50 μl of PD-L1-IFN-α2 molecules (final conc. 10 nM) were added to the plate. The plate was transferred to the IncuCyte and green and red fluorescence was measured every 2 hours (10× objective). Data analysis was performed with the IncuCyte® Software 2022A (basic analysis, Spectral unmixing % Red contributes to Green value: 2.8%).

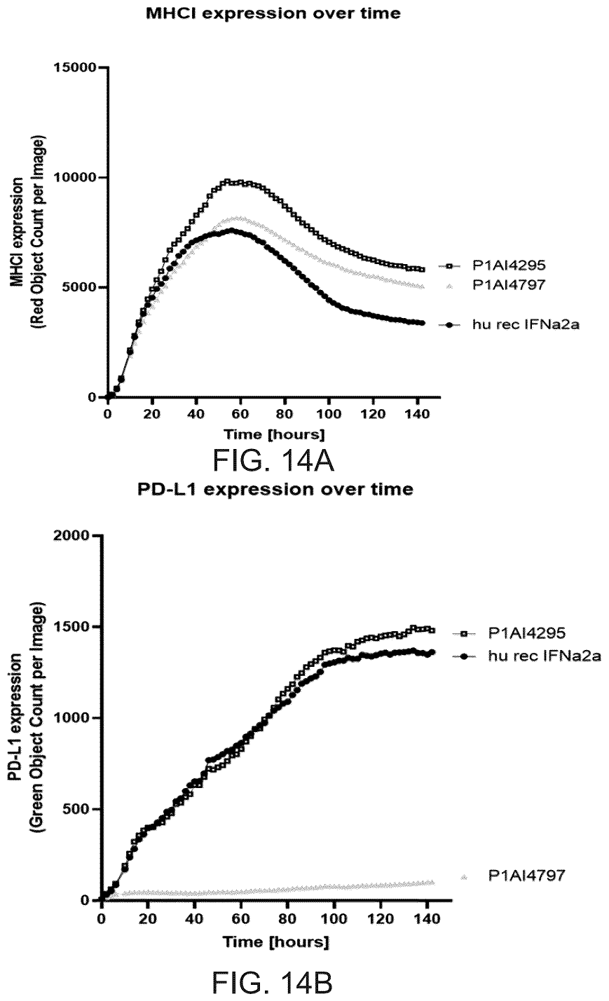

As can be seen from FIG. 14A, the PD-L1-targeted IFN-α2a Dutaflip fusion proteins induce MHCI expression of HCC1954 tumor cells to a comparable extent as recombinant human IFN-α2a, indicating increased tumor immune recognition and enhanced PD-L1-targeting. FIG. 14 B shows, that P1AI4295 is also able to induce PD-L1 expression. Since the PD-L1-targeting domain of the PD-L1-targeted IFN-α2a Dutaflip fusion protein P1AI4797 was derived from Atezolizumab, it blocked the binding of the PD-L1 staining antibody used in the assay. Thus, PD-L1 expression levels for this molecule were not detectable in this assay.

### Example 16: NK Cell Activation by PD-L1-Targeted IFN-α2a Dutaflip Fusion Proteins

NK-92 (ATCC No. CRL-2407) was cultivated in 80% MEM alpha w/o nucleosides (Gibco #12561-056), 12,5% FCS (Gibco #10500-064), 12.5% Horse Serum (Anprotech AC-SM-0059), 2 mM L-Glutamine (Anprotech #AN-182008), 0.1 mM β-Mercaptoethanol (Gibco #31350-010), 0.2 mM Myo-Inositol (Sigma #7508) and 0.02 mM Folic Acid (Sigma #8758). 100 U/ml IL-2 (Roche #11147528001) were added directly to the cell culture. 100,000 NK-92 cells per well (150 μl/well) were seeded in corresponding medium without IL-2 and 50 μl of respective fusion protein or recombinant human IFN-α2a (PBL Assay Science #11101-2, Lot #7191) dilutions (100 nM to 0.001 nM, 1:10 dilution) were added per well. After 48 hours of incubation, plates were centrifuged and supernatants for an IFN-y ELISA (Human IFN-γ DuoSet ELISA, R&D #DY285B and DuoSet Ancillary Reagent Kit2, R&D #DY008) collected. The IFN-γ ELISA was performed as described in the protocol provided by the manufacturer. Undiluted samples or standards (600 μg/ml diluted in Reagent Diluent 1:2 until 9.38 pg/ml) were added (100 μl per well), sealed and incubated for two hours at room temperature. Afterwards the supernatant was aspirated, wells were washed and detection antibody (200 ng per ml diluted in Reagent Diluent) was added to each well (100 μl per well), sealed and incubated for 2 hours at room temperature. After another aspiration and wash step, streptavidin-HRP (40-fold dilution in Reagent Diluent) was added to each well (100 μl per well) and incubated for 20 minutes at room temperature. The aspiration and wash step was then repeated. Substrate Solution (equal volumes of Color Reagent A and B) was added to each well (100 μl per well) and incubated for 20 minutes in the dark at room temperature. Finally the stop solution (50 μl per well) was added to each well and the optical density using a microplate reader (set to 450 nm, wavelength correction 570 nm) was determined.

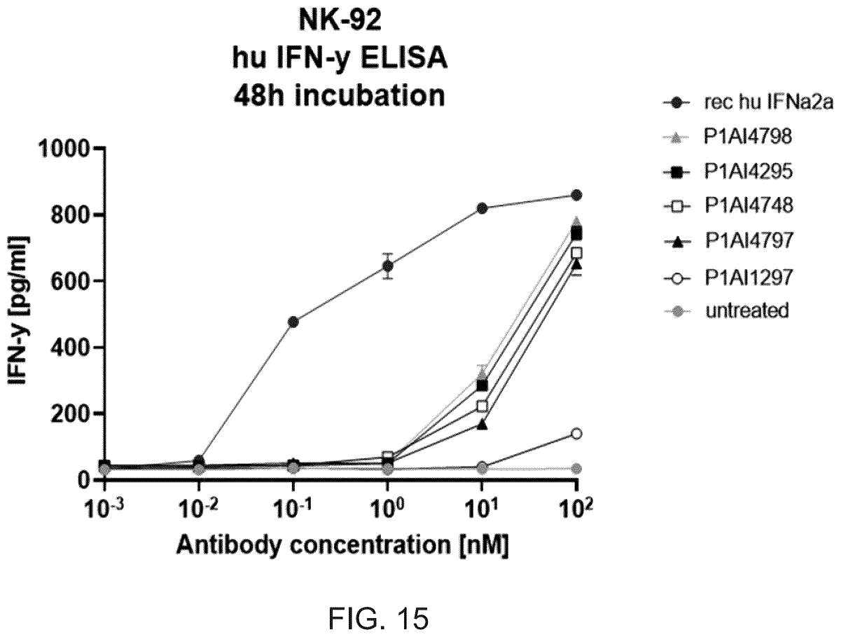

As shown in FIG. 15, the PD-L1-targeted IFN-α2a Dutaflip fusion proteins P1AI4797, P1AI4798, P1AI4748 and P1AI4295 induce lower IFNγ expression of human NK-92 cells which show a low PD-L1 expression of about 500 to 1000 PD-L1 molecules per cell, compared to recombinant IFN-α2, indicating lower periphery NK cell activation, indicating a potential improved safety profile.

### Example 17: PD-L1-Targeted IFN-α2a Stimulated Human Primary DC Subsets

4× 50 ml falcon tubes containing 1 ml sterile 1:2 dilution of Heparin (Ratiopharm #03029843) and 0.9% Sodium Chloride solution (Fresenius Kabi #0809078) were prepared. 200 ml blood from one Donor was washed with RPMI1640 (PAN #P04-17500) 1:2. The blood mix was filled into Lymphocyte Separating Pancoll tubes (PAN Biotech #P04-60125) and centrifuged (800 g, 15 min at room temperature (RT)) without brake. The plasma layer above the lymphocyte ring was aspirated and the ring was transferred into tubes containing 25 ml RPMI1640 (RT). After centrifugation (300 g, 10 min at RT) pellets were washed and resuspended for counting. The Isolation of dendritic cells (DCs) was performed as described in the protocol (human Pan-Dendritic Cell Enrichment Kit, Miltenyi #130-100-777) provided by the manufacturer. In brief, the cells were centrifuged (300 g, 10 min at 4° C.) and resuspended in MACS Buffer (350 μl/10⁸ cells). FcR Blocking Reagent (50 μl/10⁸ cells) and Pan DC Biotin-Antibody Cocktail (100 μl/10⁸ cells) were added for 5 min at RT. MACS buffer (400 μl/10⁸ cells) and Pan DC MicroBead-Antibody Cocktail (100 μl/10⁸ cells) were added for 5 min at RT. The cells were washed with MACS Buffer (10 ml/10⁸ cells), centrifuged and resuspended in MACS Buffer (500 μl/10⁸ cells). For the Magnetic separation LS columns (Miltenyi #130-042-401) were prepared by placing them into a magnet and rinsing with 3 ml MACS Buffer. After applying the cell suspension onto the column they were washed once with 3 ml MACS Buffer. Unlabeled cells (enriched dendritic cell fraction) that pass through were collected, centrifuged and resuspended for counting. Dendritic cells were seeded in 96-u-bottom-plates (Costar #3799) with 1×10⁵ cells/well in media (180 μl) containing RPMI 1640 (PAN #P04-17500), 10% FCS (Anprotech, Cat: AC-SM-0014Hi), 2 mM L-Glutamine (Sigma, CatNr: G7513), 0.1 mM 2-ME (Gibco #31350-010), 1 mM Na-Pyruvate (Anprotech Cat: AC-DS-0023), 1 mM MEM NEAA (PAN Cat #P08-32100), 1× Vitamine (PAN #P08-41100), 10 mM HEPES (Anprotech #AC-DS-0007), 100 μg/ml Pen/Strep (PAN #P06-07100-100 ml) and 20 μl of respective fusion proteins or 10 nM recombinant human IFN-α2a (PBL Assay Science #11101-2, Lot #7191) were added per well. After 24 hours incubation, plates were stained for FACS. All cells were transferred to 96-v-bottom-plates (Costar #3357) and centrifuged twice (5 min, 400 g, RT) by washing with 200 μl PBS (Anprotec #AC-BS-0002). Zombie UV dye (Biolegend #423107) was dissolved in PBS (1:400) and pellets were resuspended in 100 μl Zombie UV dye in PBS solution. After incubation (20 min, 4° C. in the dark) 150 μl FACS buffer containing PBS, 3% FCS (Gibco #10500-064) and 2 mM EDTA (Gibco #15575) was pipetted. Then cells were centrifuged, washed (1× 250 μl FACS-buffer), taken in FcBlock (BD #564220, c=0.5 mg/ml, final conc. 1:100 in FACS buffer, 50 μl/well, 2× Fc block) and incubated 10 min at RT. Anti-human extracellular stain was added (50 μl/well, 2× antibody) containing Linage Cocktail CD3,14,16,19,20,56-FITC (Biolegend #348801, clone UCHT1,HCD14,3G8,HIB19, 2H7,HCD56), CD141-BV605 (Biolegend #344118, clone M80), CD11c-BUV395 (BD Biosciences #748289, clone 3.9), CD1c-BV421 (Biolegend #331526, clone L161), CD123-BUV661 (BD Biosciences #741541, clone 7G3), CD123-BUV661 (BD Biosciences #741541, clone 7G3), CD303-APC (Biolegend #354206, clone 201A), PD-L1-PE/Cy7 (Biolegend #329718, clone 29E.2A3), CD86-BV786 (Biolegend #305442, clone IT2.2), CD80-BV711 (Biolegend #305236, clone 2D10) and CLEC9A-PE (Biolegend #353804, clone 8F9). All antibodies were diluted 1:100 in FACS buffer, except of CLEC9A-PE, which was diluted 1:50. Also corresponding isotypes (containing mouse IgG2bk-PD-L1 from Biolegend #400326 clone MPC-11-, mouse IgG2bk-CD86 from Biolegend #400356 clone MPC-11-, mouse IgG1k-CD80 from Biolegend #400168 clone MOPC-21-isotypes and the remaining markers) or FACS buffer for unstained sample were added, resuspended and incubated 20 min. at 4° C. in the dark. After that cells were washed 1× with FACS-buffer (150 μl/well), resuspended, centrifuged and collected in 200 μl FACS-buffer for measurement at FACS-Fortessa.

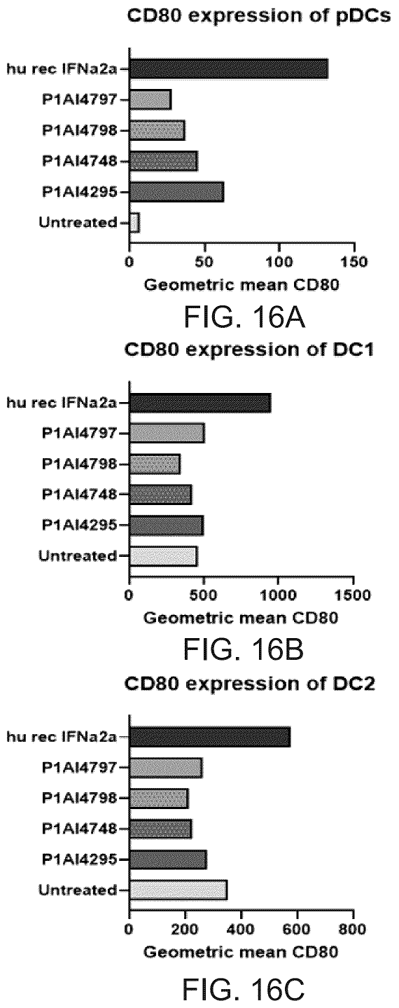

As can be seen from FIGS. 16A-16C, the PD-L1-targeted IFN-α2a Dutaflip fusion proteins P1AI4295, P1AI4798, P1AI4797 and P1AI4748 induce lower CD80 expression on human primary DC subsets compared to recombinant IFN-α2, indicating lower periphery DC activation, indicating improved safety.

### Example 18: PD-L1-Targeted IFN-α2a Fusion Protein Stimulated Human Primary T Cells

Human primary CD4 and CD8 T cells (characterized by low surface PD-L1 expression levels, 50-200 PD-L1 proteins per cells) were stimulated with PD-L1-targeted IFN-α2a Dutaflip fusion proteins or recombinant human IFN-α2a and CD69 expression of human primary CD4 and CD8 T cells was measured by flow cytometry. Therefore blood was obtained from blood donors via the Blutspendedienst of the medical service Penzberg in 50 ml tubes (Greiner Bio-One, Cat.No. 227261) containing 1 ml of 1:2 dilution of Heparin (ratiopharm, Cat.No. 03029843) and 0.9% Sodium Chloride solution (Fresenius Kabi, Cat.No. 0809078). In order to isolate peripheral blood mononuclear cells (PBMCs), the blood was diluted in the same volume of RPMI1640 (PAN Biotech, Cat. No. P04-17000) and 30 ml of the blood mix were carefully poured into Pancoll tubes (PAN Biotech, Cat.No. P04-60225). The tubes were centrifuged at 800 g for 15 minutes at room temperature with low acceleration and without break. Afterwards the PBMCs were collected from the interface, washed twice with RPMI1640 and resuspended in 30-50 mL of RPMI1640. The cells were counted using a Neubauer chamber and 1:10 dilution of Trypan blue 0.4% (Invitrogen, Cat.No. T10282). Subsequently, T-cells were isolated from the PBMCs with the Miltenyi Pan T cell isolation kit (Cat.No. 130-095-535) according to manufacturer's instructions and frozen with 90% FCS and 10% DMSO. After thawing, T-cells were seeded into a 96 well round-bottom plate and Dutaflip PD-L1-targeted IFN-α2a molecules were added at a concentration of 10 nM in 20 μl of RPMI1640 (PAN Biotech, Cat. No. P04-17000) medium containing 10% FCS (Gibco, Cat.No. 10500-064), 0.1 mM 2-Mercaptoethanol (Gibco, Cat.No. 31350-010), 2 mM L-Glutamine (Sigma, Cat.No. G7513)), 1 mM Na Pyruvate (PAN, Cat.No. P04-43100), 100 μg/ml PenStrep (PAN, Cat.No. P06-07100), MEM NEAA (Pan, Cat.No. P08-32100) and 10 mM HEPES (Anprotech, Cat.No. AC-Ds-0007). As positive control, rec. huIFN-α2a (PBL Assay Science, Cat.No. 11101-2) was used.

After 72 hours cells were washed once with FACS-Buffer (PBS containing 3% FCS, 2 mM EDTA) and incubated with 20 μl of 1:50 diluted Fc receptor blocking (BD Pharmingen, Cat.No. 564219) in FACS Buffer. After 15 minutes of incubation at 4° C., cells were washed with FACS buffer and 20 μl of a mixture of fluorescently labeled antibodies in FACS Buffer was added to the cells. The following fluorescently labeled antibodies were used: anti-human CD3 FITC (Biolegend, Cat. No. 100204), anti-human CD8 PE-Cy7 (Biolegend, Cat. No. 980910), anti-human CD4 BUV395 (BD Biosciences, Cat.No. 564724), anti-human CD69 APC (Biolegend, Cat. No. 310910). After 20 minutes of incubation at 4° C., cells were washed twice with FACS Buffer and then resuspended in 200 μl of FACS Buffer containing the viability dye DAPI to distinguish between live and dead cells.

Cells were analyzed the same day using 5-laser LSR-Fortessa (BD Bioscience with DIVA software). Data analysis was performed using the FlowJo version 10 software (FlowJo LLC). Live (DAPI negative) cells, positive for CD3 and positive for CD4 or CD8 cells were analyzed for CD69 expression.

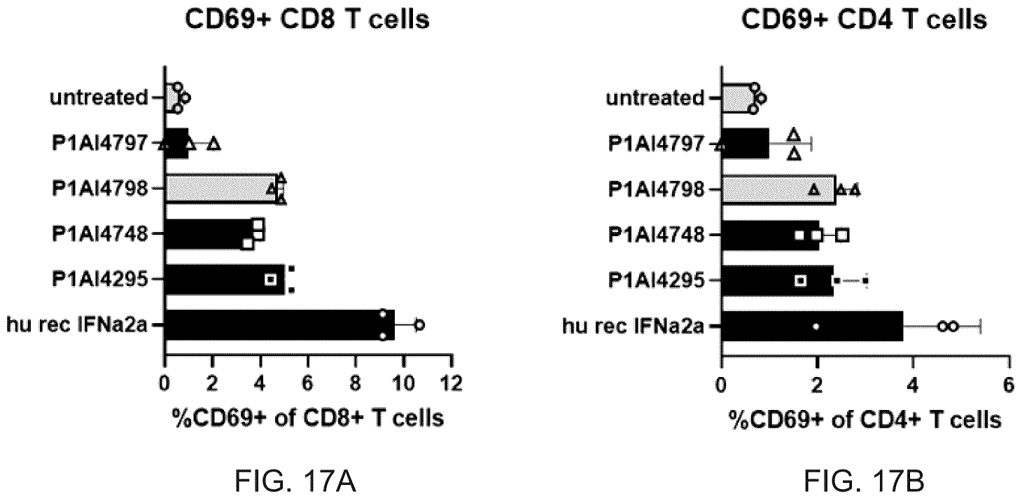

As can be seen from FIG. 17A-17B, the Dutaflip PD-L1-targeted IFN-α2a molecules P1AI4295, P1AI4798, P1AI4797 and P1AI4748 induce lower levels of CD69 expression on naive CD4+ (FIG. 17A) and CD8+ (FIG. 17B) T cells compared to recombinant IFN-α2a, indicating lower periphery T cell activation and thus a potential improved safety profile.

### Example 19: PD-L1-Targeted IFN-α2a Whole Blood Cytokine Assay

Fresh human whole blood (which has a naturally low PD-L1 expression) was incubated with indicated PD-L1-targeted IFN-α2a Dutaflip fusion proteins at a concentration of 4 nM for 24 h, and IL6 and CXCL10 levels were measured. This was done by using the enzyme-linked immunosorbent assay (ELISA).

Blood was obtained from human donors via the Blutspendedienst of the medical service Penzberg. 100 μl blood was transferred into a 96 well round-bottom plate containing 80 μl RPMI1640 (PAN Biotech, Cat. No. P04-17000) medium supplemented with 10% FCS (Gibco, Cat.No. 10500-064), 0.1 mM 2-Mercaptoethanol (Gibco, Cat.No. 31350-010), 2 mM L-Glutamine (Sigma, Cat.No. G7513)), 1 mM Pyruvate (PAN, Cat.No. P04-43100), 100 μg/ml PenStrep (PAN, Cat.No. P06-07100), MEM NEAA (Pan, Cat.No. P08-32100) and 10 mM HEPES (Anprotech, Cat.No. AC-Ds-0007) per well. PD-L1-targeted IFN-α2a Dutaflip fusion proteins were added at a concentration of 4 nM in 20 μl of RPMI1640 with supplements. As positive control, rec. huIFN-α2a (PBL Assay Science, Cat.No. 11101-2) was used. After 24 hours, plates were centrifuged and supernatants were used for the CXCL10 or IL-6 ELISA (R&D, Cat.No.DY266, Cat.No.DY206, Cat.No. DY008). Supernatant for the CXCL10 ELISA was diluted 1:10, for the IL-6 ELISA 1:2. The ELISA was performed as described in the manufacturer's protocol. The optical density was determined using a microplate reader (set to 450 nm, wavelength correction 570 nm).

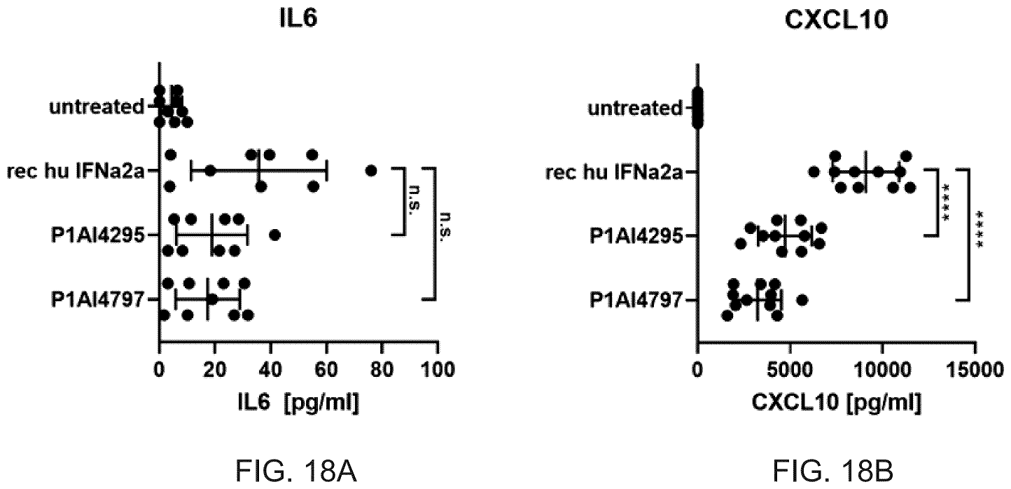

As can be seen from FIG. 18A-18B, the PD-L1-targeted IFN-α2a Dutaflip fusion proteins P1AI4295 and P1AI4797 induce lower CXCL10 (FIG. 18A) and IL6 (FIG. 18B) compared to recombinant IFN-α2 in vitro in human whole blood, potentially indicating lower periphery activity and thus improved safety.

### Example 20: PD-L1-Targeted IFN-α2a Internalization Assay

Internalization of PD-L1-targeted IFN-α2 Dutaflip fusion proteins into the PD-L1 expressing human tumor cell line HCC1954 was measured using the live cell imaging of the IncuCyte SX5 (Sartorius). Cells were cultivated with 90% RPMI 1640, 2 mM L-Glutamine, 1 mM Sodium Pyruvate, 10 mM HEPES, 4.5 g/L Glucose, 1.5 g/L NaHCO₃(PAN Biotech, Cat.No.P04-18047) supplemented with 10% Fetal Bovine Serum (Anprotec, Cat.No. AC-SM-0014Hi)) and Penicillin/Streptomycin (Roche, Cat.no 11074440001). 15,000 tumor cells were seeded in 200 μl cultivation medium in a flat-bottom 96-well plate (Corning, Cat.No. 3585) and incubated overnight at 37° C. to adhere. PD-L1-targeted IFN-α2 antibodies and the IncuCyte Human/Mouse Fabfluor-pH Red Antibody Labeling Reagent (Sartorius, Cat.No. 4722/4750) were mixed at a molar ratio of 1:3 in media and incubated for 15 min. at 37° C. to allow conjugation. The supernatant of the cells were removed and 100 μl of the Antibody-Fabfluor Mix was added. The plate was transferred to the IncuCyte and imaged every 2 hours (10× objective). Data analysis was performed with the IncuCyte® Software 2022A (Basic analysis, Red object count per Image).

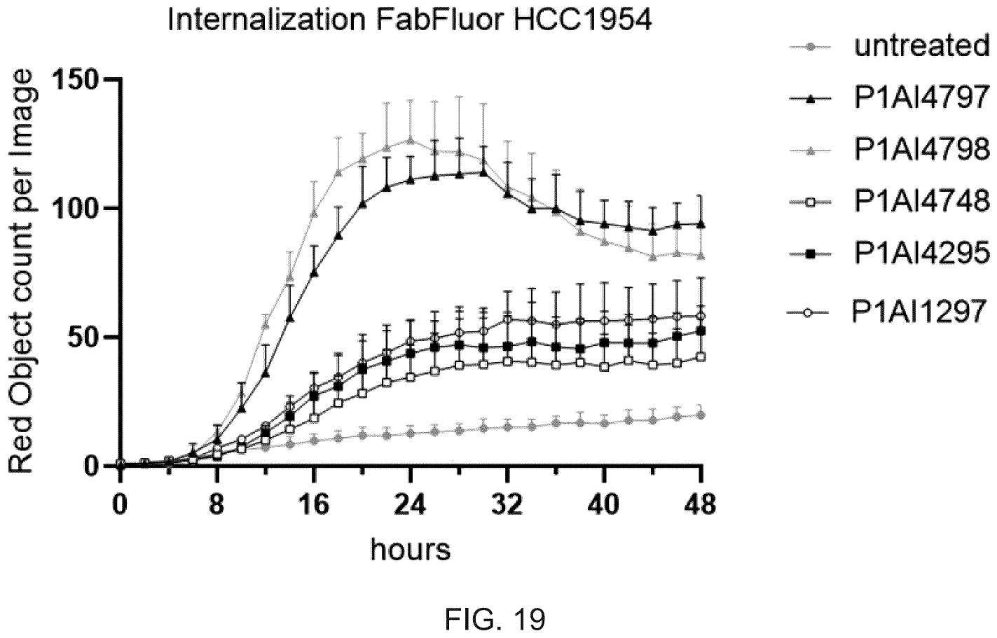

As can be seen from FIG. 19, the PD-L1 (M14)-Dutaflip fusion proteins P1AI4748 and P1AI4295 show the lowest HCC1954 internalization rate over time compared to the other Dutaflip PD-L1-targeted IFN-α2a molecules, leading to potentially lower clearance levels (CL) in vivo.

### Example 21: Efficacy of PD-L1-Targeted IFN-α2a Dutaflip Fusion Proteins in Tumor-Bearing Hu NSG Mice

The anti-tumor activity of PD-L1-targeted IFN-α2a Dutaflip fusion proteins P1AI4295 and P1AI4798 was assessed in vivo in MDA-MB-231 tumor-bearing humanized NSG mice (i.e. immunodeficient mice humanized by engraftment with human hematopoietic stem cells (“HSC”)) engrafted with the human breast cancer cell line MDA-MB-231, injected into the intramammary fat pad. The efficacy of this treatment was compared to treatment with Pegasys (Pegylated interferon alfa-2a) as a control. Vehicle was used as negative control.

For the study, MDA-MB-231 human breast carcinoma cell lines were obtained from the American Type Culture Collection (Manassas, VA) and maintained according to the supplier's instructions. Cells were harvested at the exponential phase of growth for injection into the mammary fat pads of mice. Production of fully humanized mice: Female 3-week old NSG (NOD/scid/IL-2Rγnull) mice were irradiated (140 cGy) and engrafted by intravenous injection of 9×104 CD34+ cord blood cells per mouse at Jackson Laboratories. After reaching a human immune infiltrate (hCD45) above 25% in blood, mice were shipped to Roche and maintained for 5 days to get accustomed to the new environment. Mice were kept under specific-pathogen-free condition with daily cycles of 12 hours light/12 hours darkness according to committed guidelines (GV-Solas; Felasa; TierschG). Continuous health monitoring was carried out on a daily basis. Experimental study protocol was reviewed and approved by local government (ROB-55.2-2532.Vet_03-20-170).

Efficacy Experiment: Humanized mice were injected with 2×10⁶ MDA-MB-231 cells in a total volume of 20 μL PBS into the mammary fat pad (i.m.f.p.). Once tumors reached an average volume of approximately 200 mm³, mice were randomized into different treatment groups based on tumor volume. The first group of mice received histidine buffer (vehicle) as control. All antibodies were prepared freshly in 20 mM Histidine, 140 mM NaCl, pH 6.0 before injection and administered intravenously (IV) biweekly, 5 applications in total, at equimolar doses according to molecular weight (P1AI4295: 1.24 mg/kg; P1AI4798: 1.25 mg/kg; Pegasys (recombinant IFN-α2a): 0.46 mg/kg). Animals were controlled daily for clinical symptoms and detection of adverse effects. Tumor volume was measured by caliper and body weight was controlled twice weekly. Termination criteria for animals were visible sickness (scruffy fur, arched back, breathing problems, impaired locomotion), body weight loss (≥25% within 7 days after first treatment or ≥20% after 7 days of treatment) or tumor size (diameter ≥2 cm, volume ≥4000 mm³). Animals were sacrificed according to the termination criteria or at the end of the experiment. The impact of the therapy was assessed by measuring the tumor size and displayed as median tumor growth over time. Animals with tumors only of a tumor volume of less than 50 mm³ were considered as tumor-free animals. Graphical analysis was performed using GraphPad Prism v6.07 (GraphPad Software, Inc).

Treatment with PD-L1-targeted IFN-α2a Dutaflip fusion proteins P1AI4295 and P1AI4798 showed an anti-tumor response in MB-231 tumor-bearing huNSG animals, as demonstrated by a decrease in tumor volume (FIG. 20). The response in animals treated with P1AI4798 was in a similar range as treatment with Pegasys, while P1AI4295 showed the superior therapeutic efficacy of all tested molecules, compared to P1AI4798 and recombinant IFN-α2a (Pegasys). Notably, six out of 10 treated mice treated with P1AI4295 showed a complete response, while in none of the other treatment arms a complete response was observed.

### Example 22: Pharmacokinetic Analysis of PD-L1-Targeted IFN-α2a Fusion Proteins in huPD1/huPD-L1 Transgenic Mice

In order to investigate the PK of molecules according to the invention, 9 weeks old C57B16N mice genetically modified to expression recombinant human PD1 and human PD-L1 (and knocked-out for murine PD-1 and murine PD-L1) are dosed with either one of two PD-L1-targeted IFN-α2a fusion proteins (P1AI4295, P1A14798), a reference molecule or atezolizumab (control), respectively (single dose, 5 mice per tested molecule), and followed up for 7 days to collect blood samples (6 samples from each mouse in total).

Briefly, compounds are injected once intravenously into the tail vein on Day 0 to anesthetized mice at a dose of 5.0 mg/kg. Body weights of mice are measured and volumes for injections are defined accordingly. The tested molecules are diluted in sterile filtered 20 mM Histidine, 140 mM NaCl pH 6.0 for injections. The blood of each mouse is sampled at 7 days before compound injection, and at 30 min, 6h, 24 h, 72h and 168 h (terminal) after compound injection. Each blood sample is limited to 20 to 25 μL sampling except for the terminal one at 168h post-injection. The time point at 168h is the terminal sampling and mice are sacrificed after blood collection. Sera are prepared for all blood samples by incubating tubes with blood samples 1 to 4 hours at room temperature to allow for coagulation. Then they are centrifuged 5 minutes at 1000g at 20° C. to separate serum. If needed a 2nd identical centrifugation is done to improve the collected serum volume. Then serum is sampled using a micropipette. The exact collected serum volume is determined using a micropipette. The serum samples are frozen and stored in cryoboxes at −80° C.

### Bioanalytical Method

Serum samples of drug-treated mice treated with are analyzed with a electrochemiluminescence immunoassay (ECLIA) method on the cobas e 411 analyzer (Roche Diagnostics GmbH) under non-GLP conditions using antibodies specifically recognizing the CH2 domain of human IgG. Briefly, test samples, the biotinylated first detection antibody, the ruthenylated second detection antibody and streptavidin-coupled beads (Roche Diagnostics GmbH) are added stepwise to a detection vessel and incubated for 9 minutes at each step. Finally, the streptavidin bead bound complex is detected by a measuring cell that numbers the counts of streptavidin beads in the assay. The counts are proportional to the analyte concentration in the test sample.

### Example 23: Evaluation of Blocking of PD1/PD-L1 Interaction by PD-L1-Targeted IFN-α2a Fusion Proteins in Different Formats

The PD1/PD-L1 blockade bioassay (Promega) was used to evaluate whether blocking of PD1/PD-L1 interaction by an anti-PD-L1 targeting antibody used in a PD-L1-targeted IFN-α2a fusion protein was dependent on the molecule format used. The assay was essentially performed as described in Example 7 e). Addition of either an anti-PD-1 or an anti-PD-L1 antibody that blocks the PD-1/PD-L1 interaction between PD-L1 aAPC/CHO-K1 cells and PD-1 effector cells releases the inhibitory signal and results in TCR activation of PD-1 effector cells followed by NFAT-RE-mediated luciferase expression. Luciferase activity can be detected and quantified using the Bio-Glo™ Luciferase Assay System.

In this assay, PD-L1-targeted IFN-α2a molecules in different formats and/or having different Dutaflip arms were tested for blocking of PD1/PD-L1 interaction (see Table 17). Atezolizumab, which is known to block PD1/PD-L1 interaction, was used as a positive control. Inhibition of PD-1/PD-L1 interaction by PD-L1-targeted IFN-α2a fusion proteins is indicated by the induction of luminescence signal compared to untreated cells (e.g. a fold change of four corresponds to a four-times induction of luminescence due to PD-L1 blockade).

TABLE 17
Overview of different molecules used for blockade assay

| Molecule ID | Format as shown in | Targeting arm |
|---|---|---|
| P1AI4295 | FIG. 5 A | M14 |
| P1AI4748 | FIG. 5 A | M14 |
| P1AI4798 | FIG. 5 A | Atezolizumab |
| P1AI4797 | FIG. 5 A | Atezolizumab |
| P1AI4853 | FIG. 5 F | Atezolizumab |
| P1AI4848 | FIG. 5 E | Atezolizumab |
| P1AH1108 | FIG. 5 A | Atezolizumab |

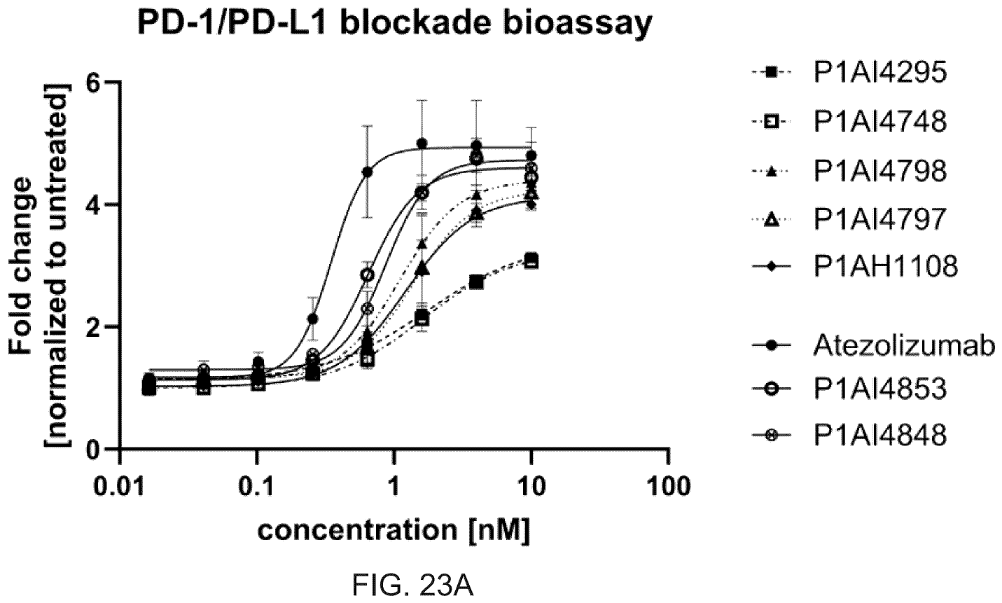

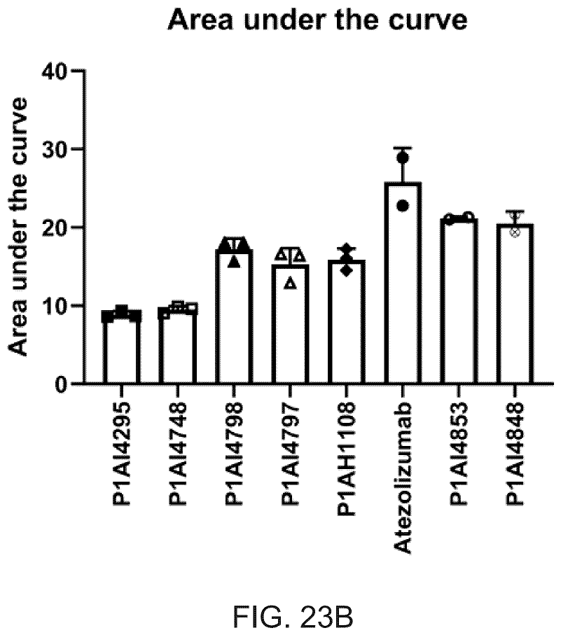

As shown in FIG. 23A, the effect of blocking of PD1/PD-L1 interaction which was shown for the M14HH17L (parental)-derived anti-PD-L1 Fabs in Example 7 e was also observed in the PD-L1-targeted IFN-α2a fusion proteins containing these anti-PD-L1 Fabs as targeting arms. As illustrated in FIG. 23A, the PD-L1 (M14)-Dutaflip fusion proteins P1AI4748 and P1AI4295 demonstrate the lowest PD-L1/PD-1 blocking efficacy. Notable blockade occurs only at elevated concentrations, and even then, the plateau reached is significantly lower than that of Atezolizumab. The limited PD-1/PD-L1 blockade observed with P1AI4748 and P1AI4295 is likely due to steric hindrance, since the anti-PD-L1-targeting binder M14 binds to a different epitope on PD-L1 than PD-1 does, as described above. FIG. 23 B shows the AUC values calculated from these curves in FIG. 23A for all molecules.

### Example 24: Analysis of PD-L1-Targeted IFN-α2a Induced Response in Human Tumor Tissue Samples

### a) Description of Assay

In order to investigate the PD-L1 target-specific mode of action (MOA) of the PD-L1-targeted IFN-α2a Dutaflip fusion protein in the most translational-relevant human tumor model for cancer immunotherapy, fresh human patient-derived tumor explants (PTE) were cultivated ex vivo with and without treatment for 18-24 h in a dynamic flow 3D cultivation system. Various functional readouts post-cultivation (including cytokine and single cell RNA Sequencing data) were compared to proteomic and transcriptomic baseline characteristics in order to analyze and study the target specific MOA on a single cell level.

### b) Functional Analysis

For this study, “fresh” (received within <24 h post resection) PTEs (e.g. NSCLC, BLCA, RCC, TNBC) were sourced from an external vendor (Fidelis) and transported as fast as possible, at 4° C. in MACS tissue storage solution (Miltenyi; 130-100-008), by a DHL or UPS to the lab in Penzberg. PTEs were manually carved into small fragments (2×2 mm²), randomly grouped in 3 fragments per condition, and cultivated for 18-24 h with the PD-L1-targeted IFN-α2a Dutaflip molecule (P1AI4295, Concept: FV023813_FV024473_h-IFNA2(24-188). Human Interferon Alpha 2a (PBL Assay Science; 11101-2) was used as a positive control. An untreated sample, cultivated in pure 3D cultivation medium [500 mL Advanced RPMI 1640 Medium (Thermo Fisher Scientific; 12633020); 1× Gibco GlutaMAX™ Supplement (Thermo Fisher Scientific; 35050-061); 1× Pen/Strep (Pan Biotech; P06-07100); 1×MEM NEAA (Pan Biotech; P08-32100); 0.5 mM Sodium Pyruvate (Anprotec; AC-DS-0023); 10 nM HEPES buffer (Anprotec; AC-DS-0007); 5% Human serum (TCS biosciences; CS100-500); 20 ng/mL EGF (Sigma Aldrich; SRP3027-500UG); 10 ng/mL FGF (Sigma Aldrich; F0291-25 μg)], was used as a negative control.

For the cultivation, the dynamic flow 3D cultivation system MIVOR Single-Organ Platform (REACT4LIFE S.p.A.) was utilized and assembled according to customer instruction. In brief, inserts were coated with 100 μL of 3% matrigel (VWR, 356231/734-1101) in 3D cultivation medium for 1 h at 37° C. and subsequently placed in the MIVO cups (REACT4LIFE S.p.A., M0001). In total, 3 PTE fragments were placed in a matrigel-coated insert (REACT4LIFE S.p.A., A0030) and transferred to a MIVO cup. The MIVO cups were filled with 2 mL medium (+/−compound) while an additional 0.5 mL of the same solution (medium+/−compound) was added to the matrigel-coated insert. The MIVO cups were closed with a lid, transferred to the incubator (37° C., 5% CO₂) and the tubes (REACT4LIFE S.p.A., A0014) connected to the syringe pump (Harvard Apparatus, D-403385) [pump settings: 1) Infusion 0.5 ml/min; 2) Delay 15 sec; 3) Withdrawal 0.1 ml/min; 4) Delay 15 sec; 5) Infusion 0.1 ml/min; 6) go to step 2].

After an overnight incubation period of 18-24 h the assay was stopped and the material was harvested for further analysis by demounting the 3D cultivation system. The supernatant was collected in a 15 mL Falcon tube (Corning, 352096), centrifuged, distributed as 200 μL per sample in a 96-well U-bottom plate (Corning, 3799) and frozen at −80° C. for long term storage.

In order to investigate the treatment-induced effects of the PD-L1-targeted IFN-α2a Dutaflip molecule, the supernatants of treated and untreated samples were assayed by BD™ Cytometric Bead Array (CBA) and ELISA to examine changes in cytokine secretion, providing insights into the immunomodulatory effects of the compound. In brief, all cytokines [Human IL6 Flex Set (BD, 558276); Human MIP1a Flex Set (BD, 558325); Human Granzyme B Flex Set (BD, 560304); Human IP10 Flex Set (BD, 558280); Human IFN-γ Enhanced Sensitivity Flex Set (BD, 561515)], released into the supernatants were detected using the CBA Soluble Protein Master Buffer Kit (BD, 558265) or Human Enhanced Sensitivity Master Buffer Kit (BD, 561521), according to manufacturer's instructions on a flow cytometer (BD Celesta). The half-maximal effective concentration (EC50) values were calculated using FCAP Array Infinite (BD) and the results were displayed using Prism 10 (GraphPad).

In addition, BLC/CXCL13 Human (Thermo Fisher Scientific, EHCXCL13X5) and ISG15 Human (Fine Test, EH1673) were assayed by ELISA (dilutions: ISG15 1:5, CXCL13 undiluted) according to the manufacturer's instructions. The absorbance at 450 nm was read on a Tecan Infinite 200 Pro M Plex microplate reader (Tecan, 30213614). EC50 values were calculated using Excel 2016 and the results were displayed using Prism 10 (GraphPad).

### c) Single Cell Nucleus RNA Sequencing Analysis

With the intent to study transcriptional changes on single cell level, the remaining fragments, post-cultivation, were chopped into small fragments and fixated overnight in accordance with the Tissue Fixation & Dissociation Protocol for Chromium Fixed RNA Profiling (CG000553|Rev B). The generation of libraries was conducted while utilizing the Chromium Fixed RNA Profiling Reagent Kits (User Guide CG000477 Rev C, 10× Genomics). The quality of indexed libraries was checked with the Agilent bioanalyzer (Agilent Technologies; 5067-4626), and libraries were pooled in an equimolar fashion and sequenced on a NovaSeq6000 (Illumina). The Cell Ranger mkfastq function pipeline was used to convert the output files into FASTQ files.

For pre-processing and quality control, raw sequencing reads were de-multiplexed and mapped to the GRCh38 genome using the Cell Ranger Single Cell software (10× Genomics). Raw gene expression matrices generated per sample were merged and analyzed with the Scanpy package (Wolf, F. et al. SCANPY: large-scale single-cell gene expression data analysis. Genome Biol 19, 15 (2018). https://doi.org/10.1186/s13059-017-1382-0). First, low quality cells and potential multiplets were excluded (minimum 200 genes, maximum 8,000 genes), resulting in ˜4,000 to 8,000 cells per sample passing quality control for downstream analysis. Filtered cells were normalized by log-transformed UMI counts per 10,000 reads [log (CP10K+1)]. After scaling the gene expression, the most variable genes per sample were calculated (minimum mean expression of 0.0125, maximum mean expression of 3 and minimum dispersion of 0.5), and were used for principal component analysis. Finally, the first 40 PCs were used as input for calculating the 10 nearest neighbors and the neighborhood graph was then embedded into the two-dimensional space using the uniform manifold approximation and projection (UMAP) algorithm⁷⁷). Cell clustering was performed using the Leiden algorithm at a resolution of 1⁷⁸.

Cell type annotation was performed based on cell type marker genes for T cells, B cells, mast cells, plasma cells, myeloid cells, endothelial cells, fibroblasts, pneumocytes and the other cells. The cells that did not express the marker genes were annotated as tumor cells.

### d) Baseline Characterization

In parallel to the 3D cultivation, small proportions of the fresh tumor were used for baseline characterization by histopathological analysis, bulk RNA Sequencing and flow cytometry (described in the following).

For histopathological analysis, a single tumor fragment (2×2 mm²) was processed as described above including 4% Formaldehyde fixation and dehydration in 70% EtOH. The FFPE embedding as well as the staining with the following antibodies: CD3 (clone SP7, DBS, RMAB005); CD8-GrB (clone SP16, Cell Marque, 108R-16; clone 11F1, Novocastra, NCL-L-GRAN-B); CD8-KI67 (clone SP16, Cell Marque, 108R-16; clone SP6, Cell Marque, 275R-16), was conducted by Sophistolab AG.

For bulk RNA Sequencing, one fresh tumor fragment (2×2 mm²) was snap frozen in liquid nitrogen and sequenced at GENEWIZ Germany GmbH. In brief, mRNA was enriched by rRNA depletion workflow (NEBNext® rRNA Depletion Kit (Human/Mouse/Rat), NEB, E7400). Samples were sequenced using paired-end reads, with sequencing coverage 30 million reads per sample using the NovaSeq 6000 or NovaSeq X plus sequencers.

In order to perform flow cytometry baseline characterization, PTE fragments were manually dissociated (scalpel) and digested [MACS Tissue Storage Solution (Miltenyi, 130-130-263); Accutase (Pan Biotech; P10-21100); Bovine serum albumin (Sigma-Aldrich, A9576); collagenase IV (Worthington, #LS004188); DNase I Type 4 (Sigma-Aldrich, D5025); Hyaluronidase (Sigma-Aldrich, H6254)] for 20-30 min at 37° C. on a thermo shaker (Thermo Scientific, SHKE4450). The single cell suspension was subsequently strained on a 70 μm cell strainer and washed twice with 30 mL and 10 mL cold RPMI media, respectively (15 min, 300×g at 4° C.). The staining was performed in a 96-well V-bottom plate (Corning, 3357). 1 Mio cells/well were stained, and 0.5 Mio cells/well were used for the Isotype control. Cells were washed with PBS (Gibco, 14190-136) and incubated with Zombie UV live/dead stain (Biolegend, 423108) for 10 min in the dark. Samples were washed with PBS and subsequently with FACS buffer [500 mL PBS; 3% FCS (Anprotec, AC-SM-0014Hi); 2 mM EDTA (Sigma, D2650)]. Human TruStain FcX Blocking Solutions (Biolegend, 422302) was added to the samples and incubated for 10 min in the dark. Subsequently 25 μL of antibody mix [HLA-DR BUV395 1:200 (clone G46-6, BD, 564040); CD45 BUV805 1:200 (clone HI30, BD, 612891); CD11b BUV661 1:50 (clone MI/70, BD, 565080); EpCAM BV421 1:50 (clone 9C4, Biolegend, 324220); CD3 BV510 1:200 (clone UCHT-1, BD, 563109); CD56 BV510 1:50 (clone NCAM16.2, BD, 563041); NKp46 BV510 1:50 (clone 9E2/NKp46, BD, 564064); CD19 BV605 1:50 (clone SJ25C1, BD, 562653); CD20 BV605 1:50 (clone L27, BD, 740333); LTBR BV650 1:50 (clone hTNFR-RP-M12, BD, 745342); CD14 BV711 1:100 (clone MφP9, BD, 563372); PD1 BV785 1:50 (clone EH12.2H7, Biolegend, 329929); PD-L1 PE 1:50 (clone 29E.2A3, Biolegend, 329706)], diluted in FACS buffer, was added to the cells and incubated for 15 min in the dark. Cells were washed twice with FACS buffer (centrifugation 300× g at 4° C.) and resuspended in 100 μL FACS buffer for acquisition.

### c) Results

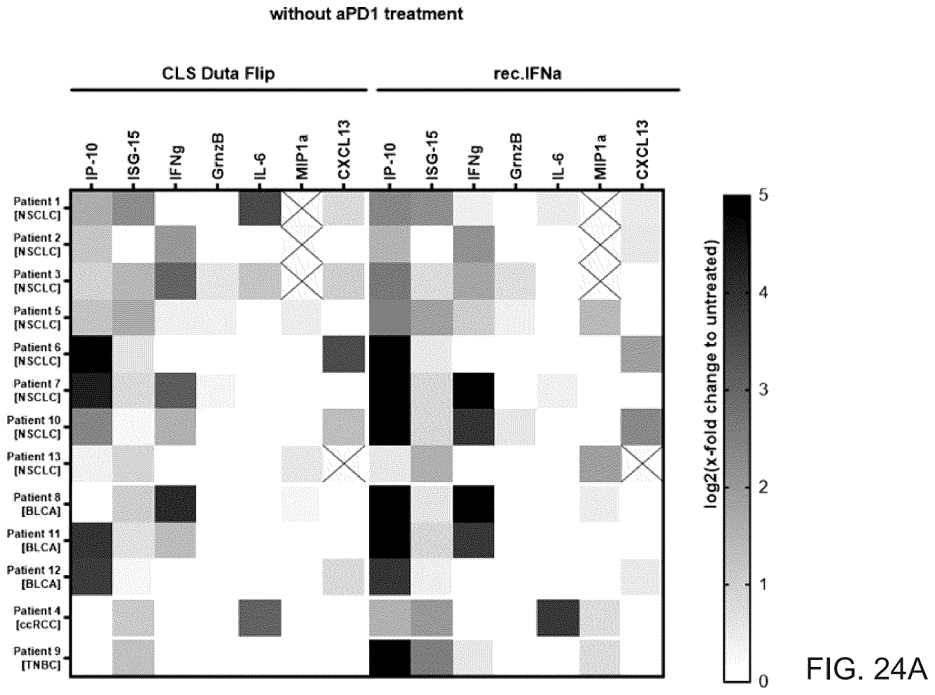

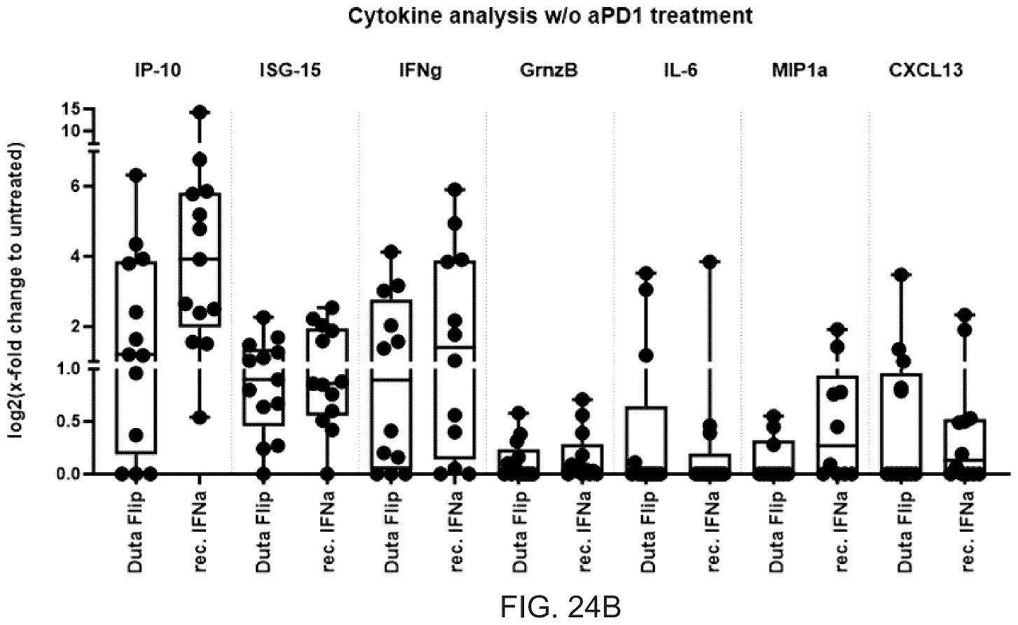

As can be seen from FIG. 24A and FIG. 24 B, the PD-L1-targeted IFN-α2a Dutaflip fusion proteins (“Duta Flip”) elicit a consistent cytokine response (as defined by log 2 x-fold change compared to untreated) for IP-10, ISG-15, and IFNγ across all NSCLC patients. This response is also observed in BLCA patients to a lesser degree, but not in ccRCC and TNBC samples (as of the current status, the study cohort with only one sample per indication for these two indications is not conclusive). The observed responses are comparable to those induced by the positive control, recombinant human IFN-α2a (“rec. IFN-α”), albeit to a slightly lesser extent (FIG. 24 B).

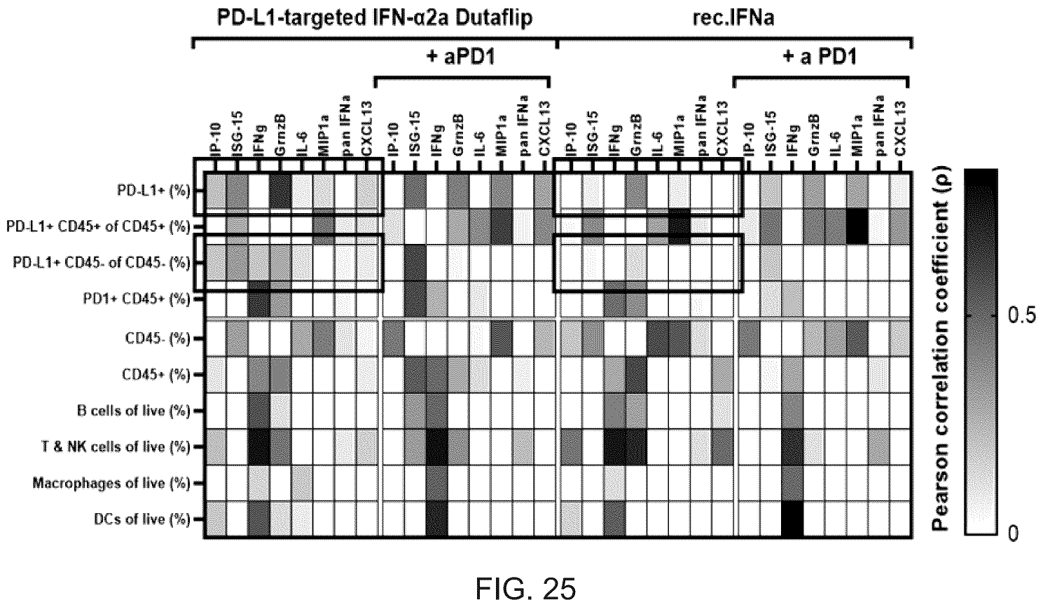

To investigate the target specificity of the PD-L1-targeted IFN-α2a Dutaflip fusion protein (“Duta Flip”), we conducted a Pearson correlation analysis to analyze strength and direction of the linear relationship between two continuous variables: e.g. the functional responses, as determined by CBA and ELISA, and the various variables assessed during baseline characterization via flow cytometry on data originating from all patients (n=13). Our investigation revealed a trend towards a positive correlation between both the frequency of PD-L1 positive cells and PD-L1 positive CD45 negative cells of all samples, and elevated cytokine response levels for IP-10, ISG-15, IFNγ, GrnzB, IL-6, MIP-1, pan IFNα, CXCL13 (highlighted by black boxes, on the left side) in samples treated with the PD-L1-targeted IFN-α2a Dutaflip fusion protein (see FIG. 25). However, this PD-L1 specific correlation was not observed for the same set of cytokines in the positive control group (highlighted by black boxes, on the right side), where recombinant human Interferon Alpha 2a (“rec.IFNa”) was utilized as a “target-independent” treatment. panIFNα response levels were also measured, but since differentiation between endogenous IFNα and added IFNα is not possible in this setup, the results were not taken into consideration.

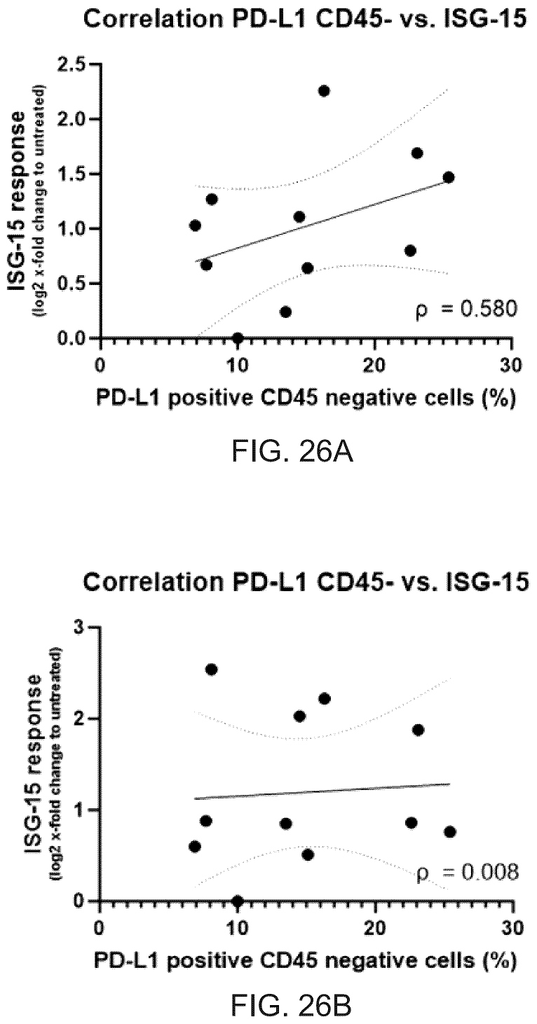

In order to enhance comprehension of the correlation visualization depicted in the heat map (FIG. 25), we deliberately chose a specific instance to illustrate the correlation between two continuous variables: the frequency of PD-L1 expressing CD45 negative cells and the ISG-15 response within the study cohort (n=13 patients). The P1AI4295-treated (“PD-L1-targeted IFN-α2a Dutaflip”) condition is shown in FIG. 26A), the condition treated with recombinant human Interferon Alpha 2a (“rec.IFNa”) is shown in FIG. 26 B. A distinct positive trend is evident by a Pearson coefficient of ρ=0.580, as represented in the heat map (FIG. 25) by an escalation of the gray scale (where white denotes no or a negative-correlation (not covered in this visualization) and black indicates a strong positive correlation). There is no correlation seen (Pearson coefficient of ρ=0.008) for the frequency of PD-L1 expressing CD45 negative cells and the ISG-15 response in the sample treated with the recombinant human Interferon Alpha 2a (FIG. 26 B).

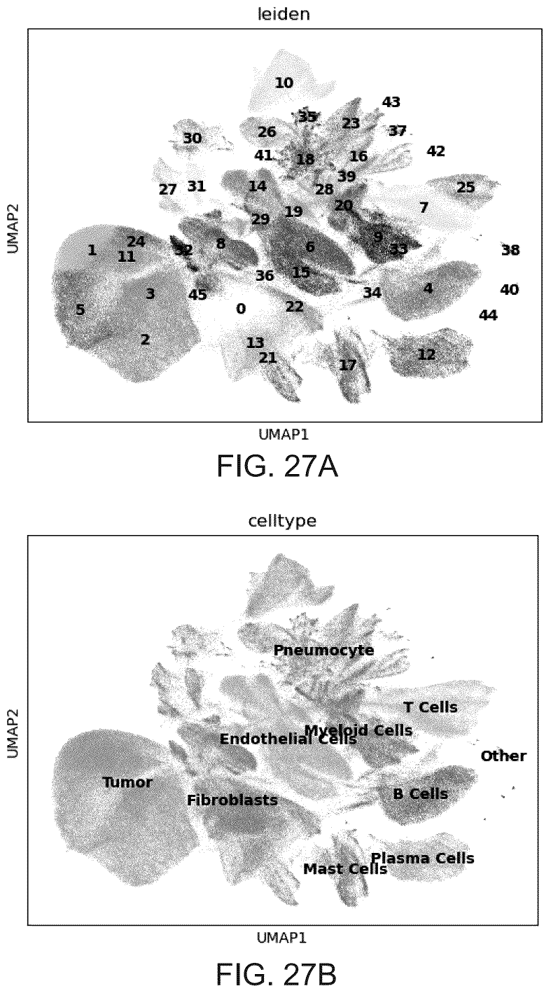

Following up the observed trend of a preferential PD-L1 target specific activation of the non-immune population (tumor cells), we performed a UMAP analysis on the snRNA-seq data obtained from the present samples post 3D cultivation and treatment (negative control; P1AI4295; positive control). UMAP visualization revealed 44 clusters representing different cellular subpopulations, based on 351,005 cells derived from 39 samples originating from 7 patients (FIG. 27A). To characterize the cellular composition of the sample, we analyzed the expression of known marker genes within each UMAP cluster (FIG. 27 B). Our analysis identified all major cell types of immune and non-immune cells, including tumor cells, T cells, myeloid cells, and further enabled us to qualify and quantify the PD-L1 expression per cell & cell population.

With a particular emphasis on the target specific activation of PD-L1-positive non-immune cells (“Tumor,” FIG. 27 B), we aimed to investigate an IFNα-targeted cis-activation pattern by examining an inflammatory gene signature [ISG15, MX1, XAF1, HERC5, STAT1, IFIT1, IF16, CXCL11, ISG20, IL10] in PD-L1-positive and -negative tumor cells (FIG. 28). In the untreated conditions, we did not observe any activation signals. However, in the positive control group treated with the recombinant IFN-α2a, both PD-L1-negative and PD-L1-positive cells demonstrated heightened frequencies of positive cells (indicated by the size of the bubble) and notable upregulation in the mean expression levels of the respective markers (indicated by the escalation of the gray scale). Particularly noteworthy, in P1AI4295-treated conditions (PD-L1-targeted), there was evidence of preferential target-specific activation of cells. Specifically, only PD-L1-expressing cells showed an IFN-α2a-induced activation, defined by an increase in the frequency of positive cells and mean expression levels of the inflammatory gene signature.

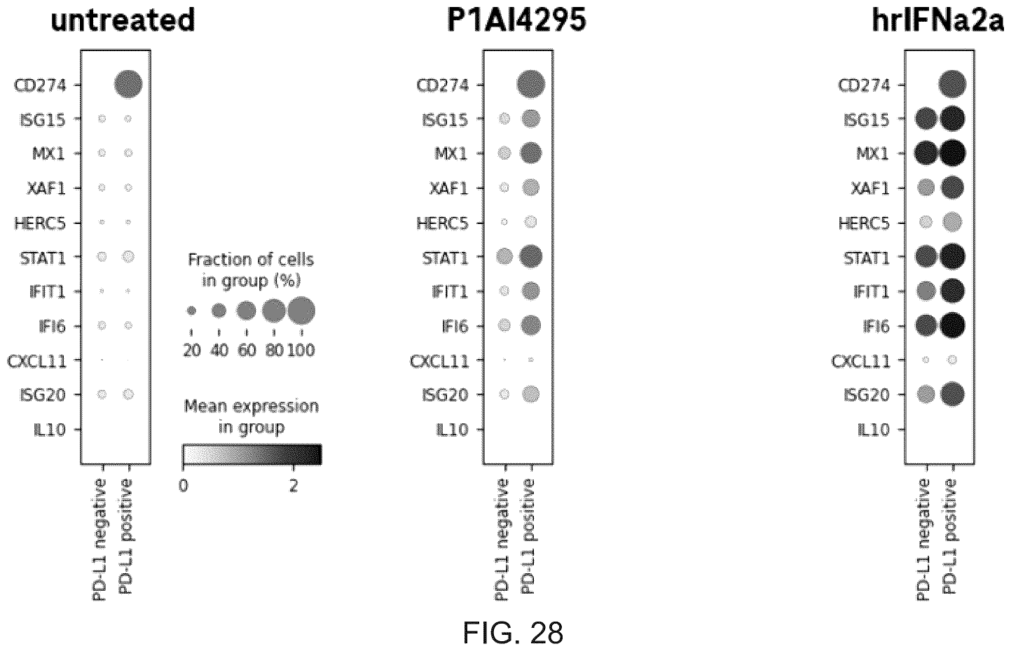

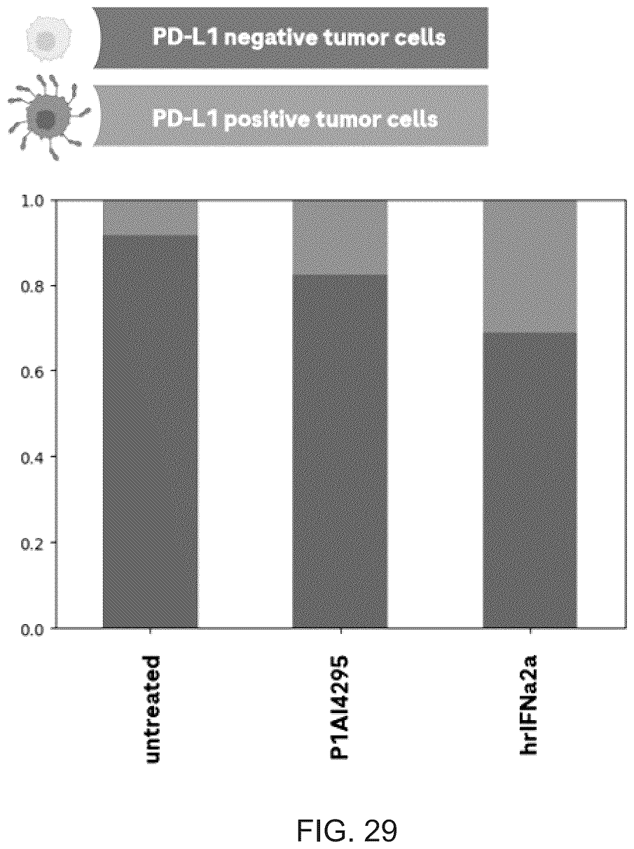

Given that PD-L1 upregulation is an immunomodulatory mechanism triggered by IFN-α treatment through the JAK-STAT signaling pathway, we proceeded to compare PD-L1 expression levels across the three test conditions (FIG. 29). We observed increased PD-L1 expression levels in the P1AI4295-treated samples compared to the untreated control. However, this elevation was even more pronounced in the recombinant IFN-α2a-treated condition.

### Example 25: Immunocompetent Hematotoxicity Assay

For the Immunocompetent Hematotoxicity Assay, 1.0E5 bone marrow mononuclear cells (BMMNCs) were seeded in SFEM II media (StemCell Technologies) for 7 days in ultra-low attachment 96-well round bottom plates (Corning). Cultured media contained titrated hematopoietic cytokines including animal component free (ACF) SCF, FLT3L, TPO, EPO, GM-CSF, G-CSF (StemCell Technologies) optimized to facilitate simultaneous multi-lineage hematopoiesis of myeloid lineages while in the context of lymphoid cells including T, B, and natural killer cells that are part of the whole BMMNC material. Titrated test article (either PD-L1-targeted IFN-α2a fusion protein P1AI4295, or recombinant IFN-α2a as control) spanning 1.0 pM to 100 nM was added on day 0 and cultured for 7 days after which cells were harvested and evaluated by flow cytometry to assess the impact of test articles on cell yields. The flow cytometry panel was composed of DAPI, DyeCycle Green (ThermoFisher), CD235ab-BV711 (HIR2 GA-R2), CD45-BV510 (HI30), and CD371-PE (50C1) (BD Biosciences) and analyzed using an LSR Fortessa X-20 fit with a high throughput sampler (BD Biosciences). Erythroblasts were defined as DAPI negative (live cells), CD235ab+45−, DyeCycle Green (DCG)+, and high FSC events (relative to DCG-CD235ab+ events). Total granulocyte/monocyte-lineage was defined by FSC/SSC characteristics, and expression of CD371. CountBright Absolute Counting Beads (ThermoFisher Scientific) were included in samples to transform raw event count information to cells per milliliter (mL). Once cell per mL information was determined, data were normalized to vehicle well counts, and plotted using GraphPad Prism with a log (inhibitor) vs. response variable slope 4-parameter curve fit.

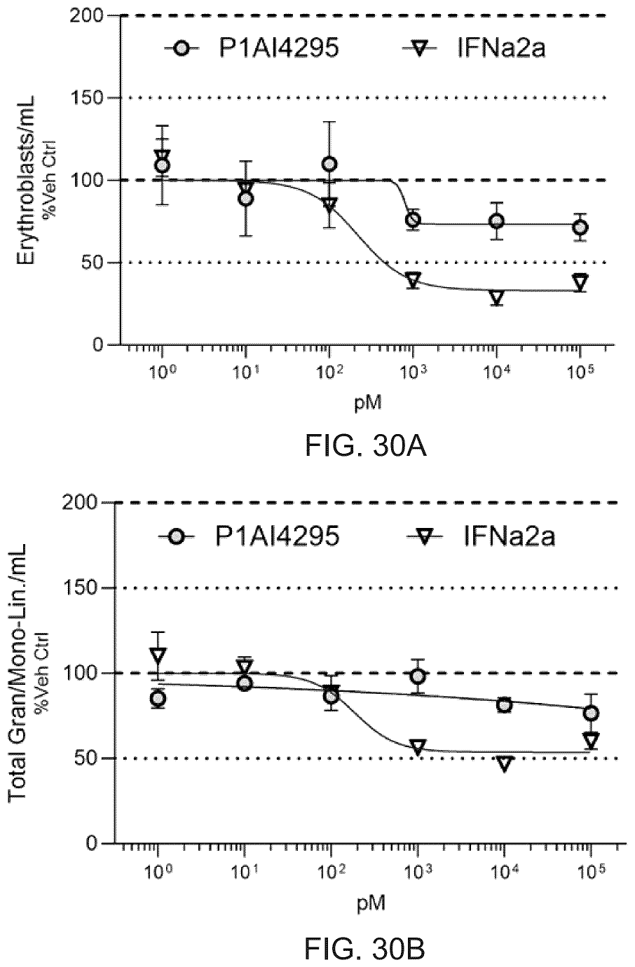

As shown in FIGS. 30A and 30B, 7 day treatment with IFN-α2a vs P1AI4295 on an immunocompetent hematotoxicity model resolves reduced toxicity of P1AI4295 as compared to IFN-α2a in both erythroid-lineage (FIG. 30A) and granulocyte/monocyte (FIG. 30 B) lineage production and survival. Error bars represent the standard error across 2 bone marrow mononuclear cell donors. Maximal reduction of Erythroblast subset resolved at 10 nM by IFN-α2a (71% suppression vs vehicle control), while P1AI4295 resolved a maximal suppression at 100 nM (29% suppression vs vehicle control). Similarly, with regards to granulocyte and monocyte lineage suppression, IFN-α2a showed a maximal suppression at 10 nM (53% suppression vs vehicle control) while P1AI4295 showed a maximal suppression at 100 nM (23% suppression vs vehicle control).

### Example 26: PBMC Immunotoxicity Assay

For the Peripheral Blood Mononuclear Cell (PBMC) Immunotoxicity Assay, 2.0E5 PBMCs were seeded in SFEM II media (StemCell Technologies) for 2 days in ultra-low attachment 96-well round bottom plates (Corning). Titrated test article (either PD-L1-targeted IFN-α2a fusion protein P1AI4295, or IFN-α2a as control) spanning 1.0 pM to 100 nM was added on day 0 and cultured for 2 days after which cells were harvested and evaluated by flow cytometry to assess the impact of test articles on cell activation profiles. The flow cytometry panel was composed of DAPI (ThermoFisher), CD45-BV510 (HI30), CD3-PEDazzle594 (OKT3), CD8-BV605 (RPA-T8), CD19-BV711 (HIB19), HLADR-APCCy7 (L243), CD69-AF488 (FN50) (BioLegend), and CD4-BUV737 (SK3) (BD Biosciences). Samples were analyzed using an LSR Fortessa X-20 fit with a high throughput sampler (BD Biosciences). Viable cells were defined as DAPI negative, and T-cell subsets were defined based on expression of CD3, after which a bivariate plot of CD4 vs CD8 was used to define CD4 from CD8 T-cells. Similarly, B-cells were defined on expression of CD19, while monocytes were defined by absence of CD3 and CD19, but high positivity for HLADR. After cell subsets were defined, CD69 expression was evaluated using gates set to include a minimum of 5% the population in vehicle-treated wells. Once percentage-based data was determined, raw values were plotted using GraphPad Prism with a log (agonist) vs. response variable slope 4-parameter curve fit.

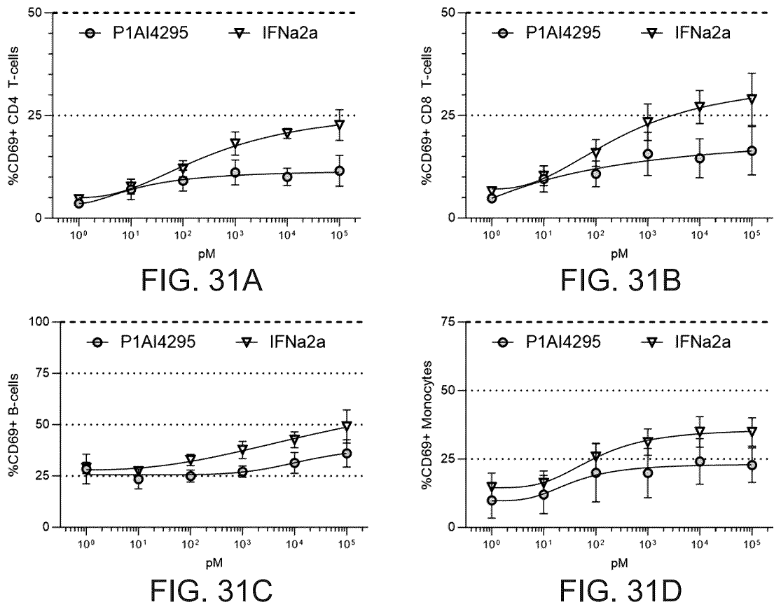

As shown in FIGS. 31A and 31B, 2-day treatment with IFN-α2a vs P1AI4295 on an immunotoxicity model resolves reduced % CD69 activation profile of P1AI4295 as compared to IFN-α2a on CD4 T-cells (FIG. 31A), CD8 T-cells (FIG. 31 B) B-cells (FIG. 31 C), and monocytes (FIG. 31 D). Error bars represent the standard error across 2 peripheral blood mononuclear cells donors.

1. A fusion protein that comprises
a. a first antigen-binding domain capable of binding to human PD-L1;
b. a second antigen-binding domain capable of binding to human PD-L1 and to human IFN-α2; and
c. a human IFN-α2;
wherein
i. the second antigen-binding domain is a bispecific Fab; and
ii. the human IFN-α2 is fused at its C-terminus to the N-terminus of the heavy chain or the light chain of the second antigen-binding domain.

2. The fusion protein of claim 1, wherein the first antigen-binding domain is monospecific for human PD-L1.

3. The fusion protein of claim 1, wherein the second antigen-binding domain is capable of blocking the human IFN-α2 from binding to IFNAR.

4. The fusion protein of claim 1, wherein the human IFN-α2 is blocked from binding to IFNAR when it is bound to the bispecific Fab and is able to bind to IFNAR when the bispecific Fab is bound to PD-L1.

5. The fusion protein claim 1, wherein the first antigen-binding domain capable of binding to human PD-L1 is a Fab.

6. The fusion protein claim 1, wherein the bispecific Fab is a DutaFab.

7. The fusion protein of claim 1, wherein the second antigen-binding domain capable of binding to human PD-L1 and to human IFN-α2 comprises a human PD-L1 paratope and a human IFN-α2 paratope within one cognate pair of a variable light chain domain (VL domain) and a variable heavy chain domain (VH domain), wherein the human IFN-α2 paratope comprises amino acid residues from CDR-H2, CDR-L1 and CDR-L3 of the antigen-binding domain, and wherein the human PD-L1 paratope comprises amino acid residues from the CDR-H1, CDR-H3 and CDR-L2 of the antigen-binding domain.

8. The fusion protein of claim 1, wherein the fusion protein further comprises an Fc domain composed of a first and a second subunit.

9. The fusion protein claim 1, wherein the second antigen-binding domain capable of binding to human PD-L1 and to human IFN-α2 comprises
a. a heavy chain variable domain (VH) comprising (a) CDR-H1 comprising the amino acid sequence of SEQ ID NO:1, (b) CDR-H2 comprising the amino acid sequence of SEQ ID NO:2, and (c) CDR-H3 comprising the amino acid sequence of SEQ ID NO:3, and a light chain variable domain (VL) comprising (d) CDR-L1 comprising the amino acid sequence of SEQ ID NO:4, (e) CDR-L2 comprising the amino acid sequence of SEQ ID NO:5, and (f) CDR-L3 comprising the amino acid sequence of SEQ ID NO:6;
b. a heavy chain variable domain (VH) comprising (a) CDR-H1 comprising the amino acid sequence of SEQ ID NO:1, (b) CDR-H2 comprising the amino acid sequence of SEQ ID NO:2, and (c) CDR-H3 comprising the amino acid sequence of SEQ ID NO:3, and a light chain variable domain (VL) comprising (d) CDR-L1 comprising the amino acid sequence of SEQ ID NO:9, (e) CDR-L2 comprising the amino acid sequence of SEQ ID NO:5, and (f) CDR-L3 comprising the amino acid sequence of SEQ ID NO:6;
c. a heavy chain variable domain (VH) comprising (a) CDR-H1 comprising the amino acid sequence of SEQ ID NO:1, (b) CDR-H2 comprising the amino acid sequence of SEQ ID NO:2, and (c) CDR-H3 comprising the amino acid sequence of SEQ ID NO:3, and a light chain variable domain (VL) comprising (d) CDR-L1 comprising the amino acid sequence of SEQ ID NO:4, (e) CDR-L2 comprising the amino acid sequence of SEQ ID NO:11, and (f) CDR-L3 comprising the amino acid sequence of SEQ ID NO: 6; or
d. a heavy chain variable domain (VH) comprising (a) CDR-H1 comprising the amino acid sequence of SEQ ID NO:13, (b) CDR-H2 comprising the amino acid sequence of SEQ ID NO:14, and (c) CDR-H3 comprising the amino acid sequence of SEQ ID NO: 15, and a light chain variable domain (VL) comprising (d) CDR-L1 comprising the amino acid sequence of SEQ ID NO:16, (e) CDR-L2 comprising the amino acid sequence of SEQ ID NO:17, and (f) CDR-L3 comprising the amino acid sequence of SEQ ID NO:18.

10. The fusion protein claim 1, wherein the second antigen-binding domain capable of binding to human PD-L1 and to human IFN-α2 comprises
a. a VH domain comprising the amino acid sequence of SEQ ID NO:7 and a VL domain comprising the amino acid sequence of SEQ ID NO:8;
b. a VH domain comprising the amino acid sequence of SEQ ID NO:7 and a VL domain comprising the amino acid sequence of SEQ ID NO:10;
c. a VH domain comprising the amino acid sequence of SEQ ID NO:7 and a VL domain comprising the amino acid sequence of SEQ ID NO: 12; or
d. a VH domain comprising the amino acid sequence of SEQ ID NO: 19 and a VL domain comprising the amino acid sequence of SEQ ID NO:20.

11. The fusion protein of claim 1, wherein the first antigen binding domain capable of binding to human PD-L1 comprises
a. a heavy chain variable domain (VH) comprising (a) CDR-H1 comprising the amino acid sequence of SEQ ID NO:21, (b) CDR-H2 comprising the amino acid sequence of SEQ ID NO:22, and (c) CDR-H3 comprising the amino acid sequence of SEQ ID NO: 23, and a light chain variable domain (VL) comprising (d) CDR-L1 comprising the amino acid sequence of SEQ ID NO:24, (e) CDR-L2 comprising the amino acid sequence of SEQ ID NO:25, and (f) CDR-L3 comprising the amino acid sequence of SEQ ID NO:26; or
b. a heavy chain variable domain (VH) comprising (a) CDR-H1 comprising the amino acid sequence of SEQ ID NO:29, (b) CDR-H2 comprising the amino acid sequence of SEQ ID NO:30, and (c) CDR-H3 comprising the amino acid sequence of SEQ ID NO: 31, and a light chain variable domain (VL) comprising (d) CDR-L1 comprising the amino acid sequence of SEQ ID NO:32, (e) CDR-L2 comprising the amino acid sequence of SEQ ID NO:33, and (f) CDR-L3 comprising the amino acid sequence of SEQ ID NO:34.

12. The fusion protein claim 1, wherein the first antigen binding domain capable of binding to human PD-L1 comprises
a. a VH domain comprising the amino acid sequence of SEQ ID NO:27 and a VL domain comprising the amino acid sequence of SEQ ID NO:28; or
b. a VH domain comprising the amino acid sequence of SEQ ID NO:35 and a VL domain comprising the amino acid sequence of SEQ ID NO:36.

13. The fusion protein of claim 1, wherein
the first antigen-binding domain capable of binding to human PD-L1 comprises a VH domain comprising the amino acid sequence of SEQ ID NO:27 and a VL domain comprising the amino acid sequence of SEQ ID NO:28, and
the second antigen-binding domain capable of binding to human PD-L1 and to human IFN-α2 comprises a VH domain comprising the amino acid sequence of SEQ ID NO:7 and a VL domain comprising the amino acid sequence of SEQ ID NO:8.

14. An antibody that binds to human PD-L1, wherein the antibody comprises a heavy chain variable domain (VH) comprising (a) CDR-H1 comprising the amino acid sequence of SEQ ID NO:21, (b) CDR-H2 comprising the amino acid sequence of SEQ ID NO: 22, and (c) CDR-H3 comprising the amino acid sequence of SEQ ID NO:23, and a light chain variable domain (VL) comprising (d) CDR-L1 comprising the amino acid sequence of SEQ ID NO:24, (e) CDR-L2 comprising the amino acid sequence of SEQ ID NO:25, and (f) CDR-L3 comprising the amino acid sequence of SEQ ID NO:26.

15. The antibody of claim 14, comprising a VH sequence of SEQ ID NO:27 and a VL sequence of SEQ ID NO:28.

16. An isolated nucleic acid encoding the fusion protein of claim 1.

17. A host cell comprising the nucleic acid of claim 16.

18. A method of producing the fusion protein of claim 1.

19. The method of claim 18, further comprising recovering the fusion protein or the antibody from the host cell.

20. A composition comprising the antibody of claim 1 and a pharmaceutically acceptable carrier.

21. (canceled)

22. (canceled)

23. An isolated nucleic acid encoding the antibody of claim 14.

24. A host cell comprising the nucleic acid of claim 23.

25. A method of producing the antibody of claim 14 under conditions suitable for the expression of the antibody.

26. The method of claim 25, further comprising recovering the antibody from the host cell.

27. A composition comprising the antibody of claim 14 and a pharmaceutically acceptable carrier.

28. A method of treating cancer in a subject, comprising: administering an effective amount of the fusion protein of claim 1 to the subject.

29. A method of treating cancer in a subject, comprising: administering an effective amount of the antibody of claim 14 to the subject.
